(19) United States
(12) Patent Application Publication
(10) Pub. No.: US 2013/0196964 A1
(43) Pub. Date: Aug. 1, 2013

(54) HETEROCYCLICALLY SUBSTITUTED ARYL COMPOUNDS AS HIF INHIBITORS

(71) Applicants: Michael Härter, Leverkusen (DE); Hartmut Beck, Koln (DE); Peter Ellinghaus, Melle (DE); Kerstin Berhörster, Essen (DE); Susanne Greschat, Wagenfeld (DE); Karl-Heinz Thierauch, Berlin (DE); Frank Süssmeier, Munchen (DE)

(72) Inventors: Michael Härter, Leverkusen (DE); Hartmut Beck, Koln (DE); Peter Ellinghaus, Melle (DE); Kerstin Berhörster, Essen (DE); Susanne Greschat, Wagenfeld (DE); Karl-Heinz Thierauch, Berlin (DE); Frank Süssmeier, Munchen (DE)

(73) Assignee: BAYER SCHERING PHARMA AKTIENGESELLSCHAFT, Berlin (DE)

(21) Appl. No.: 13/129,407

(22) PCT Filed: Oct. 31, 2009
(86) PCT No.: PCT/EP2009/007806

§ 371 Date: Aug. 8, 2011

(30) Foreign Application Priority Data
Nov. 14, 2008 (DE) 10 2008 057 343.4
Sep. 11, 2009 (DE) 10 2009 041 242.5

(51) Int. Cl. C07D 487/04; A61K 31/496; A61K 31/4545; A61K 31/4439; A61K 45/06; A61K 31/498; A61K 31/5377; A61K 31/551; A61K 31/4245; A61K 31/454; C07D 413/14; A61K 31/541

(52) U.S. Cl. CPC C07D 487/04; C07D 413/14; A61K 31/496; A61K 31/4545; A61K 31/4439; A61K 31/541; A61K 31/498; A61K 31/5377; A61K 31/551; A61K 31/4245; A61K 31/454; A61K 45/06
USPC 514/210.18; 544/364; 514/253.1; 546/194; 514/318; 546/269.1; 514/341; 544/60; 514/227.8; 514/210.2; 544/349; 514/249; 544/124; 514/236.2; 540/575; 514/218; 548/131; 514/364; 546/209; 514/326

(57) ABSTRACT

The present application relates to novel aryl compounds with heterocyclic substituents, processes for their preparation, their use for treatment and/or prevention of diseases and their use for the preparation of medicaments for treatment and/or prevention of diseases, in particular for treatment and/or prevention of hyperproliferative and angiogenic diseases and those diseases which arise from metabolic adaptation to hypoxic states. Such treatments can be carried out as monotherapy or also in combination with other medicaments or further therapeutic measures.

The present application relates to novel aryl compounds with heterocyclic substituents, processes for their preparation, their use for treatment and/or prevention of diseases and their use for the preparation of medicaments for treatment and/or prevention of diseases, in particular for treatment and/or prevention of hyperproliferative and angiogenic diseases and those diseases which arise from metabolic adaptation to hypoxic states. Such treatments can be carried out as monotherapy or also in combination with other medicaments or further therapeutic measures.

Cancer diseases are the consequence of uncontrolled cell growth of the most diverse tissue. In many cases the new cells penetrate into existing tissue (invasive growth), or they metastase into remote organs. Cancer diseases occur in the most diverse organs and often have tissue-specific courses of the disease. The term cancer as a generic term therefore describes a large group of defined diseases of various organs, tissue and cell types.

In the year 2002 4.4 million people worldwide were diagnosed with tumour diseases of the breast, intestine, ovaries, lung or prostate. In the same year, approx. 2.5 million deaths were assumed to be a consequence of these diseases (Globocan 2002 Report). In the USA alone, for the year 2005 over 1.25 million new cases and over 500,000 deaths were predicted from cancer diseases. The majority of these new cases concern cancer diseases of the intestine (˜100,000), lung (˜170,000), breast (˜210,000) and prostate (˜230,000). A further increase in cancer diseases of approx. 15% over the next 10 years is assumed (American Cancer Society, Cancer Facts and Figures 2005).

Tumours in early stages can possibly be removed by surgical and radiotherapy measures. Metastased tumours as a rule can only be treated palliatively by chemotherapeutics. The aim here is to achieve the optimum combination of an improvement in the quality of life and prolonging of life.

Chemotherapies are often composed of combinations of cytotoxic medicaments. The majority of these substances have as their action mechanism bonding to tubulin, or they are compounds which interact with the formation and processing of nucleic acids. More recently these also include enzyme inhibitors, which interfere with epigenetic DNA modification or cell cycle progression (e.g. histone deacetylase inhibitors, aurora kinase inhibitors). Since such therapies are toxic, more recently the focus has increasingly been on targeted therapies in which specific processes in the cell are blocked without there being a high toxic load. These include in particular inhibitors of kinases which inhibit the phosphorylation of receptors and signal transmission molecules. An example of these is imatinib, which is employed very successfully for treatment of chronic myeloid leukaemia (CML) and gastrointestinal stromal tumours (GIST). Further examples are substances which block EGFR kinase and HER2, such as erlotinib, and VEGFR kinase inhibitors, such as sorafenib and sunitinib, which are employed on kidney cell carcinomas, liver carcinomas and advanced stages of GIST.

The life expectancy of colorectal carcinoma patients has been successfully prolonged with an antibody directed against VEGF. Bevacizumab inhibits growth of blood vessels, which obstructs rapid expansion of tumours since this requires connection to the blood vessel system for a continuously functioning supply and disposal.

One stimulus of angiogenesis is hypoxia, which occurs again and again with solid tumours since the blood supply is inadequate because of the unregulated growth. If there is a lack of oxygen, cells switch their metabolism from oxidative phosphorylation to glycolysis so that the ATP level in the cell is stabilized. This process is controlled by a transcription factor, which is regulated upwards depending on the oxygen content in the cell. This transcription factor, called “hypoxia-induced factor” (HIF) is normally removed posttranslationally by rapid degradation and prevented from transportation into the cell nucleus. This is effected by hydroxylation of two proline units in the oxygen degradable domain (ODD) and an asparagine unit in the vicinity of the C terminus by the enzymes prolyl dehydrogenase and FIH (“factor inhibiting HIF”). After the modification of the proline units, HIF can be degraded with mediation by the Hippel-Lindau protein (part of a ubiquitin-E3-ligase complex) via the proteasome apparatus (Maxwell, Wiesener et al, 1999). In the event of oxygen deficiency, the degradation does not take place and the protein is regulated upwards and leads to transcription or blockade of the transcription of numerous (more than 100) other proteins (Semenza and Wang, 1992; Wang and Semenza, 1995).

The transcription factor HIF is formed by the regulated α-subunit and a constitutively present (3-subunit (ARNT, aryl hydrocarbon receptor nuclear translocator). There are three different species of the α-subunit, 1α, 2α and 3α, the last being rather to be assumed as a suppressor (Makino, Cao et al, 2001) The HIF subunits are bHLH (basic helix loop helix) proteins, which dimerize via their HLH and PAS (Per-Arnt-Sim) domain, which starts their transactivation activity (Jiang, Rue et al., 1996).

In the most important tumour entities, overexpression of the HIF1α protein is correlated with increasing density of blood vessels and enhanced VEGF expression (Hirota and Semenza, 2006). At the same time glucose metabolism is changed to glycolysis, and the Krebs cycle is reduced in favour of the production of cell units. This also implies a change in fat metabolism. Such changes appear to guarantee the survival of the tumours. On the other hand, if the activity of HIF is now inhibited, the development of tumours could consequently be suppressed. This has already been observed in various experimental models (Chen, Zhao et al., 2003; Stoeltzing, McCarty et al., 2004; Li, Lin et al., 2005; Mizukami, Jo et al., 2005; Li, Shi et al., 2006). Specific inhibitors of the metabolism controlled by HIF should therefore be suitable as tumour therapeutics.

The object of the present invention was therefore to provide novel compounds which act as inhibitors of the transactivating action of the transcription factor HIF and can be employed as such for treatment and/or prevention of diseases, in particular of hyperproliferative and angiogenic diseases, such as cancer diseases.

Substituted multicyclic heteroaryl compounds with pyrrole, pyrazole and/or oxadiazole partial structures and the use of these compounds for treatment of diverse diseases are described in numerous forms in the patent literature, thus inter alia in EP 0 908 456-A1, WO 97/36881-A1, WO 01/12627-A1, WO 01/85723-A1, WO 02/100826-A2, WO 2004/014370-A2, WO 2004/014881-A2, WO 2004/014902-A2, WO 2004/035566-A1, WO 2004/058176-A2, WO 2004/089303-A2, WO 2004/089308-A2, WO 2005/070925-A1, WO 2006/114313-A1, WO 2007/002559-A1, WO 2007/034279-A2, WO 2008/004096-A1, WO 2008/024390-A2 and WO 2008/114157-A1. WO 2005/030121-A2 and WO 2007/065010-A2 claim the use of certain pyrazole derivatives for inhibition of the expression of HIF and HIF-regulated genes in tumour cells. WO 2008/141731-A2 describes heteroaryl-substituted N-benzylpyrazoles as inhibitors of the HIF regulation pathway for treatment of cancer diseases. Heteroaryl-substituted 5-(1H-pyrazol-3-yl)-1,2,4-oxadiazoles as cannabinoid receptor modulators for treatment of diverse diseases are disclosed in US 2008/0255211-A1. Further diaryl-substituted isoxazole and 1,2,4-oxadiazole derivatives are described in WO 2009/029632-A1 as inhibitors of monoamine oxidase B for treatment of psychiatric diseases.

The present invention provides compounds of the general formula (I)

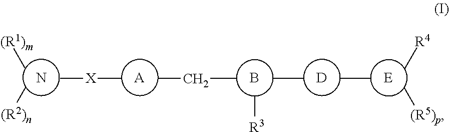

in which
the ring

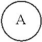

represents a phenyl or pyridyl ring,
the ring

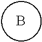

with the substituent R³ represents a heteroaryl ring of the formula

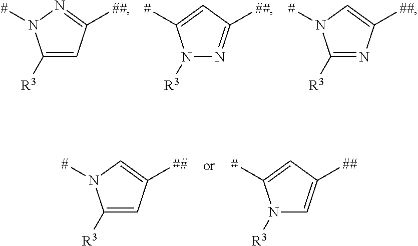

wherein
# designates the linkage point with the adjacent CH₂ group
and
## designates the linkage point with the ring

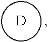

the ring

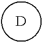

represents a heteroaryl ring of the formula

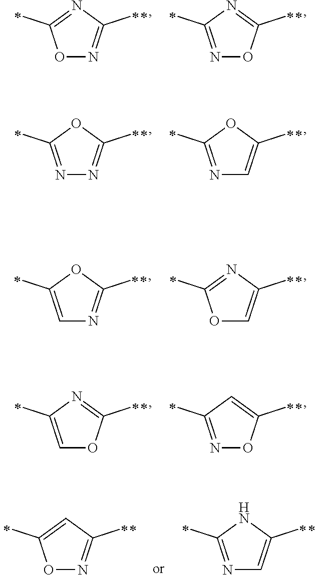

wherein
* designates the linkage point with the ring

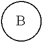

and
** designates the linkage point with the ring

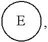

the ring

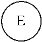

represents a phenyl or pyridyl ring,
the ring

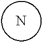

represents a saturated 4- to 10-membered aza-heterocycle, which contains at least one N atom as a ring member and in addition can contain one or two further hetero ring members from the series N, O, S and/or S(O)₂,
X represents a bond or ♦—(CH₂)_{q}—N(R⁶)—♦♦, ♦—N(R⁶)—(CH₂)_{q}—♦♦, —O—, —S—, —C(═O)—, —S(═O)₂—, ♦—C(═O)—N(R⁶)—♦♦ or ♦—N(R⁶)—C(═O)—♦♦, wherein
   ♦ designates the linkage point with the ring

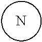

and
♦♦ designates the linkage point with the ring

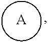

q denotes the number 0, 1 or 2
and
R⁶ denotes hydrogen, (C₁-C₆)-alkyl or (C₃-C₆)-cycloalkyl,
   wherein (C₁-C₆)-alkyl and (C₃-C₆)-cycloalkyl can each be substituted by hydroxyl or (C₁-C₄)-alkoxy,
R¹ represents a substituent bonded to a carbon atom of the ring

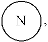

chosen from the series fluorine, cyano, (C₁-C₆)-alkyl, hydroxyl, (C₁-C₆)-alkoxy, oxo, amino, mono-(C₁-C₆)-alkylamino, di-(C₁-C₆)-alkylamino and (C₃-C₆)-cycloalkyl,
wherein (C₁-C₆)-alkyl in its turn can be substituted up to three times by fluorine and up to two times in an identical or different manner by a radical chosen from the series hydroxyl, (C₁-C₄)-alkoxy, amino, mono-(C₁-C₄)-alkylamino and di-(C₁-C₄)-alkylamino
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
m represents the number 0, 1, 2, 3 or 4,
   wherein in the case where the substituent R¹ occurs several times, its meanings can be identical or different,
R² represents a substituent bonded to a nitrogen atom of the ring

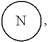

chosen from the series (C₁-C₆)-alkyl, (C₁-C₆)-alkylcarbonyl, (C₁-C₆)-alkoxycarbonyl, (C₁-C₆)-alkylsulfonyl and (C₃-C₆)-cycloalkyl,
wherein the alkyl group in (C₁-C₆)-alkyl, (C₁-C₆)-alkylcarbonyl, (C₁-C₆)-alkoxycarbonyl and (C₁-C₆)-alkylsulfonyl in its turn can be substituted up to three times by fluorine and up to two times in an identical or different manner by a radical chosen from the series hydroxyl, (C₁-C₄)-alkoxy, amino, mono-(C₁-C₄)-alkylamino, di-(C₁-C₄)-alkylamino (C₃-C₆)-cycloalkyl and 4- to 6-membered heterocyclyl
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
n represents the number 0 or 1 or also, if the aza-heterocycle

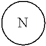

contains further N atoms as ring members, the number 2,
wherein in the case where the substituent R² occurs twice, its meanings can be identical or different,
R³ represents methyl, ethyl or trifluoromethyl,
R⁴ represents hydrogen or a substituent chosen from the series halogen, cyano, pentafluorothio, (C₁-C₆)-alkyl, tri-(C₁-C₄)-alkylsilyl, —OR⁷, —NR⁷R⁸, —N(R⁷)—C(═O)—R⁸, —N(R⁷)—C(═O)—OR⁸, —N(R⁷)—S(═O)₂—R⁸, —C(═O)—OR⁷, —C(═O)—NR⁷R⁸, —SR⁷, —S(═O)—R⁷, —S(═O)₂—R⁷, —S(═O)₂—NR⁷R⁸, —S(═O)(═NH)—R⁷, —S(═O)(═NCH₃)—R⁷, (C₃-C₆)-cycloalkyl, 4- to 6-membered heterocyclyl and 5- or 6-membered heteroaryl,
   wherein (C₁-C₆)-alkyl in its turn can be substituted up to three times by fluorine and up to two times in an identical or different manner by a radical chosen from the series —OR⁷, —NR⁷R⁸, —N(R⁷)—C(═O)—R⁸, —N(R⁷)—C(═O)—OR⁸, —C(═O)—OR⁷, —C(═O)—NR⁷R⁸, (C₃-C₆)-cycloalkyl, 4- to 6-membered heterocyclyl and 5- or 6-membered heteroaryl
   and wherein
   the cycloalkyl and heterocyclyl groups mentioned in their turn can be substituted up to two times in an identical or different manner by a radical chosen from the series fluorine, (C₁-C₄)-alkyl, hydroxyl, (C₁-C₄)-alkoxy, trifluoromethoxy, oxo, amino, mono-(C₁-C₄)-alkyl-amino, di-(C₁-C₄)-alkylamino, (C₁-C₄)-alkylcarbonylamino, (C₁-C₄)-alkoxycarbonylamino, (C₁-C₄)-alkylcarbonyl and (C₁-C₄)-alkoxycarbonyl
   and
   the heteroaryl groups mentioned in their turn can be substituted up to two times in an identical or different manner by a radical chosen from the series fluorine, chlorine, cyano, (C₁-C₄-alkyl, (C₁-C₄)-alkoxy and trifluoromethoxy
      wherein the (C₁-C₄)-alkyl substituents mentioned herein in their turn can be substituted by hydroxyl, (C₁-C₄)-alkoxy, trifluoromethoxy, (C₁-C₄)-alkylcarbonyloxy, aminocarbonyl, mono-(C₁-C₄)-alkylaminocarbonyl or di-(C₁-C₄)-alkylaminocarbonyl or up to three times by fluorine,
   and wherein
   R⁷ and R⁸ independently of each other for each individual occurrence denote hydrogen, (C₁-C₆)-alkyl, (C₃-C₆)-cycloalkyl or 4- to 6-membered heterocyclyl,
      wherein (C₁-C₆)-alkyl can be substituted up to three times by fluorine and up to two times in an identical or different manner by a radical chosen from the series hydroxyl, (C₁-C₄)-alkoxy, trifluoromethoxy, amino, mono-(C₁-C₄)-alkylamino, di-(C₁-C₄)-alkylamino, (C₁-C₄)-alkoxycarbonyl, (C₃-C₆)-cycloalkyl and 4- to 6-membered heterocyclyl
      and
      the cycloalkyl and heterocyclyl groups mentioned can be substituted up to two times in an identical or different manner by a radical chosen from the series fluorine, (C₁-C₄-alkyl, trifluoromethyl, hydroxyl, (C₁-C₄)-alkoxy, trifluoromethoxy, oxo, amino, mono-(C₁-C₄)-alkylamino, di-(C₁-C₄)-alkylamino, (C₁-C₄)-alkylcarbonyl and (C₁-C₄)-alkoxycarbonyl,
   or
   R⁷ and R⁸ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo, amino, mono-(C₁-C₄)-alkylamino, di-(C₁-C₄)-alkylamino, (C₁-C₄)-alkylcarbonyl and (C₁-C₄)-alkoxycarbonyl,
R⁵ represents a substituent chosen from the series fluorine, chlorine, cyano, methyl, trifluoromethyl and hydroxyl
   and
p represents the number 0, 1 or 2,
   wherein in the case where the substituent R⁵ occurs twice, its meanings can be identical or different,
      and their salts, solvates and solvates of the salts.

An alternative embodiment within the subject matter of the invention described above comprises compounds of the formula (I) in which
R¹ represents a substituent bonded to a carbon atom of the ring

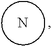

chosen from the series fluorine, (C₁-C₆)-alkyl, hydroxyl, (C₁-C₆)-alkoxy, oxo, amino, mono-(C₁-C₆)-alkylamino, di-(C₁-C₆)-alkylamino and (C₃-C₆)-cycloalkyl,
wherein (C₁-C₆)-alkyl in its turn can be substituted up to three times by fluorine and up to two times in an identical or different manner by a radical chosen from the series hydroxyl, (C₁-C₄)-alkoxy, amino, mono-(C₁-C₄)-alkylamino and di-(C₁-C₄)-alkylamino
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
m represents the number 0, 1, 2, 3 or 4,
   wherein in the case where the substituent R¹ occurs several times, its meanings can be identical or different,
      and
R⁴ represents hydrogen or a substituent chosen from the series halogen, cyano, pentafluorothio, (C₁-C₆)-alkyl, tri-(C₁-C₄)-alkylsilyl, —OR⁷, —NR⁷R⁸, —N(R⁷)—C(═O)—R⁸, —N(R⁷)—C(═O)—OR⁸, —N(R⁷)—S(═O)₂—R⁸, —C(═O)—OR⁷, —C(═O)—NR⁷R⁸, —SR⁷, —S(═O)—R⁷, —S(═O)₂—R⁷, —S(═O)₂—NR⁷R⁸, (C₃-C₆)-cycloalkyl, 4- to 6-membered heterocyclyl and 5- or 6-membered heteroaryl,
   wherein (C₁-C₆)-alkyl in its turn can be substituted up to three times by fluorine and up to two times in an identical or different manner by a radical chosen from the series —OR⁷, —NR⁷R⁸, —N(R⁷)—C(═O)—R⁸, —N(R⁷)—C(═O)—OR⁸, —C(═O)—OR⁷, —C(═O)—NR⁷R⁸, (C₃-C₆)-cycloalkyl, 4- to 6-membered heterocyclyl and 5- or 6-membered heteroaryl
   and wherein
   the cycloalkyl and heterocyclyl groups mentioned in their turn can be substituted up to two times in an identical or different manner by a radical chosen from the series fluorine, (C₁-C₄)-alkyl, trifluoromethyl, hydroxyl, (C₁-C₄)-alkoxy, trifluoromethoxy, oxo, amino, mono-(C₁-C₄)-alkylamino, di-(C₁-C₄)-alkylamino, (C₁-C₄)-alkylcarbonylamino, (C₁-C₄)-alkoxycarbonylamino, (C₁-C₄)-alkylcarbonyl and (C₁-C₄)-alkoxycarbonyl
   and
   the heteroaryl groups mentioned in their turn can be substituted up to two times in an identical or different manner by a radical chosen from the series fluorine, chlorine, cyano, (C₁-C₄)-alkyl, trifluoromethyl, (C₁-C₄)-alkoxy and trifluoromethoxy
   and wherein R⁷ and R⁸ have the meanings given above
      and their salts, solvates and solvates of the salts.

Compounds according to the invention are the compounds of the formula (I) and their salts, solvates and solvates of the salts, the compounds included in the formula (I) of the formulae mentioned in the following and their salts, solvates and solvates of the salts, and the compounds included in the formula (I) and mentioned in the following as embodiment examples and their salts, solvates and solvates of the salts, where the compounds included in the formula (I) and mentioned in the following are not already salts, solvates and solvates of the salts.

The compounds according to the invention can exist in stereoisomeric forms (enantiomers, diastereomers), depending on their structure. The invention therefore includes the enantiomers or diastereomers and their particular mixtures. The stereoisomerically uniform constituents can be isolated from such mixtures of enantiomers and/or diastereomers in a known manner; chromatography processes are preferably used for this, in particular HPLC chromatography on an achiral or chiral phase.

Where the compounds according to the invention can occur in tautomeric forms, the present invention includes all the tautomeric forms.

Preferred salts in the context of the present invention are physiologically acceptable salts of the compounds according to the invention. Salts which are not themselves suitable for pharmaceutical uses but can be used, for example, for isolation or purification of the compounds according to the invention are also included.

Physiologically acceptable salts of the compounds according to the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, benzenesulfonic acid, toluenesulfonic acid, naphthalenedisulfonic acid, formic acid, acetic acid, trifluoroacetic acid, propionic acid, lactic acid, tartaric acid, malic acid, citric acid, fumaric acid, maleic acid, benzoic acid and 4-sulfamoylbenzoic acid.

Physiologically acceptable salts of the compounds according to the invention also include salts of conventional bases, such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

Solvates in the context of the invention are described as those forms of the compounds according to the invention which form a complex in the solid or liquid state by coordination with solvent molecules. Hydrates are a specific form of solvates, in which the coordination takes place with water. Hydrates are preferred solvates in the context of the present invention.

The N-oxides of pyridyl rings and tertiary cyclic amine groupings contained in compounds according to the invention are similarly included in the present invention.

The present invention moreover also includes prodrugs of the compounds according to the invention. The term “prodrugs” here designates compounds which themselves can be biologically active or inactive, but are converted (for example metabolically or hydrolytically) into compounds according to the invention during their dwell time in the body.

In the context of the present invention, the substituents have the following meaning, unless specified otherwise:

(C₁-C₆)-Alkyl and (C₁-C₄)-alkyl in the context of the invention represent a straight-chain or branched alkyl radical having 1 to 6 or, respectively, 1 to 4 carbon atoms. A straight-chain or branched alkyl radical having 1 to 4 carbon atoms is preferred. There may be mentioned by way of example and preferably: methyl, ethyl, n-propyl, isopropyl, n-butyl, iso-butyl, sec-butyl, tert-butyl, n-pentyl, 2-pentyl, 3-pentyl, neopentyl, n-hexyl, 2-hexyl and 3-hexyl.

(C₁-C₆)-Alkylcarbonyl and (C₁-C₄)-alkylcarbonyl in the context of the invention represent a straight-chain or branched alkyl radical having 1 to 6 or, respectively, 1 to 4 carbon atoms which is linked via a carbonyl group [—C(═O)—]. A straight-chain or branched alkylcarbonyl group having 1 to 4 carbon atoms in the alkyl radical is preferred. There may be mentioned by way of example and preferably: acetyl, propionyl, n-butyryl, iso-butyryl, n-pentanoyl, pivaloyl, n-hexanoyl and n-heptanoyl.

(C₁-C₆)-Alkylsulfonyl and (C₁-C₄)-alkylsulfonyl in the context of the invention represent a straight-chain or branched alkyl radical having 1 to 6 or, respectively, 1 to 4 carbon atoms which is linked via a sulfonyl group [—S(═O)₂-]. A straight-chain or branched alkylsulfonyl group having 1 to 4 carbon atoms in the alkyl radical is preferred. There may be mentioned by way of example and preferably: methylsulfonyl, ethylsulfonyl, n-propylsulfonyl, isopropylsulfonyl, n-butylsulfonyl, tert-butylsulfonyl, n-pentylsulfonyl and n-hexylsulfonyl.

Tri-(C₁-C₄)-alkylsilyl in the context of the invention represents a silyl group with three identical or different straight-chain or branched alkyl substituents, each of which contains 1 to 4 carbon atoms. There may be mentioned by way of example and preferably: trimethylsilyl, tert-butyl-dimethylsilyl and triisopropylsilyl.

(C₁-C₆)-Alkoxy and (C₁-C₄)-alkoxy in the context of the invention represent a straight-chain or branched alkoxy radical having 1 to 6 or, respectively, 1 to 4 carbon atoms. A straight-chain or branched alkoxy radical having 1 to 4 carbon atoms is preferred. There may be mentioned by way of example and preferably: methoxy, ethoxy, n-propoxy, isopropoxy, n-butoxy, iso-butoxy, sec-butoxy, tert-butoxy, n-pentoxy, 2-pentoxy, 3-pentoxy, neopentoxy, n-hexoxy, 2-hexoxy and 3-hexoxy.

(C₁-C₆)-Alkoxycarbonyl and (C₁-C₄)-alkoxycarbonyl in the context of the invention represent a straight-chain or branched alkoxy radical having 1 to 6 or, respectively, 1 to 4 carbon atoms which is linked via a carbonyl group [—C(═O)—]. A straight-chain or branched alkoxycarbonyl group having 1 to 4 carbon atoms in the alkoxy radical is preferred. There may be mentioned by way of example and preferably: methoxycarbonyl, ethoxycarbonyl, n-propoxycarbonyl, isopropoxycarbonyl, n-butoxycarbonyl, tert-butoxycarbonyl, n-pentoxycarbonyl and n-hexoxycarbonyl.

Mono-(C₁-C₆)-alkylamino and mono-(C₁-C₄)-alkylamino in the context of the invention represent an amino group with a straight-chain or branched alkyl substituent which contains 1 to 6 or, respectively, 1 to 4 carbon atoms. A straight-chain or branched monoalkylamino radical having 1 to 4 carbon atoms is preferred. There may be mentioned by way of example and preferably: methylamino, ethylamino, n-propylamino, isopropylamino, n-butylamino, tert-butylamino, n-pentylamino and n-hexylamino.

Di-(C₁-C₆)-alkylamino and di-(C₁-C₄)-alkylamino in the context of the invention represent an amino group with two identical or different straight-chain or branched alkyl substituents which each contain 1 to 6 or, respectively, 1 to 4 carbon atoms. Straight-chain or branched dialkylamino radicals having in each case 1 to 4 carbon atoms are preferred. There may be mentioned by way of example and preferably: N,N-dimethylamino, N,N-diethylamino, N-ethyl-N-methylamino, N-methyl-N-n-propylamino, N-isopropyl-N-methylamino, N-isopropyl-N-n-propylamino, N,N-diisopropylamino, N-n-butyl-N-methylamino, N-tert-butyl-N-methylamino, N-methyl-N-n-pentyl-amino and N-n-hexyl-N-methylamino.

Mono- or di-(C₁-C₄)-alkylaminocarbonyl in the context of the invention represents an amino group which is linked via a carbonyl group [—C(═O)—] and which has a straight-chain or branched or, respectively, two identical or different straight-chain or branched alkyl substituents having in each case 1 to 4 carbon atoms. There may be mentioned by way of example and preferably: methylaminocarbonyl, ethylaminocarbonyl, n-propylaminocarbonyl, isopropylaminocarbonyl, n-butylaminocarbonyl, tert-butylaminocarbonyl, N,N-dimethylaminocarbonyl, N,N-diethylamino-carbonyl, N-ethyl-N-methylaminocarbonyl, N-methyl-N-n-propylaminocarbonyl, N-isopropyl-N-methylaminocarbonyl, N,N-diisopropylaminocarbonyl, N-n-butyl-N-methylaminocarbonyl and N-tert-butyl-N-methylaminocarbonyl.

(C₁-C₄)-Alkylcarbonylamino in the context of the invention represents an amino group with a straight-chain or branched alkylcarbonyl substituent which contains 1 to 4 carbon atoms in the alkyl radical and is linked to the N atom via the carbonyl group. There may be mentioned by way of example and preferably: acetylamino, propionylamino, n-butyrylamino, iso-butyrylamino, n-pentanoylamino and pivaloylamino.

(C₁-C₄)-Alkylcarbonyloxy in the context of the invention represents an oxy radical with a straight-chain or branched alkylcarbonyl substituent which contains 1 to 4 carbon atoms in the alkyl radical and is linked to the O atom via the carbonyl group. There may be mentioned by way of example and preferably: acetoxy, propionoxy, n-butyroxy, iso-butyroxy, n-pentanoyloxy and pivaloyloxy.

(C₁-C₄)-Alkoxycarbonylamino in the context of the invention represents an amino group with a straight-chain or branched alkoxycarbonyl substituent which contains 1 to 4 carbon atoms in the alkoxy radical and is linked to the N atom via the carbonyl group. There may be mentioned by way of example and preferably: methoxycarbonylamino, ethoxycarbonylamino, n-propoxycarbonyl-amino, isopropoxycarbonylamino, n-butoxycarbonylamino and tert-butoxycarbonylamino.

C₃-C₆)-Cycloalkyl and C₃-C₅)-cycloalkyl in the context of the invention represent a monocyclic, saturated cycloalkyl group having 3 to 6 or, respectively, 3 to 5 ring carbon atoms. There may be mentioned by way of example and preferably: cyclopropyl, cyclobutyl, cyclopentyl and cyclohexyl.

4- to 6-membered heterocyclyl and 4- or 5-membered heterocyclyl in the context of the invention represent a monocyclic, saturated heterocycle with 4 to 6 or, respectively, 4 or 5 ring atoms in total, which contains one or two ring hetero atoms from the series N, O, S and/or S(O)₂ and is linked via a ring carbon atom or optionally via a ring nitrogen atom. 4- or 5-membered heterocyclyl with a ring hetero atom from the series N, O or S and 6-membered heterocyclyl with one or two ring hetero atoms from the series N, O and/or S is preferred. There may be mentioned by way of example: azetidinyl, oxetanyl, thietanyl, pyrrolidinyl, pyrazolidinyl, tetrahydrofuranyl, thiolanyl, 1,1-dioxidothiolanyl, 1,3-oxazolidinyl, 1,3-thiazolidinyl, piperidinyl, piperazinyl, tetrahydropyranyl, tetrahydrothiopyranyl, 1,3-dioxanyl, 1,4-dioxanyl, morpholinyl, thiomorpho-linyl and 1,1-dioxidothiomorpholinyl. Azetidinyl, oxetanyl, pyrrolidinyl, tetrahydrofuranyl, piperi-dinyl, piperazinyl, tetrahydropyranyl, morpholinyl and thiomorpholinyl are preferred.

A 4- to 10-membered aza-heterocycle in the context of the invention represent a mono- or optionally bicyclic, saturated heterocycle with 4 to 10 ring atoms in total, which contains at least one ring nitrogen atom and in addition can contain one or two further ring hetero atoms from the series N, O, S and/or S(O)₂ and is linked via a ring carbon atom or optionally via a ring nitrogen atom. A 4- to 10-membered aza-heterocycle which contains at least one ring nitrogen atom and in addition can contain a further ring hetero atom from the series N, O, S or S(O)₂ is preferred. There may be mentioned by way of example and preferably: azetidinyl, pyrrolidinyl, pyrazolidinyl, 1,3-oxazolidinyl, 1,3-thiazolidinyl, piperidinyl, piperazinyl, morpholinyl, thiomorpholinyl, 1,1-dioxidothiomorpholinyl, hexahydroazepinyl, hexahydro-1,4-diazepinyl, octahydroazocinyl, octa-hydropyrrolo[3,4-b]pyrrolyl, octahydroisoindolyl, octahydropyrrolo[3,2-b]pyridyl, octahydro-pyrrolo[3,4-b]pyridyl, octahydropyrrolo[3,4-c]pyridyl, octahydropyrrolo[1,2-a]pyrazinyl, deca-hydroisoquinolinyl, octahydropyrido[1,2-a]pyrazinyl, 7-azabicyclo[2.2.1]heptyl, 3-azabicyclo-[3.2.0]heptyl, 2-oxa-6-azaspiro[3.3]heptyl, 3-azabicyclo[3.2.1]octyl, 8-azabicyclo[3.2.1]octyl, 8-oxa-3-azabicyclo[3.2.1]octyl and 9-azabicyclo[3.3.1]nonyl.

5- or 6-membered heteroaryl in the context of the invention represents an aromatic heterocyclic radical (heteroaromatic) having 5 or, respectively, 6 ring atoms in total which contains up to three identical or different ring hetero atoms from the series N, O and/or S and is linked via a ring carbon atom or optionally via a ring nitrogen atom. There may be mentioned by way of example: furyl, pyrrolyl, thienyl, pyrazolyl, imidazolyl, thiazolyl, oxazolyl, isoxazolyl, isothiazolyl, triazolyl, oxadiazolyl, thiadiazolyl, pyridyl, pyrimidinyl, pyridazinyl, pyrazinyl and triazinyl. 5- or 6-membered heteroaryl radicals having up to two ring hetero atoms from the series N, O and/or S, such as, for example, furyl, pyrrolyl, thienyl, thiazolyl, oxazolyl, isothiazolyl, isoxazolyl, pyrazolyl, imidazolyl, pyridyl, pyrimidinyl, pyridazinyl and pyrazinyl, are preferred.

Halogen in the context of the invention includes fluorine, chlorine, bromine and iodine. Chlorine, fluorine or bromine are preferred, and fluorine or chlorine are particularly preferred.

An oxo substituent in the context of the invention represents an oxygen atom, which is bonded to a carbon atom via a double bond.

If radicals in the compounds according to the invention are substituted, the radicals can be mono- or polysubstituted, unless specified otherwise. In the context of the present invention, for all the radicals which occur several times, the meaning thereof is independent of each other. Substitution by one or by two or three identical or different substituents is preferred. Substitution by one or by two identical or different substituents is particularly preferred.

The present invention provides in particular those compounds of the general formula (I) in which
the ring

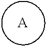

represents a phenyl or pyridyl ring and the adjacent groups X and CH₂ are bonded to ring carbon atoms

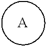

in 1, 3 or 1,4 relation to one another
and
the ring

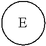

with the substituents R⁴ and R⁵ represents a phenyl ring of the formula

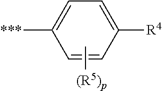

wherein
*** designates the linkage point with the ring

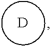

and their salts, solvates and solvates of the salts.

Compounds of the formula (I) which are preferred in the context of the present invention are those in which
the ring

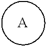

represents a pyridyl ring and the adjacent groups X and CH₂ are bonded to ring carbon atoms of this pyridyl ring in 1, 3 or 1,4 relation to one another
and
the ring

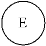

with the substituents R⁴ and R⁵ represents a phenyl ring of the formula

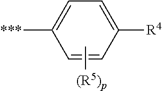

wherein
*** designates the linkage point with the ring

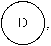

and their salts, solvates and solvates of the salts.

Preferred compounds of the formula (I) are also those in which
the ring

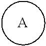

represents a phenyl ring and the adjacent groups X and CH₂ are bonded to this phenyl ring in 1, 3 or 1,4 relation to one another,
the ring

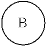

with the substituent R³ represents a heteroaryl ring of the formula

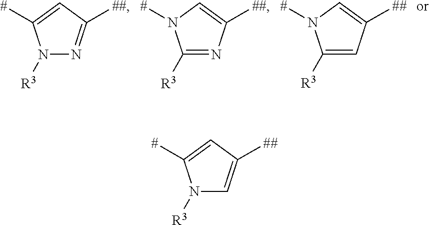

wherein
# designates the linkage point with the adjacent CH₂ group
and
## designates the linkage point with the ring

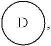

and
the ring

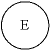

with the substituents R⁴ and R⁵ represents a phenyl ring of the formula

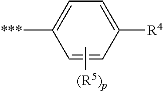

wherein
*** designates the linkage point with the ring

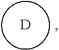

and their salts, solvates and solvates of the salts.

Compounds of the formula (I) which are likewise preferred are those in which
the ring

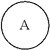

represents a phenyl ring and the adjacent groups X and CH₂ are bonded to this phenyl ring in 1, 3 or 1,4 relation to one another,
the ring

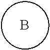

with the substituent R³ represents a heteroaryl ring of the formula

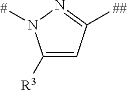

wherein
# designates the linkage point with the adjacent CH₂ group
and
## designates the linkage point with the ring

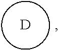

the ring

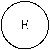

with the substituents R⁴ and R⁵ represents a phenyl ring of the formula

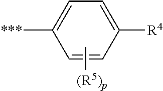

wherein
*** designates the linkage point with the ring

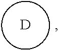

R¹ represents a substituent bonded to a carbon atom of the ring

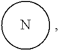

chosen from the series cyano, (C₁-C₆)-alkyl, oxo and (C₃-C₆)-cycloalkyl,
wherein (C₁-C₆)-alkyl in its turn can be substituted up to three times by fluorine and up to two times in an identical or different manner by a radical chosen from the series hydroxyl, (C₁-C₄)-alkoxy, amino, mono-(C₁-C₄)-alkylamino and di-(C₁-C₄)-alkylamino
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
R² represents a substituent bonded to a nitrogen atom of the ring

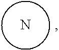

chosen from the series (C₁-C₆)-alkyl, (C₁-C₆)-alkylcarbonyl, (C₁-C₆)-alkoxycarbonyl, (C₁-C₆)-alkylsulfonyl and (C₃-C₆)-cycloalkyl,
wherein the alkyl group in (C₁-C₆)-alkyl, (C₁-C₆)-alkylcarbonyl, (C₁-C₆)-alkoxycarbonyl and (C₁-C₆)-alkylsulfonyl in its turn can be substituted up to three times by fluorine and up to two times in an identical or different manner by a radical chosen from the series hydroxyl, (C₁-C₄)-alkoxy, amino, mono-(C₁-C₄)-alkylamino, di-(C₁-C₄)-alkylamino (C₃-C₆)-cycloalkyl and 4- to 6-membered heterocyclyl
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
m represents the number 0, 1, 2, 3 or 4,
   wherein in the case where the substituent R¹ occurs several times, its meanings can be identical or different,
      and
n represents the number 0 or 1 or also, if the aza-heterocycle

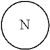

contains further N atoms as ring members, the number 2,
wherein in the case where the substituent R² occurs twice, its meanings can be identical or different,
   wherein the sum of m and n does not equal the number 0,
   and their salts, solvates and solvates of the salts.

An alternative embodiment within the embodiment described last comprises compounds of the formula (I) in which
R¹ represents a substituent bonded to a carbon atom of the ring

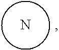

chosen from the series (C₁-C₆)-alkyl, oxo and (C₃-C₆)-cycloalkyl,
wherein (C₁-C₆)-alkyl in its turn can be substituted up to three times by fluorine and up to two times in an identical or different manner by a radical chosen from the series hydroxyl, (C₁-C₄)-alkoxy, amino, mono-(C₁-C₄)-alkylamino and di-(C₁-C₄)-alkylamino
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
m represents the number 0, 1, 2, 3 or 4,
   wherein in the case where the substituent R¹ occurs several times, its meanings can be identical or different,
      and
n represents the number 0 or 1 or also, if the aza-heterocycle

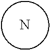

contains further N atoms as ring members, the number 2,
wherein in the case where the substituent R² occurs twice, its meanings can be identical or different,
   wherein the sum of m and n does not equal the number 0,
   and their salts, solvates and solvates of the salts.

Compounds of the formula (I) which are particularly preferred in the context of the present invention are those in which
the ring

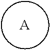

represents a pyridyl ring and the adjacent groups X and CH₂ are bonded to ring carbon atoms of this pyridyl ring in 1, 3 or 1,4 relation to one another,
the ring

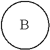

with the substituent R³ represents a heteroaryl ring of the formula

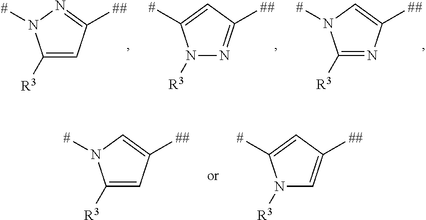

wherein
# designates the linkage point with the adjacent CH₂ group
and
## designates the linkage point with the ring

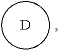

the ring

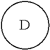

represents a heteroaryl ring of the formula

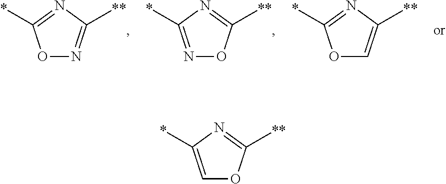

wherein
* designates the linkage point with the ring

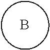

and
** designates the linkage point with the ring

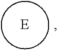

the ring

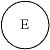

with the substituents R⁴ and R⁵ represents a phenyl ring of the formula

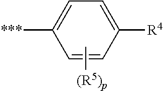

wherein
*** designates the linkage point with the ring

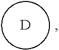

the ring

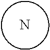

represents a saturated 4- to 10-membered aza-heterocycle, which contains at least one N atom as a ring member and in addition can contain a further hetero ring member from the series N, O, S or S(O)₂,
X represents a bond or ♦—(CH₂)_{q}—N(R⁶)—♦♦, —O—, —S—, —C(═O)—, —S(═O)₂— or ♦—N(R⁶)—C(═O)—♦♦, wherein
   ♦ designates the linkage point with the ring

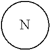

and
└♦ designates the linkage point with the ring

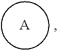

q denotes the number 0, 1 or 2
and
R⁶ denotes hydrogen, (C₁-C4)-alkyl or (C₃-C₆)-cycloalkyl,
R¹ represents a substituent bonded to a carbon atom of the ring

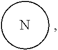

chosen from the series fluorine, cyano, (C₁-C₄)-alkyl, hydroxyl, (C₁-C₄)-alkoxy, oxo, amino, mono-(C₁-C₄)-alkylamino, di-(C₁-C₄)-alkylamino and (C₃-C₆)-cycloalkyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
m represents the number 0, 1 or 2,
   wherein in the case where the substituent R¹ occurs twice, its meanings can be identical or different,
R² represents a substituent bonded to a nitrogen atom of the ring

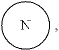

chosen from the series (C₁-C₄)-alkyl, (C₁-C₄)-alkylcarbonyl, (C₁-C₄)-alkoxycarbonyl, (C₁-C₄)-alkylsulfonyl and (C₃-C₆)-cycloalkyl,
wherein the alkyl group in (C₁-C₄)-alkyl, (C₁-C₄)-alkylcarbonyl, (C₁-C₄)-alkoxycarbonyl and (C₁-C₄)-alkylsulfonyl in its turn can be substituted by a radical chosen from the series hydroxyl, (C₁-C₄)-alkoxy, amino, mono-(C₁-C₄)-alkylamino, di-(C₁-C₄)-alkylamino (C₃-C₆)-cycloalkyl and 4- to 6-membered heterocyclyl and up to three times by fluorine
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
n represents the number 0 or 1,
R³ represents methyl, ethyl or trifluoromethyl,
R⁴ represents a substituent chosen from the series fluorine, chlorine, cyano, pentafluorothio, (C₁-C₆)-alkyl, tri-(C₁-C₄)-alkylsilyl, —OR⁷, —NR⁷R⁸, —SR⁷, —S(═O)—R⁷, —S(═O)₂—R⁷, —S(═O)(═NH)—R⁷, —S(═O)(═NCH₃)—R⁷, (C₃-C₆)-cycloalkyl and 4- to 6-membered heterocyclyl,
   wherein (C₁-C₆)-alkyl in its turn can be substituted up to three times by fluorine and up to two times in an identical or different manner by a radical chosen from the series —OR⁷, —NR⁷R⁸, —N(R⁷)—C(═O)—R⁸, —C(═O)—NR⁷R⁸, (C₃-C₆)-cycloalkyl, 4- to 6-membered heterocyclyl and 5- or 6-membered heteroaryl
   and wherein
   the cycloalkyl and heterocyclyl groups mentioned in their turn can be substituted up to two times in an identical or different manner by a radical chosen from the series fluorine, (C₁-C₄)-alkyl, hydroxyl, (C₁-C₄)-alkoxy, trifluoromethoxy, oxo and (C₁-C₄)-alkylcarbonyl
   and
   the heteroaryl group mentioned in its turn can be substituted up to two times in an identical or different manner by a radical chosen from the series fluorine, chlorine, cyano, (C₁-C₄)-alkyl, (C₁-C₄)-alkoxy and trifluoromethoxy
      wherein the (C₁-C₄)-alkyl substituents mentioned herein in their turn can be substituted by hydroxyl, methoxy, trifluoromethoxy, ethoxy, acetoxy, aminocarbonyl, methylaminocarbonyl or dimethylaminocarbonyl or up to three times by fluorine,
   and wherein
   R⁷ and R⁸ independently of each other for each individual occurrence denote hydrogen, (C₁-C₄)-alkyl, (C₃-C₆)-cycloalkyl or 4- to 6-membered heterocyclyl,
      wherein (C₁-C₄)-alkyl can be substituted up to three times by fluorine and up to two times in an identical or different manner by a radical chosen from the series hydroxyl, (C₁-C₄)-alkoxy, trifluoromethoxy, (C₃-C₆)-cycloalkyl and 4- to 6-membered heterocyclyl
      and
      the cycloalkyl and heterocyclyl groups mentioned can be substituted up to two times in an identical or different manner by a radical chosen from the series fluorine, (C₁-C₄)-alkyl, trifluoromethyl, hydroxyl, (C₁-C₄)-alkoxy, trifluoromethoxy, oxo and (C₁-C₄)-alkylcarbonyl
   or
   R⁷ and R⁸ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo and (C₁-C₄)-alkylcarbonyl,
R⁵ represents a substituent chosen from the series fluorine, chlorine and methyl
   and
p represents the number 0 or 1,
   and their salts, solvates and solvates of the salts.

An alternative embodiment within the embodiment described last comprises compounds of the formula (I) in which
R¹ represents a substituent bonded to a carbon atom of the ring

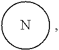

chosen from the series fluorine, (C₁-C₄)-alkyl, hydroxyl, (C₁-C₄)-alkoxy, oxo, amino, mono-(C₁-C₄)-alkylamino, di-(C₁-C₄)-alkylamino and (C₃-C₆)-cycloalkyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
m represents the number 0, 1 or 2,
   wherein in the case where the substituent R¹ occurs twice, its meanings can be identical or different,
      and
R⁴ represents a substituent chosen from the series fluorine, chlorine, cyano, pentafluorothio, (C₁-C₆)-alkyl, tri-(C₁-C₄)-alkylsilyl, —OR⁷, —NR⁷R⁸, —SR⁷, —S(═O)—R⁷, —S(═O)₂—R⁷, (C₃-C₆)-cycloalkyl and 4- to 6-membered heterocyclyl,
   wherein (C₁-C₆)-alkyl in its turn can be substituted up to three times by fluorine and up to two times in an identical or different manner by a radical chosen from the series —OR⁷, —NR⁷R⁸, —N(R⁷)—C(═O)—R⁸, —C(═O)—NR⁷R⁸, (C₃-C₆)-cycloalkyl, 4- to 6-membered heterocyclyl and 5- or 6-membered heteroaryl
   and wherein
   the cycloalkyl and heterocyclyl groups mentioned in their turn can be substituted up to two times in an identical or different manner by a radical chosen from the series fluorine, (C₁-C₄)-alkyl, trifluoromethyl, hydroxyl, (C₁-C₄)-alkoxy, trifluoromethoxy, oxo and (C₁-C₄)-alkylcarbonyl
   and
   the heteroaryl group mentioned in its turn can be substituted up to two times in an identical or different manner by a radical chosen from the series fluorine, chlorine, cyano, (C₁-C₄)-alkyl, trifluoromethyl, (C₁-C₄)-alkoxy and trifluoromethoxy
   and wherein R⁷ and R⁸ have the meanings given in this embodiment last described,
      and their salts, solvates and solvates of the salts.

Compounds of the formula (I) which are also particularly preferred are those in which
the ring

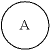

represents a phenyl ring and the adjacent groups X and CH₂ are bonded to this phenyl ring in 1, 3 or 1,4 relation to one another,
the ring

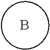

with the substituent R³ represents a heteroaryl ring of the formula

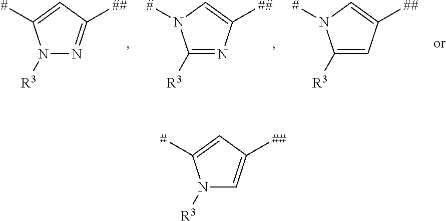

wherein
# designates the linkage point with the adjacent CH₂ group
and
## designates the linkage point with the ring

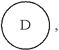

the ring

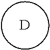

represents a heteroaryl ring of the formula

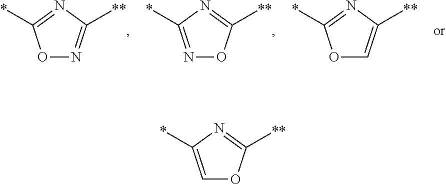

wherein
* designates the linkage point with the ring

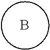

and
** designates the linkage point with the ring

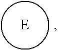

the ring

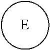

with the substituents R⁴ and R⁵ represents a phenyl ring of the formula

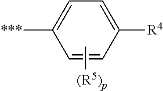

wherein
*** designates the linkage point with the ring

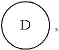

the ring

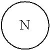

represents a saturated 4- to 10-membered aza-heterocycle, which contains at least one N atom as a ring member and in addition can contain a further hetero ring member from the series N, O, S or S(O)₂,
X represents a bond or ♦—(CH₂)_{q}—N(R⁶)—♦♦, —O—, —S—, —C(═O)—, —S(═O)₂— or ♦—N(R⁶)—C(═O)—♦♦, wherein
   ♦ designates the linkage point with the ring

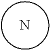

and
♦♦ designates the linkage point with the ring

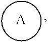

q denotes the number 0, 1 or 2
and
R⁶ denotes hydrogen, (C₁-C₄)-alkyl or (C₃-C₆)-cycloalkyl,
R¹ represents a substituent bonded to a carbon atom of the ring

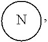

chosen from the series fluorine, cyano, (C₁-C₄)-alkyl, hydroxyl, (C₁-C₄)-alkoxy, oxo, amino, mono-(C₁-C₄)-alkylamino, di-(C₁-C₄)-alkylamino and (C₃-C₆)-cycloalkyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
m represents the number 0, 1 or 2,
   wherein in the case where the substituent R¹ occurs twice, its meanings can be identical or different,
R² represents a substituent bonded to a nitrogen atom of the ring

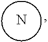

chosen from the series (C₁-C₄)-alkyl, (C₁-C₄)-alkylcarbonyl, (C₁-C₄)-alkoxycarbonyl, (C₁-C₄)-alkylsulfonyl and (C₃-C₆)-cycloalkyl,
wherein the alkyl group in (C₁-C₄)-alkyl, (C₁-C₄)-alkylcarbonyl, (C₁-C₄)-alkoxycarbonyl and (C₁-C₄)-alkylsulfonyl in its turn can be substituted by a radical chosen from the series hydroxyl, (C₁-C₄)-alkoxy, amino, mono-(C₁-C₄)-alkylamino, di-(C₁-C₄)-alkylamino (C₃-C₆)-cycloalkyl and 4- to 6-membered heterocyclyl and up to three times by fluorine
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
n represents the number 0 or 1,
R³ represents methyl, ethyl or trifluoromethyl,
R⁴ represents a substituent chosen from the series fluorine, chlorine, cyano, pentafluorothio, (C₁-C₆)-alkyl, tri-(C₁-C₄)-alkylsilyl, —OR⁷, —NR⁷R⁸, —SR⁷, —S(═O)—R⁷, —S(═O)₂—R⁷, —S(═O)(═NH)—R⁷, —S(═O)(═NCH₃)—R⁷, (C₃-C₆)-cycloalkyl and 4- to 6-membered heterocyclyl,
   wherein (C₁-C₆)-alkyl in its turn can be substituted up to three times by fluorine and up to two times in an identical or different manner by a radical chosen from the series —OR⁷, —NR⁷R⁸, —N(R⁷)—C(═O)—R⁸, —C(═O)—NR⁷R⁸, (C₃-C₆)-cycloalkyl, 4- to 6-membered heterocyclyl and 5- or 6-membered heteroaryl
   and wherein
   the cycloalkyl and heterocyclyl groups mentioned in their turn can be substituted up to two times in an identical or different manner by a radical chosen from the series fluorine, (C₁-C₄)-alkyl, hydroxyl, (C₁-C₄)-alkoxy, trifluoromethoxy, oxo and (C₁-C₄)-alkylcarbonyl
   and
   the heteroaryl group mentioned in its turn can be substituted up to two times in an identical or different manner by a radical chosen from the series fluorine, chlorine, cyano, (C₁-C₄)-alkyl, (C₁-C₄)-alkoxy and trifluoromethoxy
      wherein the (C₁-C₄)-alkyl substituents mentioned herein in their turn can be substituted by hydroxyl, methoxy, trifluoromethoxy, ethoxy, acetoxy, aminocarbonyl, methylaminocarbonyl or dimethylaminocarbonyl or up to three times by fluorine,
   and wherein
   R⁷ and R⁸ independently of each other for each individual occurrence denote hydrogen, (C₁-C₄)-alkyl, (C₃-C₆)-cycloalkyl or 4- to 6-membered heterocyclyl,
      wherein (C₁-C₄)-alkyl can be substituted up to three times by fluorine and up to two times in an identical or different manner by a radical chosen from the series hydroxyl, (C₁-C₄)-alkoxy, trifluoromethoxy, (C₃-C₆)-cycloalkyl and 4- to 6-membered heterocyclyl
      and
      the cycloalkyl and heterocyclyl groups mentioned can be substituted up to two times in an identical or different manner by a radical chosen from the series fluorine, (C₁-C₄)-alkyl, trifluoromethyl, hydroxyl, (C₁-C₄)-alkoxy, trifluoromethoxy, oxo and (C₁-C₄)-alkylcarbonyl
   or
   R⁷ and R⁸ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo and (C₁-C₄)-alkylcarbonyl,
R⁵ represents a substituent chosen from the series fluorine, chlorine and methyl
   and
p represents the number 0 or 1,
   and their salts, solvates and solvates of the salts.

An alternative embodiment within the embodiment described last comprises compounds of the formula (I) in which
R¹ represents a substituent bonded to a carbon atom of the ring

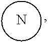

chosen from the series fluorine, (C₁-C₄)-alkyl, hydroxyl, (C₁-C₄)-alkoxy, oxo, amino, mono-(C₁-C₄)-alkylamino, di-(C₁-C₄)-alkylamino and (C₃-C₆)-cycloalkyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
m represents the number 0, 1 or 2,
   wherein in the case where the substituent R¹ occurs twice, its meanings can be identical or different,
      and
R⁴ represents a substituent chosen from the series fluorine, chlorine, cyano, pentafluorothio, (C₁-C₆)-alkyl, tri-(C₁-C₄)-alkylsilyl, —OR⁷, —NR⁷R⁸, —SR⁷, —S(═O)—R⁷, —S(═O)₂—R⁷, (C₃-C₆)-cycloalkyl and 4- to 6-membered heterocyclyl,
   wherein (C₁-C₆)-alkyl in its turn can be substituted up to three times by fluorine and up to two times in an identical or different manner by a radical chosen from the series —OR⁷, —NR⁷R⁸, —N(R⁷)—C(═O)—R⁸, —C(═O)—NR⁷R⁸, (C₃-C₆)-cycloalkyl, 4- to 6-membered heterocyclyl and 5- or 6-membered heteroaryl
   and wherein
   the cycloalkyl and heterocyclyl groups mentioned in their turn can be substituted up to two times in an identical or different manner by a radical chosen from the series fluorine, (C₁-C₄)-alkyl, trifluoromethyl, hydroxyl, (C₁-C₄)-alkoxy, trifluoromethoxy, oxo and (C₁-C₄)-alkylcarbonyl
   and
   the heteroaryl group mentioned in its turn can be substituted up to two times in an identical or different manner by a radical chosen from the series fluorine, chlorine, cyano, (C₁-C₄)-alkyl, trifluoromethyl, (C₁-C₄)-alkoxy and trifluoromethoxy
   and wherein R⁷ and R⁸ have the meanings given in this embodiment last described,
      and their salts, solvates and solvates of the salts.

Compounds of the formula (I) which are likewise particularly preferred are those in which
the ring

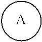

represents a phenyl ring and the adjacent groups X and CH₂ are bonded to this phenyl ring in 1, 3 or 1,4 relation to one another,
the ring

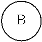

with the substituent R³ represents a heteroaryl ring of the formula

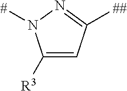

wherein
# designates the linkage point with the adjacent CH₂ group
and
## designates the linkage point with the ring

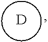

the ring

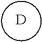

represents a heteroaryl ring of the formula

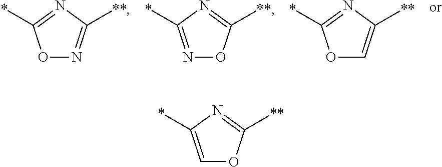

wherein
* designates the linkage point with the ring

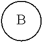

and
** designates the linkage point with the ring

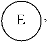

the ring

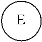

with the substituents R⁴ and R⁵ represents a phenyl ring of the formula

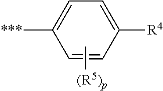

wherein
*** designates the linkage point with the ring

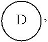

the ring

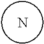

represents a saturated 4- to 10-membered aza-heterocycle, which contains at least one N atom as a ring member and in addition can contain a further hetero ring member from the series N, O, S or S(O)₂,
X represents a bond or ♦—(CH₂)_{q}—N(R⁶)—♦♦, —O—, —S—, —C(═O)—, —S(═O)₂— or ♦—N(R⁶)—C(═O)—♦♦, wherein
   ♦ designates the linkage point with the ring

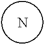

and
♦♦ designates the linkage point with the ring

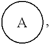

q denotes the number 0, 1 or 2
and
R⁶ denotes hydrogen, (C₁-C₄)-alkyl or (C₃-C₆)-cycloalkyl,
R¹ represents a substituent bonded to a carbon atom of the ring

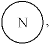

chosen from the series cyano, (C₁-C₄)-alkyl, oxo and (C₃-C₆)-cycloalkyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
R² represents a substituent bonded to a nitrogen atom of the ring

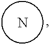

chosen from the series (C₁-C₄)-alkyl, (C₁-C₄)-alkylcarbonyl, (C₁-C₄)-alkoxycarbonyl, (C₁-C₄)-alkylsulfonyl and (C₃-C₆)-cycloalkyl,
wherein the alkyl group in (C₁-C₄)-alkyl, (C₁-C₄)-alkylcarbonyl, (C₁-C₄)-alkoxycarbonyl and (C₁-C₄)-alkylsulfonyl in its turn can be substituted by a radical chosen from the series hydroxyl, (C₁-C₄)-alkoxy, amino, mono-(C₁-C₄)-alkylamino, di-(C₁-C₄)-alkylamino (C₃-C₆)-cycloalkyl and 4- to 6-membered heterocyclyl and up to three times by fluorine
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
m represents the number 0, 1 or 2,
   wherein in the case where the substituent R¹ occurs twice, its meanings can be identical or different,
n represents the number 0 or 1,
   wherein the sum of m and n equals the number 1, 2 or 3,
R³ represents methyl, ethyl or trifluoromethyl,
R⁴ represents a substituent chosen from the series fluorine, chlorine, cyano, pentafluorothio, (C₁-C₆)-alkyl, tri-(C₁-C₄)-alkylsilyl, —OR⁷, —NR⁷R⁸, —SR⁷, —S(═O)—R⁷, —S(═O)₂—R⁷, —S(═O)(═NH)—R⁷, —S(═O)(═NCH₃)—R⁷, (C₃-C₆)-cycloalkyl and 4- to 6-membered heterocyclyl,
   wherein (C₁-C₆)-alkyl in its turn can be substituted up to three times by fluorine and up to two times in an identical or different manner by a radical chosen from the series —OR⁷, —NR⁷R⁸, —N(R⁷)—C(═O)—R⁸, —C(═O)—NR⁷R⁸, (C₃-C₆)-cycloalkyl, 4- to 6-membered heterocyclyl and 5- or 6-membered heteroaryl
   and wherein
   the cycloalkyl and heterocyclyl groups mentioned in their turn can be substituted up to two times in an identical or different manner by a radical chosen from the series fluorine, (C₁-C₄)-alkyl, hydroxyl, (C₁-C₄)-alkoxy, trifluoromethoxy, oxo and (C₁-C₄)-alkylcarbonyl
   and
   the heteroaryl group mentioned in its turn can be substituted up to two times in an identical or different manner by a radical chosen from the series fluorine, chlorine, cyano, (C₁-C₄)-alkyl, (C₁-C₄)-alkoxy and trifluoromethoxy
      wherein the (C₁-C₄)-alkyl substituents mentioned herein in their turn can be substituted by hydroxyl, methoxy, trifluoromethoxy, ethoxy, acetoxy, aminocarbonyl, methylaminocarbonyl or dimethylaminocarbonyl or up to three times by fluorine,
   and wherein
   R⁷ and R⁸ independently of each other for each individual occurrence denote hydrogen, (C₁-C₄)-alkyl, (C₃-C₆)-cycloalkyl or 4- to 6-membered heterocyclyl,
      wherein (C₁-C₄)-alkyl can be substituted up to three times by fluorine and up to two times in an identical or different manner by a radical chosen from the series hydroxyl, (C₁-C₄)-alkoxy, trifluoromethoxy, (C₃-C₆)-cycloalkyl and 4- to 6-membered heterocyclyl
      and
      the cycloalkyl and heterocyclyl groups mentioned can be substituted up to two times in an identical or different manner by a radical chosen from the series fluorine, (C₁-C₄)-alkyl, trifluoromethyl, hydroxyl, (C₁-C₄)-alkoxy, trifluoromethoxy, oxo and (C₁-C₄)-alkylcarbonyl
   or
   R⁷ and R⁸ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo and (C₁-C₄)-alkylcarbonyl,
R⁵ represents a substituent chosen from the series fluorine, chlorine and methyl
   and
p represents the number 0 or 1,
   and their salts, solvates and solvates of the salts.

An alternative embodiment within the embodiment described last comprises compounds of the formula (I) in which
R¹ represents a substituent bonded to a carbon atom of the ring

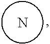

chosen from the series (C₁-C₄)-alkyl, oxo and (C₃-C₆)-cycloalkyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
m represents the number 0, 1 or 2,
   wherein in the case where the substituent R¹ occurs twice, its meanings can be identical or different,
n represents the number 0 or 1,
   wherein the sum of m and n equals the number 1, 2 or 3,
   and
R⁴ represents a substituent chosen from the series fluorine, chlorine, cyano, pentafluorothio, (C₁-C₆)-alkyl, tri-(C₁-C₄)-alkylsilyl, —OR⁷, —NR⁷R⁸, —SR⁷, —S(═O)—R⁷, —S(═O)₂—R⁷, (C₃-C₆)-cycloalkyl and 4- to 6-membered heterocyclyl,
   wherein (C₁-C₆)-alkyl in its turn can be substituted up to three times by fluorine and up to two times in an identical or different manner by a radical chosen from the series —OR⁷, —NR⁷R⁸, —N(R⁷)—C(═O)—R⁸, —C(═O)—NR⁷R⁸, (C₃-C₆)-cycloalkyl, 4- to 6-membered heterocyclyl and 5- or 6-membered heteroaryl
   and wherein
   the cycloalkyl and heterocyclyl groups mentioned in their turn can be substituted up to two times in an identical or different manner by a radical chosen from the series fluorine, (C₁-C₄)-alkyl, trifluoromethyl, hydroxyl, (C₁-C₄)-alkoxy, trifluoromethoxy, oxo and (C₁-C₄)-alkylcarbonyl
   and
   the heteroaryl group mentioned in its turn can be substituted up to two times in an identical or different manner by a radical chosen from the series fluorine, chlorine, cyano, (C₁-C₄)-alkyl, trifluoromethyl, (C₁-C₄)-alkoxy and trifluoromethoxy
   and wherein R⁷ and R⁸ have the meanings given in this embodiment last described,
      and their salts, solvates and solvates of the salts.

Compounds of the formula (I) which are very particularly preferred in the context of the present invention are those in which
the ring

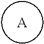

represents a pyridyl ring of the formula

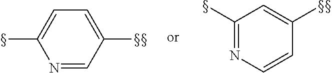

wherein
§ designates the linkage point with the adjacent group X
and
§§ designates the linkage point with the adjacent CH₂ group,
the ring

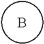

with the substituent R³ represents a heteroaryl ring of the formula

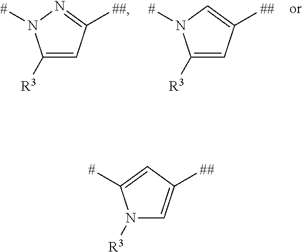

wherein
# designates the linkage point with the adjacent CH₂ group
and
## designates the linkage point with the ring

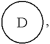

the ring

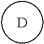

represents a heteroaryl ring of the formula

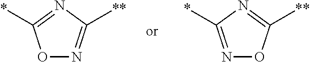

wherein
* designates the linkage point with the ring

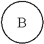

and
** designates the linkage point with the ring

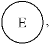

the ring

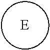

with the substituents R⁴ and R⁵ represents a phenyl ring of the formula

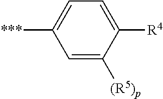

wherein
*** designates the linkage point with the ring

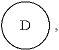

the ring

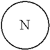

represents a saturated 4- to 10-membered aza-heterocycle, which contains at least one N atom as a ring member and in addition can contain a further hetero ring member from the series N, O, S or S(O)₂,
X represents a bond or ♦—(CH₂)_{q}—N(R⁶)—♦♦, —C(═O)— or ♦—N(R⁶)—C(═O)—♦♦, wherein
   ♦ designates the linkage point with the ring

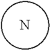

and
♦♦ designates the linkage point with the ring

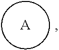

q denotes the number 0, 1 or 2
and
R⁶ denotes hydrogen, methyl, ethyl, isopropyl, cyclopropyl or cyclobutyl,
R¹ represents a substituent bonded to a carbon atom of the ring

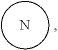

chosen from the series fluorine, cyano, (C₁-C₄)-alkyl, hydroxyl, (C₁-C₄)-alkoxy, oxo, amino, mono-(C₁-C₄)-alkylamino, di-(C₁-C₄)-alkylamino, cyclopropyl and cyclobutyl, wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine,
m represents the number 0 or 1,
R² represents a substituent bonded to a nitrogen atom of the ring

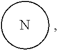

chosen from the series (C₁-C₄)-alkyl, (C₁-C₄)-alkylcarbonyl, (C₁-C₄)-alkoxycarbonyl, (C₁-C₄)-alkylsulfonyl, cyclopropyl and cyclobutyl,
wherein the alkyl group in (C₁-C₄)-alkyl, (C₁-C₄)-alkylcarbonyl, (C₁-C₄)-alkoxycarbonyl and (C₁-C₄)-alkylsulfonyl in its turn can be substituted by a radical chosen from the series hydroxyl, (C₁-C₄)-alkoxy, amino, mono-(C₁-C₄)-alkylamino, di-(C₁-C₄)-alkylamino (C₃-C₅)-cycloalkyl and 4- or 5-membered heterocyclyl and up to three times by fluorine
n represents the number 0 or 1,
R³ represents methyl,
R⁴ represents a substituent chosen from the series chlorine, pentafluorothio, (C₁-C₆)-alkyl, trimethylsilyl, —OR⁷, —SR⁷, —S(═O)—R⁷, —S(═O)₂—R⁷, —S(═O)(═NCH₃)—CF₃, (C₃-C₆)-cycloalkyl and 4- to 6-membered heterocyclyl,
   wherein (C₁-C₆)-alkyl in its turn can be substituted by a radical chosen from the series —OR⁷, —NR⁷R⁸, —C(═O)—NR⁷R⁸, (C₃-C₆)-cycloalkyl and 4- to 6-membered heterocyclyl and up to three time by fluorine
   and
   the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
      wherein the (C₁-C₄)-alkyl substituent in its turn can be substituted by methoxy, trifluoromethoxy or ethoxy,
   and wherein
   R⁷ and R⁸ independently of each other for each individual occurrence denote hydrogen, (C₁-C₄)-alkyl or (C₃-C₆)-cycloalkyl,
      wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
      and
      the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy and trifluoromethoxy,
   or
   R⁷ and R⁸ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo and (C₁-C₄)-alkylcarbonyl,
R⁵ represents fluorine,
   and
p represents the number 0 or 1,
   and their salts, solvates and solvates of the salts.

An alternative embodiment within the embodiment described last comprises compounds of the formula (I) in which
R¹ represents a substituent bonded to a carbon atom of the ring

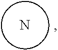

chosen from the series fluorine, (C₁-C₄)-alkyl, hydroxyl, (C₁-C₄)-alkoxy, oxo, amino, mono-(C₁-C₄)-alkylamino, di-(C₁-C₄)-alkylamino, cyclopropyl and cyclobutyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine,
m represents the number 0 or 1,
   and
R⁴ represents a substituent chosen from the series chlorine, pentafluorothio, (C₁-C₆)-alkyl, trimethylsilyl, —OR⁷, —SR⁷, —S(═O)—R⁷, —S(═O)₂—R⁷, (C₃-C₆)-cycloalkyl and 4- to 6-membered heterocyclyl,
   wherein (C₁-C₆)-alkyl in its turn can be substituted by a radical chosen from the series —OR′, —NR⁷R⁸, —C(═O)—NR⁷R⁸, (C₃-C₆)-cycloalkyl and 4- to 6-membered heterocyclyl and up to three time by fluorine
   and
   the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
   and wherein R⁷ and R⁸ have the meanings given in this embodiment last described,
      and their salts, solvates and solvates of the salts.

Compounds of the formula (I) which are also very particularly preferred are those in which
the ring

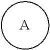

represents a phenyl ring and the adjacent groups X and CH₂ are bonded to this phenyl ring in 1, 3 or 1,4 relation to one another,
the ring

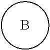

with the substituent R³ represents a heteroaryl ring of the formula

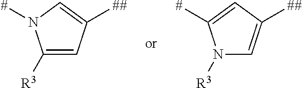

wherein
# designates the linkage point with the adjacent CH₂ group
and
## designates the linkage point with the ring

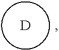

the ring

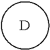

represents a heteroaryl ring of the formula

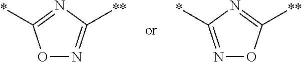

wherein
* designates the linkage point with the ring

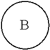

and
** designates the linkage point with the ring

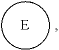

the ring

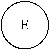

with the substituents R⁴ and R⁵ represents a phenyl ring of the formula

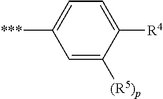

wherein
*** designates the linkage point with the ring

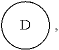

the ring

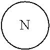

represents a saturated 4- to 10-membered aza-heterocycle, which contains at least one N atom as a ring member and in addition can contain a further hetero ring member from the series N, O, S or S(O)₂,
X represents a bond or ♦—(CH₂)_{q}—N(R⁶)—♦♦, —C(═O)— or ♦—N(R⁶)—C(═O)—♦♦, wherein
   ♦ designates the linkage point with the ring

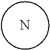

and
♦♦ designates the linkage point with the ring

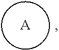

q denotes the number 0, 1 or 2
and
R⁶ denotes hydrogen, methyl, ethyl, isopropyl, cyclopropyl or cyclobutyl,
R¹ represents a substituent bonded to a carbon atom of the ring

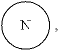

chosen from the series fluorine, cyano, (C₁-C₄)-alkyl, hydroxyl, (C₁-C₄)-alkoxy, oxo, amino, mono-(C₁-C₄)-alkylamino, di-(C₁-C₄)-alkylamino, cyclopropyl and cyclobutyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine,
m represents the number 0 or 1,
R² represents a substituent bonded to a nitrogen atom of the ring

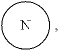

chosen from the series (C₁-C₄)-alkyl, (C₁-C₄)-alkylcarbonyl, (C₁-C₄)-alkoxycarbonyl, (C₁-C₄)-alkylsulfonyl, cyclopropyl and cyclobutyl,
wherein the alkyl group in (C₁-C₄)-alkyl, (C₁-C₄)-alkylcarbonyl, (C₁-C₄)-alkoxycarbonyl and (C₁-C₄)-alkylsulfonyl in its turn can be substituted by a radical chosen from the series hydroxyl, (C₁-C₄)-alkoxy, amino, mono-(C₁-C₄)-alkylamino, di-(C₁-C₄)-alkylamino (C₃-C₅)-cycloalkyl and 4- or 5-membered heterocyclyl and up to three times by fluorine,
n represents the number 0 or 1,
R³ represents methyl,
R⁴ represents a substituent chosen from the series chlorine, pentafluorothio, (C₁-C₆)-alkyl, trimethylsilyl, —OR⁷, —SR⁷, —S(═O)—R⁷, —S(═O)₂—R⁷, —S(═O)(═NCH₃)—CF₃, (C₃-C₆)-cycloalkyl and 4- to 6-membered heterocyclyl,
   wherein (C₁-C₆)-alkyl in its turn can be substituted by a radical chosen from the series —OR′, —NR⁷R⁸, —C(═O)—NR⁷R⁸, (C₃-C₆)-cycloalkyl and 4- to 6-membered heterocyclyl and up to three time by fluorine
   and
   the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
      wherein the (C₁-C₄)-alkyl substituent mentioned in its turn can be substituted by methoxy, trifluoromethoxy or ethoxy,
   and wherein
   R⁷ and R⁸ independently of each other for each individual occurrence denote hydrogen, (C₁-C₄)-alkyl or (C₃-C₆)-cycloalkyl,
      wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
      and
      the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy and trifluoromethoxy,
   or
   R⁷ and R⁸ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo and (C₁-C₄)-alkylcarbonyl,
R⁵ represents fluorine,
   and
p represents the number 0 or 1,
   and their salts, solvates and solvates of the salts.

An alternative embodiment within the embodiment described last comprises compounds of the formula (I) in which
R¹ represents a substituent bonded to a carbon atom of the ring

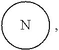

chosen from the series fluorine, (C₁-C₄)-alkyl, hydroxyl, (C₁-C₄)-alkoxy, oxo, amino, mono-(C₁-C₄)-alkylamino, di-(C₁-C₄)-alkylamino, cyclopropyl and cyclobutyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine,
m represents the number 0 or 1,
   and
R⁴ represents a substituent chosen from the series chlorine, pentafluorothio, (C₁-C₆)-alkyl, trimethylsilyl, —OR⁷, —SR⁷, —S(═O)—R⁷, —S(═O)₂—R⁷, (C₃-C₆)-cycloalkyl and 4- to 6-membered heterocyclyl,
   wherein (C₁-C₆)-alkyl in its turn can be substituted by a radical chosen from the series —OR′, —NR⁷R⁸, —C(═O)—NR⁷R⁸, (C₃-C₆)-cycloalkyl and 4- to 6-membered heterocyclyl and up to three times by fluorine
   and
   the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
   and wherein R⁷ and R⁸ have the meanings given in this embodiment last described,
      and their salts, solvates and solvates of the salts.

Compounds of the formula (I) which are likewise very particularly preferred are those in which
the ring

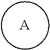

represents a phenyl ring and the adjacent groups X and CH₂ are bonded to this phenyl ring in 1, 3 or 1,4 relation to one another,
the ring

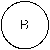

with the substituent R³ represents a heteroaryl ring of the formula

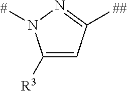

wherein
# designates the linkage point with the adjacent CH₂ group
and
## designates the linkage point with the ring

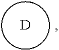

the ring

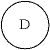

represents a heteroaryl ring of the formula

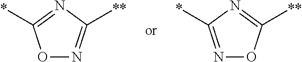

wherein
* designates the linkage point with the ring

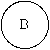

and
** designates the linkage point with the ring

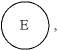

the ring

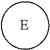

with the substituents R⁴ and R⁵ represents a phenyl ring of the formula

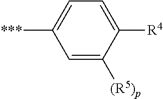

wherein
*** designates the linkage point with the ring

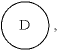

the ring

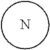

represents a saturated 4- to 10-membered aza-heterocycle, which contains at least one N atom as a ring member and in addition can contain a further hetero ring member from the series N, O, S or S(O)₂,
X represents a bond or ♦—(CH₂)_{q}—N(R⁶)—♦♦, —C(═O)— or ♦—N(R⁶)—C(═O)—♦♦, wherein
   ♦ designates the linkage point with the ring

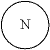

and
♦♦ designates the linkage point with the ring

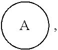

q denotes the number 0, 1 or 2
and
R⁶ denotes hydrogen, methyl, ethyl, isopropyl, cyclopropyl or cyclobutyl,
R¹ represents a substituent bonded to a carbon atom of the ring

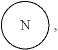

chosen from the series cyano, (C₁-C₄)-alkyl, oxo, cyclopropyl and cyclobutyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine,
R² represents a substituent bonded to a nitrogen atom of the ring

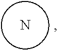

chosen from the series (C₁-C₄)-alkyl, (C₁-C₄)-alkylcarbonyl, (C₁-C₄)-alkoxycarbonyl, (C₁-C₄)-alkylsulfonyl, cyclopropyl and cyclobutyl,
wherein the alkyl group in (C₁-C₄)-alkyl, (C₁-C₄)-alkylcarbonyl, (C₁-C₄)-alkoxycarbonyl and (C₁-C₄)-alkylsulfonyl in its turn can be substituted by a radical chosen from the series hydroxyl, (C₁-C₄)-alkoxy, amino, mono-(C₁-C₄)-alkylamino, di-(C₁-C₄)-alkylamino (C₃-C₅)-cycloalkyl and 4- or 5-membered heterocyclyl and up to three times by fluorine,
m represents the number 0 or 1,
n represents the number 0 or 1,
   wherein the sum of m and n equals the number 1 or 2,
R³ represents methyl,
R⁴ represents a substituent chosen from the series chlorine, pentafluorothio, (C₁-C₆)-alkyl, trimethylsilyl, —OR⁷, —SR⁷, —S(═O)—R⁷, —S(═O)₂—R⁷, —S(═O)(═NCH₃)—CF₃, (C₃-C₆)-cycloalkyl and 4- to 6-membered heterocyclyl,
   wherein (C₁-C₆)-alkyl in its turn can be substituted by a radical chosen from the series —OR⁷, —NR⁷R⁸, —C(═O)—NR⁷R⁸, (C₃-C₆)-cycloalkyl and 4- to 6-membered heterocyclyl and up to three times by fluorine
   and
   the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
      wherein the (C₁-C₄)-alkyl substituent mentioned in its turn can be substituted by methoxy, trifluoromethoxy or ethoxy,
   and wherein
   R⁷ and R⁸ independently of each other for each individual occurrence denote hydrogen, (C₁-C₄)-alkyl or (C₃-C₆)-cycloalkyl,
      wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
      and
      the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy and trifluoromethoxy,
   or
   R⁷ and R⁸ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo and (C₁-C₄)-alkylcarbonyl,
R⁵ represents fluorine,
   and
p represents the number 0 or 1,
   and their salts, solvates and solvates of the salts.

An alternative embodiment within the embodiment described last comprises compounds of the formula (I) in which
R¹ represents a substituent bonded to a carbon atom of the ring

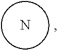

chosen from the series (C₁-C₄)-alkyl, oxo, cyclopropyl and cyclobutyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine,
m represents the number 0 or 1,
n represents the number 0 or 1,
   wherein the sum of m and n equals the number 1 or 2,
   and
R⁴ represents a substituent chosen from the series chlorine, pentafluorothio, (C₁-C₆)-alkyl, trimethylsilyl, —OR⁷, —SR⁷, —S(═O)—R⁷, —S(═O)₂—R⁷, (C₃-C₆)-cycloalkyl and 4- to 6-membered heterocyclyl,
   wherein (C₁-C₆)-alkyl in its turn can be substituted by a radical chosen from the series —OR⁷, —NR⁷R⁸, —C(═O)—NR⁷R⁸, (C₃-C₆)-cycloalkyl and 4- to 6-membered heterocyclyl and up to three times by fluorine
   and
   the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
   and wherein R⁷ and R⁸ have the meanings given in this embodiment last described,
      and their salts, solvates and solvates of the salts.

A particular embodiment of the present invention relates to compounds of the formula (I) in which, in the definition of the group X,
q represents the number 0 or 1,
   and their salts, solvates and solvates of the salts.

Compounds of the formula (I) which are preferred in particular in the context of the present invention are those in which
the ring

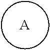

represents a pyridyl ring of the formula

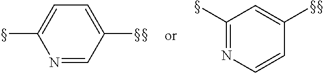

wherein
§ designates the linkage point with the adjacent group X
and
§§ designates the linkage point with the adjacent CH₂ group,
the ring

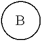

with the substituent R³ represents a heteroaryl ring of the formula

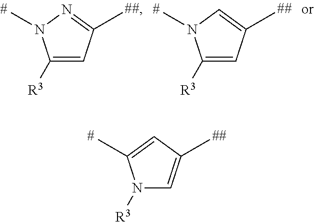

wherein
# designates the linkage point with the adjacent CH₂ group
and
## designates the linkage point with the ring

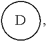

the ring

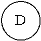

represents a heteroaryl ring of the formula

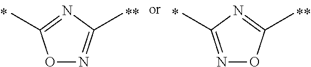

wherein
* designates the linkage point with the ring

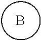

and
** designates the linkage point with the ring

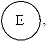

the ring

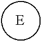

with the substituents R⁴ and R⁵ represents a phenyl ring of the formula

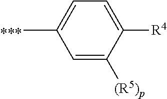

wherein
*** designates the linkage point with the ring

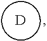

the ring

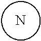

represents a saturated 4- to 10-membered aza-heterocycle, which contains at least one N atom as a ring member and in addition can contain a further hetero ring member from the series N, O, S or S(O)₂,
X represents a bond or ♦—(CH₂)_{q}—N(R⁶)—♦♦, —C(═O)— or ♦—N(R⁶)—C(═O)—♦♦, wherein
   ♦ designates the linkage point with the ring

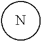

and
♦♦ designates the linkage point with the ring

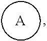

q denotes the number 0 or 1
and
R⁶ denotes hydrogen, methyl, ethyl, isopropyl, cyclopropyl or cyclobutyl,
R¹ represents a substituent bonded to a carbon atom of the ring

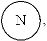

chosen from the series fluorine, cyano, (C₁-C₄)-alkyl, hydroxyl, (C₁-C₄)-alkoxy, oxo, amino, mono-(C₁-C₄)-alkylamino, di-(C₁-C₄)-alkylamino, cyclopropyl and cyclobutyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine,
m represents the number 0 or 1,
R² represents a substituent bonded to a nitrogen atom of the ring

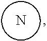

chosen from the series (C₁-C₄)-alkyl, (C₁-C₄)-alkylcarbonyl, (C₁-C₄)-alkoxycarbonyl, (C₁-C₄)-alkylsulfonyl, cyclopropyl and cyclobutyl,
wherein the alkyl group in (C₁-C₄)-alkyl, (C₁-C₄)-alkylcarbonyl, (C₁-C₄)-alkoxycarbonyl and (C₁-C₄)-alkylsulfonyl in its turn can be substituted by a radical chosen from the series hydroxyl, (C₁-C₄)-alkoxy, amino, mono-(C₁-C₄)-alkylamino, di-(C₁-C₄)-alkylamino (C₃-C₅)-cycloalkyl and 4- or 5-membered heterocyclyl and up to three times by fluorine,
n represents the number 0 or 1,
R³ represents methyl,
R⁴ represents a substituent chosen from the series chlorine, pentafluorothio, (C₁-C₆)-alkyl, trimethylsilyl, —OR⁷, —SR⁷, —S(═O)—R⁷, —S(═O)₂—R⁷, —S(═O)(═NCH₃)—CF₃, (C₃-C₆)-cycloalkyl and 4- to 6-membered heterocyclyl,
   wherein (C₁-C₆)-alkyl in its turn can be substituted by a radical chosen from the series —OR⁷, —NR⁷R⁸, —C(═O)—NR⁷R⁸, (C₃-C₆)-cycloalkyl and 4- to 6-membered heterocyclyl and up to three times by fluorine
   and
   the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
      wherein the (C₁-C₄)-alkyl substituent mentioned in its turn can be substituted by methoxy, trifluoromethoxy or ethoxy,
   and wherein
   R⁷ and R⁸ independently of each other for each individual occurrence denote hydrogen, (C₁-C₄)-alkyl or (C₃-C₆)-cycloalkyl,
      wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
      and
      the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy and trifluoromethoxy,
   or
   R⁷ and R⁸ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo and (C₁-C₄)-alkylcarbonyl,
R⁵ represents fluorine,
   and
p represents the number 0 or 1,
   and their salts, solvates and solvates of the salts.

Compounds of the formula (I) which are also preferred in particular are those in which
the ring

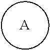

represents a phenyl ring and the adjacent groups X and CH₂ are bonded to this phenyl ring in 1, 3 or 1,4 relation to one another,
the ring

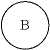

with the substituent R³ represents a heteroaryl ring of the formula

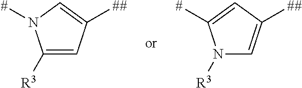

wherein
# designates the linkage point with the adjacent CH₂ group
and
## designates the linkage point with the ring

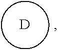

the ring

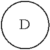

represents a heteroaryl ring of the formula

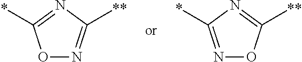

wherein
* designates the linkage point with the ring

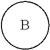

and
** designates the linkage point with the ring

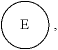

the ring

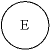

with the substituents R⁴ and R⁵ represents a phenyl ring of the formula

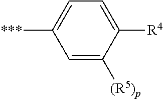

wherein
*** designates the linkage point with the ring

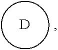

the ring

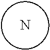

represents a saturated 4- to 10-membered aza-heterocycle, which contains at least one N atom as a ring member and in addition can contain a further hetero ring member from the series N, O, S or S(O)₂,
X represents a bond or ♦—(CH₂)_{q}—N(R⁶)—♦♦, —C(═O)— or ♦—N(R⁶)—C(═O)—♦♦, wherein
   ♦ designates the linkage point with the ring

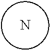

and
♦♦ designates the linkage point with the ring

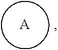

q denotes the number 0 or 1
and
R⁶ denotes hydrogen, methyl, ethyl, isopropyl, cyclopropyl or cyclobutyl,
R¹ represents a substituent bonded to a carbon atom of the ring

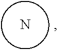

chosen from the series fluorine, cyano, (C₁-C₄)-alkyl, hydroxyl, (C₁-C₄)-alkoxy, oxo, amino, mono-(C₁-C₄)-alkylamino, di-(C₁-C₄)-alkylamino, cyclopropyl and cyclobutyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine,
m represents the number 0 or 1,
R² represents a substituent bonded to a nitrogen atom of the ring

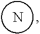

chosen from the series (C₁-C₄)-alkyl, (C₁-C₄)-alkylcarbonyl, (C₁-C₄)-alkoxycarbonyl, (C₁-C₄)-alkylsulfonyl, cyclopropyl and cyclobutyl,
wherein the alkyl group in (C₁-C₄)-alkyl, (C₁-C₄)-alkylcarbonyl, (C₁-C₄)-alkoxycarbonyl and (C₁-C₄)-alkylsulfonyl in its turn can be substituted by a radical chosen from the series hydroxyl, (C₁-C₄)-alkoxy, amino, mono-(C₁-C₄)-alkylamino, di-(C₁-C₄)-alkylamino (C₃-C₅)-cycloalkyl and 4- or 5-membered heterocyclyl and up to three times by fluorine,
n represents the number 0 or 1,
R³ represents methyl,
R⁴ represents a substituent chosen from the series chlorine, pentafluorothio, (C₁-C₆)-alkyl, trimethylsilyl, —OR⁷, —SR⁷, —S(═O)—R⁷, —S(═O)₂—R⁷, —S(═O)(═NCH₃)—CF₃, (C₃-C₆)-cycloalkyl and 4- to 6-membered heterocyclyl,
   wherein (C₁-C₆)-alkyl in its turn can be substituted by a radical chosen from the series —OR⁷, —NR⁷R⁸, —C(═O)—NR⁷R⁸, (C₃-C₆)-cycloalkyl and 4- to 6-membered heterocyclyl and up to three times by fluorine
   and
   the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
      wherein the (C₁-C₄)-alkyl substituent mentioned in its turn can be substituted by methoxy, trifluoromethoxy or ethoxy,
   and wherein
   R⁷ and R⁸ independently of each other for each individual occurrence denote hydrogen, (C₁-C₄)-alkyl or (C₃-C₆)-cycloalkyl,
      wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
      and
      the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy and trifluoromethoxy,
   or
   R⁷ and R⁸ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo and (C₁-C₄)-alkylcarbonyl,
R⁵ represents fluorine,
   and
p represents the number 0 or 1,
   and their salts, solvates and solvates of the salts.

Compounds of the formula (I) which are also preferred in particular are those in which
the ring

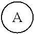

represents a phenyl ring and the adjacent groups X and CH₂ are bonded to this phenyl ring in 1, 3 or 1,4 relation to one another,
the ring

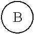

with the substituent R³ represents a heteroaryl ring of the formula

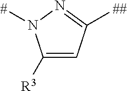

wherein
# designates the linkage point with the adjacent CH₂ group
and
## designates the linkage point with the ring

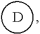

the ring

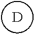

represents a heteroaryl ring of the formula

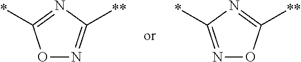

wherein
* designates the linkage point with the ring

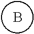

and
** designates the linkage point with the ring

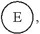

the ring

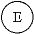

with the substituents R⁴ and R⁵ represents a phenyl ring of the formula

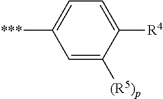

wherein
*** designates the linkage point with the ring

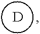

the ring

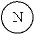

represents a saturated 4- to 10-membered aza-heterocycle, which contains at least one N atom as a ring member and in addition can contain a further hetero ring member from the series N, O, S or S(O)₂,
X represents a bond or ♦—(CH₂)_{q}—N(R⁶)—♦♦, —C(═O)— or ♦—N(R⁶)—C(═O)—♦♦, wherein
   ♦ designates the linkage point with the ring

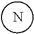

and
♦♦ designates the linkage point with the ring

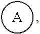

q denotes the number 0 or 1
and
R⁶ denotes hydrogen, methyl, ethyl, isopropyl, cyclopropyl or cyclobutyl,
R¹ represents a substituent bonded to a carbon atom of the ring

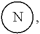

chosen from the series cyano, (C₁-C₄)-alkyl, oxo, cyclopropyl and cyclobutyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine,
R² represents a substituent bonded to a nitrogen atom of the ring

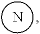

chosen from the series (C₁-C₄)-alkyl, (C₁-C₄)-alkylcarbonyl, (C₁-C₄)-alkoxycarbonyl, (C₁-C₄)-alkylsulfonyl, cyclopropyl and cyclobutyl,
wherein the alkyl group in (C₁-C₄)-alkyl, (C₁-C₄)-alkylcarbonyl, (C₁-C₄)-alkoxycarbonyl and (C₁-C₄)-alkylsulfonyl in its turn can be substituted by a radical chosen from the series hydroxyl, (C₁-C₄)-alkoxy, amino, mono-(C₁-C₄)-alkylamino, di-(C₁-C₄)-alkylamino (C₃-C₅)-cycloalkyl and 4- or 5-membered heterocyclyl and up to three times by fluorine
m represents the number 0 or 1,
n represents the number 0 or 1,
   wherein the sum of m and n equals the number 1 or 2,
R³ represents methyl,
R⁴ represents a substituent chosen from the series chlorine, pentafluorothio, (C₁-C₆)-alkyl, trimethylsilyl, —OR⁷, —SR⁷, —S(═O)—R⁷, —S(═O)₂—R⁷, —S(═O)(═NCH₃)—CF₃, (C₃-C₆)-cycloalkyl and 4- to 6-membered heterocyclyl,
   wherein (C₁-C₆)-alkyl in its turn can be substituted by a radical chosen from the series —OR′, —NR⁷R⁸, —C(═O)—NR⁷R⁸, (C₃-C₆)-cycloalkyl and 4- to 6-membered heterocyclyl and up to three time by fluorine
   and
   the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
      wherein the (C₁-C₄)-alkyl substituent mentioned in its turn can be substituted by methoxy, trifluoromethoxy or ethoxy,
   and wherein
   R⁷ and R⁸ independently of each other for each individual occurrence denote hydrogen, (C₁-C₄)-alkyl or (C₃-C₆)-cycloalkyl,
      wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
      and
      the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy and trifluoromethoxy,
   or
   R⁷ and R⁸ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo and (C₁-C₄)-alkylcarbonyl,
R⁵ represents fluorine,
   and
p represents the number 0 or 1,
   and their salts, solvates and solvates of the salts.

The radical definitions given in detail in the particular combinations or preferred combinations of radicals are also replaced as desired by radical definitions of other combinations, independently of the particular combinations of radicals given.

Combinations of two or more of the abovementioned preferred ranges are very particularly preferred.

The compounds according to the invention can be prepared in many ways. The main methods which are called process A, B, C and D in the following and can be carried out in various variants were used here in particular.

For all the processes and variants of processes described in the following, if the radical R² bonded to a nitrogen atom of the ring N represents hydrogen (i.e. n=0), instead of this hydrogen atom an amino-protective group is employed, depending on the reaction type, and is split off again when the reaction has been carried out or at the end of the reaction sequence in order to obtain the target compounds of the formula (I). This is always the case if a hydrogen atom bonded to a nitrogen atom is not compatible with the reaction conditions used. This procedure, including the knowledge of when corresponding reaction conditions are not compatible, and the amino-protective groups expedient for this, including the processes for their introduction and splitting off, are known per se and familiar to the person skilled in the art. Examples of such amino-protective groups are tert-butoxycarbonyl and benzyloxycarbonyl. Detailed descriptions of such protective group operations are to be found in the experiment instructions for the preparation of the starting materials and intermediates and the embodiment examples in the experimental part. For easier understanding, protective groups and protective group operations of this type are not dealt with further in the following description of the preparation processes to give the compounds according to the invention.

Process A (with variants A.1, A.2 and A.3; see equations 1-3) is characterized in that compounds of the formula (V), in which B, D, E, R³, R⁴, R⁵ and p have the meanings described above and in which the hydrogen atom shown is bonded to a nitrogen atom of the ring B, are reacted with a compound of the formula (II), (III) or (IV), in which A, N, X, R¹, R², m and n have the meanings described above and in which Y quite generally represents an atom or a grouping with the aid of which the connecting group X can be built up and the ring N (including its substituents R¹ and R²) can be linked, and in which Z represents a leaving group. Examples of Y are chlorine, bromine, iodine, cyano, nitro, hydroxyl, formyl, carboxyl and alkoxycarbonyl; examples of Z are chlorine, bromine, iodine, methanesulfonate (mesylate), trifluoromethanesulfonate (triflate) and 4-methylbenzenesulfonate (tosylate).

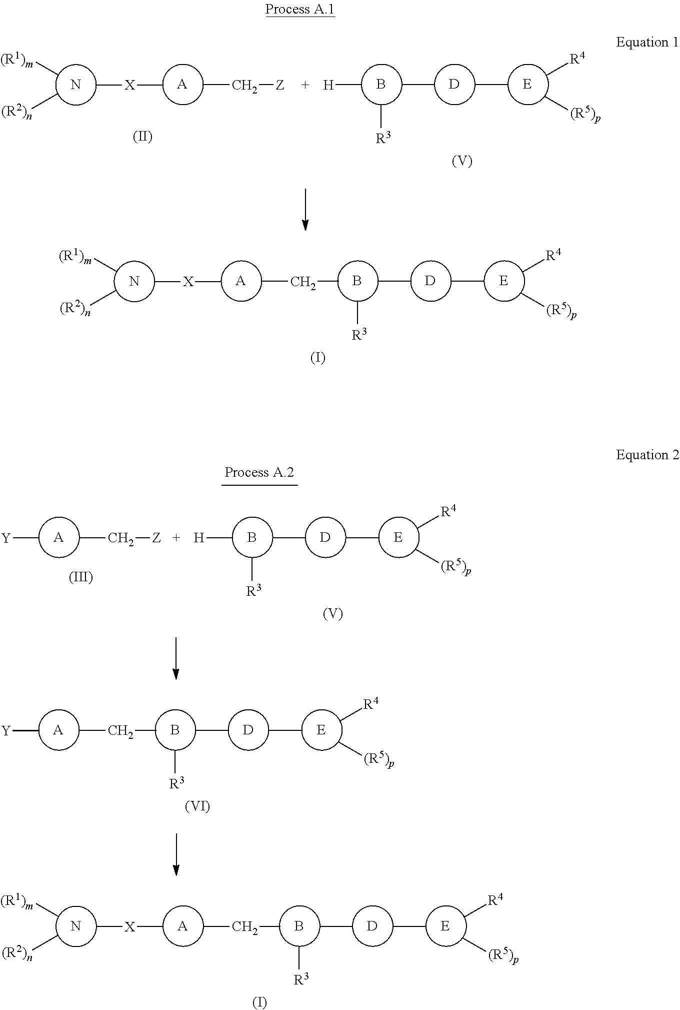

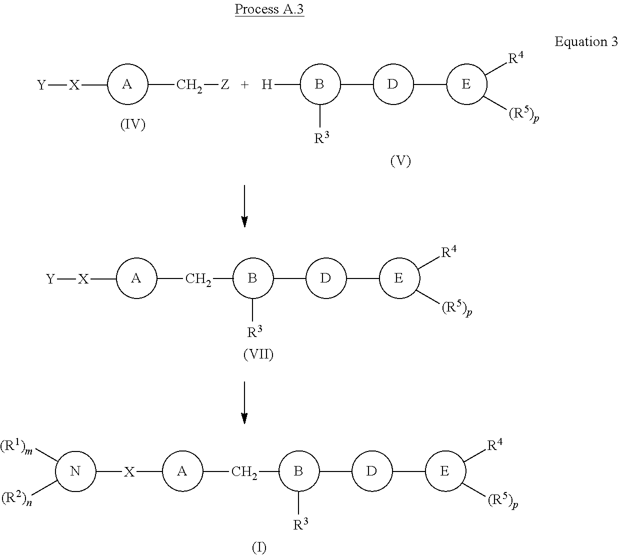

The reaction of the compounds of the formula (II), (III) or (IV) with the compounds of the formula (V) is carried out in the presence of a strong base, such as, for example and preferably, potassium tert-butylate, in a suitable solvent, such as, for example and preferably, tetrahydrofuran, in a temperature range of between −10° C. and +50° C., preferably between 0° C. and room temperature. The subsequent reaction of the intermediates of the formulae (VI) and (VIII) to give the products of the formula (I) varies and depends in particular on the nature of the group X and the ring A. These subsequent reactions are described below.

In process B the ring D is built up, the ring D representing a 1,2,4-oxadiazole here. Process B is also used in various modifications. The variants of process B (variants B.1, B.2 and B.3) are similar to the various variants of process A with respect to the educts used and the part reactions which follow the ring closure to the oxadiazole. Only variant B.1 is therefore to be described in detail in the following (equation 4). Compounds of the formula (VIII), in which A, B, N, X, R¹, R², R³, m and n have the meanings described above, are reacted here with hydroxyamidines of the formula (IX), in which E, R⁴, R⁵ and p have the meanings given above, to give the products of the formula (I-A).

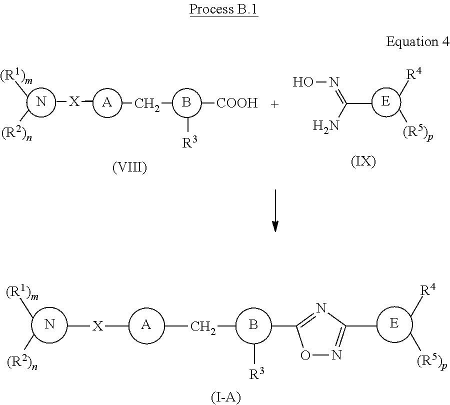

The reaction of the compounds of the formula (VIII) with the compounds of the formula (IX) is carried out in the presence of coupling reagents, such as, for example, 1H-benzotriazol-1-ol and N-[3-(dimethylamino)propyl]-N′-ethylcarbodiimide hydrochloride, in the presence of tertiary amine bases, such as, for example, triethylamine, in suitable solvents, such as, for example, N,N-dimethylformamide. The reaction partners are first reacted with one another at room temperature for some time, before the mixture is then heated to temperatures in the range of from +80° C. to +140° C. Alternatively, the compounds of the formula (VIII) can first be converted into the corresponding carboxylic acid chlorides. Chlorinating reagents, such as, for example, oxalyl chloride or thionyl chloride, in inert solvents, such as, for example, methylene chloride or chloroform, are employed for this. The reaction is preferably carried out at room temperature and in the presence of a catalytic amount of N,N-dimethylformamide. The acid chloride obtained in this way is then reacted with the compounds of the formula (IX). The product of this reaction is then heated to temperatures in the range of from +80° C. to +140° C. in inert solvents, such as, for example, dimethylsulfoxide or N,N-dimethylformamide.

In the remaining variants of process B, instead of compounds of the formula (VIII), carboxylic acids of the formula (X) (process B.2) or (XI) (process B.3), in which A, B, X, Y and R³ in each case have the meanings described above, are employed.

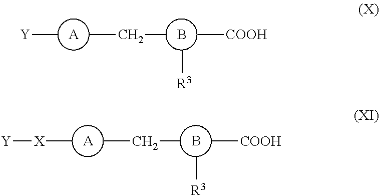

If the ring D represents a 1,3-oxazole, process C can be used, which can be carried out analogously to processes A and B in various variants C.1, C.2 and C.3. As is the case for process B, only variant C.1 is explained in more detail in the following (equation 5). In process C.1 compounds of the formula (VIII) are reacted with compounds of the formula (XII) to give intermediates of the formula (XIII), which, after cyclization, are oxidized to the products of the formula (I-B). A, B, E, N, X, R¹, R², R³, R⁴, R⁵, m, n and p in each case have the meanings described above.

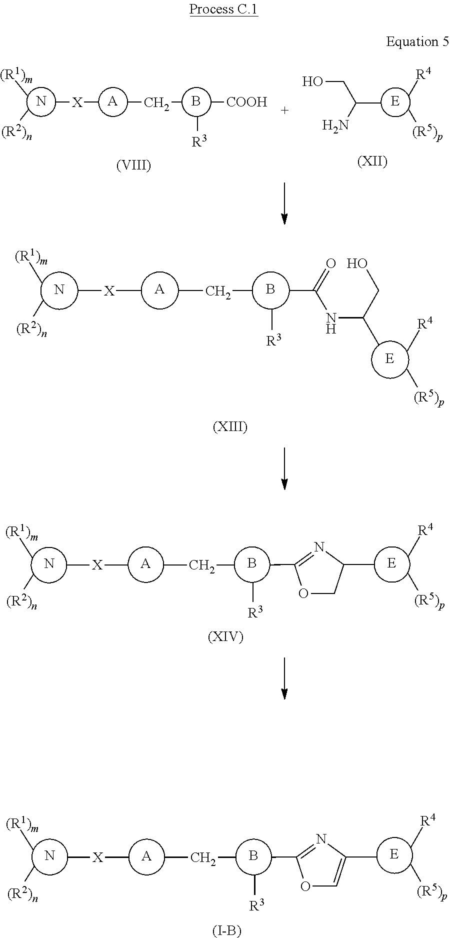

The compounds of the formula (VIII) are reacted with the amino alcohols of the formula (XII) in the presence of coupling reagents, such as, for example, O-(7-azabenzotriazol-1-yl)-N,N,N′,N′-tetramethyluronium hexafluorophosphate. The reaction is carried out at room temperature in the presence of tertiary amine bases, such as, for example, triethylamine, in polar aprotic solvents, such as, for example, N,N-dimethylformamide. Subsequent cyclization to give the compounds of the formula (XIV) is achieved with the aid of a cyclizing reagent, such as, for example and preferably, with Burgess reagent (carbomethoxysulfamoyl-triethylammonium hydroxide). The reaction is carried out in suitable solvents, such as, for example, tetrahydrofuran, at the boiling point of the solvent. The final oxidation can be carried out with various oxidizing agents. Oxidation with activated manganese dioxide in tetrahydrofuran at the boiling point of the solvent is preferred.

In the other variants of process C, the 1,3-oxazole ring is built up in the same manner. Instead of compounds of the formula (VIII), carboxylic acids of the formula (X) (process C.2) or (XI) (process C.3), in which A, B, X, Y and R³ have the meanings described above, are employed.

Process D describes the preparation of compounds of the formula (I) in which the ring D represents a 1,2,4-oxadiazole which, in contrast to the oxadiazole derivatives described in process B, is linked to the adjacent groups in a manner in which the sides are switched. Analogously to processes A, B and C, process D can be carried out in the various variants D.1, D.2 and D.3; as is the case for processes B and C, only variant D.1 is explained in more detail in the following (equation 6). The carboxylic acids of the formula (VIII) are first converted here into the primary amides of the formula (XV), from which the nitriles of the formula (XVI) are then prepared. By reaction with hydroxylamine, these are converted into the hydroxyamidines of the formula (XVII), from which the products of the formula (I-C) are obtained by coupling with the acid chlorides of the formula (XVIII) and subsequent cyclization. A, B, E, N, X, R¹, R², R³, R⁴, R⁵, m, n and p in each case have the meanings described above.

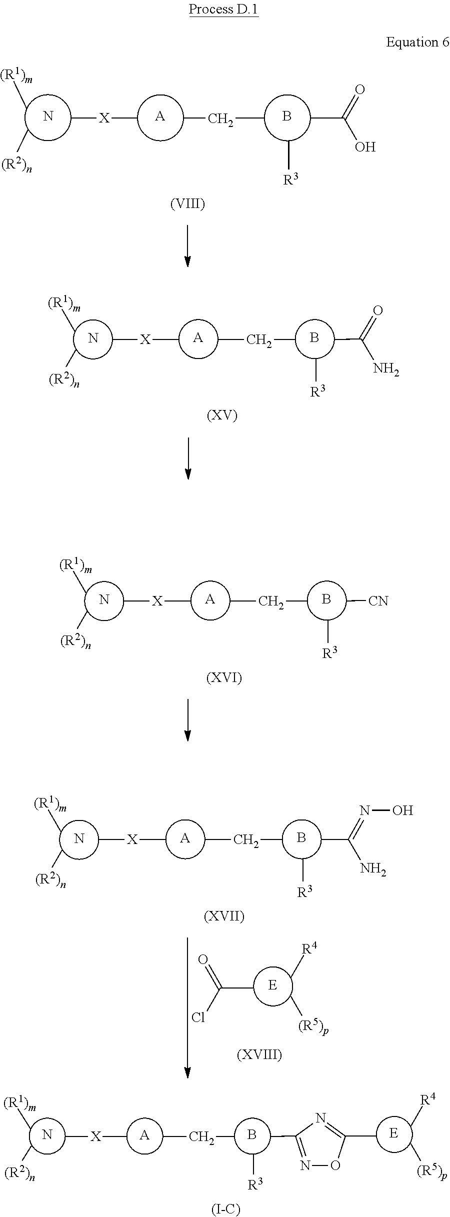

The reaction of the carboxylic acids of the formula (VIII) to give the amides of the formula (XV) is carried out in two stages: First by reaction with chlorinating reagents, such as, for example, oxalyl chloride or thionyl chloride, in inert solvents, such as, for example, methylene chloride or chloroform, and then by reaction of the carboxylic acid chlorides obtained in this way with solutions of ammonia in methanol or water in a suitable co-solvent, such as, for example, tetrahydrofuran or 1,4-dioxane. The dehydration of the primary amides of the formula (XV) to give the nitriles of the formula (XVI) is carried out by reaction with anhydrides or chlorides of strong acids, such as, for example and preferably, of trifluoromethanesulfonic acid or trifluoroacetic acid, in the presence of an excess of a base, such as, for example, triethylamine or N,N-diisopropylethylamine, in inert solvents, such as, for example, methylene chloride. The reaction is preferably carried out in the temperature range of between 0° C. and room temperature. The subsequent reaction with hydroxylamine is preferably carried out in alcoholic solvents, such as, for example, ethanol, at the boiling point of the solvent. The hydroxyamidines of the formula (XVII) obtained in this way are reacted with the acid chlorides of the formula (XVIII) in the presence of bases, such as, for example, triethylamine or N,N-diisopropylethylamine, in inert solvents, such as, for example, methylene chloride or ethyl acetate, at temperatures of between −10° C. and room temperature. The intermediate products thereby obtained are cyclized to the products of the formula (I-C) in inert solvents, such as, for example, dimethylsulfoxide or N,N-dimethylformamide, at temperatures of between +80° C. and +160° C.

The reactions which lead from the intermediates of the formula (VI) (process A.2, equation 2) to the products of the formula (I), depending on the group X and the nature of the ring A, are described in the following. These reactions are also used correspondingly in processes B.2, C.2 and D.2.

a) If X represents ♦—(CH₂)_{q}—NR⁶—♦♦, O or S, wherein R⁶, q, ♦ and ♦♦ have the meanings described above, and the ring A represents a pyridine ring, and the group Y is bonded to a carbon atom of this pyridine ring which is in the direct neighbourhood of the pyridine nitrogen atom, and Y represents halogen or a sulfonate, according to equation 7 compounds of the formula (VI) are reacted with corresponding compounds of the formula (XIX). The reaction is carried out in the presence of an excess of the compound of the formula (XIX), and if X represents O or S additionally in the presence of a base, such as, for example, sodium hydride. The reaction takes place in solvents, such as diethylene glycol dimethyl ether or N-methylpyrrolidinone, or, if X represents ♦—(CH₂)_{q}—NR⁶—♦♦, in tertiary amine bases, such as N,N-diisopropylethylamine, or the compounds of the formula (XIX) themselves serve as solvents. The reaction is carried out at elevated temperature, preferably in a temperature range of between +80° C. and +200° C. Reactions in the upper region of the temperature interval mentioned are preferably carried out in closed pressure vessels in a microwave apparatus.

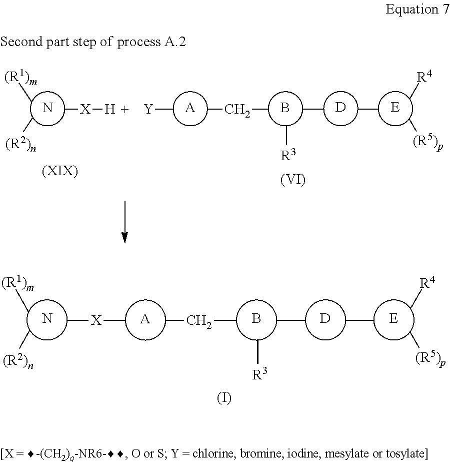

b) If X represents ♦—(CH₂)_{q}—NR⁶—♦♦, O or S and the group Y represents halogen or a sulfonate and is bonded to a carbon atom of a pyridine ring A which is in any desired position in relation to the pyridine nitrogen atom, or ring A is a phenyl ring, the compounds of the formula (VI) and the compounds of the formula (XIX) are reacted with one another according to equation 7 in the presence of palladium catalysts. Suitable palladium sources are, for example, palladium(II) acetate or tris(dibenzylidene-acetone)dipalladium(0). Ligands which can be used are, for example, 2,2′-bis(diphenylphosphino)-1,1′-binaphthyl, 1-[2-(dicyclohexylphosphino)ferrocenyl]ethyldi-tert-butylphosphine or bis(diphenylphosphino)ferrocene. The reactions proceed in the presence of bases, such as, for example, triethylamine or sodium tert-butylate. Suitable solvents are, for example, toluene, N-methylpyrrolidinone or 1,2-dimethoxyethane. The reactions are usually carried out in the temperature interval of between +60° C. and the particular boiling point of the solvent.
c) If X represents ♦—NR⁶—C(═O)—♦♦, wherein R⁶, ♦ and ♦♦ have the meanings described above, compounds of the formula (VI) in which Y represents an alkoxycarbonyl group or cyano are first converted into the corresponding carboxylic acids by treatment with aqueous alkali, and these are then reacted with compounds of the formula (XX) to give the products of the formula (I) (see equation 8). This reaction is carried out either directly from the carboxylic acid in the presence of coupling reagents, such as, for example, 1H-benzotriazol-1-ol and N-[3-(dimethylamino)propyl]-N-ethylcarbodiimide hydrochloride, or by converting the carboxylic acid into the corresponding acid chloride, for example with the aid of thionyl chloride or oxalyl chloride, and then reacting this with the amine component (XX).

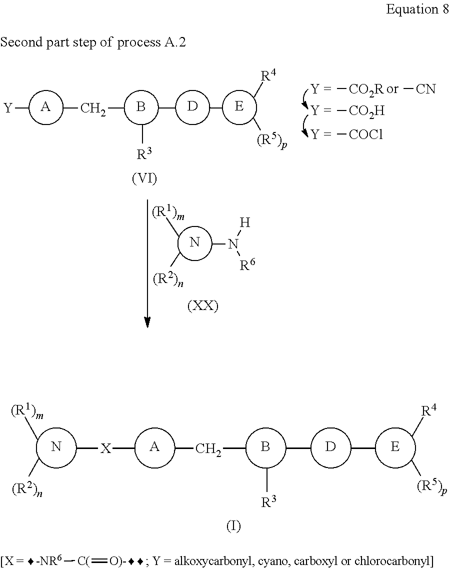

The hydrolysis of the esters (VI) [Y=alkoxycarbonyl] is preferably carried out with aqueous solutions of lithium hydroxide, sodium hydroxide or potassium hydroxide in the presence of water-miscible inert solvents, such as, for example, methanol, ethanol or tetrahydrofuran. The reaction is in general carried out in the temperature interval of between room temperature and +60° C., preferably at room temperature. The hydrolysis of the nitriles (VI) [Y=cyano] is likewise carried out with aqueous alkali, preferably with aqueous potassium hydroxide, in ethanol at the boiling point of the solvent. The subsequent conversion of the carboxylic acids obtained in this way into the corresponding acid chlorides is carried out with chlorinating reagents, such as, for example and preferably, oxalyl chloride or thionyl chloride, in inert solvents, such as, for example, methylene chloride. The reaction is carried out in the temperature range of between 0° C. and the boiling point of the solvent, preferably at room temperature. The final reaction of the amines of the formula (XX) with the acid chlorides of the formula (VI) [Y=chlorocarbonyl] is carried out in the presence of bases, such as, for example, triethylamine, N,N-diisopropylethylamine or potassium carbonate, in inert solvents, such as, for example, methylene chloride or ethyl acetate. The reaction is carried out in the temperature range of from 0° C. to room temperature. The reaction of the amines of the formula (XX) with the carboxylic acids of the formula (VI) [Y=carboxyl] is carried out with the aid of conventional coupling reagents, such as, for example, 1H-benzotriazol-1-ol and N-[3-(dimethylamino)propyl]-N-ethylcarbodiimide hydrochloride, in suitable solvents, such as, for example, N,N-dimethylformamide, and in the presence of tertiary amine bases, such as, for example, triethylamine. The reaction is preferably carried out at room temperature.

d) If X represents —C(═O)— and the ring N is bonded to X via a nitrogen atom, compounds of the formula (VI) in which Y represents carboxyl or chlorocarbonyl (see equation 8) are reacted with compounds of the formula (XXI) in which the hydrogen atom shown is bonded to a nitrogen atom of the ring N, to give the products of the formula (I).

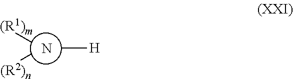

The reaction conditions for this are completely analogous to those described under section c).

e) If X represents ♦—C(═O)—NH—♦♦, wherein ♦ and ♦♦ have the meanings described above, compounds of the formula (VI) in which Y represents a nitro group are first reduced to the corresponding amines [Y═NH₂] and these are then reacted with compounds of the formula (XXII) or (XXIII) to give the products of the formula (I) (see equation 9). This reaction is carried out in the presence of conventional coupling reagents, such as, for example, 1H-benzotriazol-1-ol and N-[3-(dimethylamino)propyl]-N-ethylcarbodiimide hydrochloride, in the case of the carboxylic acids (XXII), and in the case of the acid chlorides (XXIII) directly in the presence of tertiary amine bases, such as triethylamine or N,N-diisopropylethylamine.

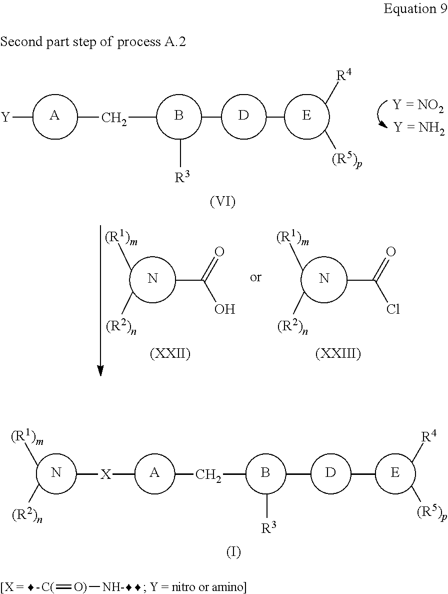

The reduction of the nitro group is achieved, for example, by catalytic hydrogenation with the aid of noble metal catalysts, such as, for example, palladium on charcoal, in inert solvents, such as, for example, ethanol, in the presence of hydrogen under a pressure of from 1 to 50 bar, preferably from 1 to 5 bar. The reaction is typically carried out at room temperature. The subsequent reaction with the carboxylic acids (XXII) or acid chlorides (XXIII) is carried out either with the aid of coupling reagents or directly in the presence of tertiary amine bases, as has already been described above.

f) If X represents oxygen, compounds of the formula (XXIV), in which Z represents a leaving group, such as, for example, chlorine, bromine or methanesulfonate, and compounds of the formula (VI) in which Y represents hydroxyl can alternatively also be reacted with one another. The latter are obtainable, for example, via corresponding silyl ethers (see equation 10).

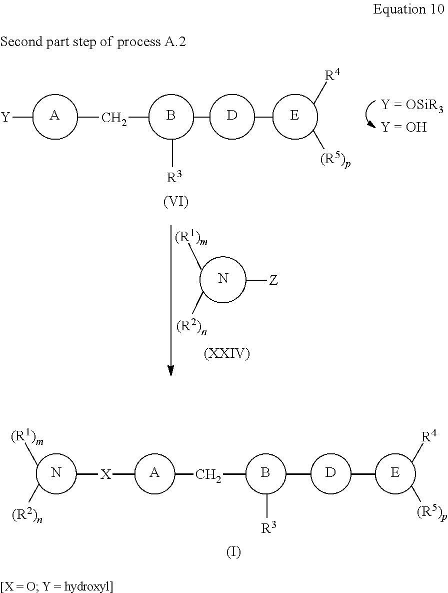

The reaction of the compounds of the formula (VI) in which Y represents a silyl ether to give the free hydroxy compounds of the formula (VI) [Y═OH] is carried out, for example, by treatment with a source of fluoride, such as tetra-n-butylammonium fluoride, in solvents, such as tetrahydrofuran, at temperatures preferably of between 0° C. and room temperature. The subsequent reaction with the compounds of the formula (XXIV) is carried out in inert solvents, such as, for example and preferably, N,N-dimethylformamide, in the presence of bases, such as, for example, sodium hydride or caesium carbonate, at temperatures of between room temperature and +140° C.

g) A process similar to that described under f) can be used if X represents NH. The compounds of the formula (VI) shown in equation 9 in which Y represents NH₂ are first converted into the corresponding carbamates, for example with di-tert-butyl dicarbonate or with benzyloxycarbonyl chloride, which are then reacted with compounds of the formula (XXIV) (see equation 10), in which Z represents a leaving group, such as chlorine, bromine or methanesulfonate. In the final reaction, the carbamate protective groups is removed again in order to obtain the products of the formula (I) in which X represents NH in this way. The processes for introduction and splitting off of the carbamate protective groups are described in the chemical literature and known to the person skilled in the art. The reaction of the compounds of the formula (XXIV) with the carbamates derived from compounds of the formula (VI) [Y═NH₂] is carried out under reaction conditions similar to those described under f).
h) If X represents a bond, the ring N is bonded to the ring A via a ring nitrogen atom, the ring

A represents a pyridine ring and the group Y is bonded to a carbon atom of this pyridine ring which is in the direct neighbourhood of the pyridine nitrogen atom, and Y represents halogen or a sulfonate, compounds of the formula (VI) are reacted with compounds of the formula (XXI) in which the hydrogen atom shown is bonded to a nitrogen atom of the ring N (see equation 11). The reaction is carried out in the presence of an excess of the compound of the formula (XXI) and optionally in the presence of a tertiary amine base, such as, for example, N,N-diisopropylethylamine. The reaction takes place in solvents, such as diethylene glycol dimethyl ether or N-methylpyrrolidinone, or the tertiary amine base or the compounds of the formula (XXI) themselves serve as solvents. The reaction is carried out at elevated temperature, preferably in a temperature range of between +80° C. and +200° C. Reactions in the upper region of the temperature interval mentioned are preferably carried out in closed pressure vessels in a microwave apparatus.

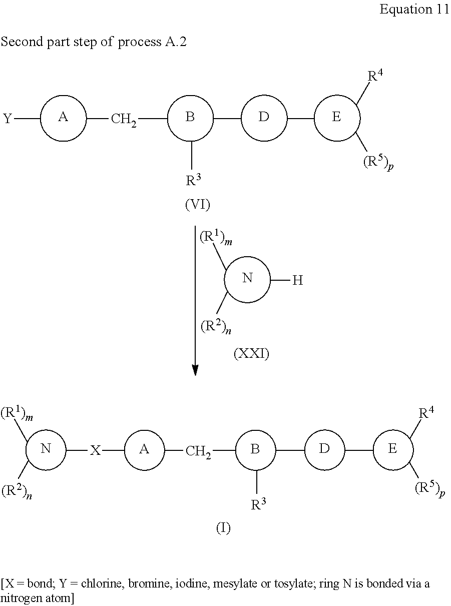

i) If X represents a bond, the ring N is bonded to the ring A via a ring nitrogen atom and the group Y represents halogen or a sulfonate and is bonded to a carbon atom of a pyridine ring A which is in any desired position in relation to the pyridine nitrogen atom, or ring A is a phenyl ring, the compounds of the formula (VI) and the compounds of the formula (XXI) are reacted with one another according to equation 11 the presence of palladium catalysts. Suitable palladium sources are, for example, palladium(II) acetate or tris(dibenzylidene-acetone)dipalladium(0). Ligands which can be used are, for example, 2,2′-bis(diphenylphosphino)-1,1′-binaphthyl, 1-[2-(dicyclo-hexylphosphino)ferrocenyl]ethyldi-tert-butylphosphine or bis(diphenylphosphino)ferrocene. The reactions are carried out in the presence of bases, such as, for example, triethylamine or sodium tert-butylate. Suitable solvents are, for example, toluene, N-methylpyrrolidinone or 1,2-dimethoxyethane. The reactions are usually carried out in the temperature interval of between +60° C. and the particular boiling point of the solvent.

The reactions which lead from the intermediates of the type of the formula (VII) (process A.3, equation 3) to the products of the formula (I), depending on the nature of the groups Y and Z, are described in the following. These reactions are also used correspondingly in processes B.3, C.3 and D.3.

j) The compounds of the formula (XXV) in which Y represents hydroxyl are first converted into compounds of the formula (XXVI), in which Z represents a leaving group, such as, for example, chlorine, bromine or methanesulfonate, and these are then reacted with amines of the formula (XX) to give the products of the formula (I) in which X represents the group ♦—NR⁶—CH₂—♦♦ (see equation 12).

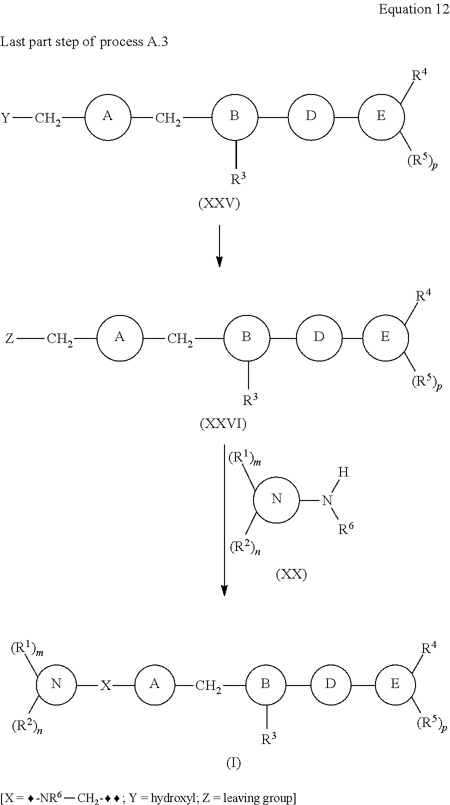

The compounds of the formula (XXV) in which Y represents hydroxyl are converted into compounds of the formula (XXVI) by reacting them, for example, with bromine in the presence of triphenylphosphine in suitable solvents, such as, for example, tetrahydrofuran, at room temperature to give the corresponding bromides (XXVI) [Z═Br]. The conversion can also be carried out, for example and preferably, with the aid of trifluoromethanesulfonic acid anhydride or methanesulfonic acid anhydride in the presence of bases, such as, for example, triethylamine or 2,6-dimethylpyridine. These reactions are preferably carried out in methylene chloride or tetrahydrofuran at low temperatures of approx. −78° C. Compounds of the formula (XXVI) in which Z represents trifluoromethanesulfonate (triflate) or methanesulfonate (mesylate) are obtained in this way. The compounds of the formula (XXVI) are then reacted with amines of the formula (XX) to give the products of the formula (I) by reacting the reactants, for example, in methylene chloride or tetrahydrofuran in the presence of tertiary amine bases, such as, for example, triethylamine or 2,6-dimethylpyridine, at temperatures of between −78° C. and room temperature. If Z represents trifluoromethanesulfonate or methanesulfonate, the reaction sequence can also be carried out as a one-pot process starting from compounds of the formula (XXV) [Y═OH].

The starting compounds of the formulae (II), (III), (IV), (V), (VIII), (IX), (X), (XI), (XII), (XVIII), (XIX), (XX), (XXI), (XXII), (XXIII) and (XXIV) are either commercially obtainable or described as such in the literature, or they can be prepared by routes evident to the person skilled in the art analogously to methods published in the literature. Thus, for example, compounds of the formula (V) in which the ring D represents a 1,2,4-oxadiazole or a 1,3-oxazole can be prepared analogously to process methods B, C and D described above, and compounds of the formulae (II), (VIII), (X) and (XI) can be obtained analogously to process variants A.1, A.2 and A.3 with the part steps described in equations 7-12.

For example, compounds of the formula (I-D) according to the invention

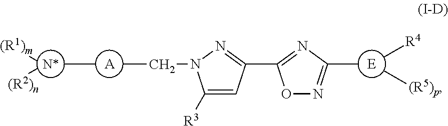

in which the rings A and E and R¹, R², R³, R⁴, R⁵, m, n and p each have the meanings given above, and
the ring N* represents a ring N which is bonded to the ring A via a ring nitrogen atom and is as defined above,
are prepared by a procedure in which an N′-hydroxyamidine of the formula (IX)

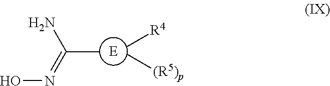

in which the ring E and R⁴, R⁵ and p have the meanings given above,
first can either be
[A] subjected to a condensation reaction with a pyrazolecarboxylic acid of the formula (XXVII)

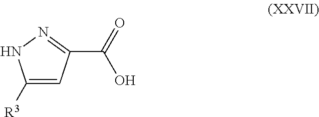

in which R³ has the meaning given above,
to give a 1,2,4-oxadiazole derivative of the formula (XXVIII)

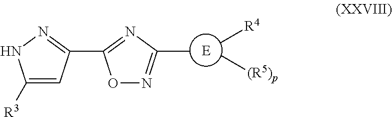

in which the ring E and R³, R⁴, R⁵ and p have the meanings given above,
and this is then alkylated in the presence of a base with a compound of the formula (III)

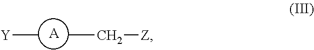

in which the ring A has the meaning given above,
Y represents chlorine, bromine or iodine
and
Z represents chlorine, bromine, iodine, mesylate, triflate or tosylate,
to give a compound of the formula (XXIX)

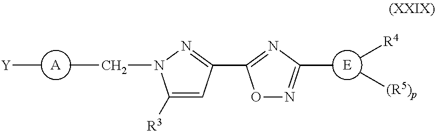

in which the rings A and E and R³, R⁴, R⁵, p and Y have the meanings given above, or
[B] subjected to a condensation reaction with a pyrazolecarboxylic acid of the formula (XXX)

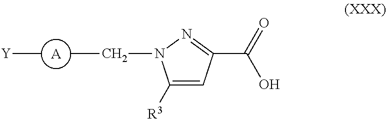

in which the ring A and R³ have the meanings given above,
and
Y represents chlorine, bromine or iodine,
to give the compound of the formula (XXIX)

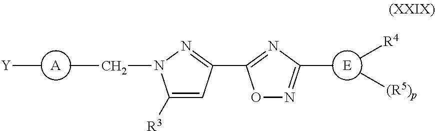

in which the rings A and E and R³, R⁴, R⁵, p and Y have the abovementioned meanings,
   and the compound of the formula (XXIX) obtained in this way is then reacted, optionally in the presence of a palladium catalyst and/or a base, with a compound of the formula (XXXI)

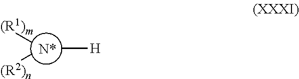

in which the ring N* and R¹, R², m and n have the meanings given above and the hydrogen atom shown is bonded to a nitrogen atom of the ring N*
(in this context cf. process A.2 described above, in combination with the variant of the second part step shown in equation 11, and processes B.1 and B.2 with the particular reaction parameters described there).

Numerous detailed instructions and literature information for the preparation of the starting materials are also to be found in the experimental part in the section for the preparation of the starting compounds and intermediates.

The compounds according to the invention have valuable pharmacological properties and can be used for prevention and treatment of diseases in humans and animals.

The compounds according to the invention are highly potent inhibitors of the HIF regulation pathway and have a good bioavailability following peroral administration.

On the basis of their action profile, the compounds according to the invention are suitable in particular for treatment of hyperproliferative diseases in humans and in mammals generally. The compounds can inhibit, block, reduce or lower cell proliferation and cell division and on the other hand increase apoptosis.

The hyperproliferative diseases for the treatment of which the compounds according to the invention can be employed include, inter alia, psoriasis, keloids, scar formation and other proliferative diseases of the skin, benign diseases, such as benign prostate hyperplasia (BPH), and in particular the group of tumour diseases. In the context of the present invention, these are understood as meaning, in particular, the following diseases, but without being limited to them: mammary carcinomas and mammary tumours (ductal and lobular forms, also in situ), tumours of the respiratory tract (parvicellular and non-parvicellular carcinoma, bronchial carcinoma), cerebral tumours (e.g. of the brain stem and of the hypothalamus, astrocytoma, medulloblastoma, ependymoma and neuro-ectodermal and pineal tumours), tumours of the digestive organs (oesophagus, stomach, gall bladder, small intestine, large intestine, rectum), liver tumours (inter alia hepatocellular carcinoma, cholangiocellular carcinoma and mixed hepatocellular and cholangiocellular carcinoma), tumours of the head and neck region (larynx, hypopharynx, nasopharynx, oropharynx, lips and oral cavity), skin tumours (squamous epithelial carcinoma, Kaposi sarcoma, malignant melanoma, Merkel cell skin cancer and nonmelanomatous skin cancer) tumours of soft tissue (inter alia soft tissue sarcomas, osteosarcomas, malignant fibrous histiocytomas, lymphosarcomas and rhabdomyosarcomas), tumours of the eyes (inter alia intraocular melanoma and retinoblastoma), tumours of the endocrine and exocrine glands (e.g. thyroid and parathyroid glands, pancreas and salivary gland), tumours of the urinary tract (tumours of the bladder, penis, kidney, renal pelvis and ureter) and tumours of the reproductive organs (carcinomas of the endometrium, cervix, ovary, vagina, vulva and uterus in women and carcinomas of the prostate and testicles in men). These also include proliferative blood diseases in solid form and as circulating blood cells, such as lymphomas, leukaemias and myeloproliferative diseases, e.g. acute myeloid, acute lymphoblastic, chronic lymphocytic, chronic myelogenic and hair cell leukaemia, and AIDS-correlated lymphomas, Hodgkin's lymphomas, non-Hodgkin's lymphomas, cutaneous T cell lymphomas, Burkitt's lymphomas and lymphomas in the central nervous system.

These well-described diseases in humans can also occur with a comparable aetiology in other mammals and can be treated there with the compounds of the present invention.

In the context of this invention the term “treatment” or “treat” is used in the conventional sense and means attending to, caring for and nursing a patient with the aim of combating, reducing, attenuating or alleviating a disease or health abnormality and improving the living conditions impaired by this disease, such as, for example, with a cancer disease.

The compounds according to the invention act as modulators of the HIF regulation pathway and are therefore also suitable for treatment of diseases associated with a harmful expression of the HIF transcription factor. This applies in particular to the transcription factors HIF-1α and HIF-2α. The term “harmful expression of HIF” here means a non-normal physiological presence of HIF protein. This can be due to excessive synthesis of the protein (mRNA- or translation-related), reduced degradation or inadequate counter-regulation in the functioning of the transcription factor.

HIF-1α and HIF-2αregulate more than 100 genes. This applies to proteins which play a role in angiogenesis and are therefore directly relevant to tumours, and also those which influence glucose, amino acid and lipid metabolism as well as cell migration, metastasis and DNA repair, or improve the survival of tumour cells by suppressing apoptosis. Others act more indirectly via inhibition of the immune reaction and upwards regulation of angiogenic factors in inflammation cells. HIF also plays an important role in stem cells, and here in particular tumour stem cells, which are reported to have increased HIF levels. By the inhibition of the HIF regulation pathway by the compounds of the present invention, tumour stem cells, which do not have a high proliferation rate and therefore are affected only inadequately by cytotoxic substances, are therefore also influenced therapeutically (cf. Semenza, 2007; Weidemann and Johnson, 2008).

Changes in cell metabolism by HIF are not exclusive to tumours, but also occur with other hypoxic pathophysiological processes, whether chronic or transient. HIF inhibitors—such as the compounds of the present invention—are therapeutically helpful in those connections in which, for example, additional damage arises from adaptation of cells to hypoxic situations, since damaged cells can cause further damage if they do not function as intended. One example of this is the formation of epileptic foci in partly destroyed tissue following strokes. A similar situation is found with cardiovascular diseases if ischaemic processes occur in the heart or in the brain as a consequence of thromboembolic events, inflammations, wounds, intoxications or other causes. These can lead to damage such as a locally retarded action potential, which in turn can bring about arrhythmias or chronic heart failure. In a transient form, e.g. due to apnoea, under certain circumstances an essential hypertension may occur, which can lead to known secondary diseases, such as, for example, stroke and cardiac infarction.

Inhibition of the HIF regulation pathway such as is achieved by the compounds according to the invention can therefore also be helpful for diseases such as cardiac insufficiency, arrhythmia, cardiac infarction, apnoea-induced hypertension, pulmonary hypertension, transplant ischaemia, reperfusion damage, stroke and macular degeneration, as well as for recovery of nerve function after traumatic damage or severance.

Since HIF is one of the factors which control the transition from an epithelial to a mesenchymal cell type, which is of importance specifically for the lung and kidney, the compounds according to the invention can also be employed for preventing or controlling fibroses of the lung and kidney associated with HIF.

Further diseases for the treatment of which the compounds according to the invention can be used are inflammatory joint diseases, such as various forms of arthritis, and inflammatory intestinal diseases, such as, for example, Crohn's disease.

Chugwash polycythaemia is mediated by HIF-2αactivity during erythropoiesis inter alia in the spleen. The compounds according to the invention, as inhibitors of the HIF regulation pathway, are therefore also suitable here for suppressing excessive erythrocyte formation and therefore for alleviating the effects of this disease.

The compounds of the present invention can furthermore be used for treatment of diseases associated with excessive or abnormal angiogenesis. These include, inter alia, diabetic retinopathy, ischaemic retinal vein occlusion and retinopathy in premature babies (cf. Aiello et al., 1994; Peer et al., 1995), age-related macular degeneration (AMD; cf. Lopex et al., 1996), neovascular glaucoma, psoriasis, retrolental fibroplasia, angiofibroma, inflammation, rheumatic arthritis (RA), restenosis, in-stent restenosis following vessel implantation.

An increased blood supply is furthermore associated with cancerous, neoplastic tissue and leads here to an accelerated tumour growth. The growth of new blood and lymph vessels moreover facilitates the formation of metastases and therefore the spread of the tumour. New lymph and blood vessels are also harmful for allografts in immunoprivileged tissues, such as the eye, which, for example, increases the susceptibility to rejection reactions. Compounds of the present invention can therefore also be employed for therapy of one of the abovementioned diseases, e.g. by an inhibition of the growth or a reduction in the number of blood vessels. This can be achieved via inhibition of endothelial cell proliferation or other mechanisms for preventing or lessening the formation of vessels and via a reduction of neoplastic cells by apoptosis.

The present invention furthermore provides the use of the compounds according to the invention for treatment and/or prevention of diseases, in particular the abovementioned diseases.

The present invention furthermore provides the use of the compounds according to the invention for the preparation of a medicament for treatment and/or prevention of diseases, in particular the abovementioned diseases.

The present invention furthermore provides the use of the compounds according to the invention in a method for treatment and/or prevention of diseases, in particular the abovementioned diseases.

The present invention furthermore provides a method for treatment and/or prevention of diseases, in particular the abovementioned diseases, using an active amount of at least one of the compounds according to the invention.

The compounds according to the invention can be employed by themselves or, if required, in combination with one or more other pharmacologically active substances, as long as this combination does not lead to undesirable and unacceptable side effects. The present invention furthermore therefore provides medicaments containing at least one of the compounds according to the invention and one or more further active compounds, in particular for treatment and/or prevention of the abovementioned diseases.

For example, the compounds of the present invention can be combined with known antihyperproliferative, cytostatic or cytotoxic substances for treatment of cancer diseases. The combination of the compounds according to the invention with other substances customary for cancer therapy or also with radiotherapy is therefore indicated in particular, since hypoxic regions of a tumour respond only weakly to the conventional therapies mentioned, whereas the compounds of the present invention display their activity there in particular.

Suitable active compounds in the combination which may be mentioned by way of example are:

aldesleukin, alendronic acid, alfaferone, alitretinoin, allopurinol, aloprim, aloxi, altretamine, aminoglutethimide, amifostine, amrubicin, amsacrine, anastrozole, anzmet, aranesp, arglabin, arsenic trioxide, aromasin, 5-azacytidine, azathioprine, BCG or tice-BCG, bestatin, betamethasone acetate, betamethasone sodium phosphate, bexarotene, bleomycin sulfate, broxuridine, bortezomib, busulfan, calcitonin, campath, capecitabine, carboplatin, casodex, cefesone, celmoleukin, cerubi-din, chlorambucil, cisplatin, cladribin, clodronic acid, cyclophosphamide, cytarabine, dacarbazine, dactinomycin, daunoxome, decadron, decadron phosphate, delestrogen, denileukin diftitox, depomedrol, deslorelin, dexrazoxane, diethylstilbestrol, diflucan, docetaxel, doxifluridine, doxo-rubicin, dronabinol, DW-166HC, eligard, elitek, ellence, emend, epirubicin, epoetin-alfa, epogen, eptaplatin, ergamisol, estrace, estradiol, estramustine sodium phosphate, ethinylestradiol, ethyol, etidronic acid, etopophos, etoposide, fadrozole, farstone, filgrastim, finasteride, fligrastim, floxuridine, fluconazole, fludarabin, 5-fluorodeoxyuridine monophosphate, 5-fluoruracil (5-FU), fluoxymesterone, flutamide, formestane, fosteabine, fotemustine, fulvestrant, gammagard, gemcitabine, gemtuzumab, gleevec, gliadel, goserelin, granisetron hydrochloride, histrelin, hycamtin, hydrocortone, erythro-hydroxynonyladenine, hydroxyurea, ibritumomab tiuxetan, idarubicin, ifosfamide, interferon-alpha, interferon-alpha-2, interferon-alpha-2a, interferon-alpha-2β, interferon-alpha-n1, interferon-alpha-n3, interferon-beta, interferon-gamma-1α, interleukin-2, intron A, iressa, irinotecan, kytril, lentinan sulfate, letrozole, leucovorin, leuprolide, leuprolide acetate, levamisole, levofolic acid calcium salt, levothroid, levoxyl, lomustine, lonidamine, mari-nol, mechlorethamine, mecobalamin, medroxyprogesterone acetate, megestrol acetate, melphalan, menest, 6-mercaptopurine, mesna, methotrexate, metvix, miltefosine, minocycline, mitomycin C, mitotane, mitoxantrone, modrenal, myocet, nedaplatin, neulasta, neumegai, neupogen, nilutamide, nolvadex, NSC-631570, OCT-43, octreotide, ondansetron hydrochloride, orapred, oxaliplatin, paclitaxel, pediapred, pegaspargase, pegasys, pentostatin, picibanil, pilocarpine hydrochloride, pirarubicin, plicamycin, porfimer sodium, prednimustine, prednisolone, prednisone, premarin, procarbazine, procrit, raltitrexed, rebif, rhenium-186 etidronate, rituximab, roferon-A, romurtide, salagen, sandostatin, sargramostim, semustine, sizofiran, sobuzoxane, solu-medrol, streptozocin, strontium-89 chloride, synthroid, tamoxifen, tamsulosin, tasonermin, tastolactone, taxoter, tece-leukin, temozolomide, teniposide, testosterone propionate, testred, thioguanine, thiotepa, thyro-tropin, tiludronic acid, topotecan, toremifen, tositumomab, tastuzumab, teosulfan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin-stimalamer, zofran; ABI-007, acolbifen, actimmune, affinitak, aminopterin, arzoxifen, asoprisnil, atamestane, atrasentan, avastin, BAY 43-9006 (sorafenib), CCI-779, CDC-501, celebrex, cetuximab, crisnatol, cyproterone acetate, decitabine, DN-101, doxorubicin-MTC, dSLIM, dutasteride, edotecarin, eflornithine, exatecan, fenretinide, histamine dihydrochloride, histrelin hydrogel implant, holmium-166 DOTMP, ibandronic acid, interferon-gamma, intron-PEG, ixabepilone, keyhole limpet hemocyanine, L-651582, lanreotide, lasofoxifen, libra, lonafarnib, miproxifen, minodronate, MS-209, liposomal MTP-PE, MX-6, nafarelin, nemorubicin, neovastat, nolatrexed, oblimersen, onko-TCS, osidem, paclitaxel polyglutamate, pamidronate disodium, PN-401, QS-21, quazepam, R-1549, raloxifen, ranpirnas, 13-cis-retic acid, satraplatin, seocalcitol, T-138067, tarceva, taxoprexin, thymosin-alpha-1, tiazofurin, tipifarnib, tirapazamine, TLK-286, toremifen, transMID-107R, valspodar, vapreotide, vatalanib, verteporfin, vinflunin, Z-100, zoledronic acid and combinations of these.

In a preferred embodiment, the compounds of the present invention can be combined with antihyperproliferative agents, which can be, by way of example—without this list being conclusive:

aminoglutethimide, L-asparaginase, azathioprine, 5-azacytidine, bleomycin, busulfan, camptothe-cin, carboplatin, carmustine, chlorambucil, cisplatin, colaspase, cyclophosphamide, cytarabine, dacarbazine, dactinomycin, daunorubicin, diethylstilbestrol, 2′,2′-difluorodeoxycytidine, docetaxel, doxorubicin (adriamycin), epirubicin, epothilone and its derivatives, erythro-hydroxynonyladenin, ethinylestradiol, etoposide, fludarabin phosphate, 5-fluorodeoxyuridine, 5-fluorodeoxyuridine monophosphate, 5-fluorouracil, fluoxymesterone, flutamide, hexamethylmelamine, hydroxyurea, hydroxyprogesterone caproate, idarubicin, ifosfamide, interferon, irinotecan, leucovorin, lomustine, mechlorethamine, medroxyprogesterone acetate, megestrol acetate, melphalan, 6-mercaptopurine, mesna, methotrexate, mitomycin C, mitotane, mitoxantrone, paclitaxel, pentostatin, N-phosphono-acetyl L-aspartate (PALA), plicamycin, prednisolone, prednisone, procarbazine, raloxifen, semustine, streptozocin, tamoxifen, teniposide, testosterone propionate, thioguanine, thiotepa, topotecan, trimethylmelamine, uridine, vinblastine, vincristine, vindesine and vinorelbine.

The compounds according to the invention can also be combined in a very promising manner with biological therapeutics, such as antibodies (e.g. avastin, rituxan, erbitux, herceptin) and recombinant proteins, which additively or synergistically intensify the effects of inhibition of the HIF signal pathway transmission.

Inhibitors of the HIF regulation pathway, such as the compounds according to the invention, can also achieve positive effects in combination with other therapies directed against angiogenesis, such as, for example, with avastin, axitinib, DAST, recentin, sorafenib or sunitinib. Combinations with inhibitors of the proteasome and of mTOR and antihormones and steroidal metabolic enzyme inhibitors are particularly suitable because of their favourable profile of side effects.

Generally, the following aims can be pursued with the combination of compounds of the present invention with other agents having a cytostatic or cytotoxic action:
an improved activity in slowing down the growth of a tumour, in reducing its size or even in its complete elimination compared with treatment with an individual active compound;
the possibility of employing the chemotherapeutics used in a lower dosage than in monotherapy;
the possibility of a more tolerable therapy with few side effects compared with individual administration;
the possibility of treatment of a broader spectrum of tumour diseases;
achievement of a higher rate of response to the therapy;
a longer survival time of the patient compared with present-day standard therapy.

The compounds according to the invention can moreover also be employed in combination with radiotherapy and/or surgical intervention.

The present invention furthermore provides medicaments which comprise at least one compound according to the invention, conventionally together with one or more inert, non-toxic, pharmaceutically suitable auxiliary substances, and the use thereof for the above-mentioned purposes.

The compounds according to the invention can act systemically and/or locally. They can be administered in a suitable manner for this purpose, such as e.g. orally, parenterally, pulmonally, nasally, sublingually, lingually, buccally, rectally, dermally, transdermally, conjunctivally, otically or as an implant or stent.

The compounds according to the invention can be administered in suitable administration forms for these administration routes.

Administration forms which function according to the prior art, release the compounds according to the invention rapidly and/or in a modified manner and contain the compounds according to the invention in crystalline and/or amorphized and/or dissolved form are suitable for oral administration, such as e.g. tablets (non-coated or coated tablets, for example withe coatings which are resistant to gastric juice or dissolve in a delayed manner or are insoluble and control the release of the compound according to the invention), tablets or films/oblates, films/lyophilisates or capsules which disintegrate rapidly in the oral cavity (for example hard or soft gelatine capsules), sugar-coated tablets, granules, pellets, powders, emulsions, suspensions, aerosols or solutions.

Parenteral administration can be effected with bypassing of an absorption step (e.g. intravenously, intraarterially, intracardially, intraspinally or intralumbally) or with inclusion of an absorption (e.g. intramuscularly, subcutaneously, intracutaneously, percutaneously or intraperitoneally). Administration forms which are suitable for parenteral administration are, inter alia, injection and infusion formulations in the form of solutions, suspensions, emulsions, lyophilisates or sterile powders.

For the other administration routes e.g. inhalation medicament forms (inter alia powder inhalers, nebulizers), nasal drops, solutions or sprays, tablets, films/oblates or capsules for lingual, sublingual or buccal administration, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, sprinkling powders, implants or stents are suitable.

Oral and parenteral administration are preferred, in particular oral and intravenous administration.

The compounds according to the invention can be converted into the administration forms mentioned. This can be effected in a manner known per se by mixing with inert, non-toxic, pharmaceutically suitable auxiliary substances. These auxiliary substances include inter alia carrier substances (for example microcrystalline cellulose, lactose, mannitol), solvents (e.g. liquid polyethylene glycols), emulsifiers and dispersing or wetting agents (for example sodium dodecyl sulfate, polyoxysorbitan oleate), binders (for example polyvinylpyrrolidone), synthetic and natural polymers (for example albumin), stabilizers (e.g. antioxidants, such as, for example, ascorbic acid), dyestuffs (e.g. inorganic pigments, such as, for example, iron oxides) and flavour and/or smell correctants.

In general, it has proved advantageous in the case of parenteral administration to administer amounts of from about 0.001 to 1 mg/kg, preferably about 0.01 to 0.5 mg/kg of body weight to achieve effective results. In the case of oral administration the dosage is about 0.01 to 100 mg/kg, preferably about 0.01 to 20 mg/kg and very particularly preferably 0.1 to 10 mg/kg of body weight.

Nevertheless it may be necessary to deviate from the amounts mentioned, and in particular depending on the body weight, administration route, individual behaviour towards the active compound, nature of the formulation and point of time or interval at which administration takes place. Thus in some cases it may be sufficient to manage with less than the abovementioned minimum amount, while in other cases the upper limit mentioned must be exceeded. In the case where relatively large amounts are administered, it may be advisable to distribute these into several individual doses over the day.

The following embodiment examples illustrate the invention. The inventions is not limited to the examples.

The percentage data in the following tests and examples are percentages by weight, unless stated otherwise; parts are parts by weight. The solvent ratios, dilution ratios and concentration data of liquid/liquid solutions in each case relate to the volume.

### A. EXAMPLES

### Abbreviations and Acronyms

abs. absolute
aq. aqueous
Boc tert-butoxycarbonyl
Ex. Example
Bu butyl
approx. circa, approximately
CI chemical ionization (in MS)
d doublet (in NMR)
d day(s)
TLC thin layer chromatography
DCI direct chemical ionization (in MS)
dd doublet of doublet (in NMR)
DMAP 4-N,N-dimethylaminopyridine
DME 1,2-dimethoxyethane
DMF dimethylformamide
DMSO dimethylsulfoxide
dt doublet of triplet (in NMR)
of th. of theory (chemical yield)
EDC N′-(3-dimethylaminopropyl)-N-ethylcarbodiimide hydrochloride
ee enantiomer excess
EI electron impact ionization (in MS)
eq. equivalent(s)
ESI electrospray ionization (in MS)
Et ethyl
GC gas chromatography
h hour(s)
HOBt 1-hydroxy-1H-benzotriazole hydrate
HPLC high pressure, high performance liquid chromatography
ⁱPr isopropyl
LC-MS liquid chromatography-coupled mass spectrometry
m multiplet (in NMR)
min minute(s)
MPLC medium pressure liquid chromatography (over silica gel; also called “flash chromatography”)
MS mass spectrometry
NMP N-methyl-2-pyrrolidone
NMR nuclear magnetic resonance spectrometry
Pd/C palladium on active charcoal
PEG polyethylene glycol
Pr propyl
quart quartet (in NMR)
quint quintet (in NMR)
R_{f} retention index (in TLC)
RT room temperature
Rₜ retention time (in HPLC)
s singlet (in NMR)
sept septet (in NMR)
t triplet (in NMR)
^{t}Bu tert-butyl
TFA trifluoroacetic acid
THF tetrahydrofuran
UV ultraviolet spectrometry
v/v volume to volume ratio (of a solution)
tog. together

### HPLC Methods:

### Method A

Instrument: HP 1100 with DAD detection; column: Kromasil 100 RP-18, 60 mm×2.1 mm, 3.5 μm; eluent A: 5 ml of perchloric acid (70% strength)/1 of water, eluent B: acetonitrile; gradient: 0 min 2% B→0.5 min 2% B→4.5 min 90% B→6.5 min 90% B→6.7 min 2% B→7.5 min 2% B; flow rate: 0.75 ml/min; column temperature: 30° C.; UV detection: 210 nm

### Method B

Instrument: HP 1100 with DAD detection; column: Kromasil 100 RP-18, 60 mm×2.1 mm, 3.5 μm; eluent A: 5 ml of perchloric acid (70% strength)/1 of water, eluent B: acetonitrile; gradient: 0 min 2% B→0.5 min 2% B→4.5 min 90% B→9 min 90% B→92 min 2% B→10 min 2% B; flow rate: 0.75 ml/min; column temperature: 30° C.; UV detection: 210 nm.

### LC/MS Methods:

### Method C

Apparatus type MS: Micromass ZQ; apparatus type HPLC: HP 1100 Series; UV DAD; column: Phenomenex Gemini 3μ, 30 mm×3.00 mm; eluent A: 1 l of water+0.5 ml of 50% strength formic acid, eluent B: 1 l of acetonitrile+0.5 ml of 50% strength formic acid; gradient 0.0 min 90% A→2.5 min 30% A→3.0 min 5% A→4.5 min 5% A; flow rate 0.0 min 1 ml/min→2.5 min/3.0 min/4.5 min 2 ml/min; oven: 50° C.; UV detection: 210 nm

### Method D

Apparatus type MS: Waters Micromass Quattro Micro; apparatus type HPLC: Agilent 1100 Series; column: Thermo Hypersil GOLD 3μ, 20 mm×4 mm; eluent A: 1 l of water+0.5 ml of 50% strength formic acid, eluent B: 1 l of acetonitrile+0.5 ml of 50% strength formic acid; gradient: 0.0 min 100% A→3.0 min 10% A→4.0 min 10% A→4.01 min 100% A→5.00 min 100% A; oven: 50° C.; flow rate: 2 ml/min; UV detection: 210 nm

### Method E

Apparatus type MS: Micromass ZQ; apparatus type HPLC: Waters Alliance 2795; column: Phenomenex Synergi 2.5μ MAX-RP 100A Mercury 20 mm×4 mm; eluent A: 1 l of water+0.5 ml of 50% strength formic acid, eluent B: 1 l of acetonitrile+0.5 ml of 50% strength formic acid; gradient: 0.0 min 90% A→0.1 min 90% A→3.0 min 5% A→4.0 min 5% A→4.01 min 90% A; flow rate: 2 ml/min; oven: 50° C.; UV detection: 210 nm

### Method F

Instrument: Micromass Quattro Premier with Waters UPLC Acquity; column: Thermo Hypersil GOLD 1.9μ, 50 mm×1 mm; eluent A: 1 l of water+0.5 ml of 50% strength formic acid, eluent B: 1 l of acetonitrile+0.5 ml of 50% strength formic acid; gradient: 0.0 min 90% A→0.1 min 90% A→1.5 min 10% A→2.2 min 10% A; flow rate: 0.33 ml/min; oven: 50° C.; UV detection: 210 nm

### Method G

Instrument: Micromass Platform LCZ with HPLC Agilent Series 1100; column: Thermo Hypersil GOLD 3μ, 20 mm×4 mm; eluent A: 1 l of water+0.5 ml of 50% strength formic acid, eluent B: 1 l of acetonitrile+0.5 ml of 50% strength formic acid; gradient: 0.0 min 100% A→0.2 min 100% A→2.9 min 30% A→3.1 min 10% A→5.5 min 10% A; oven: 50° C.; flow rate: 0.8 ml/min; UV detection: 210 nm

### Method H

Instrument: Micromass Quattro LCZ with HPLC Agilent Series 1100; column: Phenomenex Syn-ergi 2.5μ MAX-RP 100A Mercury 20 mm×4 mm; eluent A: 1 l of water+0.5 ml of 50% strength formic acid, eluent B: 1 l of acetonitrile+0.5 ml of 50% strength formic acid; gradient 0.0 min 90% A→0.1 min 90% A→3.0 min 5% A→4.0 min 5% A→4.1 min 90% A; flow rate: 2 ml/min; oven: 50° C.; UV detection: 208-400 nm

### Method I

Instrument: Waters Acquity SQD UPLC System; column: Waters Acquity UPLC HSS T3 1.8 μm, 50 mm×1 mm; Eluent A: 1 l of water+0.25 ml of 99% strength formic acid, eluent B: 1 l of acetonitrile+0.25 ml of 99% strength formic acid; gradient: 0.0 min 90% A→1.2 min 5% A→2.0 min 5% A; flow rate: 0.40 ml/min; oven: 50° C.; UV detection: 210-400 nm

### Method J

Instrument MS: Waters ZQ 2000; instrument HPLC: Agilent 1100, 2-column circuit; autosampler: HTC PAL; column: YMC-ODS-AQ, 50 mm×4.6 mm, 3.0 μm; eluent A: water+0.1% formic acid, eluent B: acetonitrile+0.1% formic acid; gradient: 0.0 min 100% A→0.2 min 95% A→1.8 min 25% A→1.9 min 10% A→2.0 min 5% A→3.2 min 5% A→3.21 min 100% A→3.35 min 100% A; oven: 40° C.; flow rate: 3.0 ml/min; UV detection: 210 nm.

### Method K

Instrument MS: Waters SQD; Instrument HPLC: Waters UPLC; column: Zorbax SB-Aq (Agilent), 50 mm×2.1 mm, 1.8 μm; Eluent A: water+0.025% formic acid, eluent B: acetonitrile+0.025% formic acid; gradient: 0.0 min 98% A→0.9 min 25% A→1.0 min 5% A→1.4 min 5% A→1.41 min 98% A→1.5 min 98% A; oven: 40° C.; flow rate: 0.60 ml/min; UV detection: DAD, 210 nm

### GC/MS Methods:

### Method L

Instrument: Micromass GCT, GC 6890; column: Restek RTX-35, 15 m×200 μm×0.33 μm; constant flow rate with helium: 0.88 ml/min; oven: 70° C.; inlet: 250° C.; gradient: 70° C., 30° C./min 310° C. (hold for 3 min)

### Method M

Instrument: Micromass GCT, GC 6890; column: Restek RTX-35, 15 m×200 μm×0.33 μm; constant flow rate with helium: 0.88 ml/min; oven: 70° C.; inlet: 250° C.; gradient: 70° C., 30° C./min→310° C. (hold for 12 min)

### Preparative HPLC Methods:

### Method N

Column: GROM-SIL 120 ODS-4 HE, 10 μm, 250 mm×30 mm; mobile phase and gradient programme: acetonitrile/0.1% aq. formic acid 10:90 (0-3 min), acetonitrile/0.1% aq. formic acid 10:90→95:5 (3-27 min), acetonitrile/0.1% aq. formic acid 95:5 (27-34 min), acetonitrile/0.1% aq. formic acid 10:90 (34-38 min); flow rate: 50 ml/min; temperature: 22° C.; UV detection: 254 nm.

### Method O

Column: Reprosil C18, 10 μm, 250 mm×30 mm; mobile phase and gradient programme: acetonitrile/0.1% aq. trifluoroacetic acid 10:90 (0-2 min), acetonitrile/0.1% aq. trifluoroacetic acid 10:90→90:10 (2-23 min), acetonitrile/0.1% aq. trifluoroacetic acid 90:10 (23-28 min), acetonitrile/0.1% aq. trifluoroacetic acid 10:90 (28-30 min); flow rate: 50 ml/min; temperature: 22° C.; UV detection: 210 nm

### Method P

Column: Reprosil C18, 10 μm, 250 mm×30 mm; mobile phase and gradient programme: acetonitrile/0.1% aq. ammonia 20:80 (0-3 min), acetonitrile/0.1% aq. ammonia 20:80→98:2 (3-35 min), acetonitrile/0.1% aq. ammonia 98:2 (35-40 min); flow rate: 50 ml/min; temperature: 22° C.; UV detection: 210 nm.

### LC/MS Method:

### Method Q

Apparatus type MS: Waters ZQ; apparatus type HPLC: Agilent 1100 Series; UV DAD; column: Thermo Hypersil GOLD 3μ, 20 mm×4 mm; eluent A: 1 l of water+0.5 ml of 50% strength formic acid, eluent B: 1 l of acetonitrile+0.5 ml of 50% strength formic acid; gradient: 0.0 min 100% A→3.0 min 10% A→4.0 min 10% A→4.1 min 100% A (flow rate 2.5 ml/min); oven: 55° C.; flow rate: 2 ml/min; UV detection: 210 nm.

For all the reactants or reagents for which the preparation is not described explicitly in the following, they were obtained commercially from generally accessible sources. For all the other reactants or reagents for which the preparation likewise is not described in the following and which were not commercially obtainable or were obtained from sources which are not generally accessible, reference is made to the published literature in which their preparation is described.

### Starting Compounds and Intermediates

### Example 1A

### N′-Hydroxy-4-(1,1,1-trifluoro-2-methylpropan-2-yl)benzenecarboximide amide

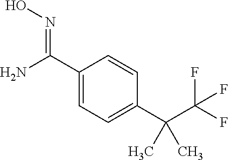 [SMILES: CC(C)(c1ccc(/C(N)=N/O)cc1)C(F)(F)F]

### Step 1: 2-(4-Bromophenyl)-1,1,1-trifluoropropan-2-ol

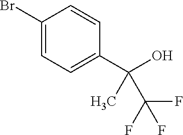

A suspension of dichloro(dimethyl)titanium in a heptane/methylene chloride mixture was first prepared as follows: 100 ml (100 mmol) of a 1 M solution of titanium tetrachloride in methylene chloride were cooled to −30° C., 100 ml (100 mmol) of a 1 M solution of dimethylzinc in heptane were added dropwise and the mixture was subsequently stirred at −30° C. for 30 min. This suspension was then cooled to −40° C. and a solution of 10 g (39.5 mmol) of 1-(4-bromophenyl)-2,2,2-trifluoroethanone in 50 ml of methylene chloride was added. The mixture was subsequently stirred at −40° C. for 5 min, the temperature was then allowed to come to RT and the mixture was stirred at RT for a further 2 h. 50 ml of water were slowly added dropwise, while cooling with ice, and the mixture was then diluted with a further 300 ml of water. It was extracted twice with methylene chloride, the combined methylene chloride phases were washed once with water, dried over anhydrous magnesium sulfate and filtered and the solvent was removed on a rotary evaporator. The residue was purified by column chromatography over silica gel (mobile phase: cyclohexane/ethyl acetate 85:15). 10.5 g (100% of th.) of the title compound were obtained which, according to ¹H-NMR, still contained residues of solvent.

¹H-NMR (400 MHz, CDCl₃, δ/ppm): 7.52 (d, 2H), 7.47 (d, 2H), 1.76 (s, 3H).

LC/MS (method C, ESIpos): Rₜ=2.27 min, m/z=268 [M+H]⁺.

### Step 2: 2-(4-Bromophenyl)-1,1,1-trifluoropropan-2-yl methanesulfonate

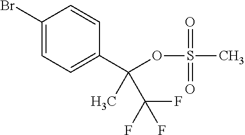 [SMILES: CC(OS(C)(=O)=O)(c1ccc(Br)cc1)C(F)(F)F]

3.12 g (78.05 mmol, 60% strength in mineral oil) of sodium hydride were initially introduced into 45 ml of THF under argon and a solution of 10.5 g (39.03 mmol) of the compound obtained in Example 1A/step 1 in 20 ml of THF was added dropwise at RT. After the mixture had been stirred at RT for 1 h and at 40° C. for 30 min, a solution of 8.94 g (78.05 mmol) of methanesulfonyl chloride in 45 ml of THF was added dropwise and the reaction mixture was stirred at 40° C. for a further 60 min. 50 ml of water were then slowly added dropwise to the mixture and the mixture was diluted with saturated aqueous sodium bicarbonate solution and extracted twice with ethyl acetate. The combined ethyl acetate phases were dried over anhydrous magnesium sulfate and filtered and the solvent was removed on a rotary evaporator. The residue was stirred in hexane and the solid obtained was filtered off and dried in vacuo. 12.4 g (92% of th.) of the title compound were obtained.

¹H-NMR (400 MHz, CDCl₃, δ/ppm): 7.58 (d, 2H), 7.43 (d, 2H), 3.16 (s, 3H), 2.28 (s, 3H).

LC/MS (method D, ESIpos): Rₜ=2.32 min, m/z=364 [M+NH₄]⁺.

### Step 3: 1-Bromo-4-(1,1,1-trifluoro-2-methylpropan-2-yl)benzene

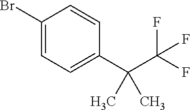

12.4 g (35.72 mmol) of the compound obtained in Example 1A/step 2 were initially introduced into 250 ml of methylene chloride and the mixture was cooled to 0° C. 35.7 ml (71.44 mmol) of a 2 M solution of trimethylaluminium were then slowly added dropwise at 0° C., while stirring, and the mixture was then allowed to come to RT and was subsequently stirred at RT for a further 1.5 h. 120 ml of a saturated aqueous sodium bicarbonate solution were slowly added dropwise to the mixture, followed by 40 ml of a saturated aqueous sodium chloride solution. The mixture was filtered over kieselguhr and the kieselguhr was rinsed twice with methylene chloride. The combined methylene chloride phases were washed once with saturated aqueous sodium chloride solution and dried over anhydrous magnesium sulfate and the solvent was removed on a rotary evaporator. 8.69 g (87% of th.) of the title compound were obtained in a purity of 95%.

¹H-NMR (400 MHz, CDCl₃, δ/ppm): 7.49 (d, 2H), 7.33 (d, 2H), 1.55 (s, 6H).

LC/MS (method E, ESIpos): Rₜ=2.54 min, no ionization.

GC/MS (method L, EI): Rₜ=3.48 min, m/z=266 [M]⁺.

### Step 4: 4-(1,1,1-Trifluoro-2-methylpropan-2-yl)benzenecarbonitrile

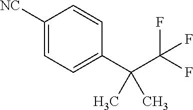

3.34 g (12.50 mmol) of the compound obtained in Example 1A/step 3 were initially introduced into 2.5 ml of degassed DMF under argon, 881 mg (7.50 mmol) of zinc cyanide and 867 mg (0.75 mmol) of tetrakis(triphenylphosphine)palladium(0) were added and the mixture was stirred at 80° C. overnight. After cooling to RT, the reaction mixture was diluted with ethyl acetate and solid constituents were filtered off. The filtrate was washed twice with 2 N aqueous ammonia solution and once with saturated aqueous sodium chloride solution, dried over anhydrous magnesium sulfate and freed from the solvent on a rotary evaporator. The residue was purified by column chromatography over silica gel (mobile phase: cyclohexane/ethyl acetate 85:15). 2.08 g (78% of th.) of the title compound were obtained.

¹H-NMR (400 MHz, CDCl₃, δ/ppm): 7.68 (d, 2H), 7.62 (d, 2H), 1.60 (s, 6H).

GC/MS (method L, EI): Rₜ=3.83 min, m/z=213 [M]⁺.

### Step 5: N′-Hydroxy-4-(1,1,1-trifluoro-2-methylpropan-2-yl)benzenecarboximide amide

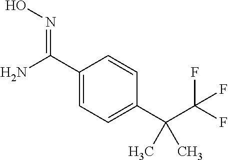 [SMILES: CC(C)(c1ccc(/C(N)=N/O)cc1)C(F)(F)F]

A mixture of 2.40 g (11.26 mmol) of the compound from Example 1A/step 4, 1.72 g (24.77 mmol) of hydroxylamine hydrochloride and 3.45 ml (24.77 mmol) of triethylamine in 60 ml of ethanol was stirred under reflux for 1 h. After cooling to RT, the solvent was removed on a rotary evaporator. Ethyl acetate was added to the residue and the solid present was filtered off. The ethyl acetate solution was washed successively with water and saturated aqueous sodium chloride solution, dried over anhydrous magnesium sulfate and filtered. After removal of the solvent, the oil obtained was triturated with petroleum ether. After the resulting solid had been filtered off with suction and dried under a high vacuum, 2.65 g (96% of th.) of the title compound were obtained.

¹H-NMR (400 MHz, CDCl₃, δ/ppm): 8.0 (s, broad, 1H), 7.62 (d, 2H), 7.52 (d, 2H), 4.88 (s, broad, 2H), 1.60 (s, 6H).

LC/MS (method D, ESIpos): Rₜ=1.34 min, m/z=247 [M+H]⁺.

### Example 2A

### 4-(2-Fluoropropan-2-yl)-N′-hydroxybenzenecarboximide amide

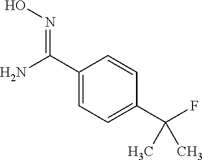

### Step 1: 4-(2-Fluoropropan-2-yl)benzenecarbonitrile

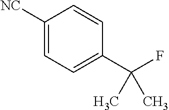

1.20 g (7.44 mmol) of diethylaminosulfur trifluoride (DAST) were added to a solution of 1.00 g (6.20 mmol) of 4-(2-hydroxypropan-2-yl)benzenecarbonitrile [obtained from 4-(propan-2-yl)benzenecarbonitrile in accordance with J. L. Tucker et al., Synth. Comm. 2006, 36 (15), 2145-2155] in 20 ml of methylene chloride at a temperature of 0° C. The reaction mixture was stirred at RT for 2 h and then diluted with water and extracted with methylene chloride. The organic phase was washed with water, dried over anhydrous magnesium sulfate and filtered. After removal of the solvent on a rotary evaporator, the residue was purified by means of MPLC (silica gel, mobile phase: cyclohexane/ethyl acetate 95:5). 675 mg (67% of th.) of the title compound were obtained.

¹H-NMR (400 MHz, CDCl₃, δ/ppm): 7.57 (d, 2H), 7.48 (d, 2H), 1.72 (s, 3H), 1.68 (s, 3H).

LC/MS (method D, ESIpos): Rₜ=2.12 min, m/z=163 [M+H]⁺.

### Step 2: 4-(2-Fluoropropan-2-yl)-N′-hydroxybenzenecarboximide amide

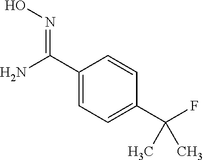

By the process described under Example 1A/step 5, 756 mg (93% of th.) of the title compound were obtained from 675 mg (4.14 mmol) of the compound from Example 2A/step 1.

¹H-NMR (400 MHz, CDCl₃, δ/ppm): 7.62 (d, 2H), 7.41 (d, 2H), 4.89 (s, broad, 2H), 1.72 (s, 3H), 1.68 (s, 3H).

LC/MS (method D, ESIpos): Rₜ=1.04 min, m/z=197 [M+H]⁺.

### Example 3A

### N′-Hydroxy-4-[(trifluoromethyl)sulfonyl]benzenecarboximide amide

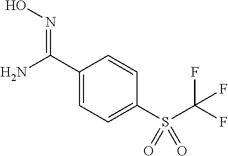 [SMILES: N/C(=N\O)c1ccc(S(=O)(=O)C(F)(F)F)cc1]

By the process described under Example 1A/step 5, 5.08 g (97% of th.) of the title compound were obtained from 4.60 g (19.56 mmol) of 4-[(trifluoromethyl)sulfonyl]benzenecarbonitrile [W. Su, Tetrahedron. Lett. 1994, 35 (28), 4955-4958].

¹H-NMR (400 MHz, DMSO-d₆, δ/ppm): 10.26 (s, 1H), 8.13 (dd, 4H), 6.12 (s, 2H).

LC/MS (method D, ESIpos): Rₜ=1.57 min, m/z=269 [M+H]⁺.

### Example 4A

### N′-Hydroxy-4-(3-methyloxetan-3-yl)benzenecarboximide amide

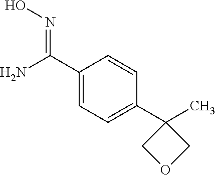

### Step 1: [4-(Dibenzylamino)phenyl]boronic acid

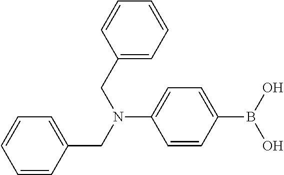 [SMILES: OB(O)c1ccc(N(Cc2ccccc2)Cc2ccccc2)cc1]

A solution of 60 g (17.03 mmol) of N,N-dibenzyl-4-bromoaniline [T. Saitoh et al., J. Am. Chem. Soc. 2005, 127 (27), 9696-9697] was initially introduced into a mixture of 75 ml of anhydrous diethyl ether and 75 ml of anhydrous THF under inert conditions. 13.9 ml (22.14 mmol) of a 1.6 M solution of n-butyllithium in hexane were added dropwise to this solution at −78° C. When the addition had ended, the mixture was stirred at −78° C. for 60 min, before 6.3 ml (27.25 mmol) of boric acid triisopropyl ester were added dropwise at the same temperature. After a further 15 min at −78° C., the reaction mixture was allowed to come to RT. After stirring at RT for 3 h, 18 ml of 2 M hydrochloric acid were added and the resulting mixture was stirred intensively at RT for 20 min. After dilution with approx. 200 ml of water, the mixture was extracted three times with approx. 200 ml of ethyl acetate each time. The combined organic extracts were washed successively with water and saturated sodium chloride solution. After drying over anhydrous magnesium sulfate, the mixture was filtered and the solvent was removed on a rotary evaporator. The oily residue obtained was triturated with a mixture of 50 ml of tert-butyl methyl ether and 50 ml of pentane. After the resulting solid had been filtered off with suction and dried under a high vacuum, 3.91 g (72% of th., purity of 90%) of the title compound were obtained, this being employed in the next stage without further purification.

¹H-NMR (400 MHz, DMSO-d₆, δ/ppm): 7.58 (d, 2H), 7.32-7.30 (m, 4H), 7.27-7.23 (m, 6H), 6.66 (d, 2H), 4.70 (s, 4H).

HPLC (method A): Rₜ=4.35 min.

MS (ESIpos): m/z=318 [M+H]⁺.

### Step 2: Ethyl {3-[4-(dibenzylamino)phenyl]oxetan-3-yl}acetate

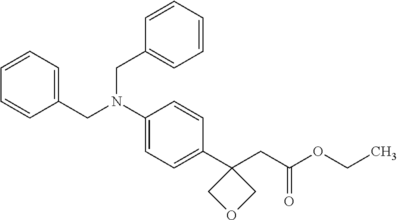 [SMILES: CCOC(=O)CC1(c2ccc(N(Cc3ccccc3)Cc3ccccc3)cc2)COC1]

10.7 ml (16.0 mmol) of a 1.5 M potassium hydroxide solution were added to a solution of 304 mg (0.616 mmol) of (1,5-cyclooctadiene)rhodium(I) chloride dimer in 30 ml of 1,4-dioxane. Solutions of 1.75 g (12.31 mmol) of ethyl oxetan-3-ylideneacetate [G. Wuitschik et al., Angew. Chem. Int. Ed. Engl. 2006, 45 (46), 7736-7739] in 1 ml of 1,4-dioxane and 3.91 g (12.31 mmol) of the compound from Example 4A/step 1 in 60 ml of 1,4-dioxane were then added successively. The reaction mixture was stirred at RT for 6 h. It was then diluted with approx. 200 ml of water and extracted three times with approx. 200 ml of ethyl acetate each time. The combined organic extracts were washed successively with water and saturated sodium chloride solution. After drying over anhydrous magnesium sulfate, the mixture was filtered and the solvent was removed on a rotary evaporator. The crude product obtained was purified by means of MPLC (silica gel, mobile phase: cyclohexane/ethyl acetate 20:1,→5:1). 3.51 g (67% of th.) of the title compound were obtained.

¹H-NMR (400 MHz, CDCl₃, δ/ppm): 7.33-7.30 (m, 4H), 7.27-7.23 (m, 6H), 6.97 (d, 2H), 6.69 (d, 2H), 4.94 (d, 2H), 4.81 (d, 2H), 4.62 (s, 4H), 4.00 (quart, 2H), 3.04 (s, 2H), 1.11 (t, 3H).

LC/MS (method E, ESIpos): Rₜ=2.57 min, m/z=416 [M+H]⁺.

### Step 3: 2-{3-[4-(Dibenzylamino)phenyl]oxetan-3-yl}ethanol

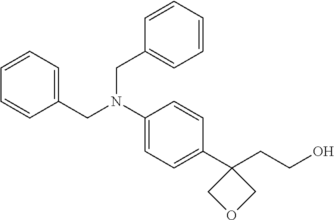 [SMILES: OCCC1(c2ccc(N(Cc3ccccc3)Cc3ccccc3)cc2)COC1]

4.9 ml (4.88 mmol) of a 1 M solution of lithium aluminium hydride in THF were added dropwise to a solution of 2.90 g (6.98 mmol) of the compound from Example 4A/step 2 in 145 ml of anhydrous THF under inert conditions and at a temperature of 0° C. When the dropwise addition had ended, the reaction mixture was stirred at 0° C. for 1.5 h. 2 g of kieselguhr and 2 ml of water were then cautiously added. The heterogeneous mixture was filtered with suction over a paper filter. The filtrate was diluted with approx. 250 ml of water and extracted three times with approx. 250 ml of ethyl acetate each time. The combined organic extracts were washed successively with water and saturated sodium chloride solution. After drying over anhydrous magnesium sulfate, the mixture was filtered and the solvent was removed on a rotary evaporator. The crude product obtained was purified by means of MPLC (silica gel, mobile phase: cyclohexane/ethyl acetate 4:1). 2.34 g (87% of th.) of the title compound were obtained.

¹H-NMR (400 MHz, CDCl₃, δ/ppm): 7.36-7.31 (m, 4H), 7.27-7.22 (m, 6H), 6.88 (d, 2H), 6.71 (d, 2H), 4.93 (d, 2H), 4.71 (d, 2H), 4.63 (s, 4H), 3.55 (quart, 2H), 2.29 (t, 2H), 1.12 (t, 1H).

HPLC (method B): Rₜ=3.98 min.

MS (DCI, NH₃): m/z=374 [M+H]⁺.

LC/MS (method E, ESIpos): Rₜ=2.15 min, m/z=374 [M+H]⁺.

### Step 4: {3-[4-(Dibenzylamino)phenyl]oxetan-3-yl}acetaldehyde

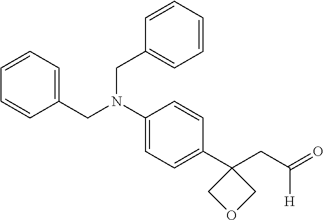 [SMILES: O=CCC1(c2ccc(N(Cc3ccccc3)Cc3ccccc3)cc2)COC1]

807 μl of anhydrous DMSO were added dropwise to a solution of 496 μl (5.68 mmol) of oxalyl chloride in 5 ml of anhydrous methylene chloride at −78° C. under inert conditions. After 20 min, a solution of 1.93 g (5.17 mmol) of the compound from Example 4A/step 3 in 5 ml of anhydrous methylene chloride was slowly added dropwise at the same temperature. After stirring at −78° C. for 60 min, 3.7 ml (26.87 mmol) of anhydrous triethylamine were added dropwise. After a further 10 min at this temperature, the reaction mixture was allowed to warm to RT. The mixture was then introduced into a suction filter filled with silica gel and elution was carried out first with cyclohexane and then with cyclohexane/ethyl acetate 7:1→1:1. The product fractions were combined and evaporated to dryness and the residue was taken up in ethyl acetate. Washing was carried out successively with saturated sodium bicarbonate solution, water and saturated sodium chloride solution. After drying over anhydrous magnesium sulfate, the mixture was filtered and the solvent was removed on a rotary evaporator. 1.81 g (92% of th.) of the title compound were obtained.

¹H-NMR (400 MHz, CDCl₃, δ/ppm): 9.69 (t, 1H), 7.34-7.31 (m, 4H), 7.28-7.23 (m, 6H), 6.97 (d, 2H), 6.70 (d, 2H), 5.00 (d, 2H), 4.72 (d, 2H), 4.63 (s, 4H), 3.18 (d, 2H).

HPLC (method B): Rₜ=4.61 min.

MS (DCI, NH₃): m/z=372 [M+H]⁺.

LC/MS (method F, ESIpos): Rₜ=1.43 min, m/z=372 [M+H]⁺.

### Step 5: N,N-Dibenzyl-4-(3-methyloxetan-3-yl)aniline

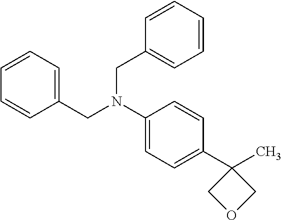 [SMILES: CC1(c2ccc(N(Cc3ccccc3)Cc3ccccc3)cc2)COC1]

A solution of 1.81 g (4.87 mmol) of the compound from Example 4A/step 4 and 13.57 g (14.62 mmol) of tris(triphenylphosphine)rhodium(I) chloride in 240 ml of toluene was heated under reflux under inert conditions for one hour. After cooling to RT, insoluble constituents were filtered off. The solvent was removed on a rotary evaporator and the residue was purified by means of MPLC (silica gel, cyclohexane/ethyl acetate 20:1→5:1). 1.36 g (73% of th., purity of approx. 90%) of the title compound were obtained.

¹H-NMR (400 MHz, CDCl₃, δ/ppm): 7.35-7.31 (m, 4H), 7.27-7.24 (m, 6H), 7.07 (d, 2H), 6.72 (d, 2H), 4.90 (d, 2H), 4.64 (s, 4H), 4.55 (d, 2H), 1.96 (s, 3H).

LC/MS (method F, ESIpos): Rₜ=1.55 min, m/z=344 [M+H]⁺.

### Step 6: 4-(3-Methyloxetan-3-yl)aniline

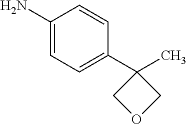

A solution of 1.35 g (3.93 mmol) of the compound from Example 4A/step 5 in 135 ml of ethanol was hydrogenated in a flow-through hydrogenation apparatus (“H-Cube” from ThalesNano, Budapest, Hungary) (conditions: 10% Pd/C catalyst, “full H₂” mode, 1 ml/min, 50° C.). After removal of the solvent on a rotary evaporator, the crude product was purified by means of MPLC (silica gel, cyclohexane/ethyl acetate 4:1→2:1). 386 mg (60% of th.) of the title compound were obtained.

¹H-NMR (400 MHz, CDCl₃, δ/ppm): 7.03 (d, 2H), 6.69 (d, 2H), 4.92 (d, 2H), 4.58 (d, 2H), 3.63 (s, broad, 2H), 1.69 (s, 3H).

LC/MS (method D, ESIpos): Rₜ=0.77 min, m/z=164 [M+H]⁺.

### Step 7: 4-(3-Methyloxetan-3-yl)benzenecarbonitrile

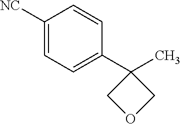

First 1.7 ml (20.7 mmol) of concentrated hydrochloric acid and then, dropwise, a solution of 159 mg (2.30 mmol) of sodium nitrite in 5 ml of water were added to a solution of 375 mg (2.30 mmol) of the compound from Example 4A/step 6 in 17 ml of water at 0° C. The mixture was stirred at 0° C. for 30 min, before 1.1 g (10.3 mmol) of solid sodium carbonate were added in portions. The solution obtained in this way was added dropwise to a solution of 257 mg (2.87 mmol) of copper(I) cyanide and 464 mg (7.12 mmol) of potassium cyanide in 16 ml of toluene/water (2:1) at 0° C. The reaction mixture was stirred at 0° C. for 1 h. The mixture was then allowed to warm to RT. The organic phase was then separated off and washed successively with water and saturated sodium chloride solution. After the solvent had been separated off on a rotary evaporator, the crude product was purified by means of MPLC (silica gel, cyclohexane/ethyl acetate 10:1→2:1). 390 mg (83% of th., purity of approx. 84%) of the title compound were obtained.

¹H-NMR (400 MHz, CDCl₃, δ/ppm): 7.66 (d, 2H), 7.31 (d, 2H), 4.92 (d, 2H), 4.68 (d, 2H), 1.73 (s, 3H).

GC/MS (method L, EIpos): Rₜ=5.45 min, m/z=173 (M)⁺.

### Step 8: N′-Hydroxy-4-(3-methyloxetan-3-yl)benzenecarboximide amide

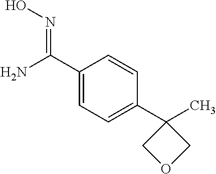

By the process described under Example 1A/step 5, 297 mg (74% of th.) of the title compound were obtained from 375 mg (1.83 mmol) of the compound from Example 4A/step 7.

¹H-NMR (400 MHz, DMSO-d₆, δ/ppm): 9.59 (s, 1H), 7.64 (d, 2H), 7.23 (d, 2H), 5.79 (s, broad, 2H), 4.80 (d, 2H), 4.53 (d, 2H), 1.62 (s, 3H).

HPLC (method A): Rₜ=2.74 min.

MS (DCI, NH₃): m/z=207 [M+H]⁺.

### Example 5A

### 4-(3-Fluoro-oxetan-3-yl)-N′-hydroxybenzenecarboximide amide

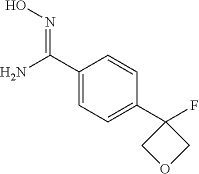 [SMILES: N/C(=N\O)c1ccc(C2(F)COC2)cc1]

### Step 1: 4-(3-Hydroxyoxetan-3-yl)benzenecarbonitrile

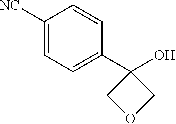 [SMILES: N#Cc1ccc(C2(O)COC2)cc1]

11 ml (21.8 mmol) of a 2 M solution of isopropylmagnesium chloride in diethyl ether were added dropwise to a solution of 5.0 g (21.8 mmol) of 4-iodobenzonitrile in 100 ml of anhydrous THF at −40° C. under inert conditions. After the mixture had been stirred at the same temperature for 1.5 h, it was cooled down to −78° C. and was slowly added to a solution, likewise cooled to −78° C., of 2.95 g (32.7 mmol, 80% in methylene chloride) of 3-oxooxetane [G. Wuitschik et al., Angew. Chem. Int. Ed. Engl. 2006, 45 (46), 7736-7739] in 100 ml of anhydrous THF with the aid of a cannula. When the addition had ended, the reaction mixture was stirred first at −78° C. for 10 min, then at 0° C. for 2 h and finally at RT for 30 min. A few ml of saturated aqueous ammonium chloride solution were then added. The solvent was then largely removed on a rotary evaporator. The residue obtained was diluted with 200 ml of water and extracted three times with approx. 200 ml of ethyl acetate each time. The combined organic extracts were washed successively with water and saturated sodium chloride solution. After drying over anhydrous magnesium sulfate, the mixture was filtered and the solvent was removed on a rotary evaporator. The crude product obtained was purified by crystallization from cyclohexane/ethyl acetate 10:1. 2.42 g (63% of th.) of the title compound were obtained.

¹H-NMR (400 MHz, DMSO-d₆, δ/ppm): 7.88 (d, 2H), 7.80 (d, 2H), 6.63 (s, 1H), 4.79 (d, 2H), 4.65 (d, 2H).

HPLC (method A): Rₜ=3.09 min.

MS (DCI, NH₃): m/z=193 [M+NH₄]⁺.

### Step 2: 4-(3-Fluoro-oxetan-3-yl)benzenecarbonitrile

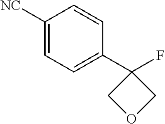 [SMILES: N#Cc1ccc(C2(F)COC2)cc1]

A solution of 662 mg (4.11 mmol) of diethylaminosulfur trifluoride (DAST) in 5 ml of methylene chloride was added dropwise to a suspension of 600 mg (3.43 mmol) of the compound from Example 5A/step 1 in 55 ml of methylene chloride at −78° C. under inert conditions. After 30 min at −78° C., the reaction mixture was warmed very rapidly to −20° C. with the aid of an ice/water bath. After approx. 30 seconds, 20 ml of 1 M sodium hydroxide solution were added and the mixture was allowed to warm to RT. After dilution with 150 ml of water, the mixture was extracted three times with approx. 50 ml of diethyl ether each time. The combined organic extracts were dried over anhydrous magnesium sulfate. After filtration, the solvent was removed on a rotary evaporator. The crude product was purified by means of MPLC (silica gel, cyclohexane/ethyl acetate 8:1). 495 mg (82% of th.) of the title compound were obtained.

¹H-NMR (400 MHz, CDCl₃, δ/ppm): 7.76 (d, 2H), 7.73 (d, 2H), 5.15 (dd, 2H), 4.81 (dd, 2H).

LC/MS (method D, ESIpos): Rₜ=1.59 min, m/z=178 [M+H]⁺.

### Step 3: 4-(3-Fluoro-oxetan-3-yl)-N′-hydroxybenzenecarboximide amide

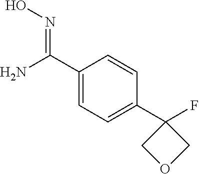 [SMILES: N/C(=N\O)c1ccc(C2(F)COC2)cc1]

By the process described under Example 1A/step 5, 470 mg (86% of th.) of the title compound were obtained from 450 mg (2.54 mmol) of the compound from Example 5A/step 2.

¹H-NMR (400 MHz, DMSO-d₆, δ/ppm): 9.71 (s, 1H), 7.77 (d, 2H), 7.54 (d, 2H), 5.87 (broad s, 2H), 4.97 (dd, 2H), 4.91 (dd, 2H).

HPLC (method A): Rₜ=2.64 min.

MS (DCI, NH₃): m/z=211 [M+H]⁺.

LC/MS (method D, ESIpos): Rₜ=0.80 min, m/z=211 [M+H]⁺.

### Example 6A

### N′-Hydroxy-4-(3-methoxyoxetan-3-yl)benzenecarboximide amide

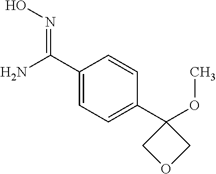 [SMILES: COC1(c2ccc(/C(N)=N/O)cc2)COC1]

### Step 1: 4-(3-Methoxyoxetan-3-yl)benzenecarbonitrile

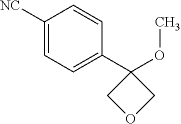

151 mg (3.77 mmol) of a 60% strength dispersion of sodium hydride in mineral oil were added to a solution of 600 mg (3.43 mmol) of the compound from Example 5A/step 1 in 12.5 ml of anhydrous DMF at 5° C. The mixture was stirred at 5° C. for 1 h, before 256 μl (4.11 mmol) of methyl iodide were added. The reaction mixture was then allowed to come to RT. After stirring for 15 h, 150 ml of water were added and the mixture was extracted twice with approx. 150 ml of diethyl ether each time. The combined organic extracts were dried over anhydrous magnesium sulfate. After filtration and removal of the solvent on a rotary evaporator, the residue obtained was purified by means of MPLC (silica gel, cyclohexane/ethyl acetate 20:1→4:1). 566 mg (87% of th.) of the title compound were obtained.

¹H-NMR (400 MHz, DMSO-d₆, δ/ppm): 7.92 (d, 2H), 7.68 (d, 2H), 4.81 (d, 2H), 4.74 (d, 2H), 3.07 (s, 3H).

HPLC (method A): Rₜ=3.63 min.

MS (DCI, NH₃): m/z=207 [M+NH₄]⁺.

LC/MS (method D, ESIpos): Rₜ=1.50 min, m/z=190 [M+H]⁺.

### Step 2: N′-Hydroxy-4-(3-methoxyoxetan-3-yl)benzenecarboximide amide

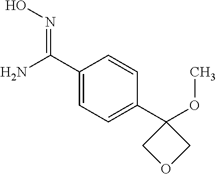 [SMILES: COC1(c2ccc(/C(N)=N/O)cc2)COC1]

By the process described under Example 1A/step 5, 520 mg (89% of th.) of the title compound were obtained from 500 mg (2.64 mmol) of the compound from Example 6A/step 1.

¹H-NMR (400 MHz, DMSO-d₆, δ/ppm): 9.67 (s, 1H), 7.73 (d, 2H), 7.43 (d, 2H), 5.83 (broad s, 2H), 4.77 (m, 4H), 3.03 (s, 3H).

HPLC (method A): Rₜ=2.54 min.

MS (DCI, NH₃): m/z=223 [M+H]⁺.

### Example 7A

### 4-(4-Fluorotetrahydro-2H-pyran-4-yl)-N′-hydroxybenzenecarboximide amide

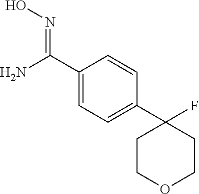 [SMILES: N/C(=N\O)c1ccc(C2(F)CCOCC2)cc1]

### Step 1: 4-(4-Hydroxytetrahydro-2H-pyran-4-yl)benzenecarbonitrile

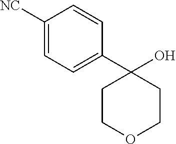 [SMILES: N#Cc1ccc(C2(O)CCOCC2)cc1]

By the process described under Example 5A/step 1, 25.0 g (109 mmol) of 4-iodobenzonitrile were reacted with 16.4 g (164 mmol) of tetrahydro-4H-pyran-4-one to give 7.56 g (34% of th.) of the title compound.

¹H-NMR (400 MHz, DMSO-d₆, δ/ppm): 7.80 (d, 2H), 7.70 (d, 2H), 5.30 (s, 1H), 3.81-3.70 (m, 4H), 2.02-1.94 (m, 2H), 1.51-1.48 (m, 2H).

HPLC (method A): Rₜ=3.35 min.

MS (DCI, NH₃): m/z=204 [M+H]⁺, 221 [M+NH₄]⁺.

### Step 2: 4-(4-Fluorotetrahydro-2H-pyran-4-yl)benzenecarbonitrile

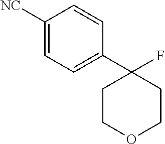 [SMILES: N#Cc1ccc(C2(F)CCOCC2)cc1]

By the process described under Example 5A/step 2, 6.5 g (31.98 mmol) of the compound from Example 7A/step 1 were reacted to give 3.73 g (57% of th.) of the title compound.

¹H-NMR (400 MHz, CDCl₃, δ/ppm): 7.68 (d, 2H), 7.50 (d, 2H), 3.98-3.83 (m, 4H), 2.23-2.05 (m, 2H), 1.91-1.85 (m, 2H).

HPLC (method A): Rₜ=4.04 min.

MS (DCI, NH₃): m/z=223 [M+NH₄]⁺.

### Step 3: 4-(4-Fluorotetrahydro-2H-pyran-4-yl)-N′-hydroxybenzenecarboximide amide

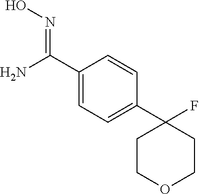 [SMILES: N/C(=N\O)c1ccc(C2(F)CCOCC2)cc1]

By the process described under Example 1A/step 5, 3.57 mg (88% of th.) of the title compound were obtained from 3.5 g (17.05 mmol) of the compound from Example 7A/step 2.

¹H-NMR (500 MHz, DMSO-d₆, δ/ppm): 9.64 (s, 1H), 7.70 (d, 2H), 7.44 (d, 2H), 5.81 (s, 2H), 3.88-3.83 (m, 2H), 3.73-3.67 (m, 2H), 2.23-2.06 (m, 2H), 1.87-1.81 (m, 2H).

HPLC (method A): Rₜ=3.06 min.

MS (DCI, NH₃): m/z=239 [M+H]⁺.

LC/MS (method F, ESIpos): Rₜ=0.40 min, m/z=239 [M+H]⁺.

### Example 8A

### N′-Hydroxy-4-(4-methoxytetrahydro-2H-pyran-4-yl)benzenecarboximide amide

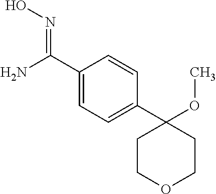 [SMILES: COC1(c2ccc(/C(N)=N/O)cc2)CCOCC1]

### Step 1: 4-(4-Methoxytetrahydro-2H-pyran-4-yl)benzenecarbonitrile

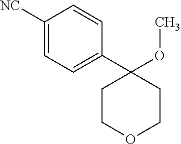 [SMILES: COC1(c2ccc(C#N)cc2)CCOCC1]

By the process described under Example 6A/step 1, 238 mg (74% of th.) of the title compound were obtained from 300 mg (1.48 mmol) of the compound from Example 7A/step 1 and 111 μl (1.77 mmol) of methyl iodide.

¹H-NMR (500 MHz, CDCl₃, δ/ppm): 7.68 (d, 2H), 7.51 (d, 2H), 3.89-3.82 (m, 4H), 2.99 (s, 3H), 2.03-1.98 (m, 2H), 1.94-1.91 (m, 2H).

HPLC (method A): Rₜ=3.99 min.

MS (DCI, NH₃): m/z=235 [M+NH₄]⁺.

GC/MS (method L, EIpos): Rₜ=6.57 min, m/z=217 (M)⁺.

### Step 2: N′-Hydroxy-4-(4-methoxytetrahydro-2H-pyran-4-yl)benzenecarboximide amide

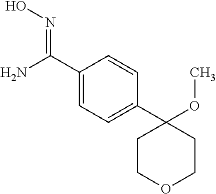 [SMILES: COC1(c2ccc(/C(N)=N/O)cc2)CCOCC1]

By the process described under Example 1A/step 5, 229 mg (99% of th.) of the title compound were obtained from 200 mg (0.921 mmol) of the compound from Example 8A/step 1.

¹H-NMR (400 MHz, DMSO-d₆, δ/ppm): 9.63 (s, 1H), 7.68 (d, 2H), 7.39 (d, 2H), 5.80 (s, 2H), 3.71-3.67 (m, 4H), 2.88 (m, 2H), 1.93-1.89 (m, 4H).

HPLC (method B): Rₜ=2.95 min.

MS (DCI, NH₃): m/z=251 [M+H]⁺.

LC/MS (method D, ESIpos): Rₜ=0.93 min, m/z=251 [M+H]⁺.

Analogously to the process described under Example 1A/step 5, the N′-hydroxybenzenecarboximide amides listed in the following table were prepared from the corresponding commercially obtainable benzonitriles. The benzonitriles which are not commercially obtainable were prepared in accordance with the following instructions in the literature: 4-cyclohexylbenzenecarbonitrile [E. Riguet et al., J. Organomet. Chem. 2001, 624 (1-2), 376-379], 4-(piperidin-1-yl)benzenecarbonitrile [A.-H. Kuthier et al., J. Org. Chem. 1987, 52 (9), 1710-1713], 4-(pentafluoro-λ⁶-sulfanyl)benzenecarbonitrile [P J. Crowley et al., Chimia 2004, 58 (3), 138-142].

| | | HPLC: | MS: m/z | LC/MS |
|---|---|---|---|---|
| Example | Structure | Rₜ [min] | [M + H]⁺ | method |
| 9A | 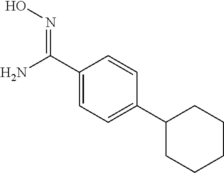 [SMILES: N/C(=N\O)c1ccc(C2CCCCC2)cc1] | 1.24 | 219 | H |
| | ¹H-NMR (400 MHz, DMSO-d₆, δ/ppm): 9.51 (s, 1H), 7.56 (d, 2H), 7.20 (d, 2H), 5.72 (s, broad, 2H), 2.52-2.48 (m, 1H), 1.81-1.74 (m, 4H), 1.73-1.67 (m, 1H), 1.45-1.31 (m, 4H), 1.28-1.19 (m, 1H). | | | |
| 10A | 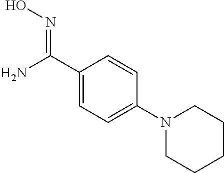 [SMILES: N/C(=N\O)c1ccc(N2CCCCC2)cc1] | 1.11 | 220 | D |
| | ¹H-NMR (400 MHz, CDCl₃, δ/ppm): 7.50 (d, 2H), 6.90 (d, 2H), 4.80 (s, broad, 2H), 3.23-3.20 (m, 4H), 1.71-1.65 (m, 4H), 1.63-1.57 (m, 2H). | | | |
| 11A | 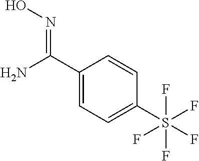 [SMILES: N/C(=N\O)c1ccc(S(F)(F)(F)(F)F)cc1] | 1.49 | 263 | D |
| | ¹H-NMR (400 MHz, DMSO-d₆, δ/ppm): 9.99 (s, 1H), 7.94-7.85 (m, 4H), 6.00 (s, 2H). | | | |
| 12A | 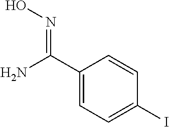 [SMILES: N/C(=N\O)c1ccc(I)cc1] | 1.98 | 263 | G |
| | ¹H-NMR (400 MHz, DMSO-d₆, δ/ppm): 9.71 (s, 1H), 7.73 (d, 2H), 7.47 (d, 2H), 5.84 (s, broad, 2H). | | | |
| 13A | 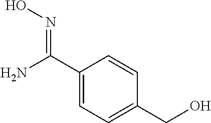 [SMILES: N/C(=N\O)c1ccc(CO)cc1] | 0.24 | 167 | D |
| | ¹H-NMR (400 MHz, DMSO-d₆, δ/ppm): 9.55 (s, 1H), 7.62 (d, 2H), 7.29 (d, 2H), 5.78 (s, 2H), 5.20 (t, 1H), 4.50 (d, 2H). | | | |
| 14A | 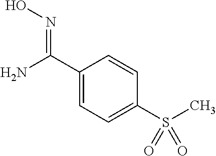 | 0.21 | 215 | F |
| | ¹H-NMR (400 MHz, DMSO-d₆, δ/ppm): 9.98 (s, 1H), 7.92 (s, 4H), 6.00 (s, broad, 2H), 3.23 (s, 3H). | | | |
| 15A | 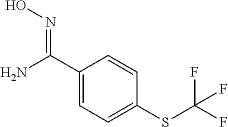 [SMILES: N/C(=N\O)c1ccc(SC(F)(F)F)cc1] | 1.42 | 237 | D |
| | ¹H-NMR (400 MHz, DMSO-d₆, δ/ppm): 9.90 (s, 1H), 7.80 (d, 2H), 7.72 (d, 2H), 5.94 (s, 2H). | | | |
| 16A | 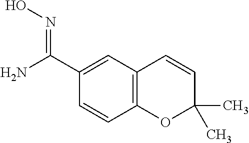 [SMILES: CC1(C)C=Cc2cc(/C(N)=N/O)ccc2O1] | 0.65 | 219 | F |
| | ¹H-NMR (400 MHz, CDCl₃, δ/ppm): 11.2 (very broad, 1H), 7.35 (dd, 1H), 7.26 (d, 1H), 6.78 (d, 1H), 6.31 (d, 1H), 5.63 (d, 1H), 4.82 (broad, 2H), 1.43 (s, 6H). | | | |
| 17A | 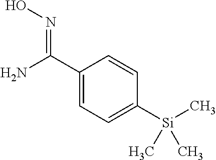 | 0.75 | 209 | I |
| | ¹H-NMR (400 MHz, CDCl₃, δ/ppm): 7.95 (s, broad, 1H), 7.60 (d, 2H), 7.55 (d, 2H), 4.86 (s, broad, 2H), 0.27 (s, 9H). | | | |
| 18A | 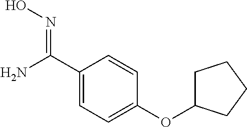 [SMILES: N/C(=N\O)c1ccc(OC2CCCC2)cc1] | 3.69 | 221 | A |
| | ¹H-NMR (400 MHz, DMSO-d₆, δ/ppm): 9.43 (s, 1H), 7.57 (d, 2H), 6.87 (d, 2H), 5.70 (s, broad, 2H), 4.84-4.81 (m, 1H), 1.97-1.88 (m, 2H), 1.73-1.66 (m, 4H), 1.62-1.53 (m, 2H). | | | |
| 19A | 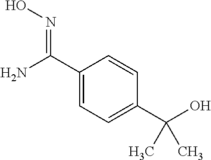 | 0.78 | 195 | D |
| | ¹H-NMR (400 MHz, DMSO-d₆, δ/ppm): 9.53 (s, 1H), 7.59 (d, 2H), 7.44 (d, 2H), 5.74 (s, broad, 2H), 5.02 (s, 1H), 1.41 (s, 6H). | | | |
| 20A | 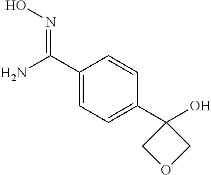 [SMILES: N/C(=N\O)c1ccc(C2(O)COC2)cc1] | 0.39 | 209 | D |
| | ¹H-NMR (400 MHz, DMSO-d₆, δ/ppm): 9.61 (s, 1H), 7.70 (d, 2H), 7.59 (d, 2H), 6.37 (s, 1H), 5.79 (s, broad, 2H), 4.76 (d, 2H), 4.68 (d, 2H). | | | |
| 21A | 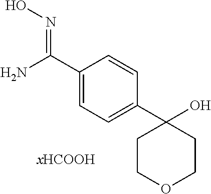 [SMILES: N/C(=N\O)c1ccc(C2(O)CCOCC2)cc1] | 0.72 | 237 | D |
| | ¹H-NMR (400 MHz, DMSO-d₆, δ/ppm): 9.71 (broad, 1H), 9.59 (s, 1H), 7.62 (d, 2H), 7.48 (d, 2H), 7.17 (broad, 1H), 5.78 (s, broad, 2H), 5.06 (s, 1H), 3.78 (dd, 2H), 3.72- 3.69 (m, 2H), 1.97 (dt, 2H), 1.52 (d, 2H). | | | |
| 22A | 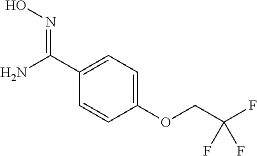 [SMILES: N/C(=N\O)c1ccc(OCC(F)(F)F)cc1] | 0.51 | 235 | I |
| | ¹H-NMR (400 MHz, DMSO-d₆, δ/ppm): 9.51 (s, 1H), 7.64 (d, 2H), 7.06 (d, 2H), 5.77 (s, broad, 2H), 4.79 (quart, 2H). | | | |
| 23A | 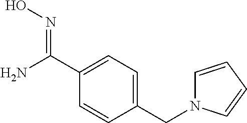 [SMILES: N/C(=N\O)c1ccc(Cn2cccc2)cc1] | 0.54 | 216 | I |

¹H-NMR (400 MHz, CDCl₃, δ/ppm): 7.76 (broad, 1H), 7.58 (d, 2H), 7.13 (d, 2H), 6.68 (dd, 2H), 6.20 (dd, 2H), 5.09 (s, 2H), 4.84 (s, broad, 2H).

### Example 24A

### N′-Hydroxy-4-(1-hydroxycyclobutyl)benzenecarboximide amide

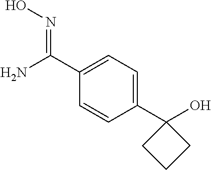 [SMILES: N/C(=N\O)c1ccc(C2(O)CCC2)cc1]

### Step 1: 4-(1-Hydroxycyclobutyl)benzenecarbonitrile

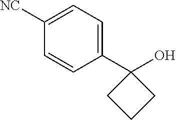 [SMILES: N#Cc1ccc(C2(O)CCC2)cc1]

Analogously to the process described under Example 5A/step 1, 9.47 g (83% of th.) of the title compound were obtained from 15.0 g (65.5 mmol) of 4-iodobenzonitrile, 34.4 ml (68.8 mmol) of isopropylmagnesium chloride solution (2 M in diethyl ether) and 7.4 ml (98.2 mmol) of cyclobutanone. The purification of the product was carried out by means of MPLC (silica gel, mobile phase: cyclohexane/ethyl acetate 10:1, 4:1).

¹H-NMR (400 MHz, CDCl₃, δ/ppm): 7.67 (d, 2H), 7.62 (d, 2H), 2.58-2.51 (m, 2H), 2.44-2.37 (m, 2H), 2.23-2.04 (m, 2H), 1.83-1.72 (m, 1H).

HPLC (method A): Rₜ=3.47 min.

MS (DCI, NH₃): m/z=191 [M+NH₄]⁺.

### Step 2: N′-Hydroxy-4-(1-hydroxycyclobutyl)benzenecarboximide amide

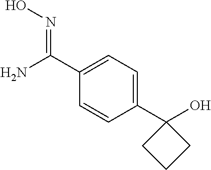 [SMILES: N/C(=N\O)c1ccc(C2(O)CCC2)cc1]

Analogously to the process described under Example 1A/step 5, 1.1 g of the title compound (92% of th.) were obtained starting from 1.0 g (5.77 mmol) of the compound from Example 24A/step 1. In contrast to that described under Example 1A/step 5, however, after removal of the solvent approx. 50 ml of water were added to the residue and the mixture was extracted three times with approx. 50 ml of ethyl acetate each time. The combined organic extracts were washed with saturated sodium chloride solution and dried over anhydrous magnesium sulfate. After filtration, the solvent was removed on a rotary evaporator and the residue obtained was purified by means of MPLC (silica gel, mobile phase: methylene chloride/methanol 50:1→10:1).

¹H-NMR (400 MHz, DMSO-d₆, δ/ppm): 9.57 (s, 1H), 7.63 (d, 2H), 7.47 (d, 2H), 5.79 (s, broad, 2H), 5.50 (s, 1H), 2.42-2.33 (m, 2H), 2.30-2.22 (m, 2H), 1.97-1.60 (m, 1H), 1.70-1.59 (m, 1H).

HPLC (method A): Rₜ=2.26 min.

MS (EIpos): m/z=207 [M+H]⁺.

LC/MS (method I, ESIpos): Rₜ=0.25 min, m/z=207 [M+H]⁺.

### Example 25A

### N′-Hydroxy-4-(1-methoxycyclobutyl)benzenecarboximide amide

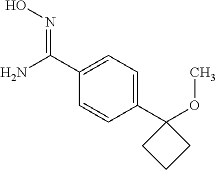 [SMILES: COC1(c2ccc(/C(N)=N/O)cc2)CCC1]

### Step 1: 4-(1-Methoxycyclobutyl)benzenecarbonitrile

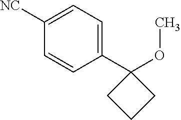

Analogously to the process described under Example 6A/step 1, 1.27 g (59% of th.) of the title compound were obtained from 2.0 g (11.5 mmol) of the compound from Example 24A/step 1, 508 mg (12.7 mmol) of a 60% strength dispersion of sodium hydride in mineral oil and 863 μl (13.9 mmol) of methyl iodide. The purification of the product was carried out by means of MPLC (silica gel; mobile phase: cyclohexane/ethyl acetate 20:1, 4:1).

¹H-NMR (400 MHz, CDCl₃, δ/ppm): 7.68 (d, 2H), 7.54 (d, 2H), 2.95 (s, 3H), 2.46-2.32 (m, 4H), 2.03-1.93 (m, 1H), 1.76-1.63 (m, 1H).

MS (DCI, NH₃): m/z=205 [M+NH₄]⁺.

### Step 2: N′-Hydroxy-4-(1-methoxycyclobutyl)benzenecarboximide amide

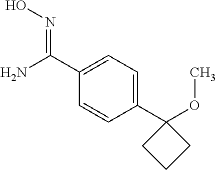 [SMILES: COC1(c2ccc(/C(N)=N/O)cc2)CCC1]

Analogously to the process described under Example 1A/step 5, 1.28 g of the title compound (98% of th.) were obtained starting from 1.1 g (5.87 mmol) of the compound from Example 25A/step 1.

¹H-NMR (400 MHz, DMSO-d₆, δ/ppm): 9.62 (s, 1H), 7.68 (d, 2H), 7.40 (d, 2H), 5.80 (s, broad, 2H), 2.83 (s, 3H), 2.37-2.24 (m, 4H), 1.91-1.81 (m, 1H), 1.65-1.53 (m, 1H).

HPLC (method A): Rₜ=3.02 min.

MS (DCI, NH₃): m/z=221 [M+H]⁺.

### Example 26A

### 4-(1-Fluorocyclobutyl)-N′-hydroxybenzenecarboximide amide

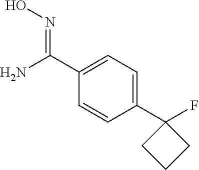 [SMILES: N/C(=N\O)c1ccc(C2(F)CCC2)cc1]

### Step 1: 4-(1-Fluorocyclobutyl)benzenecarbonitrile

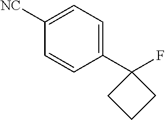 [SMILES: N#Cc1ccc(C2(F)CCC2)cc1]

Analogously to the process described under Example 5A/step 2, 1.39 g (69% of th.) of the title compound were obtained from 2.0 g (11.5 mmol) of the compound from Example 24A/step 1 and 1.8 ml (13.9 mmol) of diethylaminosulfur trifluoride (DAST). The purification of the product was carried out by means of MPLC (silica gel, mobile phase: cyclohexane/ethyl acetate 10:1,→5:1).

¹H-NMR (400 MHz, CDCl₃, δ/ppm): 7.69 (d, 2H), 7.57 (d, 2H), 2.78-2.62 (m, 2H), 2.58-2.48 (m, 2H), 2.20-2.09 (m, 1H), 1.87-1.75 (m, 1H).

GC/MS (method L, EIpos): Rₜ=4.71 min, m/z=155 [M−HF]±.

### Step 2: 4-(1-Fluorocyclobutyl)-N′-hydroxybenzenecarboximide amide

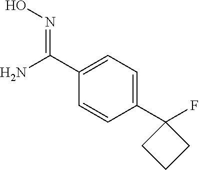 [SMILES: N/C(=N\O)c1ccc(C2(F)CCC2)cc1]

Analogously to the process described under Example 1A/step 5, 1.16 g of the title compound (78% of th.) were obtained starting from 1.25 g (7.13 mmol) of the compound from Example 26A/step 1.

¹H-NMR (400 MHz, CDCl₃, δ/ppm): 7.67 (d, 2H), 7.50 (d, 2H), 4.87 (s, broad, 2H), 2.72-2.52 (m, 5H), 2.16-2.05 (m, 1H), 1.82-1.71 (m, 1H).

HPLC (method A): Rₜ=3.17 min.

MS (DCI, NH₃): m/z=209 [M+H]⁺.

### Example 27A

### 2-Amino-2-[4-(trifluoromethoxy)phenyl]ethanol

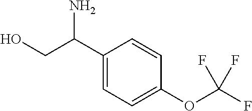 [SMILES: NC(CO)c1ccc(OC(F)(F)F)cc1]

834 mg (38.3 mmol) of lithium borohydride and 1 ml (19.1 mmol) of concentrated sulfuric acid, dissolved in 1 ml of THF, were added successively to a solution of 3.0 g (12.8 mmol) of racemic 4-(trifluoromethoxy)phenylglycine in 20 ml of THF. The reaction mixture was stirred at RT for 24 h. 15 ml of methanol were then added and the mixture was stirred until a clear solution formed. 20 ml of 4 M sodium hydroxide solution were then added dropwise to this solution. A precipitate thereby precipitated out, and was filtered off with suction and discarded. The filtrate was freed from the organic solvents on a rotary evaporator. The residue was extracted three times with approx. 20 ml of toluene each time. The combined organic extracts were concentrated on a rotary evaporator. 2.25 g (80% of th.) of the title compound were obtained.

¹H-NMR (400 MHz, DMSO-d₆, δ/ppm): 7.48 (d, 2H), 7.31 (d, 2H), 5.63 and 5.51 (each broad, tog. 2H), 4.91 (broad, 1H), 3.71-3.67 (m, 1H), 3.66-3.59 (m, 2H).

MS (DCI, NH₃): m/z=222 [M+H]⁺.

### Example 28A

### 5-(5-Methyl-1H-pyrazol-3-yl)-3-[4-(trifluoromethoxy)phenyl]-1,2,4-oxadiazole

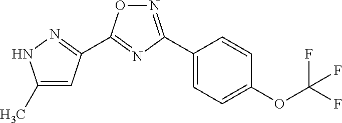 [SMILES: Cc1cc(-c2nc(-c3ccc(OC(F)(F)F)cc3)no2)n[nH]1]

23.3 g (0.121 mol) of EDC, 16.4 g (0.121 mol) of HOBt and 26.7 g (0.121 mol) of N′-hydroxy-4-(trifluoromethoxy)benzenecarboximide amide were added successively to a solution of 15.3 g (0.121 mol) of 5-methyl-1H-pyrazole-3-carboxylic acid in 600 ml of anhydrous DMF at RT. The mixture was stirred first at RT for 2 h and then at 140° C. for 5 h. After cooling, the mixture was diluted with 2 litres of water and extracted three times with 1 litre of ethyl acetate each time. The combined organic extracts were washed successively with water and saturated sodium chloride solution. After drying over anhydrous magnesium sulfate, the mixture was filtered and the solvent was removed on a rotary evaporator. The crude product obtained was purified by means of filtration with suction over a suction filter filled with silica gel (eluent: cyclohexane/ethyl acetate 5:1→1:1). The product fractions were combined and the solvent was removed on a rotary evaporator to such an extent that the product just started to precipitate out. The precipitation was brought to completion at RT. By filtration and further concentration of the mother liquor, two fractions of solid were obtained, which were combined and dried under a high vacuum. 19.7 g (52% of th.) of the title compound were obtained in total in this way.

¹H-NMR (400 MHz, CDCl₃, δ/ppm): 10.75 (broad, 1H), 8.24 (d, 2H), 7.34 (d, 2H), 6.81 (s, 1H), 2.46 (s, 3H).

HPLC (method A): Rₜ=4.72 min.

MS (DCI, NH₃): m/z=311 [M+H]⁺.

LC/MS (method F, ESIpos): Rₜ=1.27 min, m/z=311 [M+H]⁺.

The compounds listed in the following table were prepared by the process described in Example 28A from 5-methyl-1H-pyrazole-3-carboxylic acid, 5-(trifluoromethyl)-1H-pyrazole-3-carboxylic acid, 5-nitro-1H-pyrazole-3-carboxylic acid or 2-methyl-1H-imidazole-4-carboxylic acid hydrate and the corresponding N′-hydroxybenzenecarboximide amides. The reaction time during which stirring was initially carried out at RT was 0.5 to 4 h, depending on the size of the batch. The mixture was subsequently heated at 140° C. for 1 to 15 h. Depending on the polarity of the product obtained, this already precipitated out on addition of water after the reaction had ended, and it was then washed and dried under a high vacuum. Alternatively, as described above, the mixture was worked up by extraction and the product was then purified by chromatography over silica gel; various mobile phases were used for the chromatography. In some cases it was possible to omit the chromatography and to purify the product directly by extraction by stirring in methylene chloride, ethyl acetate, acetonitrile or tert-butyl methyl ether. The compound in Example 41A was purified by means of preparative HPLC (method N).

| | | HPLC: | MS: m/z | LC/MS |
|---|---|---|---|---|
| Example | Structure | Rₜ [min] | [M + H]⁺ | method |
| 29A | 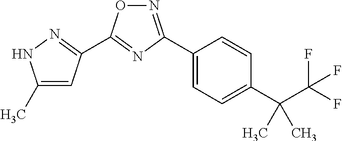 [SMILES: Cc1cc(-c2nc(-c3ccc(C(C)(C)C(F)(F)F)cc3)no2)n[nH]1] | 1.34 | 337 | F |
| | ¹H-NMR (400 MHz, DMSO-d₆, δ/ppm): 11.80 (s, broad, 1H), 8.17 (d, 2H), 7.63 (d, 2H), 6.83 (s, 1H), 2.46 (s, 3H), 1.63 (s, 6H). | | | |
| 30A | 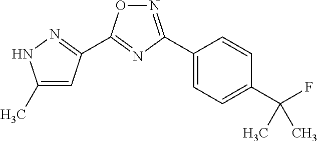 [SMILES: Cc1cc(-c2nc(-c3ccc(C(C)(C)F)cc3)no2)n[nH]1] | 2.19 | 287 | D |
| | ¹H-NMR (400 MHz, DMSO-d₆, δ/ppm): 13.54 (s, broad, 1H), 8.08 (d, 2H), 7.62 (d, 2H), 6.81 (s, 1H), 2.33 (s, 3H), 1.72 (s, 3H), 1.68 (s, 3H). | | | |
| 31A | 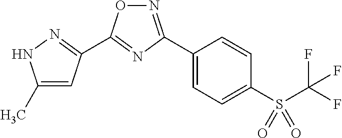 [SMILES: Cc1cc(-c2nc(-c3ccc(S(=O)(=O)C(F)(F)F)cc3)no2)n[nH]1] | 1.25 | 359 | F |
| | ¹H-NMR (400 MHz, DMSO-d₆, δ/ppm): 13.62 (s, broad, 1H), 8.49 (d, 2H), 8.38 (d, 2H), 6.83 (s, 1H), 2.34 (s, 3H). | | | |
| 32A | 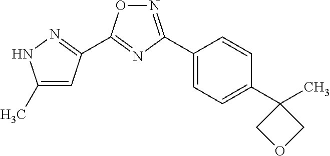 | 1.98 | 297 | C |
| | ¹H-NMR (400 MHz, CDCl₃, δ/ppm): 8.17 (d, 2H), 7.33 (d, 2H), 6.82 (s, 1H), 5.00 (d, 2H), 4.68 (d, 2H), 2.45 (s, 3H), 1.77 (s, 3H). | | | |
| 33A | 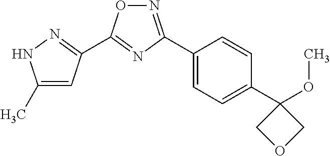 | 0.99 | 313 | F |
| | ¹H-NMR (400 MHz, DMSO-d₆, δ/ppm): 13.54 (s, broad, 1H), 8.14 (d, 2H), 7.69 (d, 2H), 6.80 (s, 1H), 4.82 (d, 2H), 4.78 (d, 2H), 3.08 (s, 3H), 2.37 (s, 3H). | | | |
| 34A | 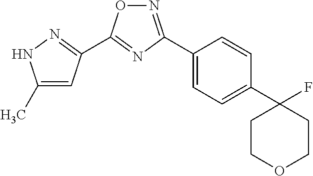 [SMILES: Cc1cc(-c2nc(-c3ccc(C4(F)CCOCC4)cc3)no2)n[nH]1] | 4.24 | 329 | C |
| | ¹H-NMR (400 MHz, CDCl₃, δ/ppm): 10.73 (broad, 1H), 8.20 (d, 2H), 7.52 (d, 2H), 6.81 (s, 1H), 4.00-3.88 (m, 4H), 2.45 (s, 3H), 2.30-2.11 (m, 2H), 1.98-1.91 (m, 2H). | | | |
| 35A | 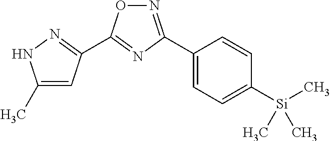 | 2.39 | 299 | E |
| | ¹H-NMR (400 MHz, CDCl₃, δ/ppm): 11.3 (s, broad, 1H), 8.12 (d, 2H), 7.63 (d, 2H), 6.81 (s, 1H), 2.43 (s, 3H), 0.31 (s, 9H). | | | |
| 36A | 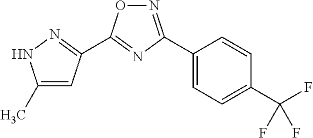 [SMILES: Cc1cc(-c2nc(-c3ccc(C(F)(F)F)cc3)no2)n[nH]1] | 1.11 | 295 | I |
| | ¹H-NMR (400 MHz, CDCl₃, δ/ppm): 10.52 (broad, 1H), 8.32 (d, 2H), 7.77 (d, 2H), 6.82 (s, 1H), 2.63 (s, 3H). | | | |
| 37A | 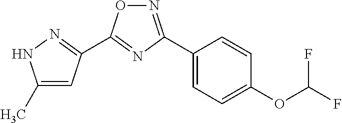 [SMILES: Cc1cc(-c2nc(-c3ccc(OC(F)F)cc3)no2)n[nH]1] | 1.02 | 293 | I |
| | ¹H-NMR (400 MHz, CDCl₃, δ/ppm): 10.85 (broad, 1H), 8.20 (d, 2H), 7.23 (d, 2H), 6.81 (s, 1H), 6.60 (t, 1H), 2.46 (s, 3H). | | | |
| 38A | 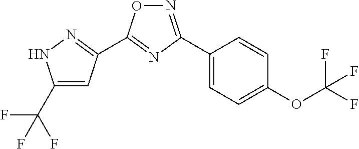 [SMILES: FC(F)(F)Oc1ccc(-c2noc(-c3cc(C(F)(F)F)[nH]n3)n2)cc1] | 2.41 | 365 | E |
| | ¹H-NMR (400 MHz, CDCl₃, δ/ppm): 11.73 (broad, 1H), 8.19 (d, 2H), 7.38 (d, 2H), 7.37 (s, 1H). | | | |
| 39A | 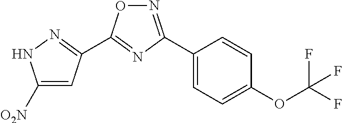 [SMILES: O=[N+]([O-])c1cc(-c2nc(-c3ccc(OC(F)(F)F)cc3)no2)n[nH]1] | 2.18 | 342 | E |
| | ¹H-NMR (500 MHz, DMSO-d₆, δ/ppm): 8.20 (d, 2H), 7.58 (d, 2H), 7.34 (s, 1H). | | | |
| 40A | 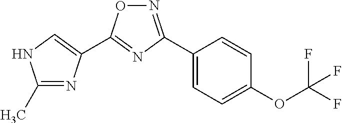 [SMILES: Cc1nc(-c2nc(-c3ccc(OC(F)(F)F)cc3)no2)c[nH]1] | 1.08 | 311 | F |
| | ¹H-NMR (400 MHz, CDCl₃, δ/ppm): 8.23 (d, 2H), 7.82 (d, 2H), 7.33 (s, 1H), 2.56 (s, 3H). | | | |
| 41A | 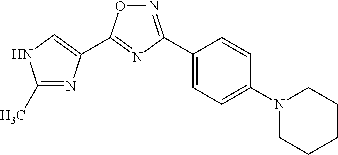 [SMILES: Cc1nc(-c2nc(-c3ccc(N4CCCCC4)cc3)no2)c[nH]1] | 0.97 | 310 | F |

¹H-NMR (400 MHz, CDCl₃, δ/ppm): 9.61 (broad, 1H), 8.02 (d, 2H), 7.79 (s, 1H), 6.96 (d, 2H), 3.31-3.27 (m, 4H), 2.54 (s, 3H), 1.73-1.61 (m, 6H).

### Example 42A

### 3-{3-[4-(Trifluoromethoxy)phenyl]-1,2,4-oxadiazol-5-yl}-1H-pyrazol-5-amine

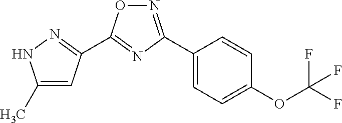 [SMILES: Cc1cc(-c2nc(-c3ccc(OC(F)(F)F)cc3)no2)n[nH]1]

A solution of 342 mg (1.0 mmol) of the compound from Example 39A in 43 ml of ethyl acetate was hydrogenated in a flow-through hydrogenation apparatus (“H-Cube” from ThalesNano, Budapest, Hungary) (conditions: 10% Pd/C catalyst, 1 bar of H₂, 25° C., 1 ml/min) After removal of the solvent on a rotary evaporator, the crude product was purified by means of MPLC (silica gel, cyclohexane/ethyl acetate 1:1). 322 mg (93% of th.) of the title compound were obtained.

¹H-NMR (400 MHz, DMSO-d₆, δ/ppm): 12.49 (s, 1H), 8.19 (d, 2H), 7.49 (d, 2H), 5.93 (s, 1H), 5.44 (s, 2H).

MS (DCI, NH₃): m/z=312 [M+H]⁺.

LC/MS (method E, ESIpos): Rₜ=1.76 min, m/z=312 [M+H]⁺.

### Example 43A

### 2-Chloro-4-(chloromethyl)pyridine

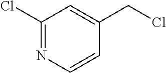

1.00 g (6.97 mmol) of (2-chloropyridin-4-yl)methanol was dissolved in 40 ml of methylene chloride, 10 ml of thionyl chloride were slowly added at RT and the mixture was stirred at RT overnight. The mixture was then concentrated on a rotary evaporator and the residue was stirred in a mixture of methylene chloride and aqueous sodium bicarbonate solution. The phases were separated and the methylene chloride phase was dried over anhydrous magnesium sulfate, filtered and concentrated on a rotary evaporator. 1.10 g (97% of th.) of the title compound were obtained.

¹H-NMR (400 MHz, CDCl₃, δ/ppm): 8.49 (d, 1H), 7.38 (s, 1H), 7.27-7.22 (m, 1H), 4.52 (s, 2H).

LC/MS (method E, ESIpos): Rₜ=1.43 min, m/z=162 [M+H]⁺.

### Example 44A

### 2-(Chloromethyl)-5-iodopyridine

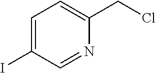

### Step 1: 2-(Hydroxymethyl)-5-iodopyridine

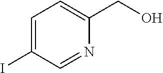

5.7 ml (9.07 mmol) of a 1.6 M solution of n-butyllithium in hexane were added dropwise to a solution of 2.50 g (7.56 mmol) of 2,5-diiodopyridine in 90 ml of toluene under inert conditions and at a temperature of −78° C. The mixture was stirred at −78° C. for 2.5 h and 756 μl of anhydrous DMF was then added at the same temperature. After a further 60 min at −78° C., the reaction mixture was allowed to warm to −10° C., 572 mg (15.11 mmol) of solid sodium borohydride were added and stirring was continued at 0° C. for 30 min. 25 ml of saturated aqueous ammonium chloride solution were then added and the mixture was warmed to RT. The organic phase was separated off and the solvent was removed on a rotary evaporator. The residue was purified by means of preparative HPLC. 890 mg (50% of th.) of the title compound (for the analytical data see below) and 243 mg (14% of th.) of the isomeric 5-(hydroxymethyl)-2-iodopyridine were obtained [preparative HPLC conditions: column: Sunfire C18 OBD 5 μm, 19 mm×150 mm; temperature: 40° C.; mobile phase: water/acetonitrile/1% strength aqueous TFA 76:5:19; flow rate: 25 ml/min; 1.3 g of crude product were dissolved in a mixture of 8 ml of 1% strength aqueous TFA and 4 ml of acetonitrile; injection volume: 1 ml].

¹H-NMR (400 MHz, CDCl₃, δ/ppm): 8.87 (d, 1H), 8.30 (dd, 1H), 7.38 (d, 1H), 5.43 (broad, 1H), 4.85 (s, 2H).

HPLC (method A): Rₜ=0.87 min.

MS (DCI, NH₃): m/z=236 [M+H]⁺.

LC/MS (method E, ESIpos): Rₜ=0.85 min, m/z=236 [M+H]⁺.

### Step 2: 2-(Chloromethyl)-5-iodopyridine

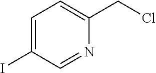

357 μl (4.88 mmol) of thionyl chloride were added dropwise to a solution of 765 mg (3.26 mmol) of the compound from Example 44A/step 1 in 12 ml of anhydrous methylene chloride at 0° C. The reaction mixture was then stirred at RT for 15 h. Approx. 50 ml of saturated aqueous sodium bicarbonate solution was then added and the mixture was extracted three times with approx. 50 ml of methylene chloride each time. The combined organic extracts were washed with saturated sodium chloride solution and dried over anhydrous magnesium sulfate. After filtration, the solvent was removed on a rotary evaporator. 541 mg (66% of th.) of the title compound were obtained.

¹H-NMR (400 MHz, CDCl₃, δ/ppm): 8.79 (d, 1H), 8.03 (dd, 1H), 7.29 (d, 1H), 4.61 (s, 2H).

MS (ESIpos): m/z=254/256 (³⁵Cl/³⁷Cl) [M+H]⁺.

LC/MS (method D, ESIpos): Rₜ=1.87 min, m/z=254/256 (³⁵Cl/³⁷Cl) [M+H]⁺.

### Example 45A

### 5-(Chloromethyl)pyridine-2-carbonitrile hydrochloride

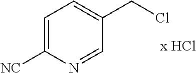

272 μl (3.73 mmol) of thionyl chloride were added to a solution of 250 mg (1.86 mmol) of 5-(hydroxymethyl)pyridine-2-carbonitrile [A. Ashimori et al., Chem. Pharm. Bull. 1990, 38 (9), 2446-2458] in 5 ml of anhydrous methylene chloride at 0° C. The reaction mixture was then stirred at RT for 6 h. All the volatile constituents were then removed on a rotary evaporator and the residue obtained was dried under a high vacuum. 263 mg (75% of th.) of the title compound were obtained.

¹H-NMR (400 MHz, CDCl₃, δ/ppm): 8.73 (d, 1H), 7.90 (dd, 1H), 7.72 (d, 1H), 4.63 (s, 2H).

MS (ESIpos): m/z=153/155 (³⁵Cl/³⁷Cl) [M+H]⁺.

LC/MS (method F, ESIpos): Rₜ=0.75 min, m/z=153/155 (³⁵Cl/³⁷Cl) [M+H]⁺.

### Example 46A

### (6-Cyanopyridin-3-yl)methyl methanesulfonate

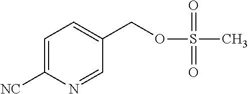

3.51 ml (27.14 mmol) of N,N-diisopropylethylamine and 2.87 ml (25.05 mmol) of methanesulfonic acid chloride were added successively to a solution of 2.8 g (20.87 mmol) of 5-(hydroxymethyl)pyridine-2-carbonitrile [A. Ashimori et al., Chem. Pharm. Bull. 1990, 38 (9), 2446-2458] in 50 ml of anhydrous methylene chloride at 0° C. The reaction mixture was then stirred at RT for 1 h. 10 ml of water were then added, the phases were separated and the aqueous phase was extracted twice with approx. 10 ml of methylene chloride each time. The combined organic extracts were washed with saturated sodium chloride solution, dried over anhydrous magnesium sulfate, filtered and freed from the solvent on a rotary evaporator. The residue obtained was separated into its components by means of MPLC (silica gel, cyclohexane/ethyl acetate 1:1). 2.12 g (48% of th.) of the title compound (for the analytical data see below) and 1.51 g (47% of th.) of the compound described in Example 45A were obtained.

¹H-NMR (400 MHz, CDCl₃, δ/ppm): 8.76 (d, 1H), 7.93 (dd, 1H), 7.78 (d, 1H), 5.32 (s, 2H), 3.10 (s, 3H).

MS (DCI, NH₃): m/z=213 [M+H]⁺, 230 [M+NH₄]⁺.

LC/MS (method F, ESIpos): Rₜ=0.57 min, m/z=213 [M+H]⁺.

### Example 47A

### [3-(Bromomethyl)phenoxy](tripropan-2-yl)silane

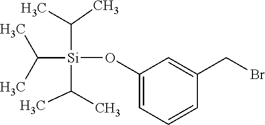 [SMILES: CC(C)[Si](Oc1cccc(CBr)c1)(C(C)C)C(C)C]

### Step 1: Ethyl 3-[(tripropan-2-ylsilyl)oxy]benzenecarboxylate

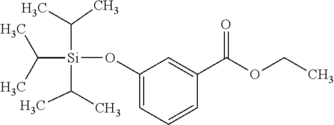 [SMILES: CCOC(=O)c1cccc(O[Si](C(C)C)(C(C)C)C(C)C)c1]

5.98 g (30.99 mmol) of triisopropylsilyl chloride were added dropwise to a solution of 5.0 g (30.09 mmol) of 3-hydroxybenzoic acid ethyl ester and 2.41 g (35.35 mmol) of imidazole in 20 ml of anhydrous DMF. After the reaction mixture had been stirred at RT for 15 h, approx. 100 ml of water were added and the mixture was extracted three times with approx. 100 ml of diethyl ether each time. The combined organic extracts were washed successively with water and saturated sodium chloride solution. After drying over anhydrous magnesium sulfate and filtration, the solvent was removed on a rotary evaporator. The residue obtained was purified by filtration with suction over silica gel with cyclohexane/ethyl acetate 10:1→1:1 as the mobile phase. 9.70 g (100% of th.) of the title compound were obtained.

¹H-NMR (400 MHz, CDCl₃, δ/ppm): 7.62 (dd, 1H), 7.53 (m, 1H), 7.28 (dd, 1H), 7.06 (dd, 1H), 4.37 (quart, 2H), 1.39 (t, 3H), 1.28 (sept, 3H), 1.10 (d, 18H).

GC/MS (method L, EI): Rₜ=6.62 min, m/z=322 (M)⁺, 279 (M−C₃H₇)⁺.

### Step 2: {3-[(Tripropan-2-ylsilyl)oxy]phenyl}methanol

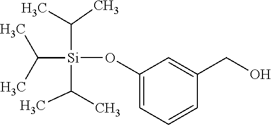 [SMILES: CC(C)[Si](Oc1cccc(CO)c1)(C(C)C)C(C)C]

Under inert conditions, 50 ml (49.61 mmol) of a 1 M solution of lithium aluminium hydride in THF were diluted with 50 ml of anhydrous diethyl ether, and a solution of 8.0 g (24.80 mmol) of the compound from Example 47A/step 1 in 50 ml of anhydrous diethyl ether was then added dropwise at 0° C. The reaction mixture was stirred at RT for 1 h. A few ml of methanol were then first added in order to solvolyse excess hydride, and then approx. 150 ml of 0.1M hydrochloric acid. The organic phase was separated off rapidly and the aqueous phase was extracted twice with approx. 50 ml of diethyl ether each time. The combined organic extracts were washed successively with water and saturated sodium chloride solution. After drying over anhydrous magnesium sulfate and subsequent filtration, the solvent was removed on a rotary evaporator. The residue obtained was purified by filtration with suction over silica gel with cyclohexane/ethyl acetate 5:1→1:1 as the mobile phase. 6.69 g (96% of th.) of the title compound were obtained.

¹H-NMR (400 MHz, CDCl₃, δ/ppm): 7.20 (dd, 1H), 6.93-6.90 (m, 2H), 6.80 (dd, 1H), 4.64 (d, 2H), 1.61 (t, 3H), 1.26 (sept, 3H), 1.09 (d, 18H).

GC/MS (method L, EI): Rₜ=6.38 min, m/z=280 (M)⁺, 237 (M−C₃H₇)⁺.

### Step 3: [3-(Bromomethyl)phenoxy](tripropan-2-yl)silane

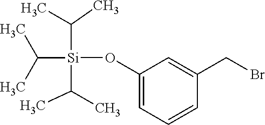 [SMILES: CC(C)[Si](Oc1cccc(CBr)c1)(C(C)C)C(C)C]

1.0 g (3.57 mmol) of the compound from Example 47A/step 2 was dissolved in 20 ml of anhydrous THF and 1.12 g (4.28 mmol) of triphenylphosphine were added. After this had dissolved, 1.42 g (4.28 mmol) of tetrabromomethane were added. The mixture was then stirred at RT for 20 h. The precipitate which had precipitated out was then filtered off and the filtrate was freed from the solvent on a rotary evaporator. The crude product was purified by means of MPLC (silica gel, cyclohexane/ethyl acetate 50:1). 1.10 g (90% of th., purity of approx. 90%) of the title compound were obtained, this being used without further purification.

¹H-NMR (400 MHz, CDCl₃, δ/ppm): 7.18 (dd, 1H), 6.95 (dd, 1H), 6.91 (m, 1H), 6.80 (dd, 1H), 4.43 (s, 2H), 1.25 (sept, 3H), 1.10 (d, 18H).

HPLC (method B): Rₜ=6.17 min.

GC/MS (method L, EI): Rₜ=6.56 min, m/z=342/344 (⁷⁹Br/⁸¹Br) (M)⁺.

### Example 48A

### Ethyl (4-{[(methylsulfonyl)oxy]methyl}phenyl)acetate

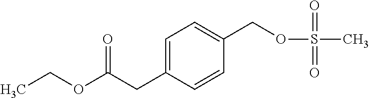 [SMILES: CCOC(=O)Cc1ccc(COS(C)(=O)=O)cc1]

A solution of 1.1 g (5.66 mmol) of [4-(hydroxymethyl)phenyl]acetic acid ethyl ester [G. Biagi et al., Farmaco Ed. Sci. 1988, 43 (7/8), 597-612] and 1.03 ml (7.36 mmol) of triethylamine in 10 ml of anhydrous THF was cooled to 0° C. A solution of 526 μl (6.80 mmol) of methanesulfonic acid chloride in 5 ml of anhydrous THF was then added dropwise. After 15 min at 0° C., the mixture was warmed to RT. After a further hour, approx. 60 ml of water were added and the mixture was extracted twice with approx. 50 ml of ethyl acetate each time. The combined organic extracts were washed with saturated sodium chloride solution. After drying over anhydrous magnesium sulfate and filtration, the solvent was removed on a rotary evaporator. The crude product was purified by means of MPLC (silica gel, cyclohexane/ethyl acetate 7:3). 1.19 g (56% of th., purity of approx. 73%) of the title compound were obtained, this being used without further purification.

MS (DCI, NH₃): m/z=290 [M+NH₄]⁺.

LC/MS (method C, ESIpos): Rₜ=1.96 min, m/z=177 (M−CH₃SO₂O)⁺.

### Example 49A

### 1-[(6-Chloropyridin-3-yl)methyl]-5-methyl-1H-pyrazole-3-carboxylic acid

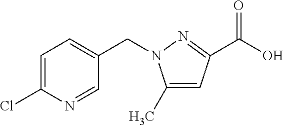 [SMILES: Cc1cc(C(=O)O)nn1Cc1ccc(Cl)nc1]

### Step 1: Ethyl 1-[(6-chloropyridin-3-yl)methyl]-5-methyl-1H-pyrazole-3-carboxylate

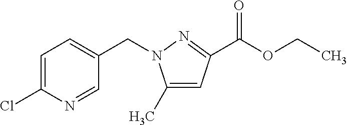 [SMILES: CCOC(=O)c1cc(C)n(Cc2ccc(Cl)nc2)n1]

9.46 g (84.3 mmol) of potassium tert-butylate were added to a solution of 10.0 g (64.9 mmol) of ethyl 3-methyl-1H-pyrazole-5-carboxylate and 13.66 g (84.3 mmol) of 2-chloro-5-(chloromethyl)pyridine in 162 ml of anhydrous THF at 0° C. The mixture was allowed to come to RT and was stirred at RT for a further 18 h. It was then diluted with 200 ml of ethyl acetate and 350 ml of water, the phases were mixed thoroughly and the aqueous phase, which was separated off, was extracted twice more with 200 ml of ethyl acetate each time. The combined organic phases were dried over anhydrous sodium sulfate, filtered and concentrated on a rotary evaporator. The residue was purified by column chromatography over silica gel (mobile phase: cyclohexane/ethyl acetate 4:1→2:1). After drying in vacuo, 12.4 g (65% of th.) of the title compound were obtained in a purity of 95%.

¹H-NMR (400 MHz, DMSO-d₆, δ/ppm): 8.30 (d, 1H), 7.58 (dd, 1H), 7.52 (d, 1H), 6.60 (s, 1H), 5.45 (s, 2H), 4.24 (quart, 2H), 2.28 (s, 3H), 1.27 (t, 3H).

LC/MS (method C, ESIpos): Rₜ=1.88 min, m/z=280 [M+H]⁺.

### Step 2: 1-[(6-Chloropyridin-3-yl)methyl]-5-methyl-1H-pyrazole-3-carboxylic acid

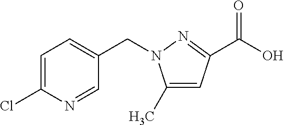 [SMILES: Cc1cc(C(=O)O)nn1Cc1ccc(Cl)nc1]

3.39 g (84.7 mmol) of sodium hydroxide, dissolved in 100 ml of water, were added to a solution of 11.85 g (42.36 mmol) of the compound from Example 49A/step 1 in 100 ml of THF and the mixture was stirred at RT for 5 h. The mixture was then diluted with 150 ml of water and washed once with 100 ml of ethyl acetate. The aqueous phase was adjusted to a pH of approx. 3 with 1 N hydrochloric acid and extracted three times with 150 ml of ethyl acetate each time. The latter ethyl acetate phases were combined, dried over anhydrous sodium sulfate, filtered and concentrated on a rotary evaporator. After the residue had been dried in vacuo, 9.72 g (91% of th.) of the title compound were obtained.

¹H-NMR (400 MHz, DMSO-d₆, δ/ppm): 12.60 (s, broad, 1H), 8.31 (d, 1H), 7.60 (dd, 1H), 7.52 (d, 1H), 6.53 (s, 1H), 5.42 (s, 2H), 2.28 (s, 3H).

LC/MS (method F, ESIpos): Rₜ=0.75 min, m/z=252 [M+H]⁺.

### Example 50A

### 1-[(6-Chloropyridin-3-yl)methyl]-5-methyl-1H-pyrrole-3-carboxylic acid

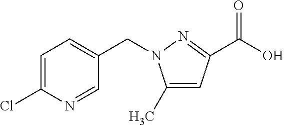 [SMILES: Cc1cc(C(=O)O)nn1Cc1ccc(Cl)nc1]

### Step 1: Methyl 2-(hydroxymethylidene)-4-oxopentanoate

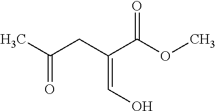

7.63 g (190.7 mmol) of a 60% strength suspension of sodium hydride in mineral oil were deoiled with pentane under inert conditions. 150 ml of anhydrous diethyl ether and, at 0° C., 138 μl (3.4 mmol) of methanol were then added. After stirring at RT for 10 min, the mixture was cooled to 0° C. again and a mixture of 12.6 ml (204.3 mmol) of formic acid methyl ester and 30.0 g (170.2 mmol) of methyl 4,4-dimethoxypentanoate [C. Meister et al., Liebigs Ann. Chem. 1983 (6), 913-921] was slowly added. The reaction mixture was stirred at RT for 16 h. Approx. 60 ml of ice-water were then added and the mixture was extracted with 100 ml of diethyl ether. The organic extract was discarded and the aqueous phase was brought to a pH of 2-3 with 3 M hydrochloric acid. It was extracted four times with approx. 50 ml of tert-butyl methyl ether each time. The combined organic extracts were dried over anhydrous magnesium sulfate, filtered and freed from the solvent on a rotary evaporator. 4.2 g (13% of th., purity of 85%) of the title compound were obtained, this being employed without further purification.

GC/MS (method L, EI): Rₜ=3.33 min, m/z=158 (M)⁺, 140 (M−H₂O)⁺.

### Step 2: Methyl 1-[(6-chloropyridin-3-yl)methyl]-5-methyl-1H-pyrrole-3-carboxylate

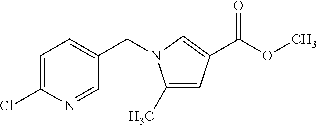 [SMILES: COC(=O)c1cc(C)n(Cc2ccc(Cl)nc2)c1]

A mixture of 4.20 g (22.73 mmol, purity of 85%) of the compound from Example 50A/step 1 and 3.24 g (22.73 mmol) of 5-(aminomethyl)-2-chloropyridine in 42 ml of methanol was stirred at RT for three days. The solvent was then removed on a rotary evaporator and the crude product was purified by means of MPLC (silica gel, cyclohexane/ethyl acetate 2:1). 3.37 g (56% of th.) of the title compound were obtained.

¹H-NMR (400 MHz, CDCl₃, δ/ppm): 8.19 (d, 1H), 7.30-7.20 (m, 3H), 6.38 (d, 1H), 5.03 (s, 2H), 3.79 (s, 3H), 2.12 (s, 3H).

HPLC (method A): Rₜ=4.10 min.

MS (DCI, NH₃): m/z=265 [M+H]⁺.

### Step 3: 1-[(6-Chloropyridin-3-yl)methyl]-5-methyl-1H-pyrrole-3-carboxylic acid

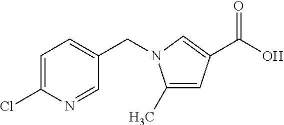 [SMILES: Cc1cc(C(=O)O)cn1Cc1ccc(Cl)nc1]

14.5 ml (14.5 mmol) of 1 M sodium hydroxide solution were added dropwise to a solution of 1.93 g (7.29 mmol) of the compound from Example 50A/step 2 in 38 ml of methanol. The reaction mixture was heated under reflux for 15 h. After cooling to RT, the methanol was mostly removed on a rotary evaporator. The residue was first diluted with 100 ml of water and then acidified with 2 M hydrochloric acid. The precipitate which had precipitated out was filtered off, rinsed with water and dried under a high vacuum. 1.41 g (76% of th.) of the title compound were obtained.

¹H-NMR (400 MHz, DMSO-d₆, δ/ppm): 11.67 (s, 1H), 8.23 (s, 1H), 7.51 (d, 2H), 7.45 (d, 2H), 6.18 (d, 1H), 5.19 (s, 2H), 2.07 (s, 3H).

HPLC (method A): Rₜ=3.59 min.

MS (ESIpos): m/z=251 [M+H]⁺.

### Example 51A

### 2-Chloro-5-[(2-methyl-4-{3-[4-(trifluoromethoxy)phenyl]-1,2,4-oxadiazol-5-yl}-1H-pyrrol-1-yl)-methyl]pyridine

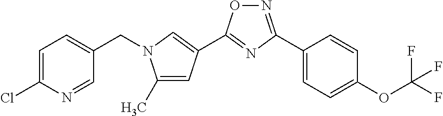 [SMILES: Cc1cc(-c2nc(-c3ccc(OC(F)(F)F)cc3)no2)cn1Cc1ccc(Cl)nc1]

418 μl (4.79 mmol) of oxalyl chloride were added to a solution of 400 mg (1.60 mmol) of the compound from Example 50A in 20 ml of anhydrous methylene chloride at 0° C. under inert conditions. The reaction mixture was stirred at RT for 2 h. All the volatile constituents were then removed on a rotary evaporator and the residue obtained in this way was dried under a high vacuum for 20 min. The residue was subsequently dissolved again in 4 ml of methylene chloride and this solution was added dropwise to a solution of 527 mg (2.39 mmol) of 4-(trifluoromethoxy)-N′-hydroxybenzenecaboximide amide and 445 μl (3.19 mmol) of triethylamine in 16 ml of methylene chloride at 0° C. After the reaction mixture had been stirred at RT for 16 h, all the volatile constituents were again removed on a rotary evaporator and the residue obtained was dissolved in 30 ml of DMSO. This solution was then heated at 140° C. in a microwave oven for 30 min (CEM Discover, initial irradiation power 250 W). After cooling to RT, the reaction mixture was purified by means of preparative HPLC (method N). 196 mg (28% of th.) of the title compound were obtained.

¹H-NMR (400 MHz, CDCl₃, δ/ppm): 8.24 (d, 1H), 8.17 (d, 2H), 7.47 (d, 1H), 7.32-7.27 (m, 4H), 6.60 (d, 1H), 5.10 (s, 2H), 2.20 (s, 3H).

LC/MS (method C, ESIpos): Rₜ=3.01 min, m/z=435 [M+H]⁺.

### Example 52A

### 2-Chloro-5-[(3-{3-[4-(2-fluoropropan-2-yl)phenyl]-1,2,4-oxadiazol-5-yl}-5-methyl-1H-pyrazol-1-yl)methyl]pyridine

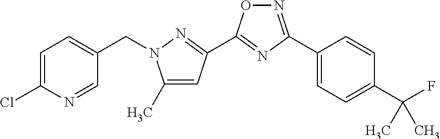 [SMILES: Cc1cc(-c2nc(-c3ccc(C(C)(C)F)cc3)no2)nn1Cc1ccc(Cl)nc1]

508 mg (2.65 mmol) of EDC and 358 mg (2.65 mmol) of HOBt were added to a solution of 667 mg (2.65 mmol) of the compound from Example 49A in 10 ml of anhydrous DMF at RT. After 30 min, 520 mg (2.65 mmol) of the compound from Example 2A, dissolved in 5 ml of DMF, were added. The mixture was stirred first at RT for 1 h and then at 140° C. for 1 h. After cooling, the majority of the solvent was removed on a rotary evaporator. 50 ml each of water and ethyl acetate were added. After separation of the phases, the organic phase was washed successively with 50 ml each of 10% strength aqueous citric acid, saturated sodium bicarbonate solution and saturated sodium chloride solution. After drying over anhydrous sodium sulfate, the mixture was filtered and the solvent was removed on a rotary evaporator. The crude product obtained was purified by means of MPLC (silica gel, cyclohexane/ethyl acetate 2:1). 418 mg (36% of th., purity of 93%) of the title compound were obtained, this being employed without further purification.

¹H-NMR (400 MHz, DMSO-d₆, δ/ppm): 8.39 (d, 1H), 8.08 (d, 2H), 7.68 (dd, 1H), 7.62 (d, 2H), 7.52 (d, 1H), 6.93 (s, 1H), 5.56 (s, 2H), 2.39 (s, 3H), 1.72 (s, 3H), 1.86 (s, 3H).

LC/MS (method F, ESIpos): Rₜ=1.43 min, m/z=412 [M+H]⁺.

The compounds in the following table were prepared from the corresponding precursors analogously to one of the processes described under Example 51A and 52A. The preparation of most of the N′-hydroxycarboximide amides (hydroxyamidines) employed has been described above; a very few were commercially obtainable or their preparation is described in the literature.

| | | HPLC: | MS: m/z | LC/MS |
|---|---|---|---|---|
| Example | Structure | Rₜ [min] | [M + H]⁺ | method |
| 53A | 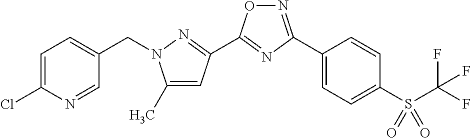 [SMILES: Cc1cc(-c2nc(-c3ccc(S(=O)(=O)C(F)(F)F)cc3)no2)nn1Cc1ccc(Cl)nc1] | 2.39 | 484 | E |
| | ¹H-NMR (400 MHz, DMSO-d₆, δ/ppm): 8.50 (d, 2H), 8.42-8.33 (m, 3H), 7.70 (dd, 1H), 7.53 (d, 2H), 6.98 (s, 1H), 5.56 (s, 2H), 2.39 (s, 3H). | | | |
| 54A | 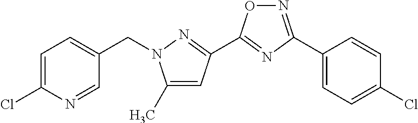 [SMILES: Cc1cc(-c2nc(-c3ccc(Cl)cc3)no2)nn1Cc1ccc(Cl)nc1] | 1.42 | 386 | F |
| | ¹H-NMR (400 MHz, CDCl₃, δ/ppm): 8.32 (d, 2H), 8.14 (d, 2H), 7.51 (dd, 1H), 7.48 (d, 2H), 7.31 (d, 1H), 6.82 (s, 1H), 5.43 (s, 2H), 2.32 (s, 3H). | | | |
| 55A | 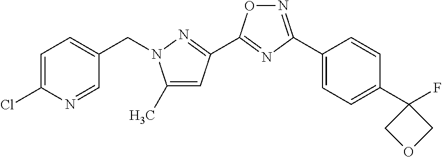 [SMILES: Cc1cc(-c2nc(-c3ccc(C4(F)COC4)cc3)no2)nn1Cc1ccc(Cl)nc1] | 1.14 | 426 | I |
| | ¹H-NMR (400 MHz, CDCl₃, δ/ppm): 8.32 (d, 1H), 8.28 (d, 2H), 7.72 (d, 2H), 7.52 (dd, 1H), 7.33 (d, 1H), 6.84 (s, 1H), 5.45 (s, 2H), 5.05 (dd, 2H), 5.00 (dd, 2H), 2.33 (s, 3H). | | | |
| 56A | 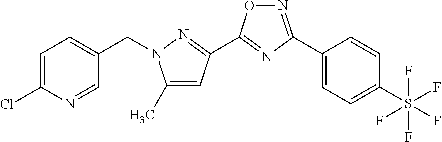 [SMILES: Cc1cc(-c2nc(-c3ccc(S(F)(F)(F)(F)F)cc3)no2)nn1Cc1ccc(Cl)nc1] | 1.50 | 478 | F |
| | ¹H-NMR (400 MHz, CDCl₃, δ/ppm): 8.31 (m, 3H), 7.89 (d, 2H), 7.52 (dd, 1H), 7.32 (d, 2H), 6.84 (s, 1H), 5.44 (s, 2H), 2.32 (s, 3H). | | | |
| 57A | 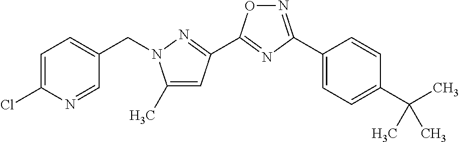 [SMILES: Cc1cc(-c2nc(-c3ccc(C(C)(C)C)cc3)no2)nn1Cc1ccc(Cl)nc1] | 5.10 | 408 | A |
| | ¹H-NMR (400 MHz, CDCl₃, δ/ppm): 8.31 (d, 1H), 8.13 (d, 2H), 7.51 (d, 2H), 7.51 (dd, 1H), 7.32 (d, 1H), 6.83 (s, 1H), 5.44 (s, 2H), 2.32 (s, 3H), 1.36 (s, 9H). | | | |
| 58A | 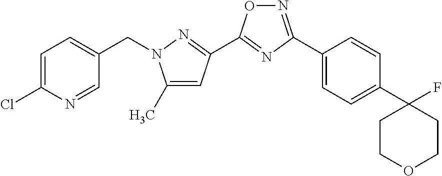 [SMILES: Cc1cc(-c2nc(-c3ccc(C4(F)CCOCC4)cc3)no2)nn1Cc1ccc(Cl)nc1] | 4.60 | 454 | A |
| | ¹H-NMR (400 MHz, CDCl₃, δ/ppm): 8.32 (d, 1H), 8.23 (d, 2H), 7.53 (d, 2H), 7.51 (dd, 1H), 7.32 (d, 1H), 6.83 (s, 1H), 5.44 (s, 2H), 4.00-3.86 (m, 4H), 2.33 (s, 3H), 2.29-2.12 (m, 2H), 1.98-1.92 (m, 2H). | | | |
| 59A | 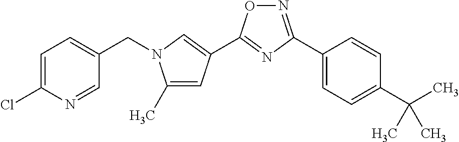 [SMILES: Cc1cc(-c2nc(-c3ccc(C(C)(C)C)cc3)no2)cn1Cc1ccc(Cl)nc1] | 5.20 | 407 | A |
| | ¹H-NMR (400 MHz, CDCl₃, δ/ppm): 8.25 (d, 1H), 8.03 (d, 2H), 7.50 (d, 2H), 7.47 (d, 1H), 7.33 (d, 1H), 7.30 (dd, 1H), 6.60 (d, 1H), 5.10 (s, 2H), 2.20 (s, 3H), 1.37 (s, 9H). | | | |
| 60A | 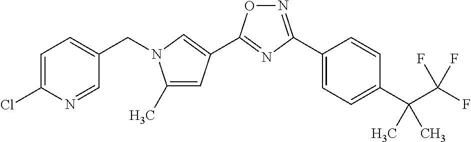 [SMILES: Cc1cc(-c2nc(-c3ccc(C(C)(C)C(F)(F)F)cc3)no2)cn1Cc1ccc(Cl)nc1] | 1.40 | 461 | I |
| | ¹H-NMR (400 MHz, CDCl₃, δ/ppm): 8.24 (d, 1H), 8.10 (d, 2H), 7.61 (d, 2H), 7.50 (d, 1H), 7.34-7.30 (m, 2H), 6.60 (d, 1H), 5.13 (s, 2H), 2.31 (s, 6H), 2.21 (s, 3H). | | | |
| 61A | 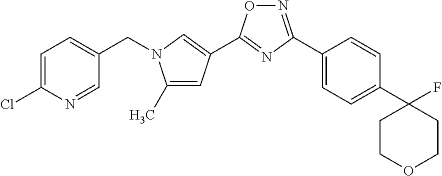 [SMILES: Cc1cc(-c2nc(-c3ccc(C4(F)CCOCC4)cc3)no2)cn1Cc1ccc(Cl)nc1] | 4.74 | 453 | A |
| | ¹H-NMR (400 MHz, CDCl₃, δ/ppm): 8.25 (d, 1H), 8.14 (d, 2H), 7.51 (d, 2H), 7.48 (d, 1H), 7.33 (d, 1H), 7.28 (dd, 1H), 6.60 (d, 1H), 5.11 (s, 2H), 4.00-3.87 (m, 4H), 2.29-2.11 (m, 2H), 2.21 (s, 3H), 1.98-1.91 (m, 2H). | | | |
| 62A | 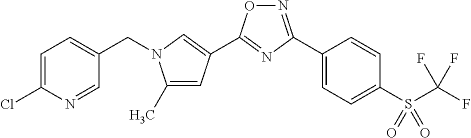 [SMILES: Cc1cc(-c2nc(-c3ccc(S(=O)(=O)C(F)(F)F)cc3)no2)cn1Cc1ccc(Cl)nc1] | 4.95 | 483 | A |
| | ¹H-NMR (400 MHz, CDCl₃, δ/ppm): 8.43 (d, 2H), 8.24 (d, 1H), 7.16 (d, 2H), 7.50 (d, 1H), 7.33 (d, 1H), 7.30 (dd, 1H), 6.61 (d, 1H), 5.12 (s, 2H), 2.22 (s, 3H). | | | |
| 63A | 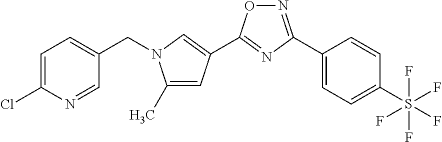 [SMILES: Cc1cc(-c2nc(-c3ccc(S(F)(F)(F)(F)F)cc3)no2)cn1Cc1ccc(Cl)nc1] | 1.40 | 477 | I |
| | ¹H-NMR (400 MHz, CDCl₃, δ/ppm): 8.25-8.20 (m, 3H), 7.87 (d, 2H), 7.49 (d, 1H), 7.33 (d, 1H), 7.29 (dd, 1H), 6.60 (d, 1H), 5.12 (s, 2H), 2.21 (s, 3H). | | | |
| 64A | 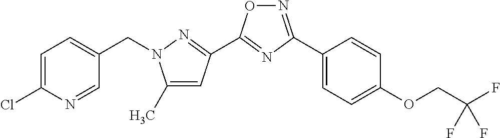 [SMILES: Cc1cc(-c2nc(-c3ccc(OCC(F)(F)F)cc3)no2)nn1Cc1ccc(Cl)nc1] | 1.23 | 450 | I |
| | ¹H-NMR (400 MHz, CDCl₃, δ/ppm): 8.32 (d, 1H), 8.18 (d, 2H), 7.51 (dd, 1H), 7.32 (d, 1H), 7.05 (d, 2H), 6.82 (s, 1H), 5.44 (s, 2H), 4.43 (quart, 2H), 2.33 (s, 3H). | | | |
| 65A | 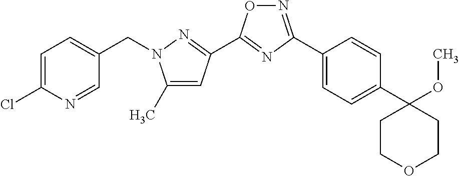 [SMILES: COC1(c2ccc(-c3noc(-c4cc(C)n(Cc5ccc(Cl)nc5)n4)n3)cc2)CCOCC1] | 1.18 | 466 | I |
| | ¹H-NMR (400 MHz, CDCl₃, δ/ppm): 8.32 (d, 1H), 8.21 (d, 2H), 7.53 (d, 2H), 7.52 (dd, 1H), 7.32 (d, 1H), 6.84 (s, 1H), 5.44 (s, 2H), 3.93-3.83 (m, 4H), 3.01 (s, 3H), 2.33 (s, 3H), 2.11-1.98 (m, 4H). | | | |
| 66A | 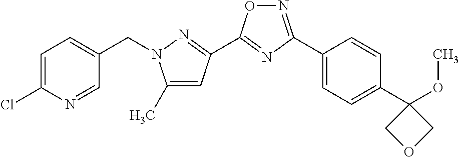 [SMILES: COC1(c2ccc(-c3noc(-c4cc(C)n(Cc5ccc(Cl)nc5)n4)n3)cc2)COC1] | 1.11 | 438 | I |
| | ¹H-NMR (400 MHz, CDCl₃, δ/ppm): 8.32 (d, 1H), 8.26 (d, 2H), 7.61 (d, 2H), 7.52 (dd, 1H), 7.32 (d, 1H), 6.84 (s, 1H), 5.45 (s, 2H), 4.97 (d, 2H), 4.85 (d, 2H), 3.19 (s, 3H), 2.33 (s, 3H). | | | |
| 67A | 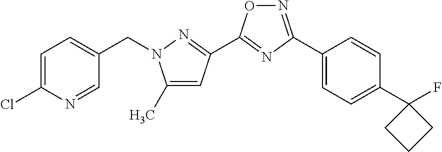 [SMILES: Cc1cc(-c2nc(-c3ccc(C4(F)CCC4)cc3)no2)nn1Cc1ccc(Cl)nc1] | 1.32 | 424 | I |
| | ¹H-NMR (400 MHz, CDCl₃, δ/ppm): 8.32 (d, 1H), 8.22 (d, 2H), 7.60 (d, 2H), 7.51 (dd, 1H), 7.32 (d, 1H), 6.84 (s, 1H), 5.44 (s, 2H), 2.77-2.55 (m, 4H), 2.33 (s, 3H), 2.20-2.08 (m, 1H), 1.87-1.75 (m, 1H). | | | |
| 68A | 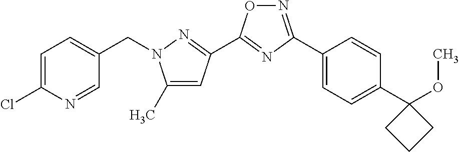 [SMILES: COC1(c2ccc(-c3noc(-c4cc(C)n(Cc5ccc(Cl)nc5)n4)n3)cc2)CCC1] | 1.30 | 436 | I |
| | ¹H-NMR (400 MHz, CDCl₃, δ/ppm): 8.32 (d, 1H), 8.20 (d, 2H), 7.56 (d, 2H), 7.52 (dd, 1H), 7.32 (d, 1H), 6.84 (s, 1H), 5.44 (s, 2H), 2.97 (s, 3H), 2.44-2.41 (m, 4H), 2.33 (s, 3H), 2.03-1.93 (m, 1H), 1.78-1.67 (m, 1H). | | | |
| 69A | 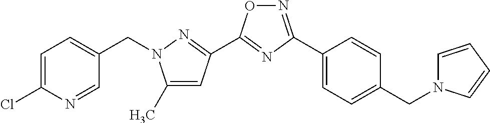 [SMILES: Cc1cc(-c2nc(-c3ccc(Cn4cccc4)cc3)no2)nn1Cc1ccc(Cl)nc1] | 1.24 | 431 | I |
| | ¹H-NMR (400 MHz, CDCl₃, δ/ppm): 8.31 (d, 1H), 8.13 (d, 2H), 7.50 (dd, 1H), 7.31 (d, 1H), 7.22 (d, 2H), 6.82 (s, 1H), 6.72 (s, 2H), 6.22 (s, 2H), 5.42 (s, 2H), 5.13 (s, 2H), 2.31 (s, 3H). | | | |
| 70A | 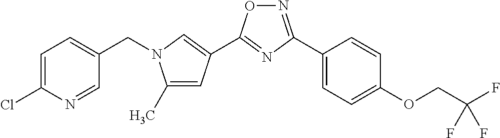 [SMILES: Cc1cc(-c2nc(-c3ccc(OCC(F)(F)F)cc3)no2)cn1Cc1ccc(Cl)nc1] | 1.30 | 449 | I |
| | ¹H-NMR (400 MHz, CDCl₃, δ/ppm): 8.25 (d, 1H), 8.10 (d, 2H), 7.46 (d, 1H), 7.33 (d, 1H), 7.27 (dd, 1H), 7.04 (d, 2H), 6.59 (d, 1H), 5.10 (s, 2H), 5.03 (s, 2H), 4.42 (quart, 2H), 2.20 (s, 3H). | | | |
| 71A | 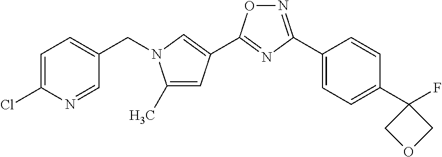 [SMILES: Cc1cc(-c2nc(-c3ccc(C4(F)COC4)cc3)no2)cn1Cc1ccc(Cl)nc1] | 1.21 | 425 | I |
| | ¹H-NMR (400 MHz, CDCl₃, δ/ppm): 8.25 (d, 1H), 8.20 (d, 2H), 7.70 (d, 2H), 7.49 (d, 1H), 7.33 (d, 1H), 7.28 (dd, 1H), 6.61 (d, 1H), 5.11 (s, 2H), 5.05 (dd, 2H), 5.00 (dd, 2H), 2.21 (s, 3H). | | | |
| 72A | 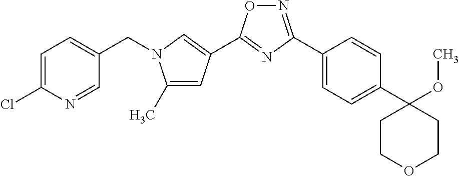 [SMILES: COC1(c2ccc(-c3noc(-c4cc(C)n(Cc5ccc(Cl)nc5)c4)n3)cc2)CCOCC1] | 1.38 | 465 | F |
| | ¹H-NMR (400 MHz, CDCl₃, δ/ppm): 8.25 (d, 1H), 8.12 (d, 2H), 7.51 (d, 2H), 7.48 (d, 1H), 7.33 (d, 1H), 7.28 (dd, 1H), 6.60 (d, 1H), 5.11 (s, 2H), 3.94-3.81 (m, 4H), 3.01 (s, 3H), 2.20 (s, 3H), 2.11-1.97 (m, 4H). | | | |
| 73A | 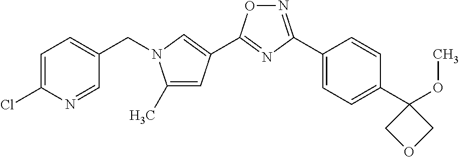 [SMILES: COC1(c2ccc(-c3noc(-c4cc(C)n(Cc5ccc(Cl)nc5)c4)n3)cc2)COC1] | 1.17 | 437 | I |
| | ¹H-NMR (400 MHz, CDCl₃, δ/ppm): 8.25 (d, 1H), 8.18 (d, 2H), 7.59 (d, 2H), 7.48 (d, 1H), 7.33 (d, 1H), 7.29 (dd, 1H), 6.61 (d, 1H), 5.11 (s, 2H), 4.96 (d, 2H), 4.85 (d, 2H), 3.17 (s, 3H), 2.21 (s, 3H). | | | |
| 74A | 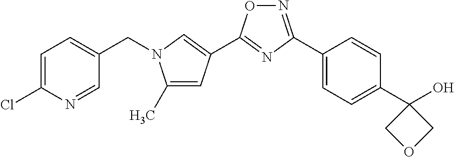 [SMILES: Cc1cc(-c2nc(-c3ccc(C4(O)COC4)cc3)no2)cn1Cc1ccc(Cl)nc1] | 4.17 | 423 | A |

¹H-NMR (400 MHz, CDCl₃, δ/ppm): 8.25 (d, 1H), 8.17 (d, 2H), 7.75 (d, 2H), 7.48 (d, 1H), 7.33 (d, 1H), 7.28 (dd, 1H), 6.61 (d, 1H), 5.11 (s, 2H), 4.97-4.94 (m, 4H), 2.78 (broad, 1H), 2.20 (s, 3H).

### Example 75A

### 2-Bromo-6-[(5-methyl-3-{3-[4-(trifluoromethoxy)phenyl]-1,2,4-oxadiazol-5-yl}-1H-pyrazol-1-yl)-methyl]pyridine

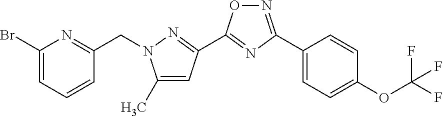 [SMILES: Cc1cc(-c2nc(-c3ccc(OC(F)(F)F)cc3)no2)nn1Cc1cccc(Br)n1]

0.73 g (6.49 mmol) of solid potassium tert-butylate was added to a solution of 1.83 g (5.90 mmol) of the compound from Example 28A and 2.04 g (7.67 mmol) of (6-bromopyridin-2-yl)methyl methanesulfonate [T. Kawano et al., Bull. Chem. Soc. Jpn. 2003, 76 (4), 709-720] in 50 ml of anhydrous THF at 0° C. The reaction mixture was subsequently allowed to come to RT. After 1.5 h, approx. 100 ml of water were added and the mixture was extracted three times with approx. 100 ml of ethyl acetate each time. The combined organic extracts were dried over anhydrous sodium sulfate and, after filtration, the solvent was removed on a rotary evaporator. The residue obtained was stirred with 30 ml of methylene chloride. After filtration and drying of the residue on the filter, a first amount of 1.21 g (43% of th.) of the title compound was obtained. The mother liquor was freed from the solvent on a rotary evaporator and the residue was purified by means of MPLC (silica gel, cyclohexane/ethyl acetate 4:1→1:1). A further 0.42 g (16% of th.) of the title compound were obtained in this manner.

¹H-NMR (400 MHz, DMSO-d₆, δ/ppm): 8.20 (d, 2H), 7.78 (t, 1H), 7.63-7.58 (m, 3H), 7.18 (d, 1H), 6.96 (s, 1H), 5.60 (s, 2H), 2.39 (s, 3H).

LC/MS (method F, ESIpos): Rₜ=1.53 min, m/z=480/482 (⁷⁹Br/⁸¹Br) [M+H]⁺.

### Example 76A

### 5-Iodo-2-[(5-methyl-3-{3-[4-(trifluoromethoxy)phenyl]-1,2,4-oxadiazol-5-yl}-1H-pyrazol-1-yl)-methyl]pyridine

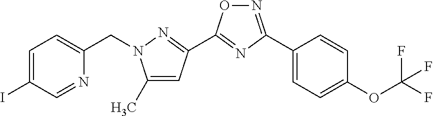 [SMILES: Cc1cc(-c2nc(-c3ccc(OC(F)(F)F)cc3)no2)nn1Cc1ccc(I)cn1]

219 mg (1.95 mmol) of solid potassium tert-butylate were added to a solution of 504 mg (1.62 mmol) of the compound from Example 28A and 535 mg (2.11 mmol) of the compound from Example 44A in 20 ml of anhydrous THF at 0° C. The reaction mixture was subsequently allowed to come to RT. After 15 h, approx. 100 ml of water were added and the mixture was extracted three times with approx. 100 ml of ethyl acetate each time. The combined organic extracts were washed with saturated sodium chloride solution and dried over anhydrous magnesium sulfate. After filtration, the solvent was removed on a rotary evaporator. The title compound was isolated by means of preparative HPLC (method N). 657 mg (77% of th.) were obtained.

¹H-NMR (400 MHz, CDCl₃, δ/ppm): 8.79 (d, 1H), 8.24 (d, 2H), 7.97 (dd, 1H), 7.33 (d, 2H), 6.86 (d, 1H), 6.83 (s, 1H), 5.50 (s, 2H), 2.36 (s, 3H).

HPLC (method B): Rₜ=5.25 min.

MS (ESIpos): m/z=528 [M+H]⁺.

The compounds in the following table were prepared from the corresponding educts analogously to the processes described in Example 75A and 76A. Depending on the polarity of the compounds, they were isolated either by extraction by stirring from methylene chloride, ethyl acetate, acetonitrile or diethyl ether, by means of preparative HPLC or by means of MPLC over silica gel with cyclohexane/ethyl acetate mixtures as the mobile phase. The arylmethyl chlorides, bromides or methanesulfonates used as educts were either commercially obtainable, or they were prepared as described above or their preparation is described in the literature: (6-chloropyridin-3-yl)methyl methanesulfonate [K. C. Iee et al., J. Org. Chem. 1999, 64 (23), 8576-8581].

| | | HPLC: | MS: m/z | LC/MS |
|---|---|---|---|---|
| Example | Structure | Rₜ [min] | [M + H]⁺ | method |
| 77A | 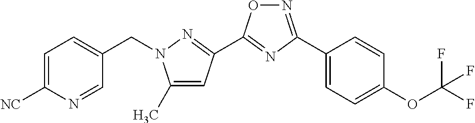 [SMILES: Cc1cc(-c2nc(-c3ccc(OC(F)(F)F)cc3)no2)nn1Cc1ccc(C#N)nc1] | 2.70 | 427 | C |
| | ¹H-NMR (400 MHz, CDCl₃, δ/ppm): 8.62 (d, 1H), 8.24 (d, 2H), 7.70 (d, 1H), 7.62 (dd, 1H), 7.34 (d, 2H), 6.88 (s, 1H), 5.52 (s, 2H), 2.34 (s, 3H). | | | |
| 78A | 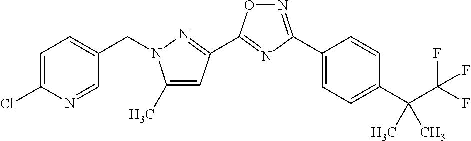 [SMILES: Cc1cc(-c2nc(-c3ccc(C(C)(C)C(F)(F)F)cc3)no2)nn1Cc1ccc(Cl)nc1] | 2.94 | 462 | C |
| | ¹H-NMR (400 MHz, CDCl₃, δ/ppm): 8.33 (d, 1H), 8.18 (d, 2H), 7.63 (d, 2H), 7.52 (dd, 1H), 7.32 (d, 1H), 6.84 (s, 1H), 5.45 (s, 2H), 2.32 (s, 3H), 1.63 (s, 6H). | | | |
| 79A | 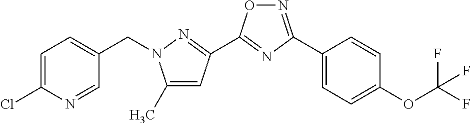 [SMILES: Cc1cc(-c2nc(-c3ccc(OC(F)(F)F)cc3)no2)nn1Cc1ccc(Cl)nc1] | 2.83 | 436 | C |
| | ¹H-NMR (400 MHz, CDCl₃, δ/ppm): 8.31 (d, 1H), 8.25 (d, 2H), 7.51 (dd, 1H), 7.36-7.30 (m, 3H), 6.82 (s, 1H), 5.43 (s, 2H), 2.32 (s, 3H). | | | |
| 80A | 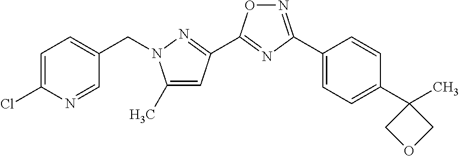 [SMILES: Cc1cc(-c2nc(-c3ccc(C4(C)COC4)cc3)no2)nn1Cc1ccc(Cl)nc1] | 1.26 | 422 | F |
| | ¹H-NMR (400 MHz, CDCl₃, δ/ppm): 8.32 (d, 1H), 8.20 (d, 2H), 7.51 (dd, 1H), 7.33 (d, 2H), 7.31 (d, 1H), 6.83 (s, 1H), 5.44 (s, 2H), 5.00 (d, 2H), 4.58 (d, 2H), 2.33 (s, 3H), 1.77 (s, 3H). | | | |
| 81A | 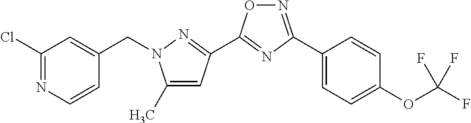 [SMILES: Cc1cc(-c2nc(-c3ccc(OC(F)(F)F)cc3)no2)nn1Cc1ccnc(Cl)c1] | 1.47 | 436 | F |
| | ¹H-NMR (400 MHz, CDCl₃, δ/ppm): 8.37 (d, 1H), 8.28-8.22 (m, 2H), 7.34 (d, 2H), 7.05 (s, 1H), 6.97 (d, 1H), 6.88 (s, 1H), 5.43 (s, 2H), 2.32 (s, 3H). | | | |
| 82A | 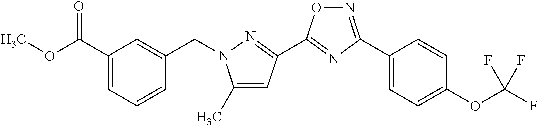 [SMILES: COC(=O)c1cccc(Cn2nc(-c3nc(-c4ccc(OC(F)(F)F)cc4)no3)cc2C)c1] | 1.55 | 459 | F |
| | ¹H-NMR (400 MHz, CDCl₃, δ/ppm): 8.25 (d, 2H), 7.99 (d, 1H), 7.89 (s, 1H), 7.42 (dd, 1H), 7.33 (d, 2H und d, 1H), 6.82 (s, 1H), 5.50 (s, 2H), 3.90 (s, 3H), 2.29 (s, 3H). | | | |
| 83A | 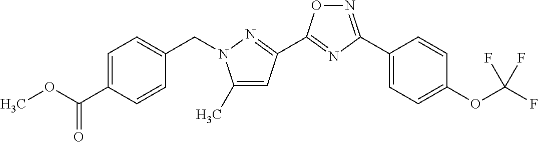 [SMILES: COC(=O)c1ccc(Cn2nc(-c3nc(-c4ccc(OC(F)(F)F)cc4)no3)cc2C)cc1] | 1.54 | 459 | F |
| | ¹H-NMR (400 MHz, CDCl₃, δ/ppm): 8.25 (d, 2H), 8.01 (d, 2H), 7.33 (d, 2H), 7.21 (d, 2H), 6.83 (s, 1H), 5.52 (s, 2H), 3.91 (s, 3H), 2.27 (s, 3H). | | | |
| 84A | 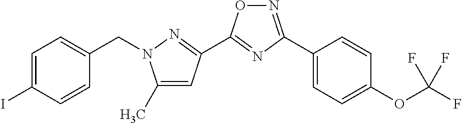 [SMILES: Cc1cc(-c2nc(-c3ccc(OC(F)(F)F)cc3)no2)nn1Cc1ccc(I)cc1] | 2.85 | 527 | E |
| | ¹H-NMR (400 MHz, CDCl₃, δ/ppm): 8.25 (d, 2H), 7.67 (d, 2H), 7.32 (d, 2H), 6.91 (d, 2H), 6.81 (s, 1H), 5.39 (s, 2H), 2.27 (s, 3H). | | | |
| 85A | 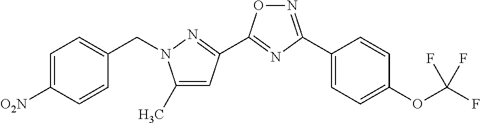 [SMILES: Cc1cc(-c2nc(-c3ccc(OC(F)(F)F)cc3)no2)nn1Cc1ccc([N+](=O)[O-])cc1] | 5.20 | 446 | B |
| | ¹H-NMR (400 MHz, CDCl₃, δ/ppm): 8.18 (d, 2H), 8.14 (d, 2H), 7.27 (d, 2H), 7.24 (d, 2H), 6.80 (s, 1H), 5.48 (s, 2H), 2.23 (s, 3H). | | | |
| 86A | 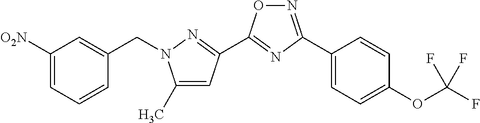 [SMILES: Cc1cc(-c2nc(-c3ccc(OC(F)(F)F)cc3)no2)nn1Cc1cccc([N+](=O)[O-])c1] | 1.52 | 446 | F |
| | ¹H-NMR (400 MHz, CDCl₃, δ/ppm): 8.25 (d, 2H), 8.19 (d, 1H), 8.04 (s, 1H), 7.54 (dd, 1H), 7.49 (d, 1H), 7.33 (d, 2H), 6.84 (s, 1H), 5.55 (s, 2H), 2.33 (s, 3H). | | | |
| 87A | 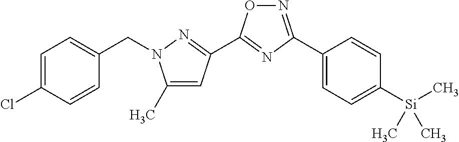 [SMILES: Cc1cc(-c2nc(-c3ccc([Si](C)(C)C)cc3)no2)nn1Cc1ccc(Cl)cc1] | 1.45 | 424 | I |
| | ¹H-NMR (400 MHz, DMSO-d₆, δ/ppm): 8.32 (dd, 1H), 8.16 (d, 2H), 7.63 (d, 2H), 7.52-7.49 (dd, 1H), 7.31 (d, 1H), 6.82 (s, 1H), 5.42 (s, 2H), 2.32 (s, 3H), 0.31 (s, 9H). | | | |
| 88A | 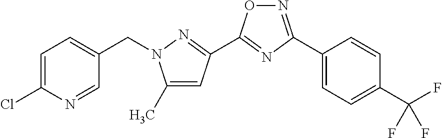 [SMILES: Cc1cc(-c2nc(-c3ccc(C(F)(F)F)cc3)no2)nn1Cc1ccc(Cl)nc1] | 1.30 | 420 | I |
| | ¹H-NMR (400 MHz, CDCl₃, δ/ppm): 8.34-8.30 (m, 3H), 7.77 (d, 2H), 7.52 (dd, 1H), 7.31 (d, 1H), 6.84 (s, 1H), 5.44 (s, 2H), 2.32 (s, 3H). | | | |
| 89A | 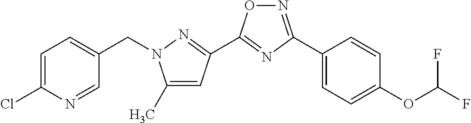 [SMILES: Cc1cc(-c2nc(-c3ccc(OC(F)F)cc3)no2)nn1Cc1ccc(Cl)nc1] | 1.21 | 418 | I |
| | ¹H-NMR (400 MHz, CDCl₃, δ/ppm): 8.32 (d, 1H), 8.21 (d, 2H), 7.51 (dd, 1H), 7.31 (d, 1H), 7.22 (d, 2H), 6.82 (s, 1H), 6.60 (t, 1H), 5.43 (s, 2H), 2.32 (s, 3H). | | | |
| 90A | 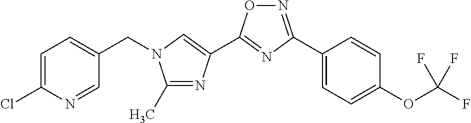 [SMILES: Cc1nc(-c2nc(-c3ccc(OC(F)(F)F)cc3)no2)cn1Cc1ccc(Cl)nc1] | 1.29 | 436 | F |

¹H-NMR (400 MHz, CDCl₃, δ/ppm): 8.33 (s, 1H), 8.22 (d, 2H), 7.71 (s, 1H), 7.39 (s, 2H), 7.32 (d, 2H), 5.18 (s, 2H), 2.50 (s, 3H).

### Example 91A

### 5-[(5-Methyl-3-{3-[4-(trifluoromethoxy)phenyl]-1,2,4-oxadiazol-5-yl}-1H-pyrazol-1-yl)methyl]-pyridine-2-carbaldehyde

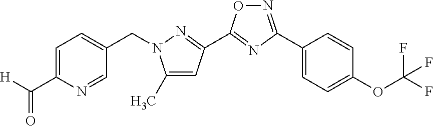 [SMILES: Cc1cc(-c2nc(-c3ccc(OC(F)(F)F)cc3)no2)nn1Cc1ccc(C=O)nc1]

3.5 ml (3.5 mmol) of a 1 M solution of diisobutylaluminium hydride (DIBAL-H) in heptane was added to a solution of 980 mg (2.30 mmol) of the compound from Example 77A in 30 ml of anhydrous THF under inert conditions and at −78° C. After the reaction mixture had been stirred at −78° C. for 3 h, 22 ml of 1 M hydrochloric acid were added. The mixture was allowed to warm to RT, while stirring. It was then extracted with ethyl acetate. The organic extracts were washed successively with water and saturated sodium chloride solution. After drying over anhydrous magnesium sulfate, the mixture was filtered and the solvent was removed on a rotary evaporator. The crude product was purified by means of MPLC (silica gel, cyclohexane/ethyl acetate 1:1). 300 mg (30% of th.) of the title compound were obtained.

¹H-NMR (400 MHz, CDCl₃, δ/ppm): 10.07 (s, 1H), 8.67 (d, 1H), 8.25 (d, 2H), 7.95 (d, 1H), 7.67 (dd, 1H), 7.34 (d, 2H), 6.87 (s, 1H), 5.57 (s, 2H), 2.35 (s, 3H).

MS (DCI, NH₃): m/z=430 [M+H]⁺.

LC/MS (method C, ESIpos): Rₜ=2.66 min, m/z=430 [M+H]⁺.

### Example 92A

### 5-[(5-Methyl-3-{3-[4-(trifluoromethoxy)phenyl]-1,2,4-oxadiazol-5-yl}-1H-pyrazol-1-yl)methyl]-pyridine-2-carboxylic acid

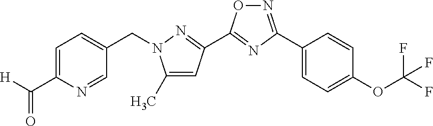 [SMILES: Cc1cc(-c2nc(-c3ccc(OC(F)(F)F)cc3)no2)nn1Cc1ccc(C=O)nc1]

5 ml of a 30% strength potassium hydroxide solution in water were added to a solution of 500 mg (1.17 mmol) of the compound from Example 77A in 5 ml of ethanol and the mixture was heated under reflux for 1 h. After cooling to RT, approx. 20 ml of water were added and the product was precipitated out with concentrated hydrochloric acid. This was filtered off, washed neutral with water and dried under a high vacuum. 448 mg (86% of th.) of the title compound were obtained.

¹H-NMR (400 MHz, CDCl₃, δ/ppm): 8.52 (d, 1H), 8.24 (d, 2H), 8.22 (d, 1H), 7.75 (dd, 1H), 7.33 (d, 2H), 6.88 (s, 1H), 5.57 (s, 2H), 2.36 (s, 3H).

MS (DCI, NH₃): m/z=446 [M+H]⁺.

LC/MS (method F, ESIpos): Rₜ=1.22 min, m/z=446 [M+H]⁺.

### Example 93A

### 3-[(5-Methyl-3-{3-[4-(trifluoromethoxy)phenyl]-1,2,4-oxadiazol-5-yl}-1H-pyrazol-1-yl)methyl]-benzenecarboxylic acid

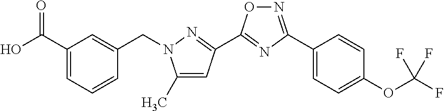 [SMILES: Cc1cc(-c2nc(-c3ccc(OC(F)(F)F)cc3)no2)nn1Cc1cccc(C(=O)O)c1]

89 ml (88.7 mmol) of a 1 M sodium hydroxide solution were added to a suspension of 8.13 g (17.7 mmol) of the compound from Example 82A in 120 ml of methanol and the mixture was heated under reflux for 1 h. The methanol was then mostly removed on a rotary evaporator. The aqueous solution which remained was acidified with 100 ml of 1 M hydrochloric acid, while stirring. The product thereby precipitated out, and was filtered off with suction, washed with water and dried under a high vacuum. 7.51 g (95% of th.) of the title compound were obtained.

¹H-NMR (400 MHz, DMSO-d₆, δ/ppm): 13.07 (s, broad, 1H), 8.20 (d, 2H), 7.40 (d, 1H), 7.78 (s, 1H), 7.59 (d, 2H), 7.51 (dd, 1H), 7.46 (d, 1H), 6.97 (s, 1H), 5.60 (s, 2H), 2.34 (s, 3H).

LC/MS (method C, ESIpos): Rₜ=2.68 min, m/z=445 [M+H]⁺.

Analogously to the process described in Example 9A, the compound in the following table was obtained by hydrolysis of the corresponding ester:

| | | HPLC: | MS: m/z | LC/MS |
|---|---|---|---|---|
| Example | Structure | Rₜ [min] | [M + H]⁺ | method |
| 94A | 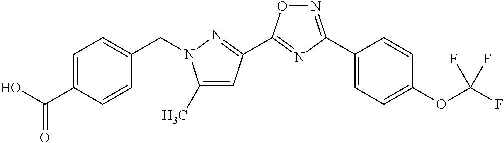 [SMILES: Cc1cc(-c2nc(-c3ccc(OC(F)(F)F)cc3)no2)nn1Cc1ccc(C(=O)O)cc1] | 2.56 | 445 | D |

¹H-NMR (400 MHz, DMSO-d₆, δ/ppm): 13.00 (broad, 1H), 8.20 (d, 2H), 7.94 (d, 2H), 7.59 (d, 2H), 7.29 (d, 2H), 6.96 (s, 1H), 5.60 (s, 2H), 2.33 (s, 3H).

### Example 95A

### 3-[(5-Methyl-3-{3-[4-(trifluoromethoxy)phenyl]-1,2,4-oxadiazol-5-yl}-1H-pyrazol-1-yl)methyl]-phenol

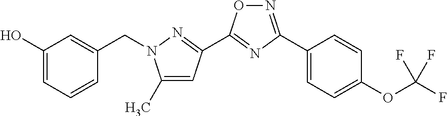 [SMILES: Cc1cc(-c2nc(-c3ccc(OC(F)(F)F)cc3)no2)nn1Cc1cccc(O)c1]

199 mg (1.77 mmol) of solid potassium tert-butylate were added to a solution of 500 mg (1.61 mmol) of the compound from Example 28A and 719 mg (2.10 mmol) of the compound from Example 47A in 10 ml of anhydrous THF at 0° C. The reaction mixture was subsequently allowed to come to RT. After 15 h, approx. 100 ml of water were added and the mixture was extracted three times with approx. 100 ml of ethyl acetate each time. The combined organic extracts were washed with saturated sodium chloride solution and dried over anhydrous magnesium sulfate. After filtration, the solvent was removed on a rotary evaporator. The residue obtained in this way was dissolved again in 20 ml of THF, and 3.2 ml (3.2 mmol) of a 1 M solution of tetra-n-butylammonium fluoride in THF were added at 0° C. After the mixture had been stirred at RT for 1 h, the batch was diluted with a few ml of methanol and separated into its components directly by means of preparative HPLC (method N). 218 mg (32% of th.) of the title compound were obtained.

¹H-NMR (400 MHz, CDCl₃, δ/ppm): 8.11 (d, 2H), 7.29 (d, 2H), 7.20 (t, 1H), 6.80 (d, 1H), 6.79 (s, 1H), 6.73 (d, 1H), 6.62 (s, 1H), 6.50 (s, 1H), 5.33 (s, 2H), 2.06 (s, 3H).

HPLC (method A): Rₜ=4.81 min.

MS (DCI, NH₃): m/z=417 [M+H]⁺.

LC/MS (method E, ESIpos): Rₜ=2.34 min, m/z=417 [M+H]⁺.

Analogously to the process described under Example 95A, the compound in the following table was obtained from the corresponding educts:

| | | HPLC: | MS: m/z | LC/MS |
|---|---|---|---|---|
| Example | Structure | Rₜ [min] | [M + H]⁺ | method |
| 96A | 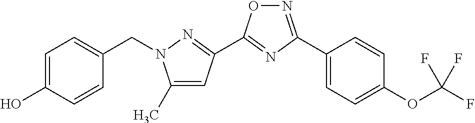 [SMILES: Cc1cc(-c2nc(-c3ccc(OC(F)(F)F)cc3)no2)nn1Cc1ccc(O)cc1] | 2.55 | 417 | D |

¹H-NMR (400 MHz, CDCl₃, δ/ppm): 8.24 (d, 2H), 7.32 (d, 2H), 7.07 (d, 2H), 6.80 (s, 1H), 6.79 (d, 2H), 5.37 (s, 2H), 5.31 (s, broad, 1H), 2.28 (s, 3H).

### Example 97A

### 4-[(5-Methyl-3-{3-[4-(trifluoromethoxy)phenyl]-1,2,4-oxadiazol-5-yl}-1H-pyrazol-1-yl)methyl]-aniline

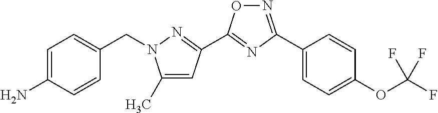 [SMILES: Cc1cc(-c2nc(-c3ccc(OC(F)(F)F)cc3)no2)nn1Cc1ccc(N)cc1]

A solution of 400 mg (0.898 mmol) of the compound from Example 85A in a mixture of 25 ml of ethanol and 25 ml of ethyl acetate was hydrogenated in a flow-through hydrogenation apparatus (“H-Cube” from ThalesNano, Budapest, Hungary) (conditions: 10% Pd/C catalyst, “full H₂” mode, 1 ml/min, 25° C.). After removal of the solvent, the residue was taken up in a few ml of ethanol and the undissolved material was filtered off. This undissolved material was educt material, which was subsequently hydrogenated once more, as described above. The crude product obtained from the two hydrogenations was combined and purified by means of preparative HPLC (method N). 229 mg (62% of th.) of the title compound were obtained.

¹H-NMR (400 MHz, CDCl₃, δ/ppm): 8.25 (d, 2H), 7.33 (d, 2H), 7.01 (d, 2H), 6.87 (s, 1H), 6.63 (d, 2H), 5.33 (s, 2H), 3.69 (broad, 2H), 2.27 (s, 3H).

LC/MS (method C, ESIpos): Rₜ=2.57 min, m/z=416 [M+H]⁺.

The compound in the following table was prepared from the corresponding nitro compound by hydrogenation analogously to the process described under Example 97A:

| | | HPLC: | MS: m/z | LC/MS |
|---|---|---|---|---|
| Example | Structure | Rₜ [min] | [M + H]⁺ | method |
| 98A | 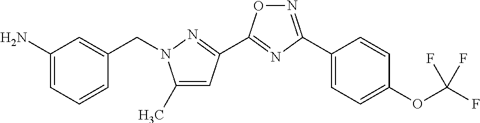 [SMILES: Cc1cc(-c2nc(-c3ccc(OC(F)(F)F)cc3)no2)nn1Cc1cccc(N)c1] | 2.63 | 416 | D |

¹H-NMR (400 MHz, CDCl₃, δ/ppm): 8.26 (d, 2H), 7.33 (d, 2H), 7.10 (dd, 1H), 6.80 (s, 1H), 6.60 (dd, 1H), 6.55 (dd, 1H), 6.44 (dd, 1H), 5.36 (s, 2H), 3.67 (s, broad, 2H), 2.27 (s, 3H).

### Example 99A

### tert-Butyl {4-[(5-methyl-3-{3-[4-(trifluoromethoxy)phenyl]-1,2,4-oxadiazol-5-yl}-1H-pyrazol-1-yl)methyl]phenyl}carbamate

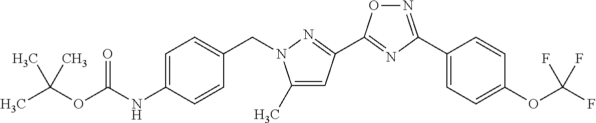 [SMILES: Cc1cc(-c2nc(-c3ccc(OC(F)(F)F)cc3)no2)nn1Cc1ccc(NC(=O)OC(C)(C)C)cc1]

134 μl (0.963 mmol) of triethylamine and 3 mg (0.024 mmol) of DMAP were added to a solution of 200 mg (0.481 mmol) of the compound from Example 97A in 10 ml of anhydrous THF. The reaction mixture was cooled to 0° C. and 132 mg (0.602 mmol) of di-tert-butyl dicarbonate were added. The mixture was stirred at 0° C. for 1 h and then at RT for a further 16 h. Thereafter, it was diluted with 5 ml of methanol and the product was isolated in two portions by means of preparative HPLC (method N). 74 mg (30% of th.) of the title compound were obtained.

¹H-NMR (400 MHz, CDCl₃, δ/ppm): 8.26 (d, 2H), 7.33 (2 d, tog. 4H), 7.12 (d, 2H), 6.79 (s, 1H), 6.49 (s, broad, 1H), 5.39 (s, 2H), 2.26 (s, 3H), 1.50 (s, 9H).

LC/MS (method E, ESIpos): Rₜ=2.74 min, m/z=516 [M+H]⁺.

### Example 100A

### 2-Bromo-5-[(5-methyl-3-{3-[4-(trifluoromethoxy)phenyl]-1,2,4-oxadiazol-5-yl}-1H-pyrazol-1-yl)-methyl]pyridine

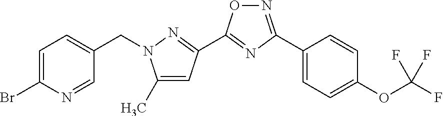 [SMILES: Cc1cc(-c2nc(-c3ccc(OC(F)(F)F)cc3)no2)nn1Cc1ccc(Br)nc1]

A mixture of 1.95 g (4.47 mmol) of the compound from Example 79A and 1.37 g (8.95 mmol) of bromo(trimethyl)silane in 0.5 ml of propionitrile was heated at 120° C. in a microwave apparatus for 70 min, while stirring (CEM Discover, initial irradiation power 250 W). During this operation a relatively marked increase in pressure and temperature was to be observed in the first 10 min. After cooling to RT, a further 350 mg (2.29 mmol) of bromo(trimethyl)silane were added and the mixture was heated at 120° C. in the microwave oven for a further 60 min. During this operation a relatively marked increase in pressure and temperature was again to be observed in the first 10 min. After cooling to RT, the mixture was diluted with 100 ml of water and 100 ml of ethyl acetate and the phases were separated. The combined organic phase was washed once with 100 ml of water, dried over sodium sulfate, filtered and concentrated on a rotary evaporator. The residue was purified by column chromatography over silica gel (mobile phase: cyclohexane/ethyl acetate 3:2). 1.45 g (65% of th.) of the title compound were obtained with a purity of 86% according to LC-MS. Approx. 10% of the educt (compound from Example 79A) was obtained as an impurity.

¹H-NMR (400 MHz, CDCl₃, δ/ppm): 8.31 (d, 1H), 8.23 (d, 2H), 7.47 (d, 1H), 7.40 (dd, 1H), 7.33 (d, 2H), 6.82 (s, 1H), 5.41 (s, 2H), 2.32 (s, 3H).

LC/MS (method E, ESIpos): Rₜ=2.54 min, m/z=480 [M+H]⁺.

### Example 101A

### 2-Iodo-5-[(5-methyl-3-{3-[4-(trifluoromethoxy)phenyl]-1,2,4-oxadiazol-5-yl}-1H-pyrazol-1-yl)-methyl]pyridine

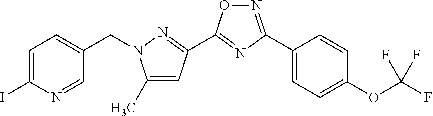 [SMILES: Cc1cc(-c2nc(-c3ccc(OC(F)(F)F)cc3)no2)nn1Cc1ccc(I)nc1]

103 mg (0.688 mmol) of sodium iodide and 27 mg (0.252 mmol) of chloro(trimethyl)silane were added to a solution of 100 mg (0.229 mmol) of the compound from Example 79A in 0.5 ml of propionitrile in a microwave reaction vessel at RT, after which the reaction mixture rapidly assumed a solid consistency. This mixture was then heated at 120° C. in a microwave apparatus for 1 h (CEM Discover, initial irradiation power 250 W). After cooling to RT, the reaction mixture was diluted with 2 ml of acetonitrile and 1 ml of water. Two liquid phases formed, which were separated from one another. The organic phase was purified directly, without further treatment, by means of preparative HPLC (method O). 61 mg (50% of th.) of the title compound were obtained.

¹H-NMR (400 MHz, CDCl₃, δ/ppm): 8.29 (d, 1H), 8.24 (d, 2H), 7.71 (d, 1H), 7.32 (d, 2H), 7.18 (dd, 1H), 6.82 (s, 1H), 5.39 (s, 2H), 2.31 (s, 3H).

LC/MS (method F, ESIpos): Rₜ=1.52 min, m/z=528 [M+H]⁺.

### Example 102A

### 4-[(5-Methyl-3-{3-[4-(trifluoromethoxy)phenyl]-1,2,4-oxadiazol-5-yl}-1H-pyrazol-1-yl)methyl]-pyridine-2-carbonitrile

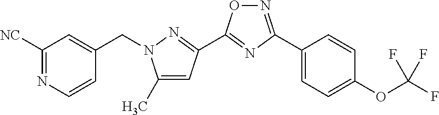 [SMILES: Cc1cc(-c2nc(-c3ccc(OC(F)(F)F)cc3)no2)nn1Cc1ccnc(C#N)c1]

200 mg (0.459 mmol) of the compound from Example 81A were initially introduced into 3.4 ml of dimethylacetamide, 31 mg (0.266 mmol) of zinc cyanide, 6.7 mg (0.020 mmol) of palladium(II) trifluoroacetate, 16 mg (0.040 mmol) of racemic 2-(di-tert-butylphosphino)-1,1′-binaphthyl and 6 mg (0.092 mmol) of zinc powder (97.5%, 325 mesh) were added successively at RT and the mixture was stirred at 90° C. overnight. After cooling to RT, a further 6.7 g (0.020 mmol) of palladium(II) trifluoroacetate were added and the mixture was stirred at 90° C. for a further 24 h. After cooling to RT, 6.7 mg (0.020 mmol) of palladium(II) trifluoroacetate, 16 mg (0.040 mmol) of racemic 2-(di-tert-butylphosphino)-1,1′-binaphthyl and 6 mg (0.092 mmol) of zinc powder (97.5%, 325 mesh) were again added and the mixture was stirred again at 90° C. overnight. After cooling to RT, the solid constituents were then filtered off and the mixture which remained was purified by means of preparative HPLC (method O). The combined product-containing fractions were concentrated on a rotary evaporator to a small residual volume and sodium bicarbonate was then added, after which a solid precipitated out. This was filtered off and dried in vacuo. 21 mg (11% of th.) of the title compound were obtained in this way.

¹H-NMR (400 MHz, CDCl₃, δ/ppm): 8.71 (d, 1H), 8.24 (d, 2H), 7.41 (s, 1H), 7.34 (d, 2H), 7.24 (s, 1H), 6.90 (s, 1H), 5.51 (s, 2H), 2.32 (s, 3H).

LC/MS (method D, ESIpos): Rₜ=2.52 min, m/z=427 [M+H]⁺.

### Example 103A

### 1-(2-Fluoroethyl)piperazine dihydrochloride

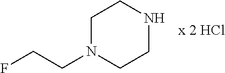

### Step 1: tert-Butyl 4-(2-fluoroethyl)piperazine-1-carboxylate

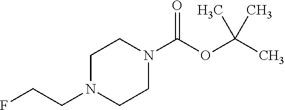 [SMILES: CC(C)(C)OC(=O)N1CCN(CCF)CC1]

A mixture of 1.00 g (5.37 mmol) of tert-butyl piperazine-1-carboxylate, 937 μl (8.05 mmol) of 1-bromo-2-fluoroethane and 1.86 g (13.4 mmol) of potassium carbonate in 15 ml of acetonitrile was heated at 60° C. for 16 h. After cooling to RT, the undissolved material was filtered off and the filtrate was freed from the solvent on a rotary evaporator. The residue obtained was purified by means of MPLC (silica gel; mobile phase: cyclohexane/ethyl acetate 1:2). 1.12 g (89% of th.) of the title compound were obtained.

¹H-NMR (400 MHz, CDCl₃, δ/ppm): 4.58 (td, 2H), 3.46 (t, 4H), 2.71 (td, 2H), 2.48 (t, 4H), 1.46 (s, 9H).

GC/MS (method L, EI): Rₜ=4.74 min, m/z=232 [M]⁺.

### Step 2: 1-(2-Fluoroethyl)piperazine dihydrochloride

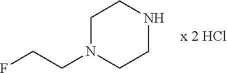

30 ml of a 4 M solution of hydrogen chloride in 1,4-dioxane were added to 1.10 g (4.72 mmol) of the compound from Example 103A/step 1 and the mixture was stirred at RT for 16 h. All the volatile constituents were then removed on a rotary evaporator. The residue obtained was stirred with diethyl ether, filtered off with suction and rinsed with diethyl ether. After drying under a high vacuum, 938 mg (97% of th.) of the title compound were obtained.

MS (DCI, NH₃): m/z=133 [M+H]⁺.

### Example 104A

### 1-(2,2-Difluoroethyl)piperazine dihydrochloride

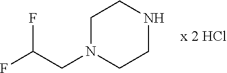

### Step 1: tert-Butyl 4-(2,2-difluoroethyl)piperazine-1-carboxylate

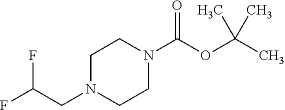 [SMILES: CC(C)(C)OC(=O)N1CCN(CC(F)F)CC1]

1.35 ml (9.66 mmol) of anhydrous triethylamine and 1.27 ml (7.52 mmol) of trifluoromethanesulfonic acid anhydride were added to a solution of 408 μl (6.44 mmol) of 2,2-difluoroethanol in 10 ml of anhydrous methylene chloride at 0° C. After stirring at 0° C. for 30 min, a solution of 1.0 g (5.37 mmol) of tert-butyl piperazine-1-carboxylate in 10 ml of anhydrous methylene chloride was added. The reaction mixture was then allowed to warm to RT. After 16 h, approx. 20 ml of water were added and the phases were separated. The organic phase was washed with water and dried over anhydrous magnesium sulfate. After filtration, the solvent was removed on a rotary evaporator and the residue was purified by means of MPLC (silica gel; mobile phase: cyclohexane/ethyl acetate 1:2). 538 mg (45% of th.) of the title compound were obtained.

¹H-NMR (400 MHz, CDCl₃, δ/ppm): 5.88 (tt, 1H), 3.43 (t, 4H), 2.75 (dt, 2H), 2.53 (t, 4H), 1.45 (s, 9H).

GC/MS (method L, EI): Rₜ=4.41 min, m/z=250 [M]⁺.

### Step 2: 1-(2,2-Difluoroethyl)piperazine dihydrochloride

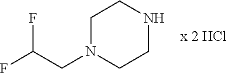

Analogously to the process described under Example 103A/step 2, 257 mg (92% of th.) of the title compound were obtained starting from 314 mg (1.26 mmol) of the compound from Example 104A/step 1.

MS (DCI, NH₃): m/z=151 [M+H]⁺.

### Example 105A

### 5-Methyl-1-{3-[(4-methylpiperazin-1-yl)carbonyl]benzyl}-1H-pyrazole-3-carboxylic acid

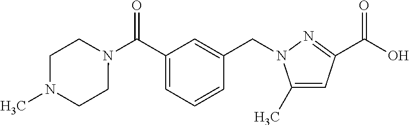 [SMILES: Cc1cc(C(=O)O)nn1Cc1cccc(C(=O)N2CCN(C)CC2)c1]

### Step 1: Ethyl 1-[3-(tert-butoxycarbonyl)benzyl]-5-methyl-1H-pyrazole-3-carboxylate

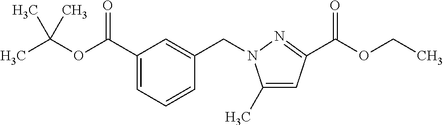 [SMILES: CCOC(=O)c1cc(C)n(Cc2cccc(C(=O)OC(C)(C)C)c2)n1]

1.90 g (17.0 mmol) of solid potassium tert-butylate were added to a solution of 2.38 g (15.4 mmol) of ethyl 5-methyl-1H-pyrazole-3-carboxylate and 4.60 g (17.0 mmol) of tert-butyl-3-(bromomethyl)benzenecarboxylate in 50 ml of anhydrous THF at 0° C. The reaction mixture was stirred at RT for 16 h. Approx. 250 ml of water were then added and the mixture was extracted three times with approx. 150 ml of ethyl acetate each time. The combined organic extracts were washed successively with water and saturated sodium chloride solution. After drying over anhydrous magnesium sulfate, the mixture was filtered and the solvent was removed on a rotary evaporator. The residue obtained was purified by means of MPLC (silica gel, mobile phase: cyclohexane/ethyl acetate 10:1→2:1). 4.45 g (84% of th.) of the title compound were obtained.

¹H-NMR (400 MHz, CDCl₃, δ/ppm): 7.90 (d, 1H), 7.77 (s, 1H), 7.36 (t, 1H), 7.19 (d, 1H), 6.63 (s, 1H), 5.42 (s, 2H), 4.42 (quart, 2H), 2.19 (s, 3H), 1.58 (s, 9H), 1.40 (t, 3H).

MS (DCI, NH₃): m/z=345 [M+H]⁺.

### Step 2: 3-{[3-(Ethoxycarbonyl)-5-methyl-1H-pyrazol-1-yl]methyl}benzenecarboxylic acid

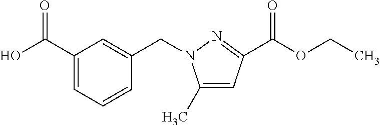 [SMILES: CCOC(=O)c1cc(C)n(Cc2cccc(C(=O)O)c2)n1]

10 ml of trifluoroacetic acid were added to a solution of 4.47 g (13.0 mmol) of the compound from Example 105A/step 1 in 50 ml of methylene chloride. After the reaction mixture had been stirred at RT for 6 h, all the volatile constituents were removed on a rotary evaporator. The residue obtained was stirred with diethyl ether and filtered off with suction. After drying under a high vacuum, 3.19 g (85% of th.) of the title compound were obtained.

¹H-NMR (400 MHz, DMSO-d₆, δ/ppm): 13.05 (s, broad, 1H), 7.87 (d, 1H), 7.69 (s, 1H), 7.49 (t, 1H), 7.37 (d, 1H), 6.60 (s, 1H), 5.49 (s, 2H), 4.25 (quart, 2H), 2.24 (s, 3H), 1.27 (t, 3H).

HPLC (method A): Rₜ=3.71 min.

MS (ESIpos): m/z=289 [M+H]⁺.

LC/MS (method F, ESIpos): Rₜ=0.94 min, m/z=289 [M+H]⁺.

### Step 3: Ethyl 5-methyl-1-{3-[(4-methylpiperazin-1-yl)carbonyl]benzyl}-1H-pyrazole-3-carboxylate

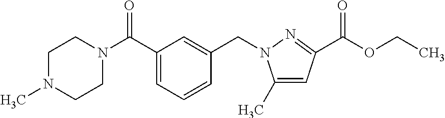 [SMILES: CCOC(=O)c1cc(C)n(Cc2cccc(C(=O)N3CCN(C)CC3)c2)n1]

Under inert conditions, 3.15 g (10.9 mmol) of the compound from Example 105A/step 2 were dissolved in 100 ml of anhydrous methylene chloride, and 4.8 ml (54.6 mmol) of oxalyl chloride and one drop of DMF were added. After the mixture had been stirred at RT for approx. 2.5 h, it was concentrated to dryness on a rotary evaporator. The residue obtained was dried under a high vacuum for approx. 1 h and then dissolved in 40 ml of anhydrous THF. This solution was added dropwise to a solution of 2.19 g (21.9 mmol) of 1-methylpiperazine and 5.7 ml (32.8 mmol) of N,N-diisopropylethylamine in 60 ml of anhydrous THF. After stirring at RT for 16 h, the reaction mixture was diluted with approx. 400 ml of water and extracted three times with approx. 100 ml of ethyl acetate each time. The combined organic extracts were washed successively with water and saturated sodium chloride solution. After drying over anhydrous magnesium sulfate, the mixture was filtered and the solvent was removed on a rotary evaporator. 4.04 g (99% of th.) of the title compound were obtained.

¹H-NMR (400 MHz, CDCl₃, δ/ppm): 7.36 (t, 1H), 7.31 (d, 1H), 7.13 (d, 1H), 7.09 (s, 1H), 6.62 (s, 1H), 5.40 (s, 2H), 4.40 (quart, 2H), 3.77 (broad, 2H), 3.36 (broad, 2H), 2.46 (broad, 2H), 2.31 (s, 3H), 2.30 (broad, 2H), 2.20 (s, 3H), 1.40 (t, 3H).

LC/MS (method I, ESIpos): Rₜ=0.61 min, m/z=371 [M+H]⁺.

### Step 4: 5-Methyl-1-{3-[(4-methylpiperazin-1-yl)carbonyl]benzyl}-1H-pyrazole-3-carboxylic acid

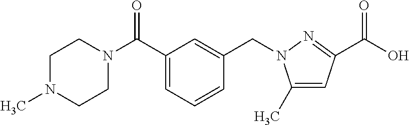 [SMILES: Cc1cc(C(=O)O)nn1Cc1cccc(C(=O)N2CCN(C)CC2)c1]

21.6 ml (21.6 mmol) of 1 M sodium hydroxide solution were added dropwise to a solution of 4.0 g (10.8 mmol) of the compound from Example 105A/step 3 in 70 ml of ethanol and the mixture was heated at 70° C. for 2 h. The ethanol was then mostly removed on a rotary evaporator. 3 M hydrochloric acid was added to the aqueous solution which remained at 0° C., while stirring, until a pH of about 4 was reached. A solid thereby precipitated out, and was removed by filtration with suction. The filtrate was evaporated to dryness on a rotary evaporator and the solid residue was then stirred with methylene chloride overnight. After filtration, the filtrate was freed from the solvent on a rotary evaporator. 2.35 g (63% of th.) of the title compound were obtained.

¹H-NMR (400 MHz, DMSO-d₆, δ/ppm): 7.43 (t, 1H), 7.30 (d, 1H), 7.21 (d, 1H), 7.07 (s, 1H), 6.51 (s, 1H), 5.76 (s, 1H), 5.41 (s, 2H), 3.57 (broad, 2H), 3.24 (broad, 2H), 2.34 (broad, 2H), 2.23 (s, 3H), 2.20 (broad, 2H), 2.18 (s, 3H).

LC/MS (method I, ESIpos): Rₜ=0.33 min, m/z=343 [M+H]⁺.

### Example 106A

### 5-Methyl-1-{3-[(4-methylpiperazin-1-yl)carbonyl]benzyl}-1H-pyrrole-3-carboxylic acid

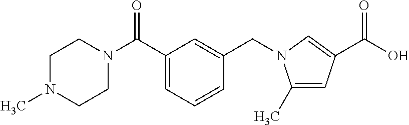 [SMILES: Cc1cc(C(=O)O)cn1Cc1cccc(C(=O)N2CCN(C)CC2)c1]

### Step 1: 3-[(4-Methylpiperazin-1-yl)carbonyl]benzenecarbonitrile

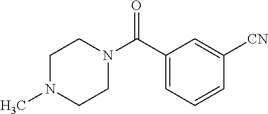 [SMILES: CN1CCN(C(=O)c2cccc(C#N)c2)CC1]

A solution of 3-cyanobenzoic acid chloride in 100 ml of methylene chloride was added dropwise to a solution of 4.57 g (45.6 mmol) of 1-methylpiperazine and 8.5 ml (60.8 mmol) of triethylamine in 100 ml of methylene chloride at 0° C. The mixture was then stirred at RT for 6 h. 200 ml of water were then added, the phases were separated and the organic phase was washed with water. After drying over anhydrous magnesium sulfate, the mixture was filtered and the solvent was removed on a rotary evaporator. 6.9 g (99% of th.) of the title compound were obtained.

¹H-NMR (400 MHz, CDCl₃, δ/ppm): 7.72 (d, 1H), 7.70 (s, 1H), 7.65 (d, 1H), 7.55 (t, 1H), 3.80 (broad, 2H), 3.40 (broad, 2H), 2.50 (broad, 2H), 2.37 (broad, 2H), 2.34 (s, 3H).

LC/MS (method I, ESIpos): Rₜ=0.21 min, m/z=230 [M+H]⁺.

GC/MS (method L, ESIpos): Rₜ=7.36 min, m/z=229 [M]⁺.

### Step 2: [3-(Aminomethyl)phenyl](4-methylpiperazin-1-yl)methanone

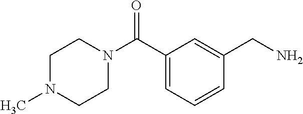 [SMILES: CN1CCN(C(=O)c2cccc(CN)c2)CC1]

A solution of 1.0 g (4.36 mmol) of the compound from Example 106A/step 1 in 100 ml of ethanol was hydrogenated in a flow-through hydrogenation apparatus (“H-Cube” from ThalesNano, Budapest, Hungary; Raney nickel catalyst, “full H₂” mode, 0.5 ml/min, 50° C.). After the solvent had been evaporated off, 1.0 g (99% of th.) of the title compound were obtained.

LC/MS (method D, ESIpos): Rₜ=0.19 min, m/z=234 [M+H]⁺.

### Step 3: Methyl 5-methyl-1-{3-[(4-methylpiperazin-1-yl)carbonyl]benzyl}-1H-pyrrole-3-carboxylate

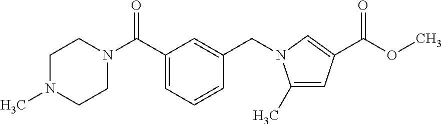

1.0 g (4.41 mmol) of the compound from Example 106A/step 2 were dissolved in 10 ml of methanol, and 698 mg (4.41 mmol) of the compound from Example 50A/step 1 were added. After the reaction mixture had been stirred at RT for 1 h, the solvent was again removed on a rotary evaporator and the residue was purified by means of MPLC (silica gel; mobile phase: methylene chloride/methanol 10:1). 1.07 g (68% of th.) of the title compound were obtained.

¹H-NMR (400 MHz, CDCl₃, δ/ppm): 7.37 (t, 1H), 7.32 (d, 1H), 7.27 (d, 1H), 7.04 (d, 1H), 7.03 (d, 1H), 5.05 (s, 2H), 3.79 (s, 3H), 3.77 (broad, 2H), 3.36 (broad, 2H), 2.48 (broad, 2H), 2.31 (s, 3H), 2.11 (s, 3H), 1.95 (broad, 2H).

LC/MS (method I, ESIpos): Rₜ=0.63 min, m/z=356 [M+H]⁺.

### Step 4: 5-Methyl-1-{3-[(4-methylpiperazin-1-yl)carbonyl]benzyl}-1H-pyrrole-3-carboxylic acid

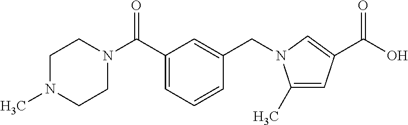 [SMILES: Cc1cc(C(=O)O)cn1Cc1cccc(C(=O)N2CCN(C)CC2)c1]

0.90 g (2.53 mmol) of the compound from Example 106A/step 3 were dissolved in 17.5 ml of methanol and 5 ml (5.0 mmol) of 1 M sodium hydroxide solution were added. The mixture was reacted in portions in a microwave oven at 80° C. for in each case 30 min (CEM Discover, initial irradiation power 250 W). The reaction mixture was subsequently adjusted to a pH of approx. 4-5 by addition of 6 M hydrochloric acid and then purified in portions by means of preparative HPLC (method N). 344 mg (39% of th.) of the title compound were obtained.

LC/MS (method I, ESIpos): Rₜ=0.53 min, m/z=342 [M+H]⁺.

### Example 107A

### N′-Hydroxy-4-(tetrahydro-2H-pyran-4-yl)benzenecarboximide amide

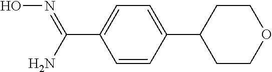 [SMILES: N/C(=N\O)c1ccc(C2CCOCC2)cc1]

### Step 1: 4-(Tetrahydro-2H-pyran-4-yl)benzonitrile

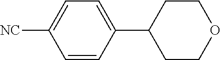 [SMILES: N#Cc1ccc(C2CCOCC2)cc1]

186 mg (0.594 mmol) of nickel (II) iodide, 90 mg (0.594 mmol) of trans-2-aminocyclohexanol hydrochloride and 3.63 g (19.8 mmol) of sodium hexamethyldisilazide were added to a solution of 2.91 g (19.8 mmol) of 4-cyanophenylboronic acid [M. Nishimura et al., Tetrahedron 2002, 58 (29), 5779-5788] in 20 ml of isopropanol. The suspension obtained in this way was stirred at RT under an argon atmosphere for 5 min 2.1 g (9.90 mmol) of 4-iodotetrahydropyran [Heuberger et al., J. Chem. Soc. 1952, 910] were then added. After the reaction mixture had been stirred at a temperature of 75° C. for 15 h, it was cooled to RT and largely freed from inorganic salts with methylene chloride by filtration over approx. 50 g of silica gel. The crude product was purified by MPLC (silica gel, mobile phase: methylene chloride). 986 mg (53% of th.) of the title compound were obtained in this way.

¹H-NMR (400 MHz, CDCl₃, δ/ppm): 7.60 (d, 2H), 7.32 (d, 2H), 4.12-4.07 (m, 2H), 3.56-3.50 (m, 2H), 2.87-2.79 (m, 1H), 1.86-1.73 (m, 4H).

GC/MS (method L, EIpos): Rₜ=5.97 min, m/z=187 [M]⁺.

### Step 2: N′-Hydroxy-4-(tetrahydro-2H-pyran-4-yl)benzenecarboximide amide

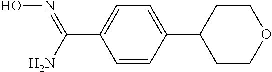 [SMILES: N/C(=N\O)c1ccc(C2CCOCC2)cc1]

Analogously to the process described under Example 1A/step 5, 480 mg (2.56 mmol) of the compound from Example 107A/step 1 were reacted to give 525 mg (93% of th.) of the title compound.

¹H-NMR (400 MHz, CDCl₃, δ/ppm): 7.58 (d, 2H), 7.26 (d, 2H), 6.79 (broad, 1H), 4.82 (s, broad, 2H), 4.11-4.05 (m, 2H), 3.57-3.50 (m, 2H), 2.83-2.74 (m, 1H), 1.87-1.73 (m, 4H).

LC/MS (method D, ESIpos): Rₜ=0.92 min, m/z=221 [M+H]⁺.

### Example 108A

### N′-Hydroxy-3-methyl-4-(tetrahydro-2H-pyran-4-yl)benzenecarboximide amide

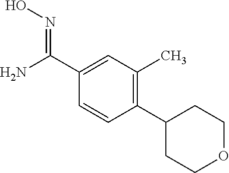 [SMILES: Cc1cc(/C(N)=N/O)ccc1C1CCOCC1]

### Step 1: 3-Methyl-4-(tetrahydro-2H-pyran-4-yl)benzonitrile

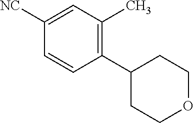

Analogously to the process described under Example 107A, step 1, 481 ml (18% of th.) of the title compound were obtained from 4.17 g (25.9 mmol) of 4-cyano-2-methylphenylboronic acid [D. Stones et al., Chem. Eur. J. 2004, 10 (1), 92-100] and 2.75 g (13.0 mmol) of 4-iodotetrahydropyran [Heuberger et al., J. Chem. Soc. 1952, 910].

¹H-NMR (400 MHz, CDCl₃, δ/ppm): 7.49 (dd, 1H), 7.43 (d, 1H), 7.31 (d, 1H), 4.12-4.09 (m, 2H), 3.59-3.52 (m, 2H), 3.05-2.97 (m, 1H), 2.39 (s, 3H), 1.86-1.75 (m, 2H), 1.69-1.64 (m, 2H).

GC/MS (method L, EIpos): Rₜ=6.31 min, m/z=201 [M]⁺.

### Step 2: N′-Hydroxy-3-methyl-4-(tetrahydro-2H-pyran-4-yl)benzenecarboximide amide

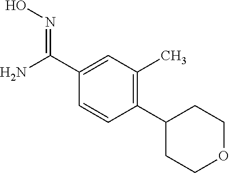 [SMILES: Cc1cc(/C(N)=N/O)ccc1C1CCOCC1]

Analogously to the process described under Example 1A/step 5, 492 mg (84% of th.) of the title compound were obtained from 500 mg (2.48 mmol) of the compound from Example 108A/step 1.

¹H-NMR (400 MHz, DMSO-d₆, δ/ppm): 9.49 (s, 1H), 7.45 (d, 1H), 7.44 (s, 1H), 7.21 (d, 1H), 5.69 (s, broad, 2H), 3.97-3.93 (m, 2H), 3.50-3.43 (m, 2H), 3.00-2.92 (m, 1H), 2.33 (s, 3H), 1.72-1.57 (m, 4H).

LC/MS (method I, ESIpos): Rₜ=0.49 min, m/z=235 [M+H]⁺.

### Example 109A

### Ethyl 1-[4-(N′-hydroxycarbamimidoyl)phenyl]cyclobutanecarboxylate

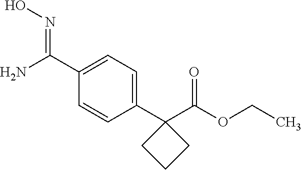 [SMILES: CCOC(=O)C1(c2ccc(/C(N)=N/O)cc2)CCC1]

### Step 1: Ethyl 1-(4-bromophenyl)cyclobutanecarboxylate

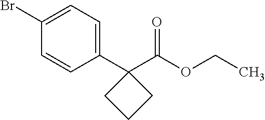 [SMILES: CCOC(=O)C1(c2ccc(Br)cc2)CCC1]

45 ml (45.2 mmol) of a 1 M solution of lithium hexamethyldisilazide in THF were added to a solution of 10.0 g (41.1 mmol) of 4-bromophenylacetic acid ethyl ester in 250 ml of anhydrous THF at 0° C. After 15 min, 5.4 ml (53.5 mmol) of 1,3-dibromopropane were added. The reaction mixture was allowed to warm to RT and was subsequently stirred at this temperature for 1 h. It was then cooled again to 0° C. and a further 45 ml (45.2 mmol) of lithium hexamethyldisilazide solution (1 M in THF) were added. Thereafter, the mixture was warmed again to RT. After 1 h, the reaction was ended by addition of approx. 10 ml of saturated aqueous ammonium chloride solution. The THF was largely removed on a rotary evaporator. The residue was diluted with water and extracted with ethyl acetate. The organic extract was washed successively with water and saturated sodium chloride solution. After drying over anhydrous magnesium sulfate, the mixture was filtered and the filtrate was freed from the solvent on a rotary evaporator. The crude product obtained in this way was coarsely purified by means of filtration with suction over approx. 300 g of silica gel with cyclohexane/ethyl acetate 3:1 as the mobile phase. 7.1 g (44% of th., purity of 73%) of the title compound were obtained, this being reacted further in this form.

¹H-NMR (400 MHz, CDCl₃, δ/ppm): 7.44 (d, 2H), 7.17 (d, 2H), 4.10 (quart, 2H), 2.85-2.79 (m, 2H), 2.49-2.41 (m, 2H), 2.10-1.98 (m, 1H), 1.91-1.81 (m, 1H), 1.18 (t, 3H).

MS (DCI, NH₃): m/z=300/302 [M+NH₄]⁺.

LC/MS (method D, ESIpos): Rₜ=2.70 min, m/z=283/285 [M+H]⁺.

### Step 2: Ethyl 1-(4-cyanophenyl)cyclobutanecarboxylate

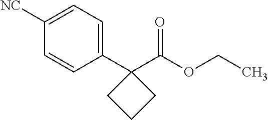 [SMILES: CCOC(=O)C1(c2ccc(C#N)cc2)CCC1]

A mixture of 160 mg (0.565 mmol) of the compound from Example 109A/step 1, 76 mg (0.644 mmol) of zinc cyanide, 26 mg (0.028 mmol) of tris(dibenzylidene-acetone)dipalladium and 23 mg (0.057 mmol) of dicyclohexyl-(2′,6′-dimethoxybiphen-2-yl)phosphane in 6 ml of DMF/water (99:1) was heated at 120° C. under oxygen-free conditions for 1 h. After cooling to RT, the mixture was diluted with approx. 30 ml of water and extracted three times with approx. 20 ml of ethyl acetate each time. The combined organic extracts were washed with saturated sodium chloride solution and dried over anhydrous magnesium sulfate. After filtration, the solvent was removed on a rotary evaporator. The residue obtained was first prepurified by means of MPLC (silica gel, mobile phase: cyclohexane/ethyl acetate 10:1). The product was then isolated in a pure form by means of preparative HPLC (method N). 110 mg (85% of th.) of the title compound were obtained.

¹H-NMR (400 MHz, CDCl₃, δ/ppm): 7.62 (d, 2H), 7.39 (d, 2H), 4.10 (quart, 2H), 2.90-2.83 (m, 2H), 2.52-2.44 (m, 2H), 2.15-2.03 (m, 1H), 1.93-1.83 (m, 1H), 1.17 (t, 3H).

LC/MS (method D, ESIpos): Rₜ=2.32 min, m/z=230 [M+H]⁺.

### Step 3: Ethyl 1-[4-(N′-hydroxycarbamimidoyl)phenyl]cyclobutanecarboxylate

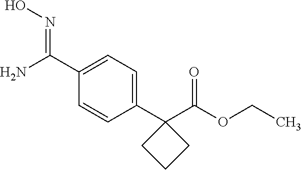 [SMILES: CCOC(=O)C1(c2ccc(/C(N)=N/O)cc2)CCC1]

Analogously to the process described under Example 1A/step 5, 122 mg (91% of th., purity of 90%) of the title compound were obtained from 105 mg (0.458 mmol) of the compound from Example 109A/step 2.

¹H-NMR (400 MHz, CDCl₃, δ/ppm): 7.59 (d, 2H), 7.33 (d, 2H), 4.84 (broad, 2H), 4.10 (quart, 2H), 2.88-2.80 (m, 2H), 2.53-2.46 (m, 2H), 2.10-1.99 (m, 1H), 1.92-1.82 (m, 1H), 1.17 (t, 3H).

LC/MS (method I, ESIpos): Rₜ=0.67 min, m/z=263 [M+H]⁺.

### Example 110A

### N′-Hydroxy-4-[1-(methoxymethyl)cyclobutyl]benzenecarboximide amide

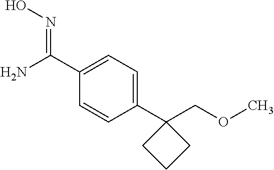 [SMILES: COCC1(c2ccc(/C(N)=N/O)cc2)CCC1]

### Step 1: [1-(4-Bromophenyl)cyclobutyl]methanol

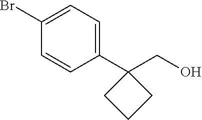 [SMILES: OCC1(c2ccc(Br)cc2)CCC1]

7.20 g (25.4 mmol) of the compound from Example 109A/step 1 were dissolved in 150 ml of anhydrous THF, and 25 ml (25 mmol) of a 1 M solution of lithium aluminium hydride in THF were added dropwise at 0° C. When the addition had ended, the ice/water bath was removed and stirring was continued at RT. After 1 h, the reaction was ended by—initially cautious—addition of approx. 450 ml of saturated aqueous ammonium chloride solution. The mixture was then extracted with ethyl acetate. After drying of the organic extracts over anhydrous magnesium sulfate and subsequent filtration, the solvent was removed on a rotary evaporator. 6.04 g (88% of th., purity of approx. 90%) of the title compound were obtained.

¹H-NMR (400 MHz, CDCl₃, δ/ppm): 7.43 (d, 2H), 7.02 (d, 2H), 3.72 (d, 2H), 2.33-2.20 (m, 4H), 2.13-2.01 (m, 1H), 1.93-1.83 (m, 1H).

MS (DCI, NH₃): m/z=258/260 [M+NH₄]⁺.

GC/MS (method L, ESIpos): Rₜ=5.77 min, m/z=240/242 [M]⁺.

### Step 2: 1-Bromo-4-[1-(methoxymethyl)cyclobutyl]benzene

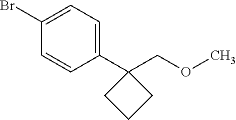

1.28 g (31.9 mmol) of a 60% strength suspension of sodium hydride in mineral oil were added to a solution of 7.0 g (29.0 mmol) of the compound from Example 110A/step 1 in 120 ml of anhydrous DMF at approx. 5° C. After the mixture had been stirred at this temperature for 1 h, 2.2 ml (34.8 mmol) of methyl iodide were added. The reaction mixture was allowed to warm to RT and stirring was continued for 15 h. The reaction mixture was then concentrated to a volume of approx. 20 ml on a rotary evaporator. Approx. 500 ml of water were added and the mixture was extracted three times with approx. 200 ml of diethyl ether each time. The combined organic extracts were washed with saturated sodium chloride solution and dried over anhydrous magnesium sulfate. After filtration and removal of the solvent on a rotary evaporator, the crude product obtained was purified by means of filtration with suction over approx. 200 g of silica gel with cyclohexane/ethyl acetate 50:1 as the mobile phase. 4.92 g (66% of th.) of the title compound were obtained.

¹H-NMR (400 MHz, CDCl₃, δ/ppm): 7.41 (d, 2H), 7.04 (d, 2H), 3.48 (s, 2H), 3.27 (s, 3H), 2.32-2.22 (m, 4H), 2.12-2.00 (m, 1H), 1.90-1.80 (m, 1H).

MS (DCI, NH₃): m/z=272/274 [M+NH₄]⁺.

GC/MS (method L, ESIpos): Rₜ=5.25 min, m/z=254/256 [M]+.

### Step 3: 4-[1-(Methoxymethyl)cyclobutyl]benzonitrile

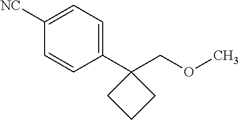

Analogously to the process described under Example 109A/step 2, 1.82 g (48% of th.) of the title compound were obtained from 4.80 g (18.8 mmol) of the compound from Example 110A/step 2.

¹H-NMR (400 MHz, CDCl₃, δ/ppm): 7.58 (d, 2H), 7.24 (d, 2H), 3.52 (s, 2H), 3.26 (s, 3H), 2.34-2.24 (m, 4H), 2.16-2.03 (m, 1H), 1.92-1.83 (m, 1H).

LC/MS (method F, ESIpos): Rₜ=1.22 min, m/z=202 [M+H]⁺.

### Step 4: N′-Hydroxy-4-[1-(methoxymethyl)cyclobutyl]benzenecarboximide amide

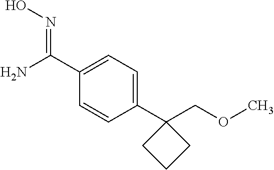 [SMILES: COCC1(c2ccc(/C(N)=N/O)cc2)CCC1]

Analogously to the process described under Example 1A/step 5, 2.04 g (96% of th.) of the title compound were obtained from 1.82 g (9.04 mmol) of the compound from Example 110A/step 3.

¹H-NMR (400 MHz, CDCl₃, δ/ppm): 7.55 (d, 2H), 7.20 (d, 2H), 7.10 (broad, 1H), 4.83 (broad, 2H), 3.51 (s, 2H), 3.27 (s, 3H), 2.36-2.25 (m, 4H), 2.12-2.01 (m, 1H), 1.90-1.81 (m, 1H).

LC/MS (method I, ESIpos): Rₜ=0.61 min, m/z=235 [M+H]⁺.

### Example 111A

### N′-Hydroxy-4-(1,1,1-trifluoro-2-methoxypropan-2-yl)benzenecarboximide amide (racemate)

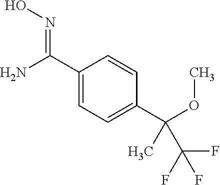 [SMILES: COC(C)(c1ccc(/C(N)=N/O)cc1)C(F)(F)F]

### Step 1: 4-(1,1,1-Trifluoro-2-hydroxypropan-2-yl)benzonitrile (racemate)

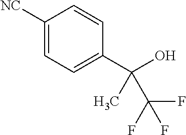

5.0 g (21.8 mmol) of 4-iodobenzonitrile were dissolved in 100 ml of anhydrous THF and the solution was cooled to −40° C. 11.5 ml (22.9 mmol) of a 2 M solution of isopropylmagnesium chloride in diethyl ether were added dropwise such that the temperature of the reaction mixture remained in the range between −30° C. and −40° C. When the addition had ended, the mixture was stirred further in this temperature interval for another 15 h, before it was cooled to −78° C. 11.2 g (100 mmol) of 1,1,1-trifluoroacetone were then added dropwise. The mixture was allowed to come to RT in the course of several hours and was stirred at RT for a further approx. 5 h. Approx. 5 ml of water were then cautiously added. The majority of the solvent was then removed on a rotary evaporator, until a residual volume of approx. 50 ml remained left. Approx. 100 ml of 0.5 M hydrochloric acid were added to this residue and the mixture was extracted three times with approx. 100 ml of ethyl acetate each time. The combined organic extracts were washed successively with water and saturated sodium chloride solution and dried over anhydrous magnesium sulfate. After filtration, the solvent was removed on a rotary evaporator and the residue obtained was purified by means of MPLC (approx. 200 g of silica gel, mobile phase: cyclohexane/ethyl acetate 20:1→5:1). 3.63 g (73% of th., purity of approx. 95%) of the title compound were obtained.

LC/MS (method I, ESIneg): Rₜ=0.89 min, m/z=214 [M−H]⁻, 260 [M−H+HCO₂H]⁻.

### Step 2: 4-(1,1,1-Trifluoro-2-methoxypropan-2-yl)benzonitrile (racemate)

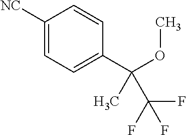 [SMILES: COC(C)(c1ccc(C#N)cc1)C(F)(F)F]

Analogously to the process described under Example 110A/step 2, 1.01 g (59% of th.) of the title compound were obtained from 1.6 g (7.44 mmol) of the compound from Example 111A/step 1 and 555 μl (8.92 mmol) of methyl iodide. Purification by chromatography was carried out with cyclohexane/ethyl acetate 100:0→20:1 as the mobile phase.

¹H-NMR (400 MHz, CDCl₃, δ/ppm): 7.71 (d, 2H), 7.63 (d, 2H), 3.27 (s, 3H), 1.80 (s, 3H).

HPLC (method A): Rₜ=4.01 min.

GC/MS (method L, EIpos): Rₜ=4.13 min, m/z=214 [M−CH₃]⁺, 160 [M−CF₃]⁺.

### Step 3: N′-Hydroxy-4-(1,1,1-trifluoro-2-methoxypropan-2-yl)benzenecarboximide amide (racemate)

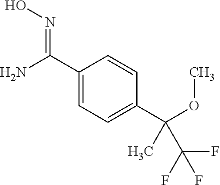 [SMILES: COC(C)(c1ccc(/C(N)=N/O)cc1)C(F)(F)F]

Analogously to the process described under Example 1A/step 5, 1.07 g (89% of th., purity of 94%) of the title compound were obtained from 990 mg (4.32 mmol) of the compound from Example 111A/step 2.

LC/MS (method D, ESIpos): Rₜ=1.23 min, m/z=263 [M+H]⁺.

### Example 112A

### 3-Fluoro-N′-hydroxy-4-(tetrahydro-2H-pyran-4-yl)benzenecarboximide amide

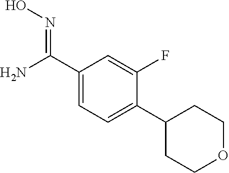 [SMILES: N/C(=N\O)c1ccc(C2CCOCC2)c(F)c1]

### Step 1: (4-Cyano-2-fluorophenyl)boronic acid

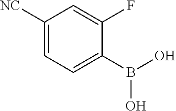 [SMILES: N#Cc1ccc(B(O)O)c(F)c1]

24.3 ml (48.6 mmol) of a 2 M solution of isopropylmagnesium chloride in diethyl ether were added dropwise to a solution of 10.0 g (40.5 mmol) of 3-fluoro-4-iodobenzonitrile in a mixture of 120 ml of anhydrous THF and 120 ml of anhydrous diethyl ether at −78° C. When the addition had ended, the mixture was stirred further at −78° C. for another 75 min. 15 ml (64.8 mmol) of triisopropyl borate were then added dropwise. The mixture was then stirred at −78° C. for a further 15 min, before the cold bath was removed and the reaction mixture was allowed to warm to RT. After 3 h at RT, 80 ml of 2 M hydrochloric acid were added and the mixture was stirred intensively at RT for 20 min. Thereafter, it was diluted with approx. 400 ml of water. The phases were separated and the aqueous phase was extracted three times with approx. 150 ml of ethyl acetate each time. The combined organic extracts were washed successively with water and saturated sodium chloride solution. After drying over anhydrous magnesium sulfate, the mixture was filtered and the filtrated was concentrated to dryness on a rotary evaporator. 3.68 g (55% of th.) of the title compound were obtained, this being employed for subsequent reactions without further purification.

LC/MS (method F, ESIneg): Rₜ=0.53 min, m/z=164 [M−H]⁻.

### Step 2: 4-Bromo-3,6-dihydro-2H-pyran

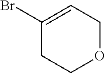

4.79 g (30.0 mmol) of bromine were added dropwise to a solution of 8.52 g (27.5 mmol) of triphenyl phosphite in 78 ml of anhydrous methylene chloride at −60° C. After addition of 4.5 ml (32.5 mmol) of triethylamine, a solution of 2.5 g (25.0 mmol) of tetrahydro-4H-pyran-4-one in 2 ml of methylene chloride was added dropwise. The reaction mixture was allowed to warm slowly (over approx. 5 h) to RT and stirring was continued at RT for a further approx. 10 h. All the volatile constituents were then removed on a rotary evaporator and the residue obtained was chromatographed coarsely by filtration with suction over approx. 100 g of silica gel with methylene chloride as the mobile phase. After renewed evaporation of the solvent, the product was isolated by means of bulb tube distillation (pressure: 8 mbar; temperature: to 120° C.). 2.51 g (62% of th.) of the title compound were obtained.

¹H-NMR (400 MHz, CDCl₃, δ/ppm): 6.07 (m, 1H), 4.13 (m, 2H), 3.83 (m, 2H), 2.55-2.50 (m, 2H).

GC/MS (method L, ESIpos): Rₜ=2.32 min, m/z=162/164 [M]⁺.

### Step 3: 4-(3,6-Dihydro-2H-pyran-4-yl)-3-fluorobenzonitrile

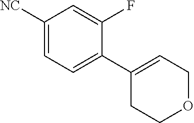 [SMILES: N#Cc1ccc(C2=CCOCC2)c(F)c1]

A mixture of 300 mg (1.84 mmol) of the compound from Example 112A/step 2, 334 mg (2.02 mmol) of the compound from Example 112A/step 1, 8 mg (0.037 mmol) of palladium(II) acetate, 1.17 g (5.52 mmol) of potassium phosphate and 44 mg (0.092 mmol) of 2-dicyclohexylphosphine-2′,2′,6′-triisopropylbiphenyl (XPhos) in 4 ml of anhydrous THF was degassed, and stirred at 80° C. under argon in a microwave oven (CEM Discover, initial irradiation power 250 W) for 1 h. After cooling to RT, all the volatile constituents were removed on a rotary evaporator. The product was isolated from the residue by means of MPLC (approx. 50 g of silica gel, mobile phase: cyclohexane/methylene chloride 100:0,→5:50→5:95). 160 mg (43% of th.) of the title compound were obtained.

¹H-NMR (400 MHz, CDCl₃, δ/ppm): 7.42 (d, 1H), 7.37 (d, 1H), 7.36 (d, 1H), 6.18 (m, 1H), 4.33 (m, 2H), 3.92 (t, 2H), 2.52-2.48 (m, 2H).

GC/MS (method L, ESIpos): Rₜ=5.79 min, m/z=203 [M]⁺.

### Step 4: 3-Fluoro-4-(tetrahydro-2H-pyran-4-yl)benzonitrile

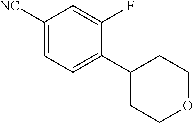 [SMILES: N#Cc1ccc(C2CCOCC2)c(F)c1]

330 mg (1.62 mmol) of the compound from Example 112A/step 3 were dissolved in a mixture of 22 ml of ethyl acetate and 22 ml of ethanol. Hydrogenation was carried out in a flow-through hydrogenation apparatus (“H-Cube” from Thales Nano, Budapest, Hungary; conditions: cartridge with 5% palladium on charcoal, hydrogen pressure of 10 bar, temperature 20° C., flow rate 1 ml/min). The solution was passed through the apparatus four times in total, until the reaction was complete. The solvent was then removed on a rotary evaporator. 211 mg (63% of th.) of the title compound were obtained.

¹H-NMR (400 MHz, CDCl₃, δ/ppm): 7.43 (d, 1H), 7.37 (d, 1H), 7.32 (d, 1H), 4.11-4.07 (m, 2H), 3.59-3.53 (m, 2H), 3.21-3.13 (m, 1H), 1.88-1.72 (m, 4H).

GC/MS (method L, EIpos): Rₜ=5.59 min, m/z=205 [M]⁺.

### Step 5: 3-Fluoro-N′-hydroxy-4-(tetrahydro-2H-pyran-4-yl)benzenecarboximide amide

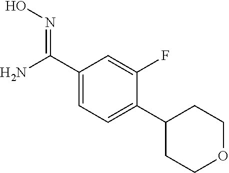 [SMILES: N/C(=N\O)c1ccc(C2CCOCC2)c(F)c1]

Analogously to the process described under Example 1A/step 5, 172 mg (85% of th.) of the title compound were obtained from 175 mg (0.853 mmol) of the compound from Example 112A/step 4.

¹H-NMR (400 MHz, CDCl₃, δ/ppm): 7.40-7.24 (m, 3H), 7.08 (broad, 1H), 4.81 (broad, 2H), 4.10-4.06 (m, 2H), 3.59-3.52 (m, 2H), 3.17-3.10 (m, 1H), 1.89-1.71 (m, 4H).

LC/MS (method F, ESIpos): Rₜ=0.46 min, m/z=239 [M+H]⁺.

### Example 113A

### 4-[1-(2-Fluoroethyl)cyclobutyl]-N′-hydroxybenzenecarboximide amide

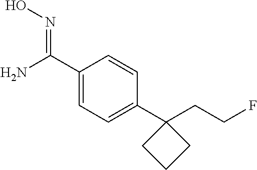 [SMILES: N/C(=N\O)c1ccc(C2(CCF)CCC2)cc1]

### Step 1: Ethyl {1-[4-(dibenzylamino)phenyl]cyclobutyl}acetate

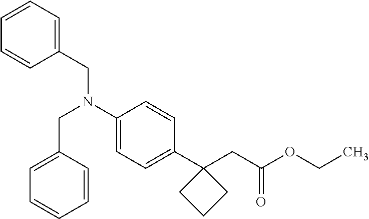 [SMILES: CCOC(=O)CC1(c2ccc(N(Cc3ccccc3)Cc3ccccc3)cc2)CCC1]

440 mg (0.892 mmol) of bis[(1,5-cyclooctadiene)rhodium(I) chloride] were initially introduced into 20 ml of 1,4-dioxane and 15.5 ml (23.2 mmol) of 1.5 M potassium hydroxide solution were added. A solution of 2.5 g (17.8 mmol) of cyclobutylidene-acetic acid ethyl ester [M. Afzal et al., J. Chem. Soc. Perkin Trans. 2, 1999 (5), 937-946] in 1 ml of 1,4-dioxane was then added. A solution of 5.66 g (17.8 mmol) of the compound from Example 4A/step 1 in 100 ml of 1,4-dioxane was then added. After the reaction mixture had been stirred at RT for 16 h, it was concentrated to dryness on a rotary evaporator. The residue obtained was dissolved in a little methylene chloride and prepurified by means of filtration with suction over approx. 100 g of silica gel with methylene chloride as the mobile phase. The product was isolated in a pure form by means of MPLC (approx. 300 g of silica gel, mobile phase: cyclohexane/methylene chloride 100:0,→50:50). 4.02 g (54% of th.) of the title compound were obtained.

¹H-NMR (400 MHz, CDCl₃, δ/ppm): 7.32-7.30 (m, 4H), 7.26-7.21 (m, 6H), 6.97 (d, 2H), 6.67 (d, 2H), 4.61 (s, 4H), 3.93 (quart, 2H), 2.70 (s, 2H), 2.43-2.28 (m, 4H), 2.07-1.96 (m, 1H), 1.88-1.78 (m, 1H).

HPLC (method A): Rₜ=4.74 min.

MS (DCI, NH₃): m/z=414 [M+H]⁺.

### Step 2: 2-{1-[4-(Dibenzylamino)phenyl]cyclobutyl}ethanol

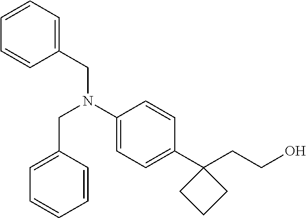 [SMILES: OCCC1(c2ccc(N(Cc3ccccc3)Cc3ccccc3)cc2)CCC1]

36.3 ml (36.3 mmol) of a 1 M solution of lithium aluminium hydride in THF were added dropwise to a solution of 15.0 g (36.3 mmol) of the compound from Example 113A/step 1 in 500 ml of anhydrous THF at 0° C. When the addition had ended, the reaction mixture was allowed to warm to RT and stirring was continued for 2 h. The reaction was then ended cautiously at 0° C. by addition of 20 g of kieselguhr and 20 ml of water. The mixture was filtered with suction over a paper filter and the residue was rinsed with tert-butyl methyl ether. The filtrate was mostly freed from the solvent on a rotary evaporator. The residue was taken up in approx. 400 ml of ethyl acetate and the mixture was washed in each case once with water and saturated sodium chloride solution. After drying over anhydrous magnesium sulfate, the mixture was filtered and the solvent was evaporated off again in vacuo. The crude product was purified by filtration with suction over approx. 250 g of silica gel with cyclohexane/ethyl acetate 10:1→3:1 as the mobile phase. 11.6 g (85% of th.) of the title compound were obtained.

¹H-NMR (400 MHz, CDCl₃, δ/ppm): 7.33-7.30 (m, 4H), 7.26-7.22 (m, 6H), 6.93 (d, 2H), 6.69 (d, 2H), 4.61 (s, 4H), 3.49-3.43 (m, 2H), 2.35-2.28 (m, 2H), 2.12-2.00 (m, 5H), 1.85-1.78 (m, 1H).

HPLC (method A): Rₜ=4.81 min.

MS (DCI, NH₃): m/z=372 [M+H]⁺.

### Step 3: N,N-Dibenzyl-4-[1-(2-fluoroethyl)cyclobutyl]aniline

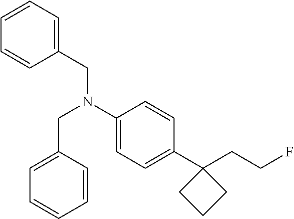 [SMILES: FCCC1(c2ccc(N(Cc3ccccc3)Cc3ccccc3)cc2)CCC1]

1.3 ml (9.69 mmol) of diethylaminosulfur trifluoride (DAST) were added dropwise to a solution of 3.0 g (8.07 mmol) of the compound from Example 113A/step 2 in 150 ml of anhydrous methylene chloride at a temperature of −78° C. After 30 min at −78° C., the reaction mixture was warmed to about −20° C. for approx. 30 seconds and the reaction vessel was then immersed again in the cold bath at −78° C. After addition of 20 ml of 1 M sodium hydroxide solution, the mixture was warmed to RT. It was diluted with 75 ml of water and extracted three times with approx. 75 ml of methylene chloride each time. The combined organic extracts were washed with water and then dried over anhydrous magnesium sulfate. After filtration, the solvent was removed on a rotary evaporator. The product was isolated by means of MPLC (approx. 300 g of silica gel, mobile phase: cyclohexane/ethyl acetate 100:0,→20:1). 1.48 g (49% of th.) of the title compound were obtained.

¹H-NMR (400 MHz, CDCl₃, δ/ppm): 7.41-7.37 (m, 4H), 7.34-7.30 (m, 6H), 7.00 (d, 2H), 6.75 (d, 2H), 4.69 (s, 4H), 4.30 (td, 2H), 2.44-2.37 (m, 2H), 2.26-2.07 (m, 5H), 1.93-1.84 (m, 1H).

HPLC (method A): Rₜ=5.31 min.

MS (DCI, NH₃): m/z=374 [M+H]⁺.

### Step 4: 4-[1-(2-Fluoroethyl)cyclobutyl]aniline

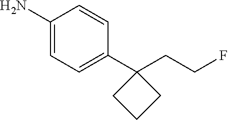 [SMILES: Nc1ccc(C2(CCF)CCC2)cc1]

Analogously to the process described under Example 4A/step 6, 460 mg (62% of th.) of the title compound were obtained from 1.43 g (3.83 mmol) of the compound from Example 113A/step 3. In this case, 180 ml of a mixture of ethanol and ethyl acetate (3:1) was used as the solvent. The crude product obtained after evaporating off the solvent was employed in the subsequent reaction without further purification by chromatography.

¹H-NMR (400 MHz, CDCl₃, δ/ppm): 6.93 (d, 2H), 6.64 (d, 2H), 4.23 (td, 2H), 3.60 (broad, 2H), 2.38-2.31 (m, 2H), 2.20-2.01 (m, 5H), 1.88-1.78 (m, 1H).

LC/MS (method I, ESIpos): Rₜ=0.77 min, m/z=194 [M+H]⁺.

### Step 5: 4-[1-(2-Fluoroethyl)cyclobutyl]benzonitrile

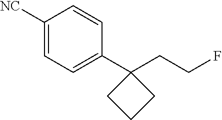 [SMILES: N#Cc1ccc(C2(CCF)CCC2)cc1]

Analogously to the process described under Example 4A/step 7, 259 mg (56% of th.) of the title compound were obtained from 440 mg (2.28 mmol) of the compound from Example 113A/step 4.

¹H-NMR (400 MHz, CDCl₃, δ/ppm): 7.61 (d, 2H), 7.24 (d, 2H), 4.23 (td, 2H), 2.44-2.36 (m, 2H), 2.31-2.10 (m, 5H), 1.91-1.82 (m, 1H).

GC/MS (method L, EIpos): Rₜ=5.63 min, m/z=203 [M]⁺.

### Step 6: 4-[1-(2-Fluoroethyl)cyclobutyl]-N′-hydroxybenzenecarboximide amide

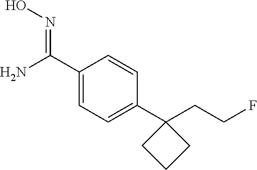 [SMILES: N/C(=N\O)c1ccc(C2(CCF)CCC2)cc1]

Analogously to the process described under Example 1A/step 5, 102 mg (35% of th.) of the title compound were obtained from 250 mg (1.23 mmol) of the compound from Example 113A/step 5. The crude product was purified by means of preparative HPLC (method N).

LC/MS (method D, ESIpos): Rₜ=1.37 min, m/z=237 [M+H]⁺.

### Example 114A

### N′-Hydroxy-4-(1,1,1-trifluoro-2-hydroxypropan-2-yl)benzenecarboximide amide (racemate)

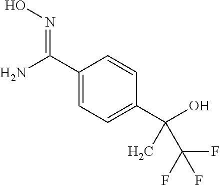 [SMILES: CC(O)(c1ccc(/C(N)=N/O)cc1)C(F)(F)F]

Analogously to the process described under Example 1A/step 5, 1.0 g (4.65 mmol) of the compound from Example 111A/step 1 were reacted to give 1.12 g (83% of th., purity of 85%) of the title compound.

LC/MS (method F, ESIpos): Rₜ=0.36 min, m/z=249 [M+H]⁺.

### Example 115A

### {1-[4-(N′-Hydroxycarbamimidoyl)phenyl]cyclobutyl}methyl acetate

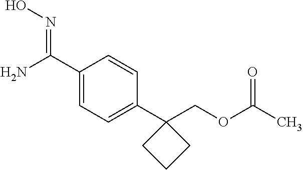 [SMILES: CC(=O)OCC1(c2ccc(/C(N)=N/O)cc2)CCC1]

### Step 1: [1-(4-Bromophenyl)cyclobutyl]methyl acetate

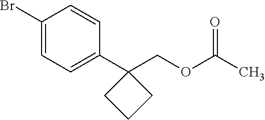 [SMILES: CC(=O)OCC1(c2ccc(Br)cc2)CCC1]

236 μl (2.50 mmol) of acetic anhydride were added to a solution of 402 mg (1.67 mmol) of the compound from Example 110A/step 1 in 6 ml of pyridine at 0° C. After the reaction mixture had been stirred at RT for 16 h, all the volatile constituents were removed on a rotary evaporator. The product was isolated from the residue obtained by means of MPLC (silica gel, mobile phase: cyclohexane/ethyl acetate 10:1). 450 mg (91% of th., purity of approx. 95%) of the title compound were obtained.

¹H-NMR (400 MHz, CDCl₃, δ/ppm): 7.42 (d, 2H), 7.02 (d, 2H), 4.21 (s, 2H), 2.38-2.30 (m, 2H), 2.29-2.21 (m, 2H), 2.16-2.03 (m, 1H), 1.98 (s, 3H), 1.93-1.83 (m, 1H).

MS (DCI, NH₃): m/z=300/302 [M+NH₄]⁺.

### Step 2: [1-(4-Cyanophenyl)cyclobutyl]methyl acetate

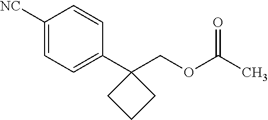 [SMILES: CC(=O)OCC1(c2ccc(C#N)cc2)CCC1]

Analogously to the process described under Example 109A/step 2, 440 mg (1.55 mmol) of the compound from Example 115A/step 1 were reacted to give 314 mg (84% of th., purity of 95%) of the title compound. The purification of the crude product was carried out directly by means of preparative HPLC (method N).

¹H-NMR (400 MHz, CDCl₃, δ/ppm): 7.60 (d, 2H), 7.25 (d, 2H), 4.26 (s, 2H), 2.42-2.26 (m, 4H), 2.20-2.09 (m, 1H), 1.98 (s, 3H), 1.98-1.87 (m, 1H).

MS (DCI, NH₃): m/z=247 [M+NH₄]⁺.

### Step 3: {1-[4-(N′-Hydroxycarbamimidoyl)phenyl]cyclobutyl}methyl acetate

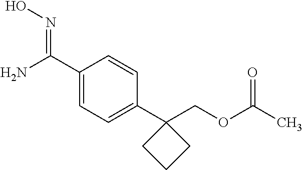 [SMILES: CC(=O)OCC1(c2ccc(/C(N)=N/O)cc2)CCC1]

Analogously to the process described under Example 1A/step 5, 260 mg (1.13 mmol) of the compound from Example 115A/step 2 were reacted to give 312 mg (94% of th., purity of 90%) of the title compound.

¹H-NMR (400 MHz, CDCl₃, δ/ppm): 7.57 (d, 2H), 7.19 (d, 2H), 4.84 (broad, 2H), 4.24 (s, 2H), 2.44-2.34 (m, 2H), 2.32-2.24 (m, 2H), 2.17-2.07 (m, 1H), 1.99 (s, 3H), 1.94-1.86 (m, 1H).

MS (DCI, NH₃): m/z=263 [M+NH₄]⁺.

LC/MS (method F, ESIpos): Rₜ=0.74 min, m/z=263 [M+H]⁺.

### Example 116A

### N′-Hydroxy-4-[1-(2-hydroxyethyl)cyclobutyl]benzenecarboximide amide

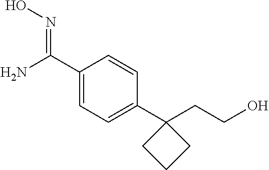 [SMILES: N/C(=N\O)c1ccc(C2(CCO)CCC2)cc1]

### Step 1: 2-{1-[4-(Dibenzylamino)phenyl]cyclobutyl}ethyl acetate

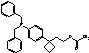 [SMILES: CC(=O)OCCC1(c2ccc(N(Cc3ccccc3)Cc3ccccc3)cc2)CCC1]

Analogously to the process described under Example 115A/step 1, 3.64 g (87% of th.) of the title compound were obtained from 3.50 g (9.42 mmol) of the compound from Example 113A/step 2. The final filtration with suction over silica gel was carried out with cyclohexane/ethyl acetate 20:1 as the mobile phase.

¹H-NMR (400 MHz, CDCl₃, δ/ppm): 7.35-7.30 (m, 4H), 7.28-7.22 (m, 6H), 6.91 (d, 2H), 6.67 (d, 2H), 4.62 (s, 4H), 3.86-3.82 (m, 2H), 2.37-2.29 (m, 2H), 2.15-2.00 (m, 5H), 1.94 (s, 3H), 1.86-1.77 (m, 1H).

HPLC (method A): Rₜ=5.22 min.

MS (DCI, NH₃): m/z=414 [M+H]⁺.

LC/MS (method I, ESIpos): Rₜ=1.51 min, m/z=414 [M+H]⁺.

### Step 2: 2-[1-(4-Aminophenyl)cyclobutyl]ethyl acetate

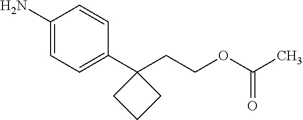 [SMILES: CC(=O)OCCC1(c2ccc(N)cc2)CCC1]

Analogously to the process described under Example 4A/step 6, 1.79 g (85% of th., purity of 94%) of the title compound were obtained from 3.50 g (8.46 mmol) of the compound from Example 116A/step 1. In this case, 300 ml of a mixture of ethanol and ethyl acetate (3:1) was used as the solvent. The crude product obtained after evaporating off the solvent was employed in the subsequent reaction without further purification by chromatography.

LC/MS (method D, ESIpos): Rₜ=1.46 min, m/z=467 [2M+H]⁺, 234 [M+H]⁺.

### Step 3: 2-[1-(4-Cyanophenyl)cyclobutyl]ethyl acetate

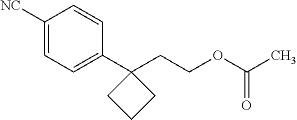 [SMILES: CC(=O)OCCC1(c2ccc(C#N)cc2)CCC1]

Analogously to the process described under Example 4A/step 7, 152 mg (29% of th.) of the title compound were prepared from 500 mg (2.14 mmol) of the compound from Example 116A/step 2.

¹H-NMR (400 MHz, CDCl₃, δ/ppm): 7.60 (d, 2H), 7.23 (d, 2H), 3.83 (t, 2H), 2.42-2.33 (m, 2H), 2.27-2.21 (m, 2H), 2.17 (t, 2H), 2.15-2.08 (m, 1H), 1.93-1.82 (m, 1H), 1.89 (s, 3H).

MS (DCI, NH₃): m/z=461 [M+NH₄]⁺.

### Step 4: N′-Hydroxy-4-[1-(2-hydroxyethyl)cyclobutyl]benzenecarboximide amide

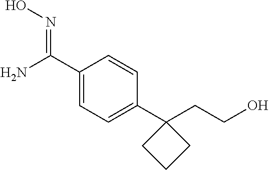 [SMILES: N/C(=N\O)c1ccc(C2(CCO)CCC2)cc1]

Analogously to the process described in Example 1A/step 5, 164 mg of a mixture of the title compound with the corresponding acetate (2-{1-[4-(N′-hydroxycarbamimidoyl)phenyl]-cyclobutyl}ethyl acetate) were obtained from 150 mg (0.617 mmol) of the compound from Example 116A/step 3. This mixture was not separated, but was employed as such in subsequent reactions.

LC/MS (method I, ESIpos): title compound: Rₜ=0.52 min, m/z=234 [M+H]⁺; corresponding acetate: Rₜ=0.70 min, m/z=277 [M+H]⁺.

### Example 117A

### 2-{1-[4-(N′-Hydroxycarbamimidoyl)phenyl]cyclobutyl}-N,N-dimethylacetamide

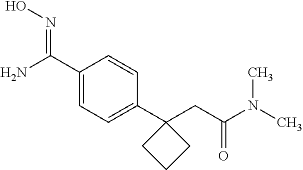 [SMILES: CN(C)C(=O)CC1(c2ccc(/C(N)=N/O)cc2)CCC1]

### Step 1: {1-[4-(Dibenzylamino)phenyl]cyclobutyl}acetic acid

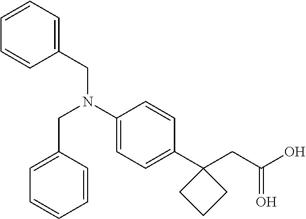 [SMILES: O=C(O)CC1(c2ccc(N(Cc3ccccc3)Cc3ccccc3)cc2)CCC1]

43.5 ml (43.5 mmol) of 1 M sodium hydroxide solution were added to a solution of 6.0 g (14.5 mmol) of the compound from Example 113A/step 1 in 90 ml of ethanol and the mixture was heated under reflux for 3 h. After cooling to RT, the mixture was neutralized with 1 M hydrochloric acid and the ethanol was mostly stripped off on a rotary evaporator. The aqueous solution obtained was extracted three times with approx. 100 ml of ethyl acetate each time. The combined organic extracts were dried over anhydrous magnesium sulfate. After filtration, the solvent was removed on a rotary evaporator. The crude product was coarsely purified by means of filtration with suction over approx. 200 g of silica gel with cyclohexane/ethyl acetate 4:1 as the mobile phase. 5.35 g (86% of th., purity of 90%) of the title compound were obtained in this way.

¹H-NMR (400 MHz, CDCl₃, δ/ppm): 10.97 (very broad, 1H), 7.33-7.28 (m, 4H), 7.25-7.22 (m, 6H), 6.99 (d, 2H), 6.66 (d, 2H), 4.60 (s, 4H), 2.73 (s, 2H), 2.43-2.27 (m, 4H), 2.04-1.96 (m, 1H), 1.88-1.78 (m, 1H).

HPLC (method A): Rₜ=4.76 min.

MS (DCI, NH₃): m/z=386 [M+H]⁺.

LC/MS (method I, ESIpos): Rₜ=1.35 min, m/z=386 [M+H]⁺.

### Step 2: 2-{1-[4-(Dibenzylamino)phenyl]cyclobutyl}-N,N-dimethylacetamide

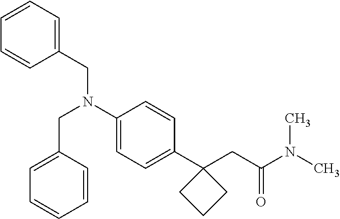 [SMILES: CN(C)C(=O)CC1(c2ccc(N(Cc3ccccc3)Cc3ccccc3)cc2)CCC1]

Under inert conditions, 2.65 g (6.87 mmol) of the compound from Example 117A/step 1 were dissolved in 70 ml of anhydrous methylene chloride, and 3 ml (34.4 mmol) of oxalyl chloride and one drop of DMF were added. After the mixture had been stirred at RT for 2 h, it was concentrated to dryness on a rotary evaporator. The residue obtained was dried under a high vacuum for approx. 1 h and then dissolved in 30 ml of anhydrous THF. This solution was added dropwise to a mixture of 10.3 ml (20.6 mmol) of a 2 M solution of dimethylamine in THF, which had been diluted beforehand with 30 ml of THF, and 3.6 ml (20.6 mmol) of N,N-diisopropylethylamine were added dropwise. After stirring at RT for 16 h, the reaction mixture was freed from all the volatile constituents on a rotary evaporator. The residue obtained was taken up in 300 ml of ethyl acetate and the mixture was washed successively with approx. 100 ml each of saturated sodium bicarbonate solution, water and saturated sodium chloride solution. After drying over anhydrous magnesium sulfate, the mixture was filtered and the filtrate was concentrated on a rotary evaporator. 1.93 g (68% of th.) of the title compound were obtained.

¹H-NMR (400 MHz, CDCl₃, δ/ppm): 7.33-7.29 (m, 4H), 7.26-7.22 (m, 6H), 6.94 (d, 2H), 6.65 (d, 2H), 4.61 (s, 4H), 2.69 (s, 2H), 2.67 (s, 3H), 2.47-2.31 (m, 4H), 2.25 (s, 3H), 2.12-2.01 (m, 1H), 1.87-1.77 (m, 1H).

LC/MS (method F, ESIpos): Rₜ=1.53 min, m/z=413 [M+H]⁺.

### Step 3: 2-[1-(4-Aminophenyl)cyclobutyl]-N,N-dimethylacetamide

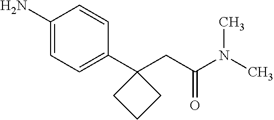 [SMILES: CN(C)C(=O)CC1(c2ccc(N)cc2)CCC1]

Analogously to the process described under Example 4A/step 6, 1.1 g (99% of th., purity of 98%) of the title compound were obtained from 1.93 g (4.68 mmol) of the compound from Example 117A/step 2. In this case, 250 ml of a mixture of ethanol and ethyl acetate (3:1) were used as the solvent. The product obtained after evaporating off the solvent was employed in the subsequent reaction without further purification by chromatography.

¹H-NMR (400 MHz, CDCl₃, δ/ppm): 6.96 (d, 2H), 6.62 (d, 2H), 3.58 (broad, 2H), 2.74 (s, 3H), 2.72 (s, 2H), 2.50-2.43 (m, 2H), 2.39-2.32 (m, 2H), 2.33 (s, 3H), 2.17-2.03 (m, 1H), 1.87-1.78 (m, 1H).

LC/MS (method F, ESIpos): Rₜ=0.55 min, m/z=233 [M+H]⁺.

### Step 4: 2-[1-(4-Cyanophenyl)cyclobutyl]-N,N-dimethylacetamide

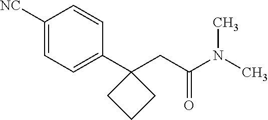 [SMILES: CN(C)C(=O)CC1(c2ccc(C#N)cc2)CCC1]

Analogously to the process described under Example 4A/step 7, 776 mg (74% of th.) of the title compound were obtained from 1.0 g (4.30 mmol) of the compound from Example 117A/step 3. The crude product was purified by means of preparative HPLC (method N).

¹H-NMR (400 MHz, CDCl₃, δ/ppm): 7.57 (d, 2H), 7.33 (d, 2H), 2.86 (s, 2H), 2.76 (s, 3H), 2.57 (s, 3H), 2.51-2.39 (m, 4H), 2.19-2.07 (m, 1H), 1.92-1.82 (m, 1H).

MS (DCI, NH₃): m/z=260 [M+NH₄]⁺, 243 [M+H]⁺.

GC/MS (method L, EIpos): Rₜ=7.39 min, m/z=242 [M]±.

### Step 5: 2-{1-[4-(N′-Hydroxycarbamimidoyl)phenyl]cyclobutyl}-N,N-dimethylacetamide

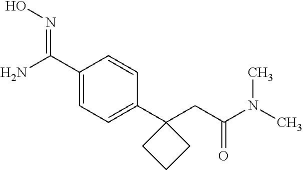 [SMILES: CN(C)C(=O)CC1(c2ccc(/C(N)=N/O)cc2)CCC1]

Analogously to the process described under Example 1A/step 5, 669 mg (73% of th., purity of 91%) of the title compound were obtained from 730 mg (3.01 mmol) of the compound from Example 117A/step 4. The final stirring of the product was carried out here not in petroleum ether but in ethanol.

¹H-NMR (400 MHz, DMSO-d₆, δ/ppm): 9.53 (s, 1H), 7.56 (d, 2H), 7.17 (d, 2H), 5.73 (s, 2H), 2.82 (s, 2H), 2.62 (s, 3H), 2.53 (s, 3H), 2.41-2.23 (m, 2H), 2.32-2.25 (m, 2H), 2.10-1.98 (m, 1H), 1.80-1.70 (m, 1H).

HPLC (method A): Rₜ=3.27 min.

MS (DCI, NH₃): m/z=551 [2M+H]⁺, 276 [M+H]⁺.

### Example 118A

### 4-[(Diisopropylamino)methyl]-N′-hydroxybenzenecarboximide amide

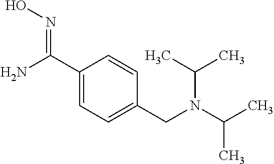 [SMILES: CC(C)N(Cc1ccc(/C(N)=N/O)cc1)C(C)C]

### Step 1: 4-[(Diisopropylamino)methyl]benzonitrile

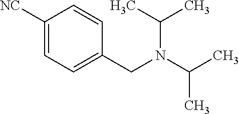 [SMILES: CC(C)N(Cc1ccc(C#N)cc1)C(C)C]

A mixture of 4.0 g (20.4 mmol) of 4-(bromomethyl)benzonitrile and 6.19 g (61.2 mmol) of diisopropylamine in 40 ml of toluene was heated in two portions at 150° C. in a microwave apparatus (CEM Discover, initial irradiation power 250 W) for in each case 3 h. After cooling to RT, the solid formed was filtered off and the filtrate was concentrated to obtain 4.52 g (92% of th., purity of 90%) of the title compound in this way.

LC/MS (method F, ESIpos): Rₜ=0.30 min, m/z=217 [M+H]⁺.

### Step 2: 4-[(Diisopropylamino)methyl]-N′-hydroxybenzenecarboximide amide

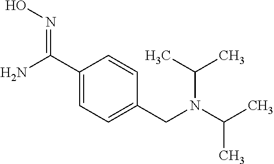 [SMILES: CC(C)N(Cc1ccc(/C(N)=N/O)cc1)C(C)C]

Analogously to the process described under Example 1A/step 5, 4.93 g (70% of th.) of the title compound were obtained from 6.80 g (28.3 mmol, purity of 90%) of the compound from Example 118A/step 1.

¹H-NMR (400 MHz, CDCl₃, δ/ppm): 7.52 (d, 2H), 7.41 (d, 2H), 4.84 (s, broad, 2H), 3.64 (s, 2H), 3.05-2.95 (m, 2H), 1.01 (d, 12H).

LC/MS (method I, ESIpos): Rₜ=0.18 min, m/z=250 [M+H]⁺.

### Example 119A

### N′-Hydroxy-4-[N-methyl-S-(trifluoromethyl)sulfonimidoyl]benzenecarboximide amide (racemate)

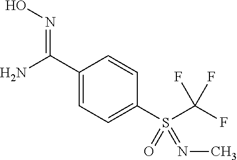 [SMILES: CN=S(=O)(c1ccc(/C(N)=N/O)cc1)C(F)(F)F]

### Step 1: 4-[S-(Trifluoromethyl)sulfonimidoyl]benzonitrile (racemate)

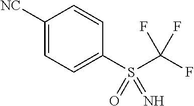 [SMILES: N#Cc1ccc(S(=N)(=O)C(F)(F)F)cc1]

150 mg (0.66 mmol) of 1-fluoro-4-[S-(trifluoromethyl)sulfonimidoyl]benzene [N. V. Kondratenko, Zhurnal Organicheskoi Khimii 1986, 22 (8), 1716-1721; ibid. 1984, 20 (10), 2250-2252] were dissolved in 20 ml DMSO, and 115 mg (0.83 mmol) of potassium carbonate, 140 mg (0.84 mmol) of potassium iodide and 130 mg (2.0 mmol) of potassium cyanide were added. The mixture was heated at 110° C. overnight, while stirring. After cooling to RT, approx. 10 ml of water were added to the mixture and the mixture was extracted with ethyl acetate. After concentration of the organic phase, the residue was purified by means of flash chromatography over silica gel. 50 mg (33% of th.) of the title compound were obtained.

### Step 2: 4-[N-Methyl-S-(trifluoromethyl)sulfonimidoyl]benzonitrile (racemate)

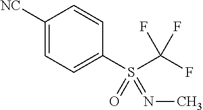 [SMILES: CN=S(=O)(c1ccc(C#N)cc1)C(F)(F)F]

400 mg (1.60 mmol) of the compound from Example 119A/step 1 were dissolved in 8 ml of THF under argon and 224 mg (2.0 mmol) of potassium tert-butylate were added. The mixture was first stirred at RT for 1 h, 283 mg (2.0 mmol) of iodomethane were then added and the mixture was stirred further at RT overnight. Water was then added to the batch and the mixture was extracted with ethyl acetate. The organic extract was washed with saturated aqueous sodium chloride solution, dried over magnesium sulfate and concentrated. The residue was purified by means of flash chromatography over silica gel. 298 mg (70% of th.) of the title compound were obtained.

¹H-NMR (400 MHz, CDCl₃, δ/ppm): 8.22 (d, 2H), 7.90 (d, 2H), 3.10 (s, 3H).

LC/MS (method D, ESIpos): Rₜ=2.17 min, m/z=249 [M+H]⁺.

### Step 3: N′-Hydroxy-4-[N-methyl-S-(trifluoromethyl)sulfonimidoyl]benzenecarboximide amide (racemate)

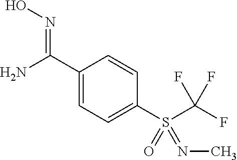 [SMILES: CN=S(=O)(c1ccc(/C(N)=N/O)cc1)C(F)(F)F]

1.00 g (4.03 mmol) of the compound from Example 119A/step 2 were initially introduced into 20 ml of ethanol. 616 mg (8.86 mmol) of hydroxylamine hydrochloride and 1.2 ml (8.86 mmol) of triethylamine were added and the mixture was heated under reflux for 1 h. It was then concentrated and the residue was taken up in a mixture of ethyl acetate and water. The phases were separated and the aqueous phase was extracted once with ethyl acetate. The combined ethyl acetate phases were washed once with saturated aqueous sodium chloride solution, dried over magnesium sulfate, filtered and concentrated. The residue was purified by means of column chromatography (silica gel, mobile phase: cyclohexane/ethyl acetate 7:3). The combined product fractions were concentrated and the residue was stirred with pentane. The resulting solid was filtered off and dried in vacuo. 775 mg (66% of th.) of the title compound were obtained.

¹H-NMR (400 MHz, CDCl₃, δ/ppm): 8.12 (d, 2H), 8.04 (s, broad, 1H), 7.87 (d, 2H), 4.93 (s, 2H), 3.10 (s, 3H).

LC/MS (method I, ESIpos): Rₜ=0.76 min, m/z=282 [M+H]⁺.

### Example 120A

### 3-Chloro-N′-hydroxy-4-(trifluoromethoxy)benzenecarboximide amide

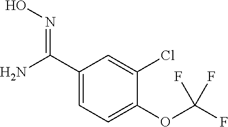 [SMILES: N/C(=N\O)c1ccc(OC(F)(F)F)c(Cl)c1]

Analogously to the process described under Example 1A/step 5, 842 mg (73% of th.) of the title compound were obtained from 1.00 g (4.51 mmol) of 3-chloro-4-(trifluoromethoxy)benzonitrile.

¹H-NMR (400 MHz, CDCl₃, δ/ppm): 7.77 (d, 1H), 7.58-7.55 (dd, 1H), 7.37-7.33 (m, 1H), 4.82 (s, broad, 1H).

LC/MS (method D, ESIpos): Rₜ=1.64 min, m/z=255/257 [M+H]⁺.

### Example 121A

### N′-Hydroxy-4-[1-(trifluoromethyl)cyclopropyl]benzenecarboximide amide

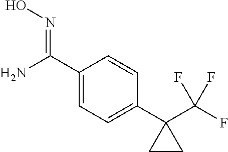 [SMILES: N/C(=N\O)c1ccc(C2(C(F)(F)F)CC2)cc1]

### Step 1: 1-Bromo-4-[1-(trifluoromethyl)cyclopropyl]benzene

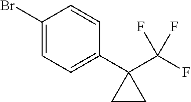 [SMILES: FC(F)(F)C1(c2ccc(Br)cc2)CC1]

Activated zinc bromide on montmorillonite was first prepared as follows: 1.40 g (6.22 mmol) of zinc bromide were initially introduced into 56 ml of methanol, 5.64 g of montmorillonite K10 were added and the mixture was stirred at RT for 1 h. After removal of the methanol, the powder which remained was heated in a sand bath at a bath temperature of 200° C. for 1 h and then allowed to cool under argon.

The title compound was then prepared as follows: 10.0 g (53.7 mmol) of 1-phenyl-1-(trifluoromethyl)cyclopropane were initially introduced into 50 ml of pentane. 6.1 g (5.37 mmol) of the activated zinc bromide on montmorillonite obtained above were added and 27.7 ml (537 mmol) of bromine were then slowly added dropwise in the dark, while stirring. The mixture was then stirred further at RT in the dark overnight. 150 ml of a saturated aqueous sodium sulfite solution were subsequently slowly added dropwise, while cooling with ice, and the mixture was stirred at RT for a further approx. 30 min until it was decolorized. The solid was filtered off and rinsed twice with pentane. After separation of the filtrate phases, the aqueous phase was extracted twice with 200 ml of pentane each time. The combined organic phases were dried over sodium sulfate, filtered and concentrated under gentle conditions (significant volatility of the target compound). 17.1 g (>100% of th.) of the title compound were obtained in this manner, which according to ¹H-NMR still contained pentane.

¹H-NMR (400 MHz, CDCl₃, δ/ppm): 7.47 (d, 2H), 7.32 (s, 2H), 1.39-1.30 (m, 2H), 1.04-0.95 (m, 2H).

GC/MS (method L, ESIpos): Rₜ=3.45 min, m/z=264/266 [M+H]⁺.

### Step 2: 4-[1-(Trifluoromethyl)cyclopropyl]benzonitrile

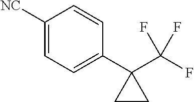 [SMILES: N#Cc1ccc(C2(C(F)(F)F)CC2)cc1]

6.00 g (22.6 mmol) of the compound from Example 121A/step 1 were initially introduced into 30 ml of DMF under argon, 1.86 g (15.8 mmol) of zinc cyanide and 1.57 g (1.36 mmol) of tetrakis(triphenylphosphine)palladium(0) were added and the mixture was stirred at 80° C. overnight. After cooling to RT, a further 4.0 g (34.1 mmol) of zinc cyanide and 3.0 g (2.56 mmol) of tetrakis(triphenylphosphine)palladium(0) were added and the mixture was heated again at 120° C. for 5 h, while stirring. After cooling to RT, the solid present was filtered off and washed once with DMF. The filtrate, combined with the wash solution, was concentrated. The residue was taken up in 200 ml of ethyl acetate and the solution obtained was washed twice with 2 M aqueous ammonia solution and once with saturated aqueous sodium chloride solution. After drying over sodium sulfate, filtration and concentration, the residue obtained was purified by flash chromatography (silica gel, mobile phase: cyclohexane/ethyl acetate 40:1). After brief drying in vacuo, 3.46 g (72% of th.) of the title compound were obtained.

¹H-NMR (400 MHz, CDCl₃, δ/ppm): 7.66 (d, 2H), 7.58 (d, 2H), 1.47-1.41 (m, 2H), 1.09-1.03 (m, 2H).

GC/MS (method L, ESIpos): Rₜ=3.81 min, m/z=212 [M+H]⁺.

### Step 3: N′-Hydroxy-4-[1-(trifluoromethyl)cyclopropyl]benzenecarboximide amide

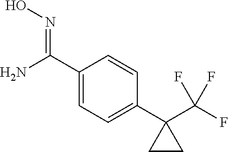 [SMILES: N/C(=N\O)c1ccc(C2(C(F)(F)F)CC2)cc1]

Analogously to the process described under Example 1A/step 5, 3.82 g of the title compound (98% of theory) were obtained from 3.40 g (16.1 mmol) of the compound from Example 121A/step 2.

¹H-NMR (400 MHz, CDCl₃, δ/ppm): 7.62 (d, 2H), 7.50 (d, 2H), 4.88 (s, broad, 2H), 1.42-1.36 (m, 2H), 1.06-1.00 (m, 2H).

LC/MS (method F, ESIpos): Rₜ=0.81 min, m/z=245 [M+H]⁺.

### Example 122A

### 3-Fluoro-N′-hydroxy-4-(trifluoromethoxy)benzenecarboximide amide

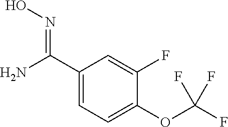 [SMILES: N/C(=N\O)c1ccc(OC(F)(F)F)c(F)c1]

Analogously to the process described under Example 1A/step 5, 5.7 g (99% of th., purity of 98%) of the title compound were obtained from 5.0 g (23.9 mmol, purity of 98%) of 3-fluoro-4-(trifluoromethoxy)benzonitrile.

¹H-NMR (400 MHz, CDCl₃, δ/ppm): 7.53-7.49 (dd, 1H), 7.45-7.41 (m, 1H), 7.37-7.31 (t, 1H), 4.87 (s, broad, 2H).

LC/MS (method I, ESIpos): Rₜ=0.74 min, m/z=239 [M+H]⁺.

### Example 123A

### 4-[4-(Fluoromethyl)tetrahydro-2H-pyran-4-yl]-N′-hydroxybenzenecarboximide amide

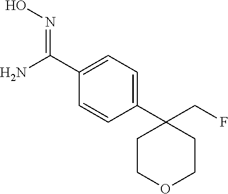 [SMILES: N/C(=N\O)c1ccc(C2(CF)CCOCC2)cc1]

### Step 1: Ethyl 4-(4-bromophenyl)tetrahydro-2H-pyran-4-carboxylate

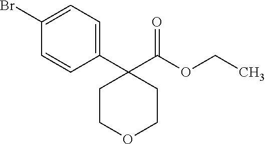 [SMILES: CCOC(=O)C1(c2ccc(Br)cc2)CCOCC1]

6.0 g (24.7 mmol) of ethyl 4-bromophenylacetate were dissolved in 120 ml of abs. DMF under argon, 1.48 g (37.0 mmol, 60% strength) of sodium hydride were added, while cooling in an ice bath, and the mixture was stirred for 30 min. 5.72 g (24.7 mmol) of bis(2-bromoethyl)ether were then added, while constantly cooling in an ice bath, and the mixture was stirred at approx. 0° C. for 1 h. After renewed addition of 1.48 g of 60% strength sodium hydride, the mixture was stirred again for 1 h, while cooling in an ice bath. Saturated aqueous ammonium chloride was then added and the mixture was extracted with ethyl acetate. The organic phase was washed with water and with saturated sodium chloride solution, dried over magnesium sulfate, filtered and concentrated on a rotary evaporator. The residue was purified by column chromatography over silica gel (mobile phase: cyclohexane/ethyl acetate 10:1). 2.62 g (33% of th.) of the title compound were obtained.

¹H-NMR (400 MHz, CDCl₃, δ/ppm): 7.47 (d, 2H), 7.25 (d, 2H), 4.14 (q, 2H), 3.93 (dt, 2H), 3.56 (td, 2H), 2.59 (dd, 2H), 1.93 (m, 2H), 1.19 (t, 3H).

MS (DCI, NH₃): m/z=329 [M+NH₄]⁺.

LC/MS (method F, ESIpos): Rₜ=1.33 min, no ionization.

### Step 2: [4-(4-Bromophenyl)tetrahydro-2H-pyran-4-yl]methanol

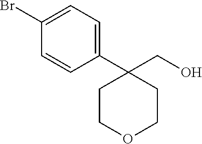 [SMILES: OCC1(c2ccc(Br)cc2)CCOCC1]

1.14 g (3.64 mmol) of the compound from Example 123A/step 1 were dissolved in 18 ml of THF, 3.64 ml (3.64 mmol) of a 1 M solution of lithium aluminium hydride in THF were added at 0° C. and the mixture was stirred for 1 h, while cooling in an ice bath. Saturated aqueous ammonium chloride solution was subsequently added dropwise and the mixture was then extracted with ethyl acetate. The organic phase was washed successively with 1 N sodium hydroxide solution, water and saturated aqueous sodium chloride solution, dried over magnesium sulfate, filtered and concentrated on a rotary evaporator. After the residue had been dried in vacuo, 780 mg (79% of th.) of the title compound were obtained.

¹H-NMR (400 MHz, CDCl₃, δ/ppm): 7.51 (d, 2H), 7.22 (d, 2H), 3.79 (m, 2H), 3.59 (d, 2H), 3.54 (t, 2H), 2.09 (d, 2H), 1.91 (m, 2H).

GC/MS (method L): Rₜ=7.09 min, m/z=252 [M−H₂O]⁺.

### Step 3: 4-(4-Bromophenyl)-4-(fluoromethyl)tetrahydro-2H-pyran

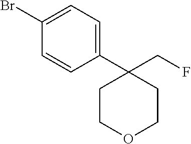 [SMILES: FCC1(c2ccc(Br)cc2)CCOCC1]

0.63 ml of a 50% strength solution of bis(2-methoxyethyl)aminosulfur trifluoride in THF was added dropwise to a solution of 400 mg (1.47 mmol) of the compound from Example 123A/step 2 in 4.0 ml of methylene chloride at −78° C. and the mixture was then stirred at RT overnight. 1 N sodium hydroxide solution was subsequently added to the batch and the mixture was diluted with methylene chloride and washed with water and saturated aqueous sodium chloride solution. The organic phase was dried over magnesium sulfate, filtered and freed from the solvent on a rotary evaporator. The residue obtained was purified by means of preparative HPLC (method P). 131 mg (33% of th.) of the title compound were obtained.

¹H-NMR (400 MHz, DMSO-d₆, δ/ppm): 7.50 (d, 2H), 7.18 (d, 2H), 3.69 (m, 2H), 3.49 (td, 2H), 2.92 (d, 2H), 1.79-1.62 (m, 2H), 1.54 (t, 2H).

GC/MS (method L): Rₜ=6.03 min, m/z=272 [M]⁺.

### Step 4: 4-[4-(Fluoromethyl)tetrahydro-2H-pyran-4-yl]benzonitrile

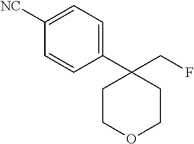 [SMILES: N#Cc1ccc(C2(CF)CCOCC2)cc1]

360 mg (1.32 mmol) of the compound obtained in Example 123A/step 3 were initially introduced into 2.1 ml of degassed DMF under argon, 93 mg (0.79 mmol) of zinc cyanide and 91 mg (0.08 mmol) of tetrakis(triphenylphosphine)palladium(0) were added and the mixture was stirred at 110° C. in a microwave oven for 1 h. After cooling to RT, the solid was filtered off and the filtrate was purified directly by means of preparative HPLC (method P). 195 mg (67% of th.) of the title compound were obtained.

¹H-NMR (400 MHz, DMSO-d₆, δ/ppm): 7.78 (d, 2H), 7.43 (d, 2H), 3.71 (m, 2H), 3.48 (td, 2H), 3.05 (d, 2H), 1.81-1.65 (m, 2H), 1.54 (t, 2H).

GC/MS (method L): Rₜ=6.32 min, m/z=199 [M−HF]⁺.

### Step 5: 4-[4-(Fluoromethyl)tetrahydro-2H-pyran-4-yl]-N′-hydroxybenzenecarboximide amide

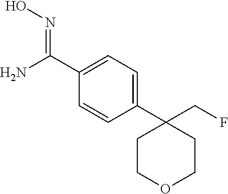 [SMILES: N/C(=N\O)c1ccc(C2(CF)CCOCC2)cc1]

A mixture of 410 mg (1.84 mmol) of the compound from Example 123A/step 4, 286 mg (4.11 mmol) of hydroxylamine hydrochloride and 0.57 ml (4.11 mmol) of triethylamine in 9.1 ml of ethanol was stirred at 80° C. for 2 h. After cooling to RT, the solvent was removed virtually completely on a rotary evaporator. The residue was suspended with a little water under ultrasound irradiation. The white solid was filtered off, washed with a little cold water and then stirred with pentane. The solid was filtered off again and dried in vacuo. 320 mg (65% of th.) of the title compound were obtained in this way.

LC/MS (method I): Rₜ=0.47 min, m/z=253 [M+H]⁺.

### Example 124A

### 5-(5-Methyl-1H-pyrazol-3-yl)-3-[4-(tetrahydro-2H-pyran-4-yl)phenyl]-1,2,4-oxadiazole

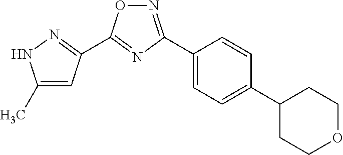 [SMILES: Cc1cc(-c2nc(-c3ccc(C4CCOCC4)cc3)no2)n[nH]1]

Analogously to the process described under Example 28A, from 469 mg (3.72 mmol) of 5-methyl-1H-pyrazole-3-carboxylic acid and 820 mg (3.72 mmol) of the compound from Example 107A, 450 mg of the title compound were obtained after extraction of the crude product by stirring in acetonitrile, and a further 97 mg of the title compound were obtained after purification of the mother liquor by preparative HPLC (method N) (yield 47% of th. in total).

¹H-NMR (400 MHz, DMSO-d₆, δ/ppm): 13.52 (s, 1H), 8.01 (d, 2H), 7.49 (d, 2H), 6.79 (s, 1H), 3.99-3.95 (m, 2H), 3.49-3.42 (m, 2H), 2.92-2.84 (m, 1H), 2.34 (s, 3H), 1.77-1.65 (m, 4H).

LC/MS (method I, ESIpos): Rₜ=0.98 min, m/z=311 [M+H]⁺.

### Example 125A

### 3-(4-tert-Butylphenyl)-5-(5-methyl-1H-pyrazol-3-yl)-1,2,4-oxadiazole

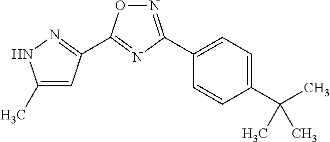 [SMILES: Cc1cc(-c2nc(-c3ccc(C(C)(C)C)cc3)no2)n[nH]1]

Analogously to the process described under Example 28A, 2.50 g (19.8 mmol) of 5-methyl-1H-pyrazole-3-carboxylic acid and 4.19 g (21.8 mmol) of 4-tert-butyl-N′-hydroxybenzenecarboximide amide were reacted to give 2.60 g (46% of th.) of the title compound. The reaction mixture was stirred here first at RT for 1 h and then at 140° C. for 30 min.

¹H-NMR (400 MHz, CDCl₃, δ/ppm): 11.08 (broad, 1H), 8.10 (d, 2H), 7.51 (d, 2H), 6.81 (s, 1H), 2.45 (s, 3H), 1.37 (s, 9H).

LC/MS (method I, ESIpos): Rₜ=1.21 min, m/z=283 [M+H]⁺, 565 [2M+H]⁺.

### Example 126A

### 3-{4-[1-(Methoxymethyl)cyclobutyl]phenyl}-5-(5-methyl-1H-pyrazol-3-yl)-1,2,4-oxadiazole

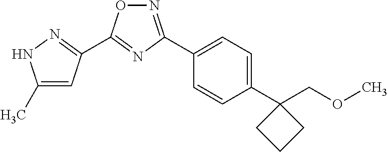

Analogously to the process described under Example 28A, 1.08 g (8.52 mmol) of 5-methyl-1H-pyrazole-3-carboxylic acid and 2.0 g (8.52 mmol) of the compound from Example 110A were reacted to give 1.87 g (46% of th.) of the title compound. The reaction mixture was stirred here first at RT for 1 h and then at 140° C. for 30 min.

¹H-NMR (400 MHz, CDCl₃, δ/ppm): 11.57 (broad, 1H), 8.10 (d, 2H), 7.30 (d, 2H), 6.81 (s, 1H), 3.57 (s, 2H), 3.29 (s, 3H), 2.45 (s, 3H), 2.41-2.28 (m, 4H), 2.15-2.03 (m, 1H), 1.93-1.84 (m, 1H).

LC/MS (method F, ESIpos): Rₜ=1.28 min, m/z=325 [M+H]⁺.

### Example 127A

### 5-(5-Methyl-1H-pyrazol-3-yl)-3-[3-methyl-4-(tetrahydro-2H-pyran-4-yl)phenyl]-1,2,4-oxadiazole

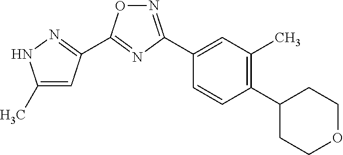 [SMILES: Cc1cc(-c2nc(-c3ccc(C4CCOCC4)c(C)c3)no2)n[nH]1]

Analogously to the process described under Example 28A, 180 mg (1.43 mmol) of 5-methyl-1H-pyrazole-3-carboxylic acid and 335 mg (1.43 mmol) of the compound from Example 108A were reacted to give 189 mg (39% of th.) of the title compound. The reaction mixture was stirred here first at RT for 16 h and then at 140° C. for 30 min. The purification of the product was carried out by means of preparative HPLC (method N).

¹H-NMR (400 MHz, CDCl₃, δ/ppm): 10.63 (broad, 1H), 8.00 (d, 1H), 7.99 (s, 1H), 7.36 (d, 1H), 6.80 (s, 1H), 4.13-4.10 (m, 2H), 3.61-3.54 (m, 2H), 3.07-3.00 (m, 1H), 2.45 (s, 3H), 2.43 (s, 3H), 1.92-1.80 (m, 2H), 1.73-1.68 (m, 2H).

LC/MS (method I, ESIpos): Rₜ=1.02 min, m/z=325 [M+H]⁺.

### Example 128A

### 3-[3-Chloro-4-(trifluoromethoxy)phenyl]-5-(5-methyl-1H-pyrazol-3-yl)-1,2,4-oxadiazole

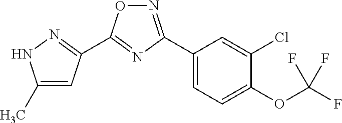 [SMILES: Cc1cc(-c2nc(-c3ccc(OC(F)(F)F)c(Cl)c3)no2)n[nH]1]

Analogously to the process described under Example 28A, 631 mg (5.00 mmol) of 5-methyl-1H-pyrazole-3-carboxylic acid and 1.27 g (5.00 mmol) of the compound from Example 120A were reacted to give 1.08 g (60% of th., purity of 95%) of the title compound. The reaction mixture was stirred here first at RT for approx. 30 min and then at 150° C. for approx. 1 h. The product was obtained by a procedure in which after the reaction had ended the solid which had precipitated out after addition of water was filtered off, washed with water and dried in vacuo.

LC/MS (method I, ESIpos): Rₜ=1.20 min, m/z=345/347 [M+H]⁺.

### Example 129A

### 5-(5-Methyl-1H-pyrazol-3-yl)-3-{4-[1-(trifluoromethyl)cyclopropyl]phenyl}-1,2,4-oxadiazole

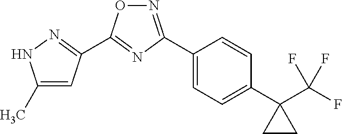 [SMILES: Cc1cc(-c2nc(-c3ccc(C4(C(F)(F)F)CC4)cc3)no2)n[nH]1]

Analogously to the process described under Example 28A, 1.19 g (9.42 mmol) of 5-methyl-1H-pyrazole-3-carboxylic acid and 2.30 g (9.42 mmol) of the compound from Example 121A were reacted to give 1.05 g (62% of th.) of the title compound. The crude product was purified by means of preparative HPLC (method O).

¹H-NMR (400 MHz, CDCl₃, δ/ppm): 11.0-10.5 (s, broad, 1H), 8.16 (d, 2H), 7.60 (d, 2H), 6.82 (s, 1H), 1.43-1.39 (m, 2H), 1.12-1.08 (m, 2H).

LC/MS (method I): Rₜ=1.17 min, m/z=335 [M+H]⁺.

### Example 130A

### 5-(5-Methyl-1H-pyrazol-3-yl)-3-{4-[N-methyl-S-(trifluoromethyl)sulfonimidoyl]phenyl}-1,2,4-oxadiazole (racemate)

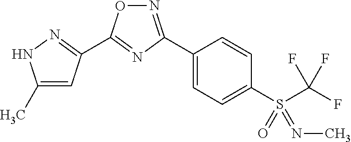 [SMILES: CN=S(=O)(c1ccc(-c2noc(-c3cc(C)[nH]n3)n2)cc1)C(F)(F)F]

Analogously to the process described under Example 28A, 89 mg (0.709 mmol) of 5-methyl-1H-pyrazole-3-carboxylic acid and 206 mg (0.709 mmol, purity of 97%) of the compound from Example 119A were reacted to give 103 mg (39% of th.) of the title compound. The crude product was purified by means of preparative HPLC (method O).

¹H-NMR (400 MHz, CDCl₃, δ/ppm): 10.59 (s, broad, 1H), 8.42 (d, 2H), 8.22 (d, 2H), 6.83 (s, 1H), 3.12 (d, 3H), 2.47 (s, 3H).

LC/MS (method I, ESIpos): Rₜ=1.10 min, m/z=372 [M+H]⁺.

### Example 131A

### 3-[3-Fluoro-4-(trifluoromethoxy)phenyl]-5-(5-methyl-1H-pyrazol-3-yl)-1,2,4-oxadiazole

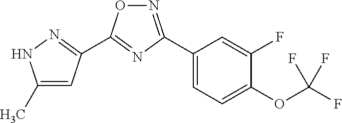 [SMILES: Cc1cc(-c2nc(-c3ccc(OC(F)(F)F)c(F)c3)no2)n[nH]1]

Analogously to the process described under Example 28A, 2.0 g (15.9 mmol) of 5-methyl-1H-pyrazole-3-carboxylic acid and 3.78 g (15.9 mmol) of the compound from Example 122A were reacted to give 3.15 g (56% of th., purity of 92%) of the title compound. In this case the product was obtained not by purification by chromatography but by washing the crude product with water and pentane and subsequent drying in vacuo.

¹H-NMR (400 MHz, CDCl₃, δ/ppm): 12.0-9.5 (s, broad, 1H), 8.10-7.97 (m, 2H), 7.46-7.41 (t, 1H), 6.81 (s, 1H), 2.47 (s, 3H).

LC/MS (method I, ESIpos): Rₜ=1.16 min, m/z=329 [M+H]⁺.

### Example 132A

### N-Isopropyl-N-{4-[5-(5-methyl-1H-pyrazol-3-yl)-1,2,4-oxadiazol-3-yl]benzyl}propan-2-amine

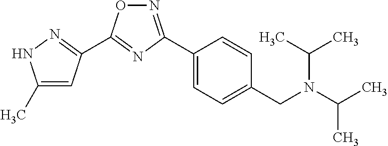 [SMILES: Cc1cc(-c2nc(-c3ccc(CN(C(C)C)C(C)C)cc3)no2)n[nH]1]

Analogously to the process described under Example 28A, 2.00 g (15.9 mmol) of 5-methyl-1H-pyrazole-3-carboxylic acid and 3.95 g (15.9 mmol) of the compound from Example 118A were reacted to give 1.49 g (26% of th., purity of 93%) of the title compound.

¹H-NMR (400 MHz, CDCl₃, δ/ppm): 11.50 (s, broad, 1H), 8.08 (d, 2H), 7.51 (d, 2H), 6.81 (s, 1H), 3.70 (s, 2H), 3.10-2.98 (m, 2H), 2.42 (s, 3H), 1.02 (d, 12H).

LC/MS (method F, ESIpos): Rₜ=0.73 min, m/z=340 [M+H]⁺.

### Example 133A

### 2-Bromo-4-(bromomethyl)pyridine

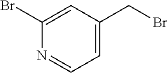

Analogously to the process described under Example 47A/step 3, 1.83 mg (95% of th.) of the title compound were prepared from 1.50 g (7.66 mmol) of 2-bromo-4-(hydroxymethyl)pyridine.

¹H-NMR (400 MHz, CDCl₃, δ/ppm): 8.36 (d, 1H), 7.52 (s, 1H), 7.27 (d, 1H), 4.32 (s, 2H).

HPLC (method A): Rₜ=3.47 min.

MS (DCI, NH₃): m/z=250/252/254 [M+H]⁺.

### Example 134A

### 3-(Bromomethyl)benzenesulfonyl chloride

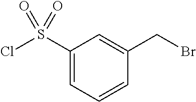 [SMILES: O=S(=O)(Cl)c1cccc(CBr)c1]

5.13 g (28.8 mmol) of N-bromosuccinimide and 43 mg (0.26 mmol) of 2,2′-azobis-2-methylpropanenitrile were added to a solution of 5.0 g (26.2 mmol) of m-toluenesulfonic acid chloride in 50 ml of acetonitrile and the mixture was heated under reflux overnight. The batch was then concentrated on a rotary evaporator and the residue was purified directly by means of MPLC (silica gel, mobile phase: cyclohexane/ethyl acetate 10:1). 4.58 g (65% of th.) of the title compound were obtained.

GC/MS (method L): Rₜ=6.14 min, m/z=189 [M−Br]⁺.

### Example 135A

### 1-[4-(Chloromethyl)pyridin-2-yl]-4-cyclopropylpiperazine

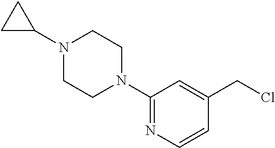 [SMILES: ClCc1ccnc(N2CCN(C3CC3)CC2)c1]

### Step 1: [2-(piperazin-1-yl)pyridin-4-yl]methanol

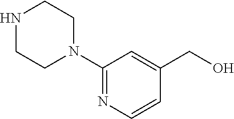 [SMILES: OCc1ccnc(N2CCNCC2)c1]

120 g (1.39 mol) of piperazine were added to 10.0 g (69.6 mmol) of (2-chloropyridin-4-yl)methanol under argon. The mixture was heated at 150° C. overnight, while stirring. After cooling to RT, part of the excess piperazine which had deposited in the upper part of the reaction vessel was removed, and the resinous contents of the flask were taken up in 700 ml of methylene chloride and the mixture was stirred at RT for 30 min. The solid present was filtered off and rinsed with methylene chloride, the solid was discarded and the filtrate was concentrated. The residue was dried in vacuo. 13.3 g (approx. 99% of th.) of the title compound were obtained, which according to ¹H-NMR still contained piperazine.

¹H-NMR (400 MHz, CDCl₃, δ/ppm): 8.14 (d, 1H), 6.67 (s, 1H), 6.58 (d, 1H), 4.64 (s, 2H), 3.55-3.45 (m, 4H), 3.01-2.94 (m, 4H).

LC/MS (method D, ESIpos): Rₜ=0.19 min, m/z=194 [M+H]⁺.

### Step 2: [2-(4-Cyclopropylpiperazin-1-yl)pyridin-4-yl]methanol

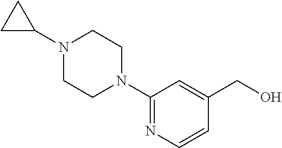 [SMILES: OCc1ccnc(N2CCN(C3CC3)CC2)c1]

13.1 g (67.9 mmol) of the compound from Example 135A/step 1 were dissolved in a mixture of 535 ml of methanol and 39 ml (679 mmol) of acetic acid. 9.2 g of molecular sieve (3 Å) and 82 ml (407 mmol) of [(1-ethoxycyclopropyl)oxy](trimethyl)silane were added. After stirring at RT for 10 min, 12.8 g (203 mmol) of sodium cyanoborohydride were added and the mixture was heated under reflux for 2 h, while stirring. After cooling to RT, the solid present was filtered off and rinsed twice with 20 ml of methanol each time. The filtrate was concentrated and the residue was taken up in 550 ml of methylene chloride. The mixture was washed twice with 500 ml of saturated aqueous sodium bicarbonate solution each time and once with 500 ml of saturated aqueous sodium chloride solution, dried over magnesium sulfate, filtered and concentrated. The residue was purified by means of column chromatography (silica gel, mobile phase: methylene chloride/methanol 95:5). After drying in vacuo, 9.59 g (61% of th.) of the title compound were obtained.

¹H-NMR (400 MHz, CDCl₃, δ/ppm): 8.13 (d, 1H), 6.67 (s, 1H), 6.57 (d, 1H), 4.63 (s, 2H), 3.58-3.46 (m, 4H), 2.77-2.66 (m, 4H), 1.70-1.60 (m, 1H), 0.55-0.41 (m, 4H).

LC/MS (method I, ESIpos): Rₜ=0.17 min, m/z=234 [M+H]⁺.

### Step 3: 1-[4-(Chloromethyl)pyridin-2-yl]-4-cyclopropylpiperazine

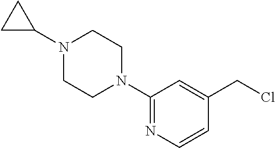 [SMILES: ClCc1ccnc(N2CCN(C3CC3)CC2)c1]

9.59 g (41.1 mmol) of the compound from Example 135A/step 2 were initially introduced into 60 ml of methylene chloride. 15 ml (205 mmol) of thionyl chloride were slowly added at RT and the mixture was stirred first at RT for 10 min, then under reflux for 4.5 h. After cooling to RT, 40 ml of water were added to the mixture and the mixture was rendered basic with 460 ml of saturated aqueous sodium bicarbonate solution and extracted three times with 500 ml of methylene chloride each time. The combined methylene chloride phases were dried over magnesium sulfate, filtered and concentrated. The residue was purified by means of column chromatography (silica gel, mobile phase: cyclohexane/ethyl acetate 7:3). After drying in vacuo, 5.47 g (53% of th.) of the title compound were obtained.

¹H-NMR (400 MHz, CDCl₃, δ/ppm): 8.16 (d, 1H), 6.68-6.56 (m, 2H), 4.45 (s, 2H), 3.61-3.45 (m, 4H), 2.79-2.67 (m, 4H), 1.69-1.62 (m, 1H), 0.58-0.35 (m, 4H).

LC/MS (method I, ESIpos): Rₜ=0.43 min, m/z=252/254 [M+H]⁺.

### Example 136A

### 1-{[3-(Bromomethyl)phenyl]sulfonyl}-4-hydroxypiperidine

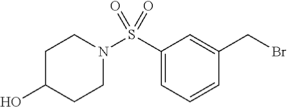 [SMILES: O=S(=O)(c1cccc(CBr)c1)N1CCC(O)CC1]

50 mg (0.5 mmol) of 4-hydroxypiperidine were initially introduced into 2 ml of THF, and 133 mg (0.5 mmol) of the compound from Example 134A and then 69 μl (0.5 mmol) of triethylamine were added, while cooling in an ice bath. After stirring at RT for two hours, the batch was diluted with 5 ml of ethyl acetate, washed with water and saturated sodium chloride solution, dried over magnesium sulfate, filtered and freed from the solvent on a rotary evaporator. The residue obtained (purity of approx. 70% according to HPLC) was employed in subsequent reactions without further purification.

### Example 137A

### 1-{[3-(Bromomethyl)phenyl]sulfonyl}-4-methylpiperazine

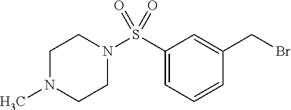 [SMILES: CN1CCN(S(=O)(=O)c2cccc(CBr)c2)CC1]

0.11 ml (1.0 mmol) of 1-methylpiperazine were initially introduced into 2 ml of THF, and 269 mg (1.0 mmol) of the compound from Example 134A and then 139 μl (1.0 mmol) of triethylamine were added, while cooling in an ice bath. After stirring at RT for 30 minutes, the precipitate was filtered off and the filtrate was further reacted directly as a solution of the title compound in THF.

### Example 138A

### 1-(2,2,2-Trifluoroethyl)piperazine dihydrochloride

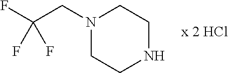 [SMILES: FC(F)(F)CN1CCNCC1]

### Step 1: tert-Butyl 4-(2,2,2-trifluoroethyl)piperazine-1-carboxylate

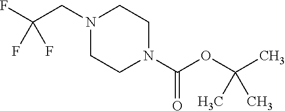 [SMILES: CC(C)(C)OC(=O)N1CCN(CC(F)(F)F)CC1]

Analogously to the process described under Example 104A/step 1, 805 mg (56% of th.) of the title compound were obtained from 1.0 g (5.37 mmol) of tert-butyl piperazine-1-carboxylate, 391 μl (5.37 mmol) of 2,2,2-trifluoroethanol, 1 ml (6.44 mmol) of trifluoromethanesulfonic acid anhydride and 1.2 ml (8.05 mmol) of triethylamine.

¹H-NMR (400 MHz, CDCl₃, δ/ppm): 3.44 (dd, 4H), 2.98 (quart, 2H), 2.61 (dd, 4H), 1.47 (s, 9H).

MS (DCI, NH₃): m/z=269 [M+H]⁺.

GC/MS (method L, EIpos): Rₜ=3.87 min, m/z=212 [M−C₄H₉+H]±.

### Step 2: 1-(2,2,2-Trifluoroethyl)piperazine dihydrochloride

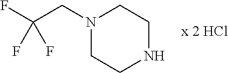 [SMILES: FC(F)(F)CN1CCNCC1]

Analogously to the process described under Example 103A/step 2, 241 mg (93% of th.) of the title compound were obtained starting from 790 mg (2.94 mmol) of the compound from Example 138A/step 1.

¹H-NMR (400 MHz, DMSO-d₆, δ/ppm): 9.14 (broad, 2H), 3.30 (quart, 2H), 3.08-3.03 (m, 4H), 2.87-2.83 (m, 4H).

MS (DCI, NH₃): m/z=169 [M+H]⁺.

### Example 139A

### 2-Bromo-4-[(5-methyl-3-{3-[4-(trifluoromethoxy)phenyl]-1,2,4-oxadiazol-6-yl}-1H-pyrazol-1-yl)-methyl]pyridine

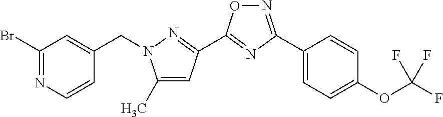 [SMILES: Cc1cc(-c2nc(-c3ccc(OC(F)(F)F)cc3)no2)nn1Cc1ccnc(Br)c1]

Analogously to the process described under Example 76A, 1.05 g (4.19 mmol) of the compound from Example 133A were reacted with 1.0 g (3.22 mmol) of the compound from Example 28A to give 0.71 g (45% of th.) of the title compound. The product was purified by means of MPLC (silica gel, mobile phase: cyclohexane/ethyl acetate 4:1).

¹H-NMR (400 MHz, CDCl₃, δ/ppm): 8.35 (d, 1H), 8.24 (d, 2H), 7.33 (d, 2H), 7.22 (d, 1H), 6.99 (dd, 1H), 6.89 (s, 1H), 5.42 (s, 2H), 2.31 (s, 3H).

LC/MS (method I, ESIpos): Rₜ=1.32 min, m/z=480/482 [M+H]⁺.

### Example 140A

### 2-Chloro-4-[(5-methyl-3-{3-[4-(tetrahydro-2H-pyran-4-yl)phenyl]-1,2,4-oxadiazol-5-yl}-1H-pyrazol-1-yl)methyl]pyridine

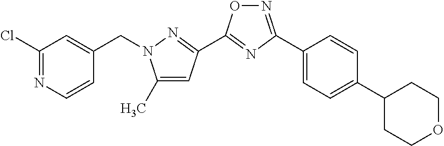 [SMILES: Cc1cc(-c2nc(-c3ccc(C4CCOCC4)cc3)no2)nn1Cc1ccnc(Cl)c1]

119 mg (1.06 mmol) of solid potassium tert-butylate were added to a solution of 204 mg (1.26 mmol) of the compound from Example 43A and 300 mg (0.967 mmol) of the compound from Example 124A in 10 ml of anhydrous THF at 0° C. The reaction mixture was then stirred first at RT for 15 h and then at the boiling point of the solvent for 4.5 h. After cooling to RT, approx. 1 ml of water and methanol in an amount such that a clear solution formed were added. This was separated directly into its components by means of preparative HPLC (method N). After removal of the solvent on a rotary evaporator, 220 mg (52% of th.) of the title compound were obtained from the combined product fractions.

¹H-NMR (400 MHz, CDCl₃, δ/ppm): 8.37 (d, 1H), 8.14 (d, 2H), 7.35 (d, 2H), 7.05 (d, 1H), 6.96 (dd, 1H), 6.88 (s, 1H), 5.43 (s, 2H), 4.12-4.08 (m, 2H), 3.58-3.52 (m, 2H), 2.88-2.79 (m, 1H), 2.31 (s, 3H), 1.92-1.79 (m, 4H).

LC/MS (method I, ESIpos): Rₜ=1.16 min, m/z=436 [M+H]⁺.

### Example 141A

### 4-({3-[3-(4-tert-Butylphenyl)-1,2,4-oxadiazol-5-yl]-5-methyl-1H-pyrazol-1-yl}methyl)-2-chloropyridine

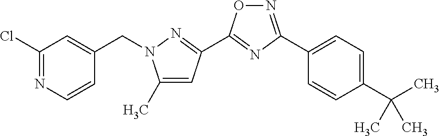 [SMILES: Cc1cc(-c2nc(-c3ccc(C(C)(C)C)cc3)no2)nn1Cc1ccnc(Cl)c1]

596 mg (5.31 mmol) of solid potassium tert-butylate were added to a solution of 1.15 g (7.08 mmol) of the compound from Example 43A and 1.00 g (3.54 mmol) of the compound from Example 125A in 30 ml of anhydrous THF at 0° C. The reaction mixture was then stirred first at RT for 15 h and then at the boiling point of the solvent for 4 h. After cooling to RT, approx. 120 ml of water were added and the mixture was extracted three times with approx. 60 ml of ethyl acetate each time. The combined organic extracts were washed with saturated sodium chloride solution and dried over anhydrous magnesium sulfate. After filtration and evaporation, the crude product obtained was purified by MPLC (silica gel, mobile phase: cyclohexane/ethyl acetate 4:1→1:2). 578 mg (40% of th.) of the title compound were obtained.

¹H-NMR (400 MHz, CDCl₃, δ/ppm): 8.37 (d, 1H), 8.12 (d, 2H), 7.51 (d, 2H), 7.06 (s, 1H), 6.97 (d, 1H), 6.88 (s, 1H), 5.43 (s, 2H), 2.31 (s, 3H), 1.37 (s, 9H).

LC/MS (method F, ESIpos): Rₜ=1.55 min, m/z=408/410 [M+H]⁺.

### Example 142A

### 2-Chloro-4-{[3-(3-{4-[1-(methoxymethyl)cyclobutyl]phenyl}-1,2,4-oxadiazol-5-yl)-5-methyl-1H-pyrazol-1-yl]methyl}pyridine

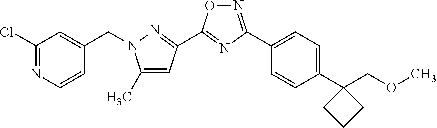 [SMILES: COCC1(c2ccc(-c3noc(-c4cc(C)n(Cc5ccnc(Cl)c5)n4)n3)cc2)CCC1]

519 mg (4.62 mmol) of solid potassium tert-butylate were added to a solution of 749 mg (4.62 mmol) of the compound from Example 43A and 750 mg (2.31 mmol) of the compound from Example 126A in 22.5 ml of anhydrous THF at 0° C. The reaction mixture was then stirred at the boiling point of the solvent for 5 h. After cooling to RT, approx. 3 drops of water were added and all the volatile constituents were removed on a rotary evaporator. The residue obtained was purified by means of MPLC (approx. 100 g of silica gel, mobile phase: cyclohexane/ethyl acetate 2:1). For further purification, the combined concentrated product fractions were then stirred with 1 ml of ethanol and the solid was filtered off with suction. 447 mg (43% of th.) of the title compound were obtained.

¹H-NMR (400 MHz, CDCl₃, δ/ppm): 8.37 (d, 1H), 8.13 (d, 2H), 7.30 (d, 2H), 7.06 (s, 1H), 6.97 (d, 1H), 6.88 (s, 1H), 5.43 (s, 2H), 3.57 (s, 2H), 3.28 (s, 3H), 2.41-2.28 (m, 4H), 2.30 (s, 3H), 2.16-2.04 (m, 1H), 1.93-1.83 (m, 1H).

LC/MS (method I, ESIpos): Rₜ=1.34 min, m/z=450/452 [M+H]⁺.

### Example 143A

### 2-Chloro-4-[(5-methyl-3-{3-[3-methyl-4-(tetrahydro-2H-pyran-4-yl)phenyl]-1,2,4-oxadiazol-5-yl}-1H-pyrazol-1-yl)methyl]pyridine

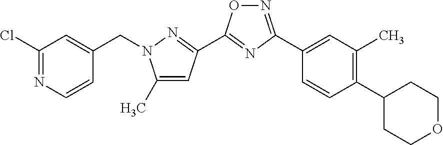 [SMILES: Cc1cc(-c2noc(-c3cc(C)n(Cc4ccnc(Cl)c4)n3)n2)ccc1C1CCOCC1]

71 mg (0.634 mmol) of solid potassium tert-butylate were added to a solution of 187 mg (0.576 mmol) of the compound from Example 127A and 121 mg (0.749 mmol) of the compound from Example 43A in 6 ml of anhydrous THF at 0° C. The reaction mixture was then stirred at RT for 16 h. Since the conversion was not complete, the mixture was heated further under reflux for another 8 h. After cooling to RT, 1 ml of water and methanol in an amount such that a clear solution formed were added. This solution was then separated directly into its components by means of preparative HPLC (method N). The product fractions were combined and freed from the solvent on a rotary evaporator. 150 mg (58% of th.) of the title compound were obtained.

¹H-NMR (400 MHz, CDCl₃, δ/ppm): 8.37 (d, 1H), 8.02 (d, 1H), 8.01 (dd, 1H), 7.34 (d, 1H), 7.05 (d, 1H), 6.96 (dd, 1H), 6.88 (s, 1H), 5.43 (s, 2H), 4.13-4.09 (m, 2H), 3.61-3.53 (m, 2H), 3.07-2.99 (m, 1H), 2.43 (s, 3H), 2.31 (s, 3H), 1.92-1.81 (m, 2H), 1.74-1.69 (m, 2H).

LC/MS (method F, ESIpos): Rₜ=1.34 min, m/z=448 [M+H]⁺.

### Example 144A

### 2-Chloro-4-[(3-{3-[3-chloro-4-(trifluoromethoxy)phenyl]-1,2,4-oxadiazol-5-yl}-5-methyl-1H-pyrazol-1-yl)methyl]pyridine

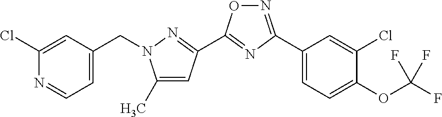 [SMILES: Cc1cc(-c2nc(-c3ccc(OC(F)(F)F)c(Cl)c3)no2)nn1Cc1ccnc(Cl)c1]

Analogously to the process described under Example 75A, 500 mg (1.38 mmol, purity of 95%) of the compound from Example 128A and 290 mg (1.79 mmol) of the compound from Example 43A were reacted to give 386 mg (57% of th., purity of 96%) of the title compound, the reaction components in this case being stirred with one another under reflux for 14 h.

¹H-NMR (400 MHz, CDCl₃, δ/ppm): 8.40-8.37 (m, 2H), 8.12 (d, 1H), 7.44 (d, 1H), 7.05 (s, 1H), 6.96 (d, 1H), 6.89 (s, 1H), 5.45 (s, 2H), 2.31 (s, 3H).

LC/MS (method I, ESIpos): Rₜ=1.36 min, m/z=469 [M+H]⁺.

### Example 145A

### 2-Chloro-4-{[5-methyl-3-(3-{4-[1-(trifluoromethyl)cyclopropyl]phenyl}-1,2,4-oxadiazol-5-yl)-1H-pyrazol-1-yl]methyl}pyridine

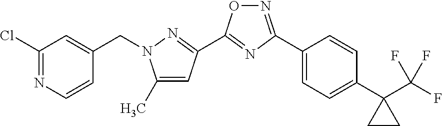 [SMILES: Cc1cc(-c2nc(-c3ccc(C4(C(F)(F)F)CC4)cc3)no2)nn1Cc1ccnc(Cl)c1]

A mixture of 450 mg (1.35 mmol) of the compound from Example 129A, 284 mg (1.75 mmol) of 2-chloro-4-(chloromethyl)pyridine and 166 mg (1.48 mmol) of potassium tert-butylate in 12 ml of THF was heated under reflux overnight, while stirring. After cooling to RT, ethyl acetate and water were added to the mixture. The phases were separated and the aqueous phase was extracted twice with ethyl acetate. The combined ethyl acetate phases were washed once with saturated sodium chloride solution, dried over magnesium sulfate, filtered and concentrated. The residue was purified by means of column chromatography (silica gel, mobile phase: cyclohexane/ethyl acetate 7:3). After drying in vacuo, 352 mg (57% of th.) of the title compound were obtained.

¹H-NMR (400 MHz, CDCl₃, δ/ppm): 8.37 (d, 1H), 8.19 (d, 2H), 7.60 (d, 2H), 7.05 (s, 1H), 6.96 (d, 1H), 6.88 (s, 1H), 5.44 (s, 2H), 2.30 (s, 3H), 1.48-1.33 (m, 2H), 1.09 (s, broad, 2H).

LC/MS (method F, ESIpos): Rₜ=1.48 min, m/z=460/462 [M+H]⁺.

### Example 146A

### tert-Butyl 4-[(5-methyl-3-{3-[4-(trifluoromethoxy)phenyl]-1,2,4-oxadiazol-5-yl}-1H-pyrazol-1-yl)methyl]-3′,6′-dihydro-2,4′-bipyridine-1′(2′H)-carboxylate

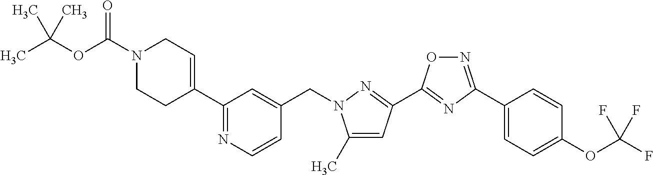 [SMILES: Cc1cc(-c2nc(-c3ccc(OC(F)(F)F)cc3)no2)nn1Cc1ccnc(C2=CCN(C(=O)OC(C)(C)C)CC2)c1]

464 mg (1.50 mmol) of tert-butyl 4-(4,4,5,5-tetramethyl-1,3,2-dioxaborolan-2-yl)-3,6-dihydro-pyridine-1(2H)-carboxylate [P. R. Eastwood, Tetrahedron Lett. 2000, 41(19), 3705-3708], 70 mg (0.10 mmol) of bis(triphenylphosphine)palladium(II) dichloride and 1.5 ml (3.0 mmol) of 2 M aqueous sodium carbonate solution were added to a solution of 480 mg (1.00 mmol) of the compound from Example 139A in 7.5 ml of DME. After the reaction mixture had been heated under reflux for 13 h, it was allowed to cool to RT and was diluted with approx. 50 ml of water. The mixture was extracted three time with approx. 20 ml of ethyl acetate each time. The combined organic extracts were washed with saturated sodium chloride solution and dried over anhydrous magnesium sulfate. After filtration, the solvent was removed on a rotary evaporator and the residue obtained was purified by means of filtration with suction over silica gel (mobile phase: methylene chloride/ethyl acetate 100:1→1:1). 318 mg (54% of th.) of the title compound were obtained.

¹H-NMR (400 MHz, CDCl₃, δ/ppm): 8.52 (d, 1H), 8.25 (d, 2H), 7.33 (d, 2H), 7.09 (s, 1H), 6.88 (d, 1H), 6.87 (s, 1H), 6.58-6.55 (m, 1H), 5.45 (s, 2H), 4.13-4.10 (m, 2H), 3.63-3.60 (m, 1H), 2.61-2.56 (m, 2H), 2.29 (s, 3H), 1.47 (s, 9H).

LC/MS (method D, ESIpos): Rₜ=2.88 min, m/z=583 [M+H]⁺.

### Example 147A

### 3-[(5-Methyl-3-{3-[4-(tetrahydro-2H-pyran-4-yl)phenyl]-1,2,4-oxadiazol-5-yl}-1H-pyrazol-1-yl)-methyl]benzoic acid

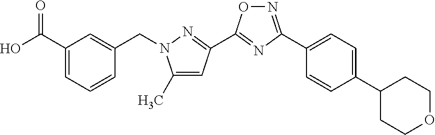 [SMILES: Cc1cc(-c2nc(-c3ccc(C4CCOCC4)cc3)no2)nn1Cc1cccc(C(=O)O)c1]

### Step 1: Methyl 3-[(5-methyl-3-{3-[4-(tetrahydro-2H-pyran-4-yl)phenyl]-1,2,4-oxadiazol-5-yl}-1H-pyrazol-1-yl)methyl]benzoate

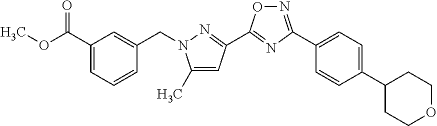 [SMILES: COC(=O)c1cccc(Cn2nc(-c3nc(-c4ccc(C5CCOCC5)cc4)no3)cc2C)c1]

Analogously to the process described under Example 76A, 168 mg (0.733 mmol) of 3-(bromomethyl)benzoic acid methyl ester and 175 mg (0.564 mmol) of the compound from Example 124A were reacted to give 208 mg (80% of th.) of the title compound.

¹H-NMR (400 MHz, CDCl₃, δ/ppm): 8.15 (d, 2H), 7.98 (d, 1H), 7.39 (s, 1H), 7.42 (t, 2H), 7.35 (d, 2H), 7.34 (d, 1H), 6.82 (s, 1H), 5.50 (s, 2H), 4.12-4.08 (m, 2H), 3.91 (s, 3H), 3.55 (dt, 2H), 2.87-2.80 (m, 1H), 2.29 (s, 3H), 1.92-1.77 (m, 4H).

LC/MS (method I, ESIpos): Rₜ=1.24 min, m/z=459 [M+H]⁺.

### Step 2: 3-[(5-Methyl-3-{3-[4-(tetrahydro-2H-pyran-4-yl)phenyl]-1,2,4-oxadiazol-5-yl}-1H-pyrazol-1-yl)methyl]benzoic acid

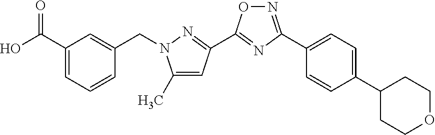 [SMILES: Cc1cc(-c2nc(-c3ccc(C4CCOCC4)cc3)no2)nn1Cc1cccc(C(=O)O)c1]

Analogously to the process described under Example 93A, 85 mg (87% of th.) of the title compound were obtained from 100 mg (0.218 mmol) of the compound from Example 147A/step 1.

¹H-NMR (400 MHz, DMSO-d₆, δ/ppm): 13.07 (broad, 1H), 8.01 (d, 2H), 7.89 (d, 1H), 7.78 (s, 1H), 7.53-7.44 (m, 4H), 6.93 (s, 1H), 5.59 (s, 2H), 3.99-3.94 (m, 2H), 3.47 (dt, 2H), 2.92-2.83 (m, 1H), 2.34 (s, 3H), 1.77-1.66 (m, 4H).

LC/MS (method F, ESIpos): Rₜ=1.22 min, m/z=445 [M+H]⁺.

### Example 148A

### N′-Hydroxy-5-methyl-1-{3-[(4-methylpiperazin-1-yl)carbonyl]benzyl}-1H-pyrazole-3-carboxamide amide

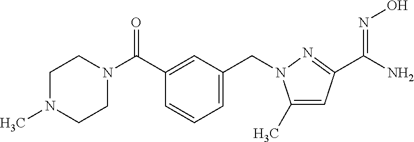 [SMILES: Cc1cc(/C(N)=N/O)nn1Cc1cccc(C(=O)N2CCN(C)CC2)c1]

### Step 1: 5-Methyl-1-{3-[(4-methylpiperazin-1-yl)carbonyl]benzyl}-1H-pyrazole-3-carboxamide

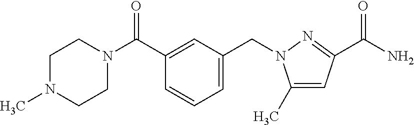 [SMILES: Cc1cc(C(N)=O)nn1Cc1cccc(C(=O)N2CCN(C)CC2)c1]

34.4 ml (0.394 mol) of oxalyl chloride were slowly added dropwise to a mixture of 27.0 g (78.9 mmol) of the compound from Example 105A and a few drops of DMF in 100 ml of methylene chloride at RT and the mixture was stirred at RT for 1 h and then concentrated. The residue was introduced in portions into 300 ml of a 33% strength aqueous ammonia solution cooled to 0° C. The mixture was stirred at RT for 1 h and the solid formed was then filtered off to obtain, after washing with water and drying in vacuo, a first batch of the title compound. The mother liquor was concentrated to dryness, toluene was added to the residue and the mixture was concentrated to dryness again. After this procedure had been repeated twice, the residue was stirred with methanol and the solid was filtered off, washed with methanol and dried in vacuo. The second batch of the title compound obtained in this way was combined with the first. 33.0 g (94% of th., purity of 77%) in total of the title compound were obtained.

LC/MS (method D, ESIpos): Rₜ=0.88 and 0.93 min, in each case m/z=342 [M+H]⁺.

### Step 2: 5-Methyl-1-{3-[(4-methylpiperazin-1-yl)carbonyl]benzyl}-1H-pyrazole-3-carbonitrile

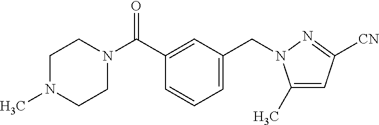 [SMILES: Cc1cc(C#N)nn1Cc1cccc(C(=O)N2CCN(C)CC2)c1]

10.9 ml (76.9 mmol) of trifluoroacetic acid anhydride were added to 21.0 g (30.7 mmol, purity of 50%) of the compound from Example 148A/step 1 and the mixture was stirred at RT for 1 h. Saturated aqueous sodium bicarbonate solution was then added and the mixture was extracted three times with ethyl acetate. The combined ethyl acetate phases were dried over magnesium sulfate, filtered and concentrated. The residue was purified by means of flash chromatography (silica gel, mobile phase: methylene chloride/methanol 95:5). After concentration and drying of the product fractions, 2.50 g (25% of th.) of the title compound were obtained.

LC/MS (method D, ESIpos): Rₜ=1.07 min, m/z=324 [M+H]⁺.

### Step 3: N′-Hydroxy-5-methyl-1-{3-[(4-methylpiperazin-1-yl)carbonyl]benzyl}-1H-pyrazole-3-carboximide amide

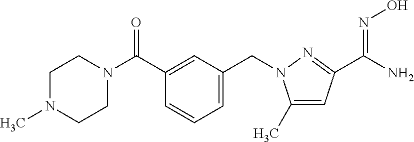 [SMILES: Cc1cc(/C(N)=N/O)nn1Cc1cccc(C(=O)N2CCN(C)CC2)c1]

8.00 g (24.7 mmol) of the compound from Example 148A/step 2 were dissolved in 320 ml of ethanol, 3.78 g (54.4 mmol) of hydroxylamine hydrochloride and 7.6 ml (54.4 mmol) of triethylamine were added and the mixture was heated under reflux for 5 h. After cooling to RT, the mixture was concentrated. The residue was stirred with a mixture of methylene chloride and methanol (8:1) and the solid formed was filtered off with suction. After drying, a first batch of the title compound was obtained in this way. The mother liquor was concentrated and the residue was purified by means of flash chromatography (silica gel, mobile phase: methylene chloride/methanol 8:2). After concentration and drying of the product fractions, a second batch of the title compound was obtained. Together, 6.70 g (68% of th., purity of 90%) of the title compound were obtained.

LC/MS (method I, ESIpos): Rₜ=0.81 min, m/z=357 [M+H]⁺.

### EMBODIMENT EXAMPLES

### Example 1

### (4-Methylpiperazin-1-yl) {5-[(5-methyl-3-{3-[4-(trifluoromethoxy)phenyl]-1,2,4-oxadiazol-5-yl}-1H-pyrazol-1-yl)methyl]pyridin-2-yl}methanone

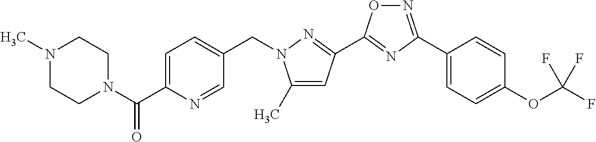 [SMILES: Cc1cc(-c2nc(-c3ccc(OC(F)(F)F)cc3)no2)nn1Cc1ccc(C(=O)N2CCN(C)CC2)nc1]

Under inert conditions, 85 mg (0.191 mmol) of the compound from Example 92A were dissolved in 3 ml of anhydrous methylene chloride, and 83 μl (0.954 mmol) of oxalyl chloride and a small drop of DMF were added. After the mixture had been stirred at RT for 1 h, it was concentrated to dryness on a rotary evaporator. The residue obtained was dried under a high vacuum for approx. 1 h and then dissolved in 2 ml of anhydrous THF. This solution was added dropwise to a solution of 29 mg (0.286 mmol) of 1-methylpiperazine and 66 μl (0.382 mmol) of N,N-diisopropylethylamine in 1 ml of anhydrous THF. After stirring at RT for 16 h, 3 ml of water were added to the reaction mixture and the mixture was separated into its components by means of preparative HPLC (method N). The product fractions were combined and freed from the solvent on a rotary evaporator. The residue was redissolved in a few ml of methanol and the solution was passed over a bicarbonate cartridge (Polymerlabs, Stratospheres SPE, PL-HCO₃ MP SPE, capacity 0.9 mmol). After renewed evaporation of the solvent, 93 mg (93% of th.) of the title compound were obtained in this way.

¹H-NMR (400 MHz, CDCl₃, δ/ppm): 8.45 (d, 1H), 8.24 (d, 2H), 7.64 (d, 1H), 7.60 (dd, 1H), 7.33 (d, 2H), 6.85 (s, 1H), 5.50 (s, 2H), 3.82 (dd, 2H), 3.60 (dd, 2H), 2.51 (dd, 2H), 2.40 (dd, 2H), 2.34 (s, 3H), 2.32 (s, 3H).

HPLC (method B): Rₜ=4.32 min.

MS (ESIpos): m/z=528 [M+H]⁺.

### Example 2

### 1-Methyl-4-[({5-[(5-methyl-3-{3-[4-(trifluoromethoxy)phenyl]-1,2,4-oxadiazol-5-yl}-1H-pyrazol-1-yl)methyl]pyridin-2-yl}amino)methyl]piperidin-4-ol

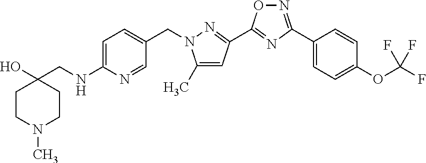 [SMILES: Cc1cc(-c2nc(-c3ccc(OC(F)(F)F)cc3)no2)nn1Cc1ccc(NCC2(O)CCN(C)CC2)nc1]

A mixture of 100 mg (0.229 mmol) of the compound from Example 79A and 249 mg (1.15 mmol) of 4-(aminomethyl)-1-methylpiperidin-4-ol was heated at 180° C. in a microwave oven (CEM Discover, initial irradiation power 250 W) for 3 h, while stirring. After cooling to RT, the liquid present was decanted off from the solid. The solid was then dissolved in a mixture of 2 ml of acetonitrile and 1 ml of water and purified by means of preparative HPLC (method O). The combined product-containing fractions were concentrated on a rotary evaporator to a residual volume, saturated aqueous sodium bicarbonate solution was added and the solid formed was filtered. After drying in vacuo, 15 mg (12% of th.) of the title compound were obtained.

¹H-NMR (400 MHz, CDCl₃, δ/ppm): 8.26 (d, 2H), 7.92 (d, 1H), 7.37-7.32 (m, 3H), 6.77 (s, 1H), 6.43 (d, 1H), 5.36 (s, broad, 1H), 5.28 (s, 2H), 4.84 (t, 1H), 3.40 (d, 2H), 2.66-2.60 (m, 2H), 2.45-2.36 (m, 2H), 2.31 (s, 6H), 1.75-1.58 (m, 4H).

LC/MS (method F, ESIpos): Rₜ=1.01 min, m/z=544 [M+H]⁺.

### Example 3

### (3R)-1-{5-[(5-Methyl-3-{3-[4-(trifluoromethoxy)phenyl]-1,2,4-oxadiazol-5-yl}-1H-pyrazol-1-yl)-methyl]pyridin-2-yl}pyrrolidin-3-amine

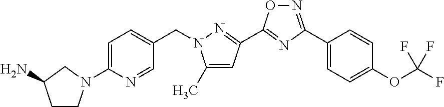 [SMILES: Cc1cc(-c2nc(-c3ccc(OC(F)(F)F)cc3)no2)nn1Cc1ccc(N2CC[C@@H](N)C2)nc1]

A mixture of 150 mg (0.344 mmol) of the compound from Example 79A and 641 mg (3.44 mmol) of tert-butyl-(3R)-pyrrolidin-3-yl carbamate in 1 ml of ethylene glycol dimethyl ether was heated at 180° C. in a microwave apparatus (CEM Discover, initial irradiation power 250 W) for 3 h, while stirring. After cooling to RT, the mixture was diluted with 3 ml of acetonitrile and 1 ml of water and then purified by means of preparative HPLC (method O). The product-containing fractions were combined, a basic pH was established with sodium bicarbonate and some of the volume of liquid was removed on a rotary evaporator. Extraction was then carried out three times with 30 ml of ethyl acetate each time, the combined ethyl acetate phases were dried over sodium sulfate and filtered and the solvent was removed on a rotary evaporator. The residue was purified again by means of preparative HPLC (modification of method O: instead of 0.1% aqueous TFA, 0.1% aqueous ammonia solution was used). 49 mg (29% of th.) of the title compound were obtained.

¹H-NMR (400 MHz, DMSO-d₆, δ/ppm): 8.20 (d, 2H), 8.07 (d, 1H), 7.60 (d, 2H), 7.40 (dd, 1H), 6.88 (s, 1H), 6.38 (d, 1H), 5.32 (s, 2H), 3.56-3.42 (m, 3H), 3.38-3.28 (m, 1H), 3.02 (dd, 1H), 2.38 (s, 3H), 2.06-1.96 (m, 1H), 1.81-1.65 (m, 3H).

LC/MS (method D, ESIpos): Rₜ=1.69 min, m/z=486 [M+H]⁺.

The compounds in the following table were prepared from the compound from Example 79A and the corresponding amine component analogously to one of the processes described under Example 2 and 3:

| | | HPLC: | MS: m/z | LC/MS |
|---|---|---|---|---|
| Example | Structure | Rₜ [min] | [M + H]⁺ | method |
| 4 | 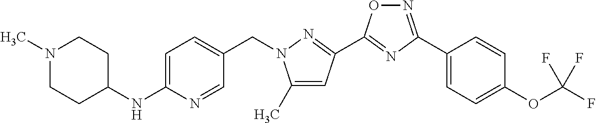 [SMILES: Cc1cc(-c2nc(-c3ccc(OC(F)(F)F)cc3)no2)nn1Cc1ccc(NC2CCN(C)CC2)nc1] | 1.51 | 514 | C |
| | ¹H-NMR (400 MHz, DMSO-d₆, δ/ppm): 8.20 (d, 2H), 7.97 (d, 1H), 7.60 (d, 2H), 7.31- | | | |
| | 7.27 (dd, 1H), 6.88 (s, 1H), 6.52 (d, 1H), 6.42 (d, 1H), 5.27 (s, 2H), 3.68-3.57 (m, 1H), | | | |
| | 2.73-2.66 (d, 2H), 2.39 (s, 3H), 2.13 (s, 3H), 2.00-1.92 (t, 2H), 1.87-1.79 (m, 2H), 1.42- | | | |
| | 1.33 (m, 2H). | | | |
| 5 | 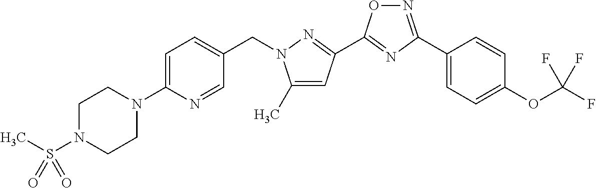 [SMILES: Cc1cc(-c2nc(-c3ccc(OC(F)(F)F)cc3)no2)nn1Cc1ccc(N2CCN(S(C)(=O)=O)CC2)nc1] | 2.23 | 564 | E |
| | ¹H-NMR (400 MHz, CDCl₃, δ/ppm): 8.25 (d, 2H), 8.12 (s, 1H), 7.44 (d, 1H), 7.33 (d, | | | |
| | 2H), 6.79 (s, 1H), 6.63 (d, 1H), 5.32 (s, 2H), 3.71-3.66 (m, 4H), 3.32-3.28 (m, 4H), 2.80 | | | |
| | (s, 3H), 2.32 (s, 3H). | | | |
| 6 | 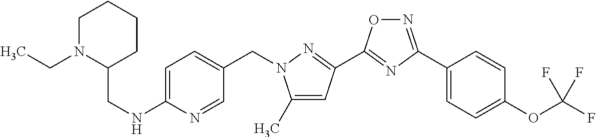 [SMILES: CCN1CCCCC1CNc1ccc(Cn2nc(-c3nc(-c4ccc(OC(F)(F)F)cc4)no3)cc2C)cn1] | 0.98 | 542 | K |
| 7 | 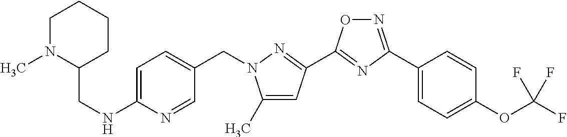 [SMILES: Cc1cc(-c2nc(-c3ccc(OC(F)(F)F)cc3)no2)nn1Cc1ccc(NCC2CCCCN2C)nc1] | 0.82 | 528 | K |
| 8 | 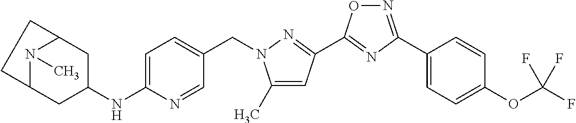 [SMILES: Cc1cc(-c2nc(-c3ccc(OC(F)(F)F)cc3)no2)nn1Cc1ccc(NC2CC3CCC(C2)N3C)nc1] | 0.86 | 540 | K |
| 9 | 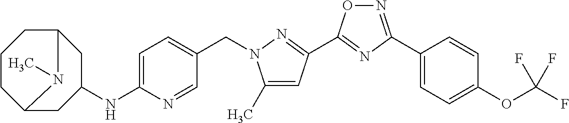 [SMILES: Cc1cc(-c2nc(-c3ccc(OC(F)(F)F)cc3)no2)nn1Cc1ccc(NC2CC3CCCC(C2)N3C)nc1] | 0.89 | 554 | K |
| 10 | 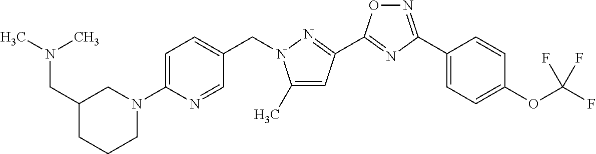 [SMILES: Cc1cc(-c2nc(-c3ccc(OC(F)(F)F)cc3)no2)nn1Cc1ccc(N2CCCC(CN(C)C)C2)nc1] | 0.94 | 542 | K |
| 11 | 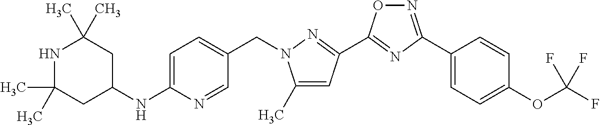 [SMILES: Cc1cc(-c2nc(-c3ccc(OC(F)(F)F)cc3)no2)nn1Cc1ccc(NC2CC(C)(C)NC(C)(C)C2)nc1] | 0.89 | 556 | K |
| 12 | 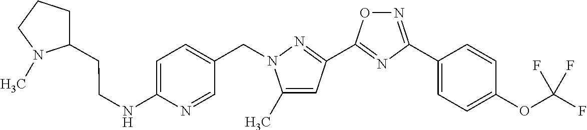 [SMILES: Cc1cc(-c2nc(-c3ccc(OC(F)(F)F)cc3)no2)nn1Cc1ccc(NCCC2CCCN2C)nc1] | 0.82 | 529 | K |
| 13 | 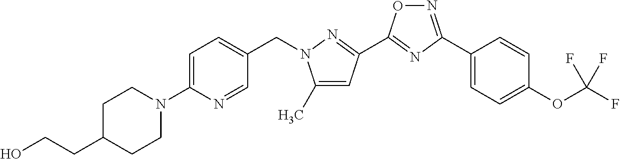 [SMILES: Cc1cc(-c2nc(-c3ccc(OC(F)(F)F)cc3)no2)nn1Cc1ccc(N2CCC(CCO)CC2)nc1] | 1.02 | 529 | K |
| 14 | 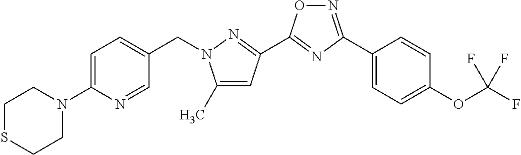 [SMILES: Cc1cc(-c2nc(-c3ccc(OC(F)(F)F)cc3)no2)nn1Cc1ccc(N2CCSCC2)nc1] | 1.26 | 503 | K |

### Example 15

### 1-Methyl-4-{4-[(5-methyl-3-{3-[4-(trifluoromethoxy)phenyl]-1,2,4-oxadiazol-5-yl}-1H-pyrazol-1-yl)methyl]pyridin-2-yl}piperazine

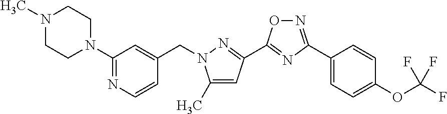 [SMILES: Cc1cc(-c2nc(-c3ccc(OC(F)(F)F)cc3)no2)nn1Cc1ccnc(N2CCN(C)CC2)c1]

A mixture of 150 mg (0.344 mmol) of the compound from Example 81A and 690 mg (6.88 mmol) of 1-methylpiperazine in 1 ml of ethylene glycol dimethyl ether was heated at 180° C. in a microwave apparatus (CEM Discover, initial irradiation power 250 W) for 3 h, while stirring. After cooling to RT, the mixture was purified directly by means of preparative HPLC (method O). The combined product-containing fractions were concentrated on a rotary evaporator to a small residual volume of liquid, adjusted to a slightly basic pH with sodium bicarbonate and then extracted three times with 30 ml of methylene chloride each time. The combined methylene chloride phases were dried over magnesium sulfate and filtered and the solvent was removed on a rotary evaporator. After crystallization from hexane, 114 mg (66% of th.) of the title compound were obtained.

¹H-NMR (400 MHz, CDCl₃, δ/ppm): 8.27 (d, 2H), 8.12 (d, 1H), 7.33 (d, 2H), 6.84 (s, 1H), 6.36-6.32 (m, 2H), 5.36 (s, 2H), 3.55-3.48 (m, 4H), 2.55-2.45 (m, 4H), 2.31 (s, 3H), 2.29 (s, 3H).

LC/MS (method F, ESIpos): Rₜ=0.97 min, m/z=500 [M+H]⁺.

### Example 16

### N,N-Dimethyl-1-{4-[(5-methyl-3-{3-[4-(trifluoromethoxy)phenyl]-1,2,4-oxadiazol-5-yl}-1H-pyrazol-1-yl)methyl]pyridin-2-yl}piperidin-4-amine

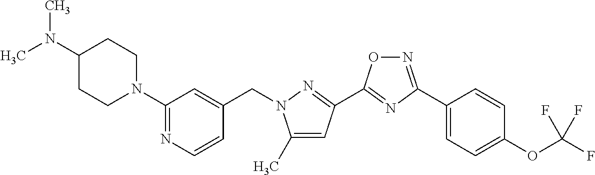 [SMILES: Cc1cc(-c2nc(-c3ccc(OC(F)(F)F)cc3)no2)nn1Cc1ccnc(N2CCC(N(C)C)CC2)c1]

294 mg (2.29 mmol) of N,N-dimethylpiperidin-4-amine were added to 200 mg (0.459 mmol) of the compound from Example 81A at RT under argon. The mixture was then stirred at a bath temperature of 150° C. overnight. After cooling to RT, the mixture was taken up in acetonitrile and purified directly by means of preparative HPLC (method O). The combined product-containing fractions were rendered basic with saturated sodium bicarbonate solution and concentrated on a rotary evaporator to a low residual volume of solvent. The solid thereby formed was filtered off, washed twice with water and twice with pentane and dried in vacuo. 137 mg (57% of th.) of the title compound were obtained.

¹H-NMR (400 MHz, CDCl₃, δ/ppm): 8.26 (d, 2H), 8.12 (d, 1H), 7.32 (d, 2H), 6.82 (s, 1H), 6.32 (d, 1H), 6.31 (s, 1H), 5.35 (s, 2H), 3.51 (t, 4H), 2.76-2.67 (m, 1H), 2.62 (t, 4H), 2.30 (s, 3H), 1.08 (d, 6H).

LC/MS (method F, ESIpos): Rₜ=1.12 min, m/z=528 [M+H]⁺.

The compounds in the following table were prepared from the compound from Example 81A and the corresponding amine component analogously to one of the processes described under Example 15 and 16:

| | | HPLC: | MS: m/z | LC/MS |
|---|---|---|---|---|
| Example | Structure | Rₜ [min] | [M + H]⁺ | method |
| 17 | 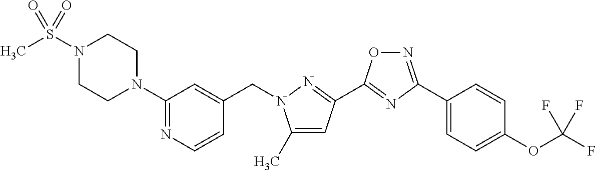 [SMILES: Cc1cc(-c2nc(-c3ccc(OC(F)(F)F)cc3)no2)nn1Cc1ccnc(N2CCN(S(C)(=O)=O)CC2)c1] | 2.14 | 564 | J |
| 18 | 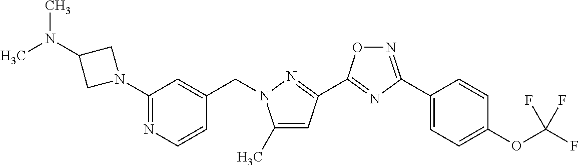 [SMILES: Cc1cc(-c2nc(-c3ccc(OC(F)(F)F)cc3)no2)nn1Cc1ccnc(N2CC(N(C)C)C2)c1] | 0.95 | 500 | I |
| | ¹H-NMR (400 MHz, CDCl₃, δ/ppm): 8.25 (d, 2H), 8.18 (d, 1H), 7.32 (d, 2H), 6.82 (s, | | | |
| | 1H), 6.32 (d, 1H), 5.95 (s, 1H), 5.32 (s, 2H), 4.06-4.02 (m, 2H), 3.83-3.80 (m, 2H), 3.25 | | | |
| | (m, 1H), 2.26 (s, 3H), 2.22 (s, 6H). | | | |
| 19 | 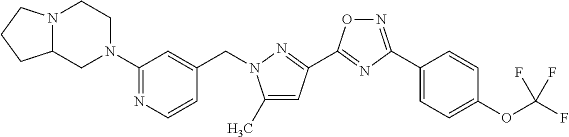 [SMILES: Cc1cc(-c2nc(-c3ccc(OC(F)(F)F)cc3)no2)nn1Cc1ccnc(N2CCN3CCCC3C2)c1] | 1.67 | 526 | J |
| 20 | 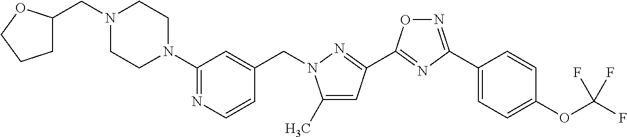 [SMILES: Cc1cc(-c2nc(-c3ccc(OC(F)(F)F)cc3)no2)nn1Cc1ccnc(N2CCN(CC3CCCO3)CC2)c1] | 1.71 | 570 | J |
| 21 | 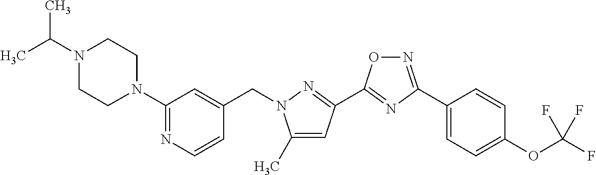 [SMILES: Cc1cc(-c2nc(-c3ccc(OC(F)(F)F)cc3)no2)nn1Cc1ccnc(N2CCN(C(C)C)CC2)c1] | 0.96 | 528 | I |
| | ¹H-NMR (400 MHz, CDCl₃, δ/ppm): 8.25 (d, 2H), 8.12 (d, 1H), 7.33 (d, 2H), 6.83 (s, | | | |
| | 1H), 6.34-6.31 (m, 2H), 5.35 (s, 2H), 3.51 (t, 4H), 2.73-2.66 (m, 1H), 2.61 (t, 4H), 2.28 | | | |
| | (s, 3H), 1.06 (d, 6H). | | | |
| 22 | 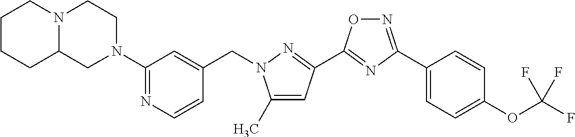 [SMILES: Cc1cc(-c2nc(-c3ccc(OC(F)(F)F)cc3)no2)nn1Cc1ccnc(N2CCN3CCCCC3C2)c1] | 1.71 | 540 | J |
| 23 | 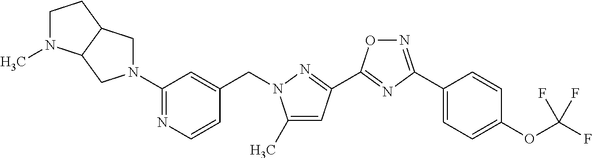 [SMILES: Cc1cc(-c2nc(-c3ccc(OC(F)(F)F)cc3)no2)nn1Cc1ccnc(N2CC3CCN(C)C3C2)c1] | 0.94 | 526 | I |
| | ¹H-NMR (400 MHz, CDCl₃, δ/ppm): 8.25 (d, 2H), 8.09 (d, 1H), 7.32 (d, 2H), 6.82 (s, | | | |
| | 1H), 6.31 (d, 1H), 6.10 (s, 1H), 5.33 (s, 2H), 3.89-3.82 (m, 1H), 3.55 (t, 1H), 3.44-3.33 | | | |
| | (m, 3H), 3.33-3.20 (m, broad, 1H), 3.07-2.98 (m, broad, 1H), 2.55 (s, 3H), 2.29 (s, 3H), | | | |
| | 2.29-2.19 (m, broad, 1H), 1.92-1.81 (m, broad, 2H). | | | |
| 24 | 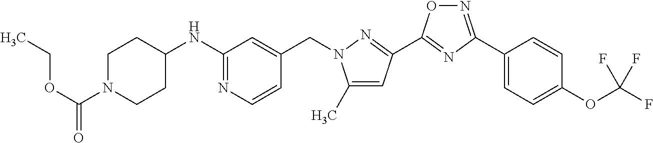 [SMILES: CCOC(=O)N1CCC(Nc2cc(Cn3nc(-c4nc(-c5ccc(OC(F)(F)F)cc5)no4)cc3C)ccn2)CC1] | 1.77 | 572 | J |
| 25 | 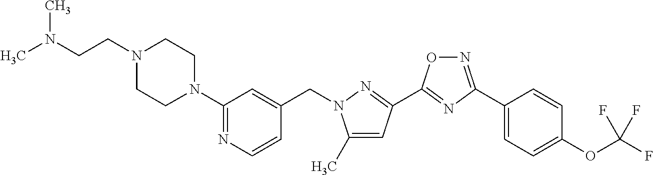 [SMILES: Cc1cc(-c2nc(-c3ccc(OC(F)(F)F)cc3)no2)nn1Cc1ccnc(N2CCN(CCN(C)C)CC2)c1] | 0.90 | 557 | F |
| | ¹H-NMR (400 MHz, CDCl₃, δ/ppm): 8.26 (d, 2H), 8.12 (d, 1H), 7.33 (d, 2H), 6.83 (s, | | | |
| | 1H), 6.35-6.30 (m, 2H), 5.36 (s, 2H), 3.55-3.49 (m, 4H), 2.60-2.55 (m, 4H), 2.55-2.42 | | | |
| | (m, 4H), 2.29 (s, 3H), 2.26 (s, 3H). | | | |
| 26 | 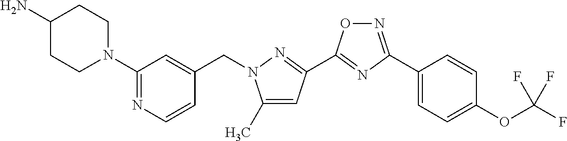 [SMILES: Cc1cc(-c2nc(-c3ccc(OC(F)(F)F)cc3)no2)nn1Cc1ccnc(N2CCC(N)CC2)c1] | 0.93 | 500 | I |
| | ¹H-NMR (400 MHz, CDCl₃, δ/ppm): 8.23 (d, 2H), 8.11 (d, 1H), 7.32 (d, 2H), 6.82 (s, | | | |
| | 1H), 6.32 (s, 1H), 6.31-6.29 (m, 1H), 5.32 (s, 2H), 4.17 (m, 2H), 2.92-2.84 (m, 3H), | | | |
| | 2.27 (s, 3H), 1.90-1.82 (m, 1H), 1.75-1.65 (m, 1H), 1.38-1.28 (m, 2H). | | | |
| 27 | 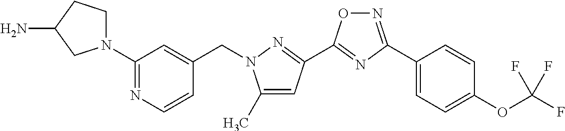 [SMILES: Cc1cc(-c2nc(-c3ccc(OC(F)(F)F)cc3)no2)nn1Cc1ccnc(N2CCC(N)C2)c1] | 1.68 | 486 | D |
| | ¹H-NMR (400 MHz, CDCl₃, δ/ppm): 8.25 (d, 2H), 8.19 (d, 1H), 7.32 (d, 2H), 6.82 (s, | | | |
| | 1H), 6.28 (d, 1H), 6.00 (s, 1H), 5.34 (s, 2H), 3.75-3.68 (m, 1H), 3.65-3.52 (m, 2H), | | | |
| | 3.48-3.38 (m, 1H), 3.18 (dd, 1H), 2.29 (s, 3H), 2.23-2.15 (m, 1H), 1.83-1.68 (m, 1H). | | | |
| 28 | 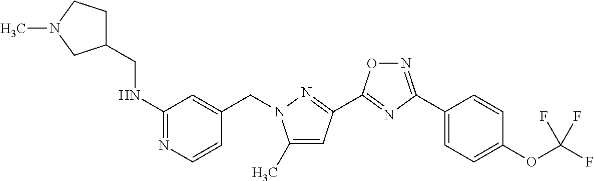 [SMILES: Cc1cc(-c2nc(-c3ccc(OC(F)(F)F)cc3)no2)nn1Cc1ccnc(NCC2CCN(C)C2)c1] | 1.41 | 513 | J |
| 29 | 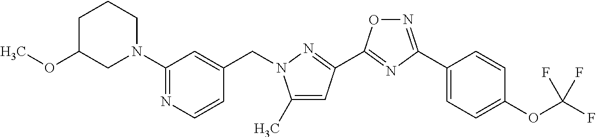 [SMILES: COC1CCCN(c2cc(Cn3nc(-c4nc(-c5ccc(OC(F)(F)F)cc5)no4)cc3C)ccn2)C1] | 1.88 | 515 | J |
| 30 | 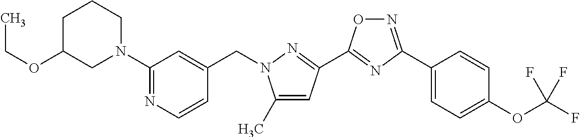 [SMILES: CCOC1CCCN(c2cc(Cn3nc(-c4nc(-c5ccc(OC(F)(F)F)cc5)no4)cc3C)ccn2)C1] | 1.90 | 529 | J |
| 31 | 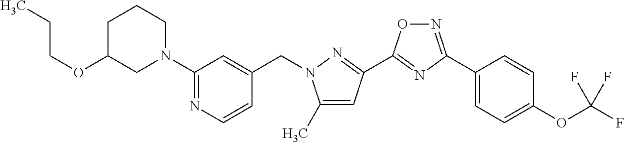 [SMILES: CCCOC1CCCN(c2cc(Cn3nc(-c4nc(-c5ccc(OC(F)(F)F)cc5)no4)cc3C)ccn2)C1] | 2.04 | 543 | J |

### Example 32

### (4-Methylpiperazin-1-yl) {3-[(5-methyl-3-{3-[4-(trifluoromethoxy)phenyl]-1,2,4-oxadiazol-5-yl}-1H-pyrazol-1-yl)methyl]phenyl}methanone

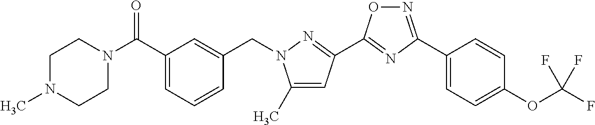 [SMILES: Cc1cc(-c2nc(-c3ccc(OC(F)(F)F)cc3)no2)nn1Cc1cccc(C(=O)N2CCN(C)CC2)c1]

Under inert conditions, 80 mg (0.180 mmol) of the compound from Example 93A were dissolved in 2 ml of anhydrous methylene chloride, and 79 μl (0.90 mmol) of oxalyl chloride and a small drop of DMF were added. After the mixture had been stirred at RT for 1 h, it was concentrated to dryness on a rotary evaporator. The residue obtained was dried under a high vacuum for approx. 1 h and then dissolved in 1 ml of anhydrous THF. This solution was added dropwise to a solution of 36 mg (0.360 mmol) of 1-methylpiperazine and 94 μl (0.540 mmol) of N,N-diisopropylethylamine in 1 ml of anhydrous THF. After stirring at RT for 16 h, 3 ml of water were added to the reaction mixture and the mixture was separated into its components by means of preparative HPLC (method N). The product fractions were combined and freed from the solvent on a rotary evaporator. The residue was redissolved in a few ml of methanol and the solution was passed over a bicarbonate cartridge (Polymerlabs, Stratospheres SPE, PL-HCO₃ MP SPE, capacity 0.9 mmol). After renewed evaporation of the solvent, 78 mg (82% of th.) of the title compound were obtained.

¹H-NMR (400 MHz, CDCl₃, δ/ppm): 8.25 (d, 2H), 7.41-7.33 (m, 4H), 7.20 (d, 1H), 7.17 (s, 1H), 6.83 (s, 1H), 5.48 (s, 2H), 3.76 (broad, 2H), 3.37 (broad, 2H), 2.44 (broad, 2H), 2.30 (s, 3H), 2.29 (broad, 2H), 2.26 (s, 3H).

HPLC (method B): Rₜ=4.45 min.

MS (DCI, NH₃): m/z=527 [M+H]⁺.

LC/MS (method C, ESIpos): Rₜ=1.71 min, m/z=527 [M+H]⁺.

The compounds in the following table were prepared from the compound from Example 93A and the corresponding amine analogously to the process described under Example 32. These amines were either commercially obtainable, or their preparation has been described above or in the literature: 1-cyclopropylpiperazine [F. Zaragoza et al., J. Med. Chem. 2004, 47 (11), 2833-2838], 1-(2,2,2-trifluoroethyl)piperazine [H.-L. Wang et al., J. Med. Chem. 2007, 50 (15), 3528-3539]. If the amines were employed in the form of their hydrochlorides or dihydrochlorides, the amount of base used (N,N-diisopropylethylamine) was in each case increased by a corresponding equivalent.

| | | HPLC: | MS: m/z | LC/MS |
|---|---|---|---|---|
| Example | Structure | Rₜ [min] | [M + H]⁺ | method |
| 33 | 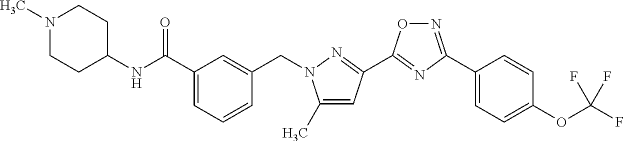 [SMILES: Cc1cc(-c2nc(-c3ccc(OC(F)(F)F)cc3)no2)nn1Cc1cccc(C(=O)NC2CCN(C)CC2)c1] | 1.75 | 541 | C |
| | ¹H-NMR (400 MHz, CDCl₃, δ/ppm): 8.25 (d, 2H), 7.65 (s, 1H), 7.63 (d, 1H), 7.40 (t, | | | |
| | 1H), 7.33 (d, 2H), 7.24 (d, 1H), 6.82 (s, 1H), 5.94 (d, broad, 1H), 5.50 (s, 2H), 4.01- | | | |
| | 3.92 (m, 1H), 2.83-2.77 (m, 2H), 2.29 (s, 3H), 2.28 (s, 3H), 2.17-2.10 (m, 2H), 2.05- | | | |
| | 2.00 (m, 2H), 1.60-1.50 (m, 2H). | | | |
| 34 | 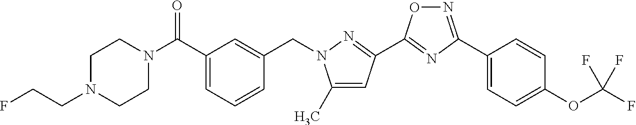 [SMILES: Cc1cc(-c2nc(-c3ccc(OC(F)(F)F)cc3)no2)nn1Cc1cccc(C(=O)N2CCN(CCF)CC2)c1] | 0.97 | 559 | I |
| | ¹H-NMR (400 MHz, CDCl₃, δ/ppm): 8.25 (d, 2H), 7.41-7.33 (m, 4H), 7.21 (dd, 1H), | | | |
| | 7.16 (d, 1H), 6.83 (s, 1H), 5.48 (s, 2H), 4.53 (td, 2H), 3.78 (broad, 2H), 3.38 (broad, | | | |
| | 2H), 2.67 (td, 2H), 2.58 (broad, 2H), 2.43 (broad, 2H), 2.31 (s, 3H). | | | |
| 35 | 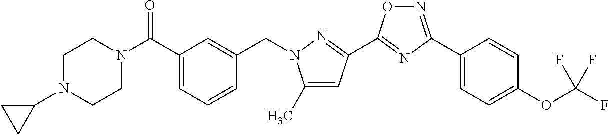 [SMILES: Cc1cc(-c2nc(-c3ccc(OC(F)(F)F)cc3)no2)nn1Cc1cccc(C(=O)N2CCN(C3CC3)CC2)c1] | 1.03 | 553 | I |
| | ¹H-NMR (400 MHz, CDCl₃, δ/ppm): 8.25 (d, 2H), 7.42-7.32 (m, 4H), 7.20 (dd, 1H), | | | |
| | 7.15 (d, 1H), 6.83 (s, 1H), 5.48 (s, 2H), 3.70 (broad, 2H), 3.29 (broad, 2H), 2.65 (broad, | | | |
| | 2H), 2.47 (broad, 2H), 2.31 (s, 3H), 1.60-1.54 (m, 1H), 0.44-0.34 (m, 4H). | | | |
| 36 | 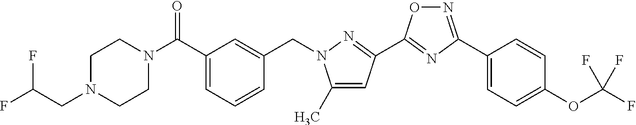 [SMILES: Cc1cc(-c2nc(-c3ccc(OC(F)(F)F)cc3)no2)nn1Cc1cccc(C(=O)N2CCN(CC(F)F)CC2)c1] | 1.23 | 577 | I |
| | ¹H-NMR (400 MHz, CDCl₃, δ/ppm): 8.25 (d, 2H), 7.42-7.33 (m, 4H), 7.21 (dd, 1H), | | | |
| | 7.16 (d, 1H), 6.83 (s, 1H), 5.84 (tt, 1H), 5.48 (s, 2H), 3.75 (broad, 2H), 3.37 (broad, | | | |
| | 2H), 2.72 (dt, 2H), 2.62 (broad, 2H), 2.48 (broad, 2H), 2.31 (s, 3H). | | | |
| 37 | 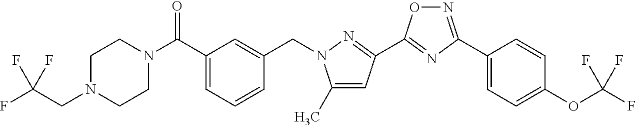 [SMILES: Cc1cc(-c2nc(-c3ccc(OC(F)(F)F)cc3)no2)nn1Cc1cccc(C(=O)N2CCN(CC(F)(F)F)CC2)c1] | 1.33 | 595 | I |

¹H-NMR (400 MHz, CDCl₃, δ/ppm): 8.24 (d, 2H), 7.42-7.33 (m, 4H), 7.21 (dd, 1H),
7.16 (d, 1H), 6.83 (s, 1H), 5.48 (s, 2H), 3.76 (broad, 2H), 3.37 (broad, 2H), 2.96 (quart,
2H), 2.72 (broad, 2H), 2.55 (broad, 2H), 2.31 (s, 3H).

### Example 38

### {3-[(5-Methyl-3-{3-[4-(trifluoromethoxy)phenyl]-1,2,4-oxadiazol-5-yl}-1H-pyrazol-1-yl)methyl]-phenyl}(piperazin-1-yl)methanone

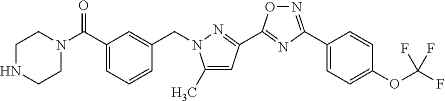 [SMILES: Cc1cc(-c2nc(-c3ccc(OC(F)(F)F)cc3)no2)nn1Cc1cccc(C(=O)N2CCNCC2)c1]

### Step 1: tert-Butyl 4-({3-[(5-methyl-3-{3-[4-(trifluoromethoxy)phenyl]-1,2,4-oxadiazol-5-yl}-1H-pyrazol-1-yl)methyl]phenyl}carbonyl)piperazine-1-carboxylate

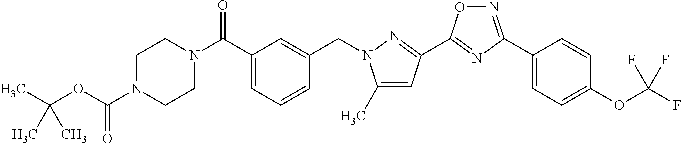 [SMILES: Cc1cc(-c2nc(-c3ccc(OC(F)(F)F)cc3)no2)nn1Cc1cccc(C(=O)N2CCN(C(=O)OC(C)(C)C)CC2)c1]

Analogously to the process described under Example 32, 110 mg (96% of th.) of the title compound were obtained from 80 mg (0.180 mmol) of the compound from Example 93A and 67 mg (0.360 mmol) of tert-butyl piperazine-1-carboxylate. In deviation from the process described under Example 32, the percolation over a sodium bicarbonate cartridge (after the HPLC purification) was omitted here.

¹H-NMR (400 MHz, CDCl₃, δ/ppm): 8.25 (d, 2H), 7.40 (t, 1H), 7.33 (2 d, tog. 3H), 7.22 (d, 1H), 7.20 (s, 1H), 6.83 (s, 1H), 5.48 (s, 2H), 3.70 (broad, 2H), 3.49 (broad, 2H), 3.35 (broad, 4H), 2.31 (s, 3H), 1.13 (s, 9H).

HPLC (method B): Rₜ=5.22 min.

MS (DCI, NH₃): m/z=613 [M+H]⁺.

LC/MS (method C, ESIpos): Rₜ=2.98 min, m/z=613 [M+H]⁺.

### Step 2: {3-[(5-Methyl-3-{3-[4-(trifluoromethoxy)phenyl]-1,2,4-oxadiazol-5-yl}-1H-pyrazol-1-yl)methyl]phenyl}(piperazin-1-yl)methanone

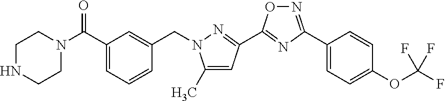 [SMILES: Cc1cc(-c2nc(-c3ccc(OC(F)(F)F)cc3)no2)nn1Cc1cccc(C(=O)N2CCNCC2)c1]

5 ml of a 4 M solution of hydrogen chloride in 1,4-dioxane were added to 70 mg (0.114 mmol) of the compound from Example 38/step 1 at RT and the mixture was stirred at RT for 15 h. Precipitation of the product was then brought to completion by addition of 10 ml of diethyl ether. The solid was filtered off with suction, washed with a little cold diethyl ether and dried under a high vacuum. The solid was then dissolved in a few ml of methanol and the solution was passed over a bicarbonate cartridge (Polymerlabs, Stratospheres SPE, PL-HCO₃ MP SPE, capacity 0.9 mmol) in order to liberate the base. After renewed evaporation of the solvent and drying under a high vacuum, 40 mg (68% of th.) of the title compound were obtained.

¹H-NMR (400 MHz, CDCl₃, δ/ppm): 8.24 (d, 2H), 7.41-7.33 (m, 4H), 7.20 (d, 1H), 7.19 (s, 1H), 6.83 (s, 1H), 5.48 (s, 2H), 3.73 (broad, 2H), 3.33 (broad, 2H), 2.92 (broad, 2H), 2.77 (broad, 2H), 2.30 (s, 3H).

HPLC (method B): Rₜ=4.40 min.

LC/MS (method C, ESIpos): Rₜ=1.68 min, m/z=513 [M+H]⁺.

The compounds in the following table were prepared from the compound from Example 93A and the corresponding mono-tert-butoxycarbonyl-protected diamine components analogously to the two-stage process described under Example 38:

| | | HPLC: | MS: m/z | LC/MS |
|---|---|---|---|---|
| Example | Structure | Rₜ [min] | [M + H]⁺ | method |
| 39 | 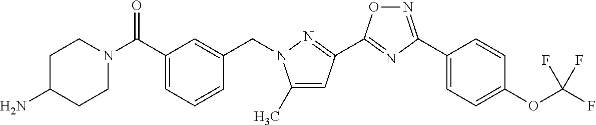 [SMILES: Cc1cc(-c2nc(-c3ccc(OC(F)(F)F)cc3)no2)nn1Cc1cccc(C(=O)N2CCC(N)CC2)c1] | 1.07 | 527 | F |
| | ¹H-NMR (400 MHz, CDCl₃, δ/ppm): 8.24 (d, 2H), 7.40-7.32 (m, 4H), 7.19 (d, 1H), 7.18 | | | |
| | (s, 1H), 6.82 (s, 1H), 5.47 (s, 2H), 4.60-4.50 (m, 1H), 3.68-3.60 (m, 1H), 3.03-2.85 (m, | | | |
| | 3H), 2.30 (s, 3H), 1.91 (broad, 1H), 1.73 (broad, 1H), 1.35 (broad, 1H), 1.20 (broad, | | | |
| | 1H). | | | |
| 40 | 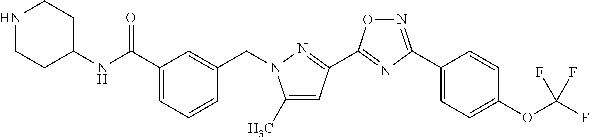 [SMILES: Cc1cc(-c2nc(-c3ccc(OC(F)(F)F)cc3)no2)nn1Cc1cccc(C(=O)NC2CCNCC2)c1] | 1.73 | 527 | C |

¹H-NMR (400 MHz, CDCl₃, δ/ppm): 8.25 (d, 2H), 7.67 (s, 1H), 7.65 (d, 1H), 7.41 (t,
1H), 7.33 (d, 2H), 7.27 (d, 1H), 6.83 (s, 1H), 6.01 (d, broad, 1H), 5.49 (s, 2H), 4.10-
4.01 (m, 1H), 3.22-3.06 (m, 2H), 2.28-2.70 (m, 2H), 2.30 (s, 3H), 2.05-1.99 (m, 2H),
1.46-1.37 (m, 2H).

### Example 41

### 4-Methylpiperazin-1-yl)(3-{[5-methyl-3-(3-{4-[(trifluoromethyl)sulfonyl]phenyl}-1,2,4-oxadiazol-5-yl)-1H-pyrazol-1-yl]methyl}phenyl)methanone

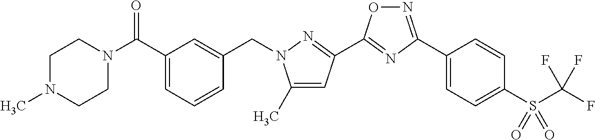 [SMILES: Cc1cc(-c2nc(-c3ccc(S(=O)(=O)C(F)(F)F)cc3)no2)nn1Cc1cccc(C(=O)N2CCN(C)CC2)c1]

76 μl (0.876 mmol) of oxalyl chloride were added to a solution of 100 mg (0.292 mmol) of the compound from Example 105A in 3 ml of anhydrous methylene chloride at 0° C. under inert conditions. The reaction mixture was stirred at RT for 2 h. All the volatile constituents were then removed on a rotary evaporator and the residue obtained in this way was dried under a high vacuum for 20 min. The residue was subsequently dissolved again in 2 ml of anhydrous methylene chloride and this solution was added dropwise to a solution of 94 mg (0.350 mmol) of the compound from Example 3A and 81 μl (0.584 mmol) of triethylamine in 1 ml of methylene chloride at 0° C. After the reaction mixture had been stirred at RT for 16 h, all the volatile constituents were again removed on a rotary evaporator and the residue was dissolved in 4 ml of DMSO. This solution was then heated at 120° C. in a microwave oven for 30 min (CEM Discover, initial irradiation power 250 W). After cooling to RT, the reaction mixture was purified directly by means of preparative HPLC (method N). The product fraction was evaporated to dryness on a rotary evaporator. The residue was dissolved in approx. 5 ml of methanol and the solution was passed over a bicarbonate cartridge (Polymerlabs, Stratospheres SPE, PL-HCO₃ MP SPE, capacity 0.9 mmol). After renewed evaporation of the solvent, 47 mg (28% of th.) of the title compound were obtained in this way.

¹H-NMR (400 MHz, CDCl₃, δ/ppm): 8.52 (d, 2H), 8.18 (d, 2H), 7.40 (t, 1H), 7.34 (d, 1H), 7.21 (d, 1H), 7.18 (s, 1H), 6.85 (s, 1H), 5.49 (s, 2H), 3.76 (broad, 2H), 3.37 (broad, 2H), 2.45 (broad, 2H), 2.31 (s, 3H), 2.29 (broad, 2H), 2.27 (s, 3H).

HPLC (method A): Rₜ=4.28 min.

MS (DCI, NH₃): m/z=575 [M+H]⁺.

Analogously to the process described under Example 41, the compounds in the following table were prepared from the compound from Example 105A or the compound from Example 106A and the corresponding N′-hydroxycarboximide amides (hydroxyamidines).

| | | HPLC: | MS: m/z | LC/MS |
|---|---|---|---|---|
| Example | Structure | Rₜ [min] | [M + H]⁺ | method |
| 42 | 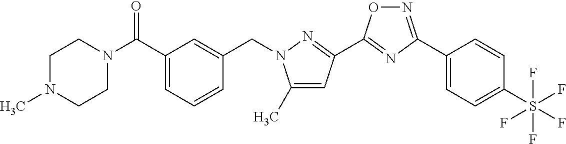 [SMILES: Cc1cc(-c2nc(-c3ccc(S(F)(F)(F)(F)F)cc3)no2)nn1Cc1cccc(C(=O)N2CCN(C)CC2)c1] | 4.39 | 569 | A |
| | ¹H-NMR (400 MHz, CDCl₃, δ/ppm): 8.31 (d, 2H), 7.89 (d, 2H), 7.39 (t, 1H), 7.34 (d, | | | |
| | 1H), 7.20 (d, 1H), 7.17 (s, 1H), 6.85 (s, 1H), 5.48 (s, 2H), 3.76 (broad, 2H), 3.37 (broad, | | | |
| | 2H), 2.45 (broad, 2H), 2.31 (s, 3H), 2.28 (broad, 2H), 2.26 (s, 3H). | | | |
| 43 | 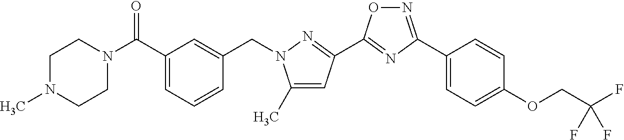 [SMILES: Cc1cc(-c2nc(-c3ccc(OCC(F)(F)F)cc3)no2)nn1Cc1cccc(C(=O)N2CCN(C)CC2)c1] | 0.93 | 541 | I |
| | ¹H-NMR (400 MHz, CDCl₃, δ/ppm): 8.18 (d, 2H), 7.39 (t, 1H), 7.34 (d, 1H), 7.20 (d, | | | |
| | 1H), 7.16 (s, 1H), 7.05 (d, 2H), 6.82 (s, 1H), 5.47 (s, 2H), 4.42 (quart, 2H), 3.76 (broad, | | | |
| | 2H), 3.36 (broad, 2H), 2.45 (broad, 2H), 2.30 (s, 3H), 2.27 (broad, 2H), 2.26 (s, 3H). | | | |
| 44 | 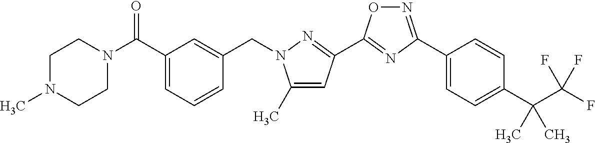 [SMILES: Cc1cc(-c2nc(-c3ccc(C(C)(C)C(F)(F)F)cc3)no2)nn1Cc1cccc(C(=O)N2CCN(C)CC2)c1] | 4.42 | 553 | A |
| | ¹H-NMR (400 MHz, CDCl₃, δ/ppm): 8.19 (d, 2H), 7.62 (d, 2H), 7.39 (t, 1H), 7.34 (d, | | | |
| | 1H), 7.20 (d, 1H), 7.16 (s, 1H), 6.83 (s, 1H), 5.48 (s, 2H), 3.76 (broad, 2H), 3.36 (broad, | | | |
| | 2H), 2.44 (broad, 2H), 2.30 (s, 3H), 2.29 (broad, 2H), 2.25 (s, 3H), 1.63 (s, 6H). | | | |
| 45 | 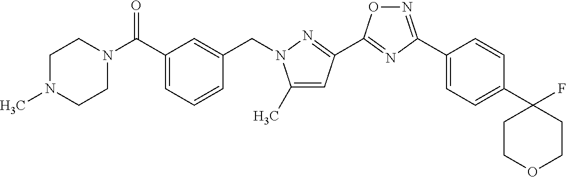 [SMILES: Cc1cc(-c2nc(-c3ccc(C4(F)CCOCC4)cc3)no2)nn1Cc1cccc(C(=O)N2CCN(C)CC2)c1] | 0.98 | 545 | F |
| | ¹H-NMR (400 MHz, CDCl₃, δ/ppm): 8.22 (d, 2H), 7.52 (d, 2H), 7.39 (t, 1H), 7.34 (d, | | | |
| | 1H), 7.20 (d, 1H), 7.16 (s, 1H), 6.84 (s, 1H), 5.48 (s, 2H), 4.00-3.87 (m, 4H), 3.75 | | | |
| | (broad, 2H), 3.36 (broad, 2H), 2.45 (broad, 2H), 2.30 (s, 3H), 2.28 (broad, 2H), 2.27- | | | |
| | 2.12 (m, 2H), 2.26 (s, 3H), 1.98-1.92 (m, 2H). | | | |
| 46 | 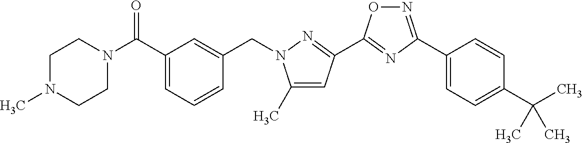 [SMILES: Cc1cc(-c2nc(-c3ccc(C(C)(C)C)cc3)no2)nn1Cc1cccc(C(=O)N2CCN(C)CC2)c1] | 1.01 | 499 | I |
| | ¹H-NMR (400 MHz, CDCl₃, δ/ppm): 8.12 (d, 2H), 7.51 (d, 2H), 7.39 (t, 1H), 7.34 (d, | | | |
| | 1H), 7.20 (d, 1H), 7.15 (s, 1H), 6.83 (s, 1H), 5.48 (s, 2H), 3.76 (broad, 2H), 3.36 (broad, | | | |
| | 2H), 2.44 (broad, 2H), 2.30 (s, 3H), 2.28 (broad, 2H), 2.25 (s, 3H), 1.36 (s, 9H). | | | |
| 47 | 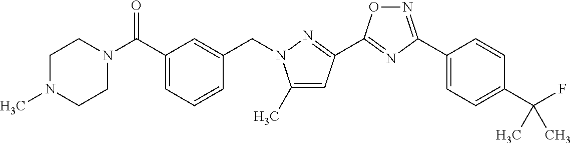 [SMILES: Cc1cc(-c2nc(-c3ccc(C(C)(C)F)cc3)no2)nn1Cc1cccc(C(=O)N2CCN(C)CC2)c1] | 1.03 | 503 | F |
| | ¹H-NMR (400 MHz, CDCl₃, δ/ppm): 8.11 (d, 2H), 7.42 (d, 1H), 7.32-7.26 (m, 2H), 7.12 | | | |
| | (d, 1H), 7.06 (s, 1H), 6.75 (s, 1H), 5.40 (s, 2H), 3.70 (broad, 2H), 3.30 (broad, 2H), | | | |
| | 2.43-2.35 (broad, 2H), 2.30-2.18 (broad, 2H), 2.22 (s, 3H), 2.20 (s, 3H), 1.66 (s, 3H), | | | |
| | 1.62 (s, 3H). | | | |
| 48 | 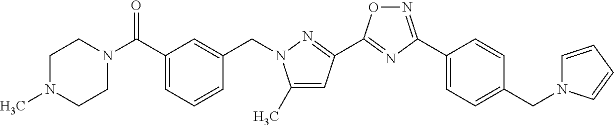 [SMILES: Cc1cc(-c2nc(-c3ccc(Cn4cccc4)cc3)no2)nn1Cc1cccc(C(=O)N2CCN(C)CC2)c1] | 0.90 | 522 | I |
| | ¹H-NMR (400 MHz, CDCl₃, δ/ppm): 8.12 (d, 2H), 7.45-7.08 (m, 6H), 6.80 (s, broad, | | | |
| | 1H), 6.71 (s, broad, 2H), 6.20 (s, broad, 2H), 5.47 (s, broad, 2H), 5.12 (s, broad, 2H), | | | |
| | 3.75 (s, broad, 2H), 3.35 (s, broad, 2H), 2.42 (s, broad, 2H), 2.28 (s, 3H), 2.25 (s, broad, | | | |
| | 2H), 2.25 (s, 3H). | | | |
| 49 | 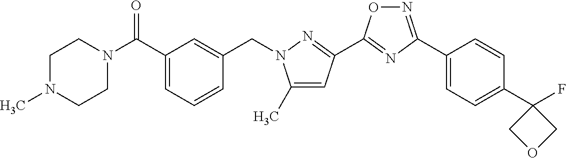 [SMILES: Cc1cc(-c2nc(-c3ccc(C4(F)COC4)cc3)no2)nn1Cc1cccc(C(=O)N2CCN(C)CC2)c1] | 3.96 | 517 | A |
| | ¹H-NMR (400 MHz, CDCl₃, δ/ppm): 8.28 (d, 2H), 7.71 (d, 2H), 7.39 (t, 1H), 7.34 (d, | | | |
| | 1H), 7.20 (d, 1H), 7.16 (s, 1H), 6.84 (s, 1H), 5.48 (s, 2H), 5.15 (dd, 2H), 4.90 (dd, 2H), | | | |
| | 3.76 (broad, 2H), 3.36 (broad, 2H), 2.44 (broad, 2H), 2.30 (s, 3H), 2.29 (broad, 2H), | | | |
| | 2.26 (s, 3H). | | | |
| 50 | 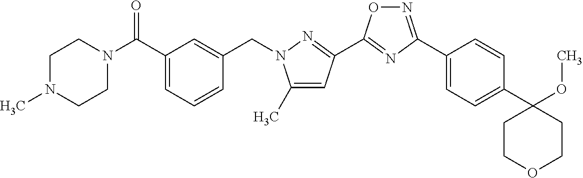 | 0.85 | 557 | I |
| | ¹H-NMR (400 MHz, CDCl₃, δ/ppm): 8.20 (d, 2H), 7.53 (d, 2H), 7.39 (t, 1H), 7.34 (d, | | | |
| | 1H), 7.21 (d, 1H), 7.16 (s, 1H), 6.83 (s, 1H), 5.48 (s, 2H), 3.93-3.82 (m, 4H), 3.76 | | | |
| | (broad, 2H), 3.37 (broad, 2H), 3.01 (s, 3H), 2.45 (broad, 2H), 2.30 (s, 3H), 2.28 (broad, | | | |
| | 2H), 2.26 (s, 3H), 2.11-1.98 (m, 4H). | | | |
| 51 | 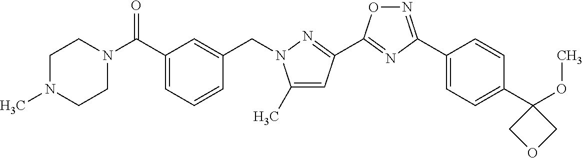 | 0.80 | 529 | I |
| | ¹H-NMR (400 MHz, DMSO-d₆, δ/ppm): 8.14 (d, 2H), 7.68 (d, 2H), 7.46 (t, 1H), 7.33 | | | |
| | (d, 1H), 7.31 (d, 1H), 7.15 (s, 1H), 6.95 (s, 1H), 5.56 (s, 2H), 4.83 (d, 2H), 4.79 (d, 2H), | | | |
| | 3.56 (broad, 2H), 3.25 (broad, 2H), 3.09 (s, 3H), 2.35 (s, 3H), 2.31 (broad, 2H), 2.18 | | | |
| | (broad, 2H), 2.12 (s, 3H). | | | |
| 52 | 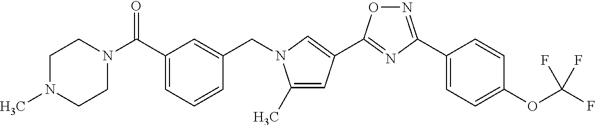 [SMILES: Cc1cc(-c2nc(-c3ccc(OC(F)(F)F)cc3)no2)cn1Cc1cccc(C(=O)N2CCN(C)CC2)c1] | 1.01 | 526 | I |

¹H-NMR (400 MHz, CDCl₃, δ/ppm): 8.16 (d, 2H), 7.49 (d, 1H), 7.40 (t, 1H), 7.35-7.28
(m, 3H), 7.10 (d, 1H), 7.09 (s, 1H), 6.58 (d, 1H), 5.13 (s, 2H), 3.76 (broad, 2H), 3.37
(broad, 2H), 2.45 (broad, 2H), 2.28 (broad, 2H), 2.27 (s, 3H), 2.19 (s, 3H).

### Example 53

### 1-Cyclopropyl-4-{5-[(5-methyl-3-{3-[4-(1,1,1-trifluoro-2-methylpropan-2-yl)phenyl]-1,2,4-oxa-diazol-5-yl}-1H-pyrazol-1-yl)methyl]pyridin-2-yl}piperazine

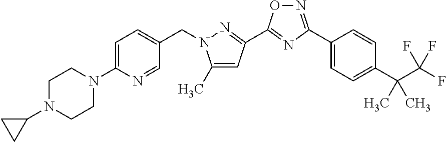 [SMILES: Cc1cc(-c2nc(-c3ccc(C(C)(C)C(F)(F)F)cc3)no2)nn1Cc1ccc(N2CCN(C3CC3)CC2)nc1]

A dispersion of 431 mg (2.17 mmol) of 1-cyclopropylpiperazine dihydrochloride and 729 mg (8.68 mmol) of sodium bicarbonate in approx. 50 ml of methanol was stirred vigorously at RT for 2 h. The undissolved material was then filtered off and the filtrate was evaporated to dryness. Half of the 1-cyclopropylpiperazine obtained in this way was dissolved in 0.5 ml of ethylene glycol dimethyl ether, and the other half was dissolved in 0.5 ml of N,N-dimethylacetamide. 50 mg (0.108 mmol) of the compound from Example 78A was added to each of the two solutions and the mixtures were then heated separately at 150° C. for 36 h. After this time, the conversion was about the same for the two batches (LC/MS control). The reaction mixtures were therefore combined, diluted with approx. 2 ml of acetonitrile and separated directly into their components by means of preparative HPLC (method N). 31 mg (26% of th.) of the title compound were obtained.

¹H-NMR (400 MHz, CDCl₃, δ/ppm): 8.19 (d, 2H), 8.10 (d, 1H), 7.61 (d, 2H), 7.41 (dd, 1H), 6.77 (s, 1H), 6.60 (d, 1H), 5.30 (s, 2H), 3.52-3.49 (m, 5H), 2.71-2.68 (m, 4H), 2.31 (s, 3H), 1.62 (s, 6H), 0.49-0.44 (m, 4H).

LC/MS (method F, ESIpos): Rₜ=1.22 min, m/z=552 [M+H]⁺.

### Example 54

### 1-{5-[(5-Methyl-3-{3-[4-(trimethylsilyl)phenyl]-1,2,4-oxadiazol-5-yl}-1H-pyrazol-1-yl)methyl]-pyridin-2-yl}piperazine

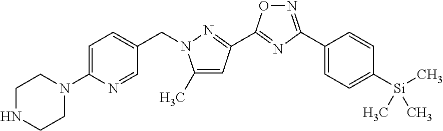 [SMILES: Cc1cc(-c2nc(-c3ccc([Si](C)(C)C)cc3)no2)nn1Cc1ccc(N2CCNCC2)nc1]

A mixture of 200 mg (0.472 mmol) of the compound from Example 87A and 813 mg (9.43 mmol) of piperazine was stirred at a temperature of 150° C. under argon for 16 h. After cooling, approx. 50 ml of water were added and the mixture was extracted three times with approx. 50 ml of ethyl acetate each time. The combined organic extracts were washed successively with water and saturated sodium chloride solution. After drying over anhydrous magnesium sulfate, the mixture was filtered and the filtrate was freed from the solvent on a rotary evaporator. The product was isolated by means of preparative HPLC (method N). 133 mg (59% of th.) of the title compound were obtained.

¹H-NMR (400 MHz, CDCl₃, δ/ppm): 8.17 (d, 2H), 8.11 (d, 1H), 7.63 (d, 2H), 7.41 (dd, 1H), 6.78 (s, 1H), 6.60 (d, 1H), 5.31 (s, 2H), 3.52-3.49 (m, 4H), 2.99-2.95 (m, 4H), 2.32 (s, 3H), 0.32 (s, 9H).

LC/MS (method F, ESIpos): Rₜ=1.23 min, m/z=474 [M+H]⁺.

### Example 55

### 1-Cyclopropyl-4-{5-[(5-methyl-3-{3-[4-(trimethylsilyl)phenyl]-1,2,4-oxadiazol-5-yl}-1H-pyrazol-1-yl)methyl]pyridin-2-yl}piperazine

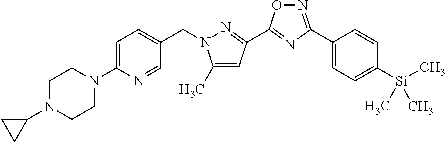 [SMILES: Cc1cc(-c2nc(-c3ccc([Si](C)(C)C)cc3)no2)nn1Cc1ccc(N2CCN(C3CC3)CC2)nc1]

121 μl (2.11 mmol) of glacial acetic acid, 30 mg of dried, powdered molecular sieve (3 Å and 255 μl (1.27 mmol) of 1-ethoxy-1-(trimethylsilyl)oxycyclopropane were added successively to a solution of 100 mg (0.211 mmol) of the compound from Example 54 in 2 ml of methanol. After stirring at RT for 10 min, 40 mg (0.633 mmol) of solid sodium cyanoborohydride were added and the mixture was heated under reflux for 2 h. After cooling to RT, the solid was first filtered off with suction and rinsed with methanol and all the volatile constituents were removed from the filtrate on a rotary evaporator. Approx. 50 ml of half-saturated sodium bicarbonate solution were added to the residue obtained and the mixture was extracted three times with approx. 50 ml of ethyl acetate each time. The combined organic extracts were washed successively with water and saturated sodium chloride solution. After drying over anhydrous sodium sulfate, the mixture was filtered and the filtrate was freed from the solvent on a rotary evaporator. The product was isolated by means of MPLC (silica gel, mobile phase: cyclohexane/ethyl acetate 1:1→1:3). 57 mg (53% of th.) of the title compound were obtained.

¹H-NMR (400 MHz, CDCl₃, δ/ppm): 8.17 (d, 2H), 8.11 (d, 1H), 7.63 (d, 2H), 7.40 (dd, 1H), 6.78 (s, 1H), 6.60 (d, 1H), 5.30 (s, 2H), 3.52-3.49 (m, 4H), 2.71-2.68 (m, 4H), 2.31 (s, 3H), 1.66-1.61 (m, 1H), 0.49-0.43 (m, 4H), 0.30 (s, 9H).

LC/MS (method I, ESIpos): Rₜ=1.08 min, m/z=514 [M+H]⁺.

### Example 56

### 5-[(5-Methyl-3-{3-[4-(trifluoromethoxy)phenyl]-1,2,4-oxadiazol-5-yl}-1H-pyrazol-1-yl)methyl]-2-(pyrrolidin-1-yl)pyridine

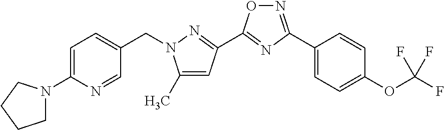 [SMILES: Cc1cc(-c2nc(-c3ccc(OC(F)(F)F)cc3)no2)nn1Cc1ccc(N2CCCC2)nc1]

A mixture of 200 mg (0.459 mmol) of the compound from Example 79A and 2 ml (23.9 mmol) of pyrrolidine was stirred at 160° C. in a microwave oven (CEM Discover, initial irradiation power 250 W) for 3 h. The excess pyrrolidine was then removed on a rotary evaporator. The crude product obtained in this way was purified by means of MPLC (silica gel, mobile phase: cyclohexane/ethyl acetate 100:0→2:1). 53 mg (24% of th.) of the title compound were obtained.

¹H-NMR (400 MHz, CDCl₃, δ/ppm): 8.25 (d, 2H), 8.09 (d, 1H), 7.38 (dd, 1H), 7.33 (d, 2H), 6.76 (s, 1H), 6.31 (d, 1H), 5.30 (s, 2H), 3.45-3.40 (m, 4H), 2.32 (s, 3H), 2.02-1.97 (m, 4H).

LC/MS (method I, ESIpos): Rₜ=1.00 min, m/z=471 [M+H]⁺.

### Example 57

### 1-Cyclopropyl-4-{5-[(5-methyl-3-{3-[4-(trifluoromethoxy)phenyl]-1,2,4-oxadiazol-5-yl}-1H-pyrazol-1-yl)methyl]pyridin-2-yl}piperazine

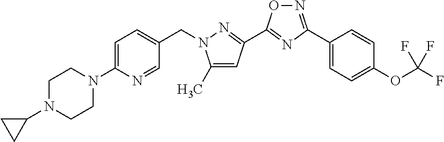 [SMILES: Cc1cc(-c2nc(-c3ccc(OC(F)(F)F)cc3)no2)nn1Cc1ccc(N2CCN(C3CC3)CC2)nc1]

Analogously to the process described under Example 53, the free base was prepared from 457 mg (2.29 mmol) of 1-cyclopropylpiperazine dihydrochloride, and was then heated at 150° C. together with 100 mg (0.229 mmol) of the compound from Example 79A for 4 days. After cooling to RT, the solidified melt was dissolved in approx. 4 ml of acetonitrile and separated into its components by means of preparative HPLC (method N). The product fractions were combined and concentrated to dryness on a rotary evaporator. The product obtained was dissolved in approx. 5 ml of methanol and the solution was passed over a bicarbonate cartridge (Polymerlabs, Stratospheres SPE, PL-HCO₃ MP SPE, capacity 0.9 mmol) in order to remove adhering formic acid from the HPLC purification. 42 mg (35% of th.) of the title compound were obtained in this way.

¹H-NMR (400 MHz, CDCl₃, δ/ppm): 8.25 (d, 2H), 8.10 (d, 1H), 7.41 (dd, 1H), 7.33 (d, 2H), 6.77 (s, 1H), 6.60 (d, 1H), 5.30 (s, 2H), 3.52-3.49 (m, 4H), 2.71-2.68 (m, 4H), 2.31 (s, 3H), 1.66-1.60 (m, 1H), 0.49-0.44 (m, 4H).

HPLC (method A): Rₜ=4.12 min.

LC/MS (method I, ESIpos): Rₜ=1.03 min, m/z=526 [M+H]⁺.

### Example 58

### 1-{5-[(5-Methyl-3-{3-[4-(trifluoromethyl)phenyl]-1,2,4-oxadiazol-5-yl}-1H-pyrazol-1-yl)methyl]-pyridin-2-yl}piperazine

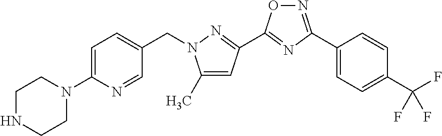 [SMILES: Cc1cc(-c2nc(-c3ccc(C(F)(F)F)cc3)no2)nn1Cc1ccc(N2CCNCC2)nc1]

Analogously to the process described under Example 54, 149 mg (67% of th.) of the title compound were obtained from 200 mg (0.476 mmol) of the compound from Example 88A and 821 mg (9.53 mmol) of piperazine. The product was isolated by means of MPLC (silica gel, mobile phase: methylene chloride/methanol 20:1→5:1).

¹H-NMR (400 MHz, CDCl₃, δ/ppm): 8.33 (d, 2H), 8.11 (d, 1H), 7.76 (d, 2H), 7.41 (dd, 1H), 6.79 (s, 1H), 6.60 (d, 1H), 5.31 (s, 2H), 3.53-3.49 (m, 4H), 2.99-2.96 (m, 4H), 2.32 (s, 3H).

LC/MS (method F, ESIpos): Rₜ=1.09 min, m/z=470 [M+H]⁺.

### Example 59

### 1-Cyclopropyl-4-{5-[(5-methyl-3-{3-[4-(trifluoromethyl)phenyl]-1,2,4-oxadiazol-5-yl}-1H-pyrazol-1-yl)methyl]pyridin-2-yl}piperazine

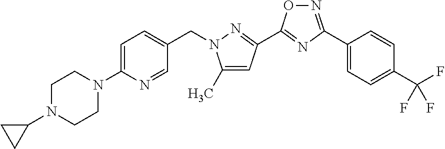 [SMILES: Cc1cc(-c2nc(-c3ccc(C(F)(F)F)cc3)no2)nn1Cc1ccc(N2CCN(C3CC3)CC2)nc1]

Analogously to the process described under Example 55, 43 mg (39% of th.) of the title compound were obtained from 100 mg (0.213 mmol) of the compound from Example 58 and 257 μl (1.28 mmol) of 1-ethoxy-1-(trimethylsilyl)oxycyclopropane.

¹H-NMR (400 MHz, CDCl₃, δ/ppm): 8.33 (d, 2H), 8.11 (d, 1H), 7.76 (d, 2H), 7.41 (dd, 1H), 6.78 (s, 1H), 6.60 (d, 1H), 5.30 (s, 2H), 3.52-3.49 (m, 4H), 2.72-2.68 (m, 4H), 2.33 (s, 3H), 1.67-1.60 (m, 1H), 0.49-0.43 (m, 4H).

LC/MS (method I, ESIpos): Rₜ=0.96 min, m/z=510 [M+H]⁺.

### Example 60

### 5-[(5-Methyl-3-{3-[4-(trifluoromethoxy)phenyl]-1,2,4-oxadiazol-5-yl}-1H-pyrazol-1-yl)methyl]-2-(piperidin-1-yl)pyridine

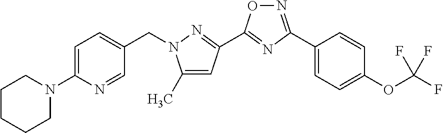 [SMILES: Cc1cc(-c2nc(-c3ccc(OC(F)(F)F)cc3)no2)nn1Cc1ccc(N2CCCCC2)nc1]

Analogously to the process described under Example 56, 154 mg (69% of th.) of the title compound were obtained from 200 mg (0.459 mmol) of the compound from Example 79A and 2.3 ml (22.9 mmol) of piperidine.

¹H-NMR (400 MHz, CDCl₃, δ/ppm): 8.25 (d, 2H), 8.09 (d, 1H), 7.39 (dd, 1H), 7.33 (d, 2H), 6.77 (s, 1H), 6.60 (d, 1H), 5.30 (s, 2H), 3.53-3.50 (m, 4H), 2.32 (s, 3H), 1.64-1.60 (m, 6H).

HPLC (method A): Rₜ=4.39 min.

MS (DCI, NH₃): m/z=485 [M+H]⁺.

LC/MS (method F, ESIpos): Rₜ=1.33 min, m/z=485 [M+H]⁺.

### Example 61

### 2-(Azetidin-1-yl)-5-[(5-methyl-3-{3-[4-(trifluoromethoxy)phenyl]-1,2,4-oxadiazol-5-yl}-1H-pyrazol-1-yl)methyl]pyridine

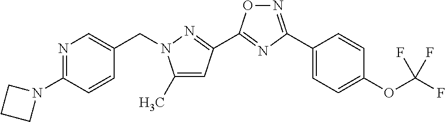 [SMILES: Cc1cc(-c2nc(-c3ccc(OC(F)(F)F)cc3)no2)nn1Cc1ccc(N2CCC2)nc1]

Analogously to the process described under Example 16, 200 mg (0.495 mmol) of the compound from Example 79A and 310 μl (4.59 mmol) of azetidine were reacted to give 81 mg (39% of th.) of the title compound.

¹H-NMR (400 MHz, CDCl₃, δ/ppm): 8.25 (d, 2H), 8.07 (d, 1H), 7.41-7.29 (m, 3H), 6.77 (s, 1H), 6.22 (d, 1H), 5.30 (s, 2H), 4.03 (t, 4H), 2.42-2.37 (m, 2H), 2.31 (s, 3H).

LC/MS (method D, ESIpos): Rₜ=2.00 min, m/z=457 [M+H]⁺.

### Example 62

tert-Butyl 4-{4-[(5-methyl-3-{3-[4-(trifluoromethoxy)phenyl]-1,2,4-oxadiazol-5-yl}-1H-pyrazol-1-yl)methyl]pyridin-2-yl}piperidine-1-carboxylate

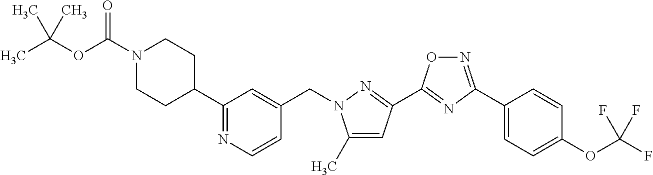 [SMILES: Cc1cc(-c2nc(-c3ccc(OC(F)(F)F)cc3)no2)nn1Cc1ccnc(C2CCN(C(=O)OC(C)(C)C)CC2)c1]

A solution of 301 mg (0.517 mmol) of the compound from Examples 146A in 30 ml of methanol was hydrogenated in a flow-through hydrogenation apparatus [“H-Cube” from Thales Nano, Budapest, Hungary; conditions: Pd cartridge (10% on charcoal), 10 bar of H₂, 25° C., flow rate 1 ml/min]. Since the reaction was not complete in the first pass, the reaction mixture was passed over the cartridge a second time. After evaporation of the solvent on a rotary evaporator, the crude product was purified by means of preparative HPLC (method N). 165 mg (53% of th., purity of 97%) of the title compound were obtained.

¹H-NMR (400 MHz, CDCl₃, δ/ppm): 8.50 (d, 1H), 8.25 (d, 2H), 7.33 (d, 2H), 6.87-6.83 (m, 3H), 5.43 (s, 2H), 4.28-4.17 (m, 2H), 2.85-2.75 (m, 3H), 2.29 (s, 3H), 1.88-1.82 (m, 2H), 1.72-1.62 (m, 2H), 1.45 (s, 9H).

LC/MS (method I, ESIpos): Rₜ=1.34 min, m/z=585 [M+H]⁺.

### Example 63

### 4-[(5-Methyl-3-{3-[4-(trifluoromethoxy)phenyl]-1,2,4-oxadiazol-5-yl}-1H-pyrazol-1-yl)methyl]-2-(piperidin-4-yl)pyridine hydrochloride

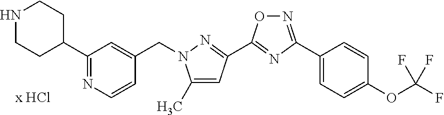 [SMILES: Cc1cc(-c2nc(-c3ccc(OC(F)(F)F)cc3)no2)nn1Cc1ccnc(C2CCNCC2)c1]

641 μl (2.57 mmol) of a 4 M solution of hydrogen chloride in dioxane were added to a solution of 150 mg (0.257 mmol) of the compound from Example 62 in 1 ml of dioxane. After the reaction mixture had been stirred at RT for 2 h, it was concentrated to dryness on a rotary evaporator. The residue obtained was triturated with approx. 5 ml of pentane/dioxane (10:1). After drying under a high vacuum, 143 mg (97% of th., purity of 91%) of the title compound were obtained.

¹H-NMR (400 MHz, DMSO-d₆, δ/ppm): 8.96 (s, broad, 1H), 8.70 (s, broad, 1H), 8.57 (d, 1H), 8.20 (d, 2H), 7.61 (d, 2H), 7.18 (d, 1H), 7.11 (dd, 1H), 7.01 (s, 1H), 5.65 (s, 2H), 3.38-3.32 (m, 2H), 3.14-3.06 (m, 1H), 3.03-2.93 (m, 2H), 2.32 (s, 3H), 2.03-1.86 (m, 4H).

LC/MS (method F, ESIpos): Rₜ=1.06 min, m/z=485 [M+H]⁺.

### Example 64

### 1-{4-[(5-Methyl-3-{3-[4-(trifluoromethoxy)phenyl]-1,2,4-oxadiazol-5-yl}-1H-pyrazol-1-yl)-methyl]pyridin-2-yl}piperazine

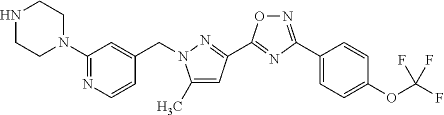 [SMILES: Cc1cc(-c2nc(-c3ccc(OC(F)(F)F)cc3)no2)nn1Cc1ccnc(N2CCNCC2)c1]

91.5 g (0.210 mol) of the compound from Example 81A and 362 g (4.20 mol) of piperazine were heated at 150° C. for 16 h without addition of solvent. After the melt had cooled to RT, 6 l of water and 4 l of ethyl acetate were added and the mixture was stirred intensively. After the organic phase had been separated off, this was washed successively with in each case approx. 2.5 l of water and saturated sodium chloride solution. After drying over anhydrous magnesium sulfate, the mixture was filtered and the filtrate was then freed from the solvent on a rotary evaporator. The residue obtained was chromatographed over approx. 3 kg of silica gel (0.04-0.06 mm) (mobile phase: methylene chloride/methanol 9:1, 12 l→8:2, 12 l→7:3, 16 l→6:1, 8 l). The product fractions were combined and concentrated on a rotary evaporator. 67.1 g (66% of th.) of the title compound were obtained.

¹H-NMR (400 MHz, CDCl₃, δ/ppm): 8.25 (d, 2H), 8.13 (d, 1H), 7.33 (d, 2H), 6.84 (s, 1H), 6.35 (d, 1H), 6.32 (s, 1H), 5.35 (s, 2H), 3.48-3.45 (m, 4H), 2.97-2.94 (m, 4H), 2.30 (s, 3H).

LC/MS (method D, ESIpos): Rₜ=1.89 min, m/z=486 [M+H]⁺.

### Example 65

### 1-Cyclopropyl-4-{4-[(5-methyl-3-{3-[4-(trifluoromethoxy)phenyl]-1,2,4-oxadiazol-5-yl}-1H-pyrazol-1-yl)methyl]pyridin-2-yl}piperazine

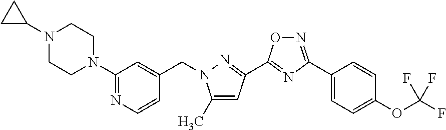 [SMILES: Cc1cc(-c2nc(-c3ccc(OC(F)(F)F)cc3)no2)nn1Cc1ccnc(N2CCN(C3CC3)CC2)c1]

66 ml (1.15 mmol) of glacial acetic acid, 13.9 g of dried, powdered molecular sieve (3 Å) and 139 ml (0.692) of 1-ethoxy-1-(trimethylsilyl)oxycyclopropane were added successively to a solution of 56.0 g (0.115 mol) of the compound from Example 64 in 1.13 l of methanol. After stirring at RT for 10 min, 21.7 g (0.346 mol) of solid sodium cyanoborohydride were added. The mixture was then heated under reflux for 1 h. After cooling to RT, the undissolved material was filtered off with suction and the filtrate was concentrated on a rotary evaporator. The residue obtained was taken up in 1 l of ethyl acetate and the mixture was washed twice with approx. 750 ml of saturated sodium bicarbonate solution each time and then with approx. 750 ml of saturated sodium chloride solution. After drying over anhydrous sodium sulfate, the mixture was filtered and the filtrate was freed from the solvent on a rotary evaporator. The residue (53 g) was recrystallized from a boiling mixture of 293 ml of ethanol and 59 ml of water. When the crystallization was complete (after approx. 20 h at RT), the mixture was filtered with suction. The solid was washed with 36 ml of ethanol/water (5:1) and then dried under a high vacuum. 26.4 g of the title compound were obtained as the first batch in this way. The mother liquor of the crystallization was concentrated on a rotary evaporator. A further 20.3 g of the product were obtained in the form of the formate salt by means of preparative HPLC (method N). For liberation of the base, a suspension of this formate in 1 l of ethyl acetate was washed successively with approx. 200 ml each of saturated sodium bicarbonate solution, water and saturated sodium chloride solution. After drying over anhydrous sodium sulfate, the mixture was filtered and the filtrate was freed from the solvent on a rotary evaporator. The residue (13 g) was recrystallized from a boiling mixture of 80 ml of ethanol and 16 ml of water. When the crystallization was complete (after approx. 4 h at RT), the mixture was filtered with suction and the solid was dried under a high vacuum. A further 11.2 g of the title compound were obtained in this manner (yield in total 37.6 g, 62% of th.).

Melting point: 140° C.

¹H-NMR (400 MHz, CDCl₃, δ/ppm): 8.26 (d, 2H), 8.13 (d, 1H), 7.33 (d, 2H), 6.83 (s, 1H), 6.33 (d, 1H), 6.32 (s, 1H), 5.35 (s, 2H), 3.47 (dd, 4H), 2.69 (dd, 4H), 2.30 (s, 3H), 1.65-1.60 (m, 1H), 0.48-0.42 (m, 4H).

LC/MS (method D, ESIpos): Rₜ=1.91 min, m/z=526 [M+H]⁺.

### Example 66

### 1-Cyclopropyl-4-{4-[(5-methyl-3-{3-[4-(trifluoromethoxy)phenyl]-1,2,4-oxadiazol-5-yl}-1H-pyrazol-1-yl)methyl]pyridin-2-yl}piperazine hydrochloride

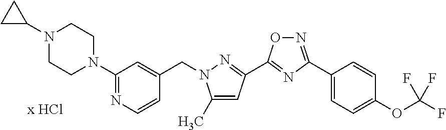 [SMILES: Cc1cc(-c2nc(-c3ccc(OC(F)(F)F)cc3)no2)nn1Cc1ccnc(N2CCN(C3CC3)CC2)c1]

690 μl of 1 M hydrochloric acid were added to a solution of 362 mg (0.690 mmol) of the compound from Example 65 in 50 ml of THF at RT and the mixture was stirred at RT for 1 h. The mixture was then concentrated completely to dryness on a rotary evaporator. The residue obtained was recrystallized from a boiling mixture of 11.5 ml of isopropanol and 5 ml of ethanol. After drying under a high vacuum, 330 mg (85% of th.) of the title compound were obtained, this being present as a solvate with one equivalent of isopropanol in the crystal.

Melting point: 206-210° C.

¹H-NMR (400 MHz, DMSO-d₆, δ/ppm): 10.40 (broad, 1H), 8.20 (d, 2H), 8.12 (d, 1H), 7.60 (d, 2H), 6.97 (s, 1H), 6.83 (d, 1H), 6.40 (s, 1H), 5.47 (s, 2H), 4.41-4.31 (m, 2H), 3.77 (sept, 1H), 3.60-3.53 (broad, 2H), 3.30-3.20 (broad, 4H), 2.90 (broad, 1H), 2.36 (s, 3H), 1.12-1.08 (broad, 2H), 1.03 (d, 6H), 0.84-0.79 (m, 2H).

LC/MS (method I, ESIpos): Rₜ=0.97 min, m/z=526 [M+H]⁺.

### Example 67

### 1-Cyclopropyl-4-{4-[(5-methyl-3-{3-[4-(trifluoromethoxy)phenyl]-1,2,4-oxadiazol-5-yl}-1H-pyrazol-1-yl)methyl]pyridin-2-yl}piperazine methanesulfonate

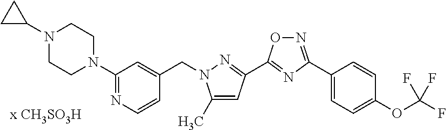 [SMILES: Cc1cc(-c2nc(-c3ccc(OC(F)(F)F)cc3)no2)nn1Cc1ccnc(N2CCN(C3CC3)CC2)c1]

91.5 mg (0.952 mmol) of methanesulfonic acid were added to a solution of 500 mg (0.952 mmol) of the compound from Example 65 in 30 ml of THF at RT and the mixture was stirred at RT for 1 h. It was then concentrated completely to dryness on a rotary evaporator. After the residue had been dried under a high vacuum, 500 mg (85% of th.) of the title compound were obtained.

¹H-NMR (400 MHz, DMSO-d₆, δ/ppm): 9.12 (broad, 1H), 8.20 (d, 2H), 8.13 (d, 1H), 7.60 (d, 2H), 6.98 (s, 1H), 6.83 (s, 1H), 6.41 (d, 1H), 5.47 (s, 2H), 4.44-4.36 (m, 2H), 3.63-3.56 (m, 2H), 3.32-3.23 (m, 2H), 3.14-3.03 (m, 2H), 2.96 (broad, 1H), 2.37 (s, 3H), 2.30 (s, 3H), 1.00-0.97 (broad, 2H), 0.89-0.82 (m, 2H).

### Example 68

### 1-Cyclopropyl-4-{4-[(5-methyl-3-{3-[4-(trifluoromethoxy)phenyl]-1,2,4-oxadiazol-5-yl}-1H-pyrazol-1-yl)methyl]pyridin-2-yl}piperazine 4-sulfamoylbenzoate

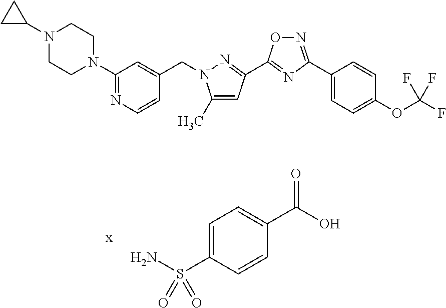 [SMILES: Cc1cc(-c2nc(-c3ccc(OC(F)(F)F)cc3)no2)nn1Cc1ccnc(N2CCN(C3CC3)CC2)c1 ; NS(=O)(=O)c1ccc(C(=O)O)cc1]

206 mg (0.994 mmol) of 4-sulfamoylbenzoic acid were added to a solution of 522 mg (0.994 mmol) of the compound from Example 65 in 20 ml of THF at RT and the mixture was stirred at RT for 1 h. The mixture was then concentrated completely to dryness on a rotary evaporator. After the residue had been dried under a high vacuum, 632 mg (88% of th.) of the title compound were obtained.

¹H-NMR (400 MHz, DMSO-d₆, δ/ppm): 13.37 (broad, 1H), 8.20 (d, 2H), 8.11 (d, 2H), 8.04 (d, 1H), 7.93 (d, 2H), 7.59 (d, 2H), 7.53 (s, 2H), 6.96 (s, 1H), 6.68 (s, 1H), 6.27 (d, 1H), 5.43 (s, 2H), 3.44-3.40 (m, 4H), 2.62-2.58 (m, 4H), 2.33 (s, 3H), 1.67-1.61 (m, 1H), 0.47-0.41 (m, 2H), 0.37-0.33 (m, 2H).

### Example 69

### 1-(2,2-Difluoroethyl)-4-{4-[(5-methyl-3-{3-[4-(trifluoromethoxy)phenyl]-1,2,4-oxadiazol-5-yl}-1H-pyrazol-1-yl)methyl]pyridin-2-yl}piperazine

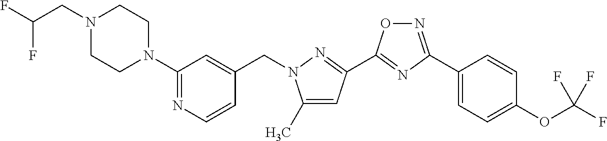 [SMILES: Cc1cc(-c2nc(-c3ccc(OC(F)(F)F)cc3)no2)nn1Cc1ccnc(N2CCN(CC(F)F)CC2)c1]

65 mg (0.149 mmol) of the compound from Example 81A and 166 mg (0.746 mmol) of the compound from Example 104A were stirred together with 260 μl (1.49 mmol) of N,N-diisopropylethylamine at 160° C. in a microwave oven (CEM Discover, initial irradiation power 250 W) for 3 h. After cooling to RT, the mixture was diluted with approx. 3 ml of methanol and the reaction mixture was separated directly into its components by means of preparative HPLC (method N). The product fractions were combined and freed from the solvent on a rotary evaporator. The residue was dissolved again in approx. 5 ml of methanol and the solution was passed over a bicarbonate cartridge (Polymerlabs, Stratospheres SPE, PL-HCO₃ MP SPE, capacity 0.9 mmol) in order to remove adhering formic acid from the HPLC purification. 65 mg (78% of th.) of the title compound were obtained.

¹H-NMR (400 MHz, CDCl₃, δ/ppm): 8.25 (d, 2H), 8.12 (d, 1H), 7.33 (d, 2H), 6.83 (s, 1H), 6.37 (d, 1H), 6.31 (s, 1H), 5.90 (ft, 1H), 5.34 (s, 2H), 3.50 (dd, 4H), 2.77 (dt, 2H), 2.66 (dd, 4H), 2.29 (s, 3H).

LC/MS (method I, ESIpos): Rₜ=1.19 min, m/z=550 [M+H]⁺.

### Example 70

### 1-{4-[(5-Methyl-3-{3-[4-(tetrahydro-2H-pyran-4-yl)phenyl]-1,2,4-oxadiazol-5-yl}-1H-pyrazol-1-yl)methyl]pyridin-2-yl}piperazine formate

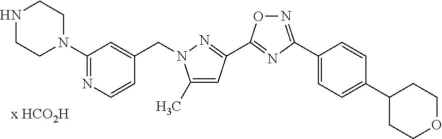 [SMILES: Cc1cc(-c2nc(-c3ccc(C4CCOCC4)cc3)no2)nn1Cc1ccnc(N2CCNCC2)c1]

A mixture of 220 mg (0.505 mmol) of the compound from Example 140A and 869 mg (10.1 mmol) of piperazine was stirred at 160° C. for 16 h without addition of solvent. After the melt had cooled to RT, approx. 50 ml of water were added and the mixture was extracted three times with approx.

20 ml of ethyl acetate each time. The combined organic extracts were washed with saturated sodium chloride solution and dried over anhydrous magnesium sulfate. After filtration, the solvent was removed on a rotary evaporator and the residue was purified by means of preparative HPLC (method N). 178 mg (66% of th.) of the title compound were obtained.

¹H-NMR (400 MHz, CDCl₃, δ/ppm): 8.33 (s, 1H), 8.15 (2d, 2H+1H), 7.35 (d, 2H), 6.83 (s, 1H), 6.43 (d, 1H), 6.34 (s, 1H), 5.37 (s, 2H), 4.13-4.08 (m, 2H), 3.70-3.67 (m, 4H), 3.58-3.52 (m, 2H), 3.15-3.11 (m, 4H), 2.88-2.80 (m, 1H), 2.29 (s, 3H), 1.92-1.78 (m, 4H).

LC/MS (method I, ESIpos): Rₜ=0.83 min, m/z=486 [M+H]⁺.

### Example 71

### 1-Cyclopropyl-4-{4-[(5-methyl-3-{3-[4-(tetrahydro-2H-pyran-4-yl)phenyl]-1,2,4-oxadiazol-5-yl}-1H-pyrazol-1-yl)methyl]pyridin-2-yl}piperazine

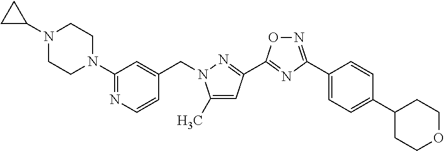 [SMILES: Cc1cc(-c2nc(-c3ccc(C4CCOCC4)cc3)no2)nn1Cc1ccnc(N2CCN(C3CC3)CC2)c1]

206 μl (3.60 mmol) of glacial acetic acid, 51 mg of dried, powdered molecular sieve (3 Å) and 435 μl (2.17 mmol) of 1-ethoxy-1-(trimethylsilyl)oxycyclopropane were added successively to a solution of 175 mg (0.360 mmol) of the compound from Example 70 in 5 ml of methanol. After stirring at RT for 10 min, 68 mg (1.08 mmol) of solid sodium cyanoborohydride were added. The mixture was then heated under reflux for 2 h. After cooling to RT, the undissolved material was filtered off with suction and the filtrate was concentrated on a rotary evaporator. The product was isolated from the residue by means of MPLC (silica gel, mobile phase: methylene chloride/methanol 30:1). By trituration with pentane, the viscous oil obtained was converted into a solid, which was stirred with acetonitrile/methanol (10:1). After drying under a high vacuum, 30 mg (15% of th., purity of 95%) of the title compound were obtained.

¹H-NMR (400 MHz, CDCl₃, δ/ppm): 8.15 (d, 2H), 8.12 (d, 1H), 7.35 (d, 2H), 6.83 (s, 1H), 6.33 (d, 1H), 6.32 (s, 1H), 5.35 (s, 2H), 4.13-4.08 (m, 2H), 3.59-3.52 (m, 2H), 3.49-3.44 (m, 4H), 2.88-2.79 (m, 1H), 2.71-2.66 (m, 4H), 2.28 (s, 3H), 1.92-1.78 (m, 4H), 1.67-1.58 (m, 1H), 0.49-0.42 (m, 4H).

LC/MS (method F, ESIpos): Rₜ=0.83 min, m/z=526 [M+H]⁺.

### Example 72

### 1-{4-[(5-Methyl-3-{3-[4-(trifluoromethoxy)phenyl]-1,2,4-oxadiazol-5-yl}-1H-pyrazol-1-yl)-methyl]pyridin-2-yl}-4-(2,2,2-trifluoroethyl)piperazine

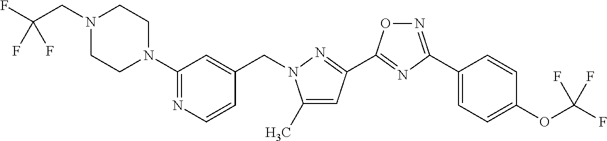 [SMILES: Cc1cc(-c2nc(-c3ccc(OC(F)(F)F)cc3)no2)nn1Cc1ccnc(N2CCN(CC(F)(F)F)CC2)c1]

100 mg (0.229 mmol) of the compound from Example 81A and 277 mg (1.15 mmol) of the compound from Example 138A were stirred together with 400 μl (2.30 mmol) of N,N-diisopropylethylamine at 160° C. in a microwave oven (CEM Discover, initial irradiation power 250 W) for 3 h. After cooling to RT, the mixture was diluted with approx. 3 ml of methanol and the reaction mixture was separated directly into its components by means of preparative HPLC (method N). The product fractions were combined and freed from the solvent on a rotary evaporator. The residue was dissolved again in approx. 5 ml of methanol and the solution was passed over a bicarbonate cartridge (Polymerlabs, Stratospheres SPE, PL-HCO₃ MP SPE, capacity 0.9 mmol) in order to remove adhering formic acid from the HPLC purification. 64 mg (49% of th.) of the title compound were obtained.

¹H-NMR (400 MHz, CDCl₃, δ/ppm): 8.24 (d, 2H), 8.12 (d, 1H), 7.33 (d, 2H), 6.83 (s, 1H), 6.37 (d, 1H), 6.31 (s, 1H), 5.35 (s, 2H), 3.51 (dd, 4H), 3.00 (quart, 2H), 2.73 (dd, 4H), 2.29 (s, 3H).

LC/MS (method I, ESIpos): Rₜ=1.31 min, m/z=568 [M+H]⁺.

### Example 73

### 1-Cyclobutyl-4-{4-[(5-methyl-3-{3-[4-(trifluoromethoxy)phenyl]-1,2,4-oxadiazol-5-yl}-1H-pyrazol-1-yl)methyl]pyridin-2-yl}piperazine

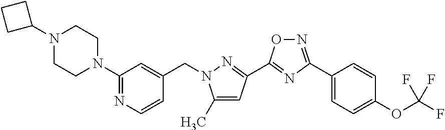 [SMILES: Cc1cc(-c2nc(-c3ccc(OC(F)(F)F)cc3)no2)nn1Cc1ccnc(N2CCN(C3CCC3)CC2)c1]

200 mg (0.412 mmol) of the compound from Example 64 and 37 μl (0.494 mmol) of cyclobutanone were dissolved in 5 ml of anhydrous ethanol and the solution was stirred at RT for 1 h. 47 mg (1.24 mmol) of solid sodium borohydride were then added dropwise. After the reaction mixture had been stirred at RT for 16 h, 25 ml of water were added and the mixture was extracted twice with approx. 20 ml of methylene chloride each time. The combined organic extracts were dried over anhydrous magnesium sulfate. After filtration, the solvent was removed on a rotary evaporator and the crude product was purified by means of preparative HPLC (method N). The product obtained was dissolved again in approx. 5 ml of methanol and the solution was passed over a bicarbonate cartridge (Polymerlabs, Stratospheres SPE, PL-HCO₃ MP SPE, capacity 0.9 mmol) in order to remove adhering formic acid from the HPLC purification. 74 mg (31% of th., purity of approx. 95%) of the title compound were obtained.

¹H-NMR (400 MHz, CDCl₃, δ/ppm): 8.25 (d, 2H), 8.12 (d, 1H), 7.33 (d, 2H), 6.83 (s, 1H), 6.33 (d, 1H), 6.31 (s, 1H), 5.35 (s, 2H), 3.50 (dd, 4H), 2.73 (quint, 1H), 2.39 (dd, 4H), 2.29 (s, 3H), 2.07-2.00 (m, 2H), 1.94-1.85 (m, 2H), 1.77-1.65 (m, 2H).

LC/MS (method I, ESIpos): Rₜ=0.93 min, m/z=540 [M+H]⁺.

### Example 74

### 1-[4-({3-[3-(4-tert-Butylphenyl)-1,2,4-oxadiazol-5-yl]-5-methyl-1H-pyrazol-1-yl}methyl)pyridin-2-yl]piperazine

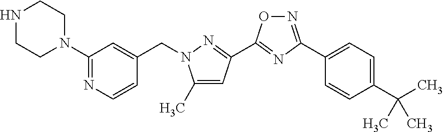 [SMILES: Cc1cc(-c2nc(-c3ccc(C(C)(C)C)cc3)no2)nn1Cc1ccnc(N2CCNCC2)c1]

A solution of 400 mg (0.981 mmol) of the compound from Example 141A and 1.69 g (10.6 mmol) of piperazine in 12 ml of ethanol was automatically controlled at 140° C. in a microwave apparatus (Biotage Initiator 2.5) and then heated manually to 190° C. in the course of 3 min. After 1 h at 190° C., the reaction mixture was allowed to cool to RT. 100 ml of water were added and the mixture was extracted three times with approx. 50 ml of ethyl acetate each time. The combined organic extracts were washed with saturated sodium chloride solution and dried over anhydrous magnesium sulfate. After filtration and evaporation, a crude product was obtained, which was purified by MPLC (approx. 30 g of silica gel, mobile phase: methylene chloride/methanol 10:1). 339 mg (76% of th.) of the title compound were obtained.

¹H-NMR (400 MHz, CDCl₃, δ/ppm): 8.12 (2d, 2H+1H), 7.51 (d, 2H), 6.83 (s, 1H), 6.37 (d, 1H), 6.32 (s, 1H), 5.36 (s, 2H), 3.50-3.47 (m, 4H), 2.98-2.95 (m, 4H), 2.29 (s, 3H), 1.37 (s, 9H).

LC/MS (method I, ESIpos): Rₜ=0.96 min, m/z=458 [M+H]⁺.

### Example 75

### 1-[4-({3-[3-(4-tert-Butylphenyl)-1,2,4-oxadiazol-5-yl]-5-methyl-1H-pyrazol-1-yl}methyl)pyridin-2-yl]-4-cyclopropylpiperazine

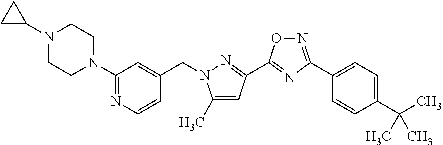 [SMILES: Cc1cc(-c2nc(-c3ccc(C(C)(C)C)cc3)no2)nn1Cc1ccnc(N2CCN(C3CC3)CC2)c1]

Analogously to the process described under Example 55, 135 mg (0.295 mmol) of the compound from Example 74 were reacted to give 60 mg (40% of th., purity of 98%) of the title compound.

¹H-NMR (400 MHz, CDCl₃, δ/ppm): 8.13 (d, 2H), 8.12 (d, 1H), 7.51 (d, 2H), 6.83 (s, 1H), 6.34 (d, 1H), 6.32 (s, 1H), 5.34 (s, 2H), 3.48-3.45 (m, 4H), 2.70-2.67 (m, 4H), 2.29 (s, 3H), 1.65-1.60 (m, 1H), 1.37 (s, 9H), 0.48-0.43 (m, 4H).

LC/MS (method D, ESIpos): Rₜ=2.04 min, m/z=498 [M+H]⁺.

### Example 76

### 1-(4-{[3-(3-{4-[1-(Methoxymethyl)cyclobutyl]phenyl}-1,2,4-oxadiazol-5-yl)-5-methyl-1H-pyrazol-1-yl]methyl}pyridin-2-yl)piperazine formate

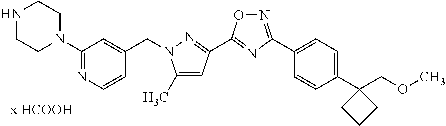 [SMILES: COCC1(c2ccc(-c3noc(-c4cc(C)n(Cc5ccnc(N6CCNCC6)c5)n4)n3)cc2)CCC1]

Analogously to the process described under Example 74, 766 mg (8.89 mmol) of piperazine and 200 mg (0.444 mmol) of the compound from Example 142A were reacted to give 198 mg (82% of th.) of the title compound. The crude product was purified by means of preparative HPLC (method N).

¹H-NMR (400 MHz, CDCl₃, δ/ppm): 8.39 (s, 1H), 8.13 (d, 1H), 8.12 (d, 2H), 7.30 (d, 2H), 6.83 (s, 1H), 6.44 (d, 1H), 6.33 (s, 1H), 5.37 (s, 2H), 3.71-3.69 (m, 4H), 3.55 (s, 2H), 3.29 (s, 3H), 3.15-3.12 (m, 4H), 2.42-2.29 (m, 4H), 2.29 (s, 3H), 2.16-2.03 (m, 1H), 1.93-1.83 (m, 1H).

LC/MS (method D, ESIpos): Rₜ=1.86 min, m/z=500 [M+H]⁺.

### Example 77

### 1-Cyclopropyl-4-(4-{[3-(3-{4-[1-(methoxymethyl)cyclobutyl]phenyl}-1,2,4-oxadiazol-5-yl)-5-methyl-1H-pyrazol-1-yl]methyl}pyridin-2-yl)piperazine

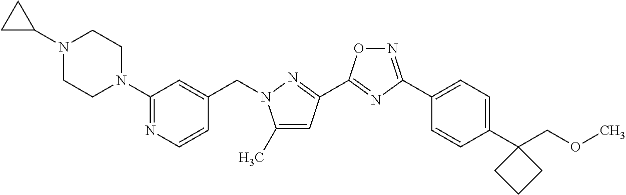 [SMILES: COCC1(c2ccc(-c3noc(-c4cc(C)n(Cc5ccnc(N6CCN(C7CC7)CC6)c5)n4)n3)cc2)CCC1]

Analogously to the process described under Example 55, 100 mg (0.183 mmol) of the compound from Example 76 were reacted to give 65 mg (65% of th., purity of 98%) of the title compound.

¹H-NMR (400 MHz, CDCl₃, δ/ppm): 8.13 (d, 2H), 8.12 (d, 1H), 7.30 (d, 2H), 6.83 (s, 1H), 6.34 (d, 1H), 6.32 (s, 1H), 5.35 (s, 2H), 3.56 (s, 2H), 3.48-3.45 (m, 4H), 3.29 (s, 3H), 2.70-2.67 (m, 4H), 2.44-2.29 (m, 4H), 2.29 (s, 3H), 2.16-2.03 (m, 1H), 1.93-1.83 (m, 1H), 1.65-1.60 (m, 1H), 0.49-0.43 (m, 4H).

LC/MS (method F, ESIpos): Rₜ=1.15 min, m/z=540 [M+H]⁺.

### Example 78

### 1-[4-({3-[3-(4-tert-Butylphenyl)-1,2,4-oxadiazol-5-yl]-5-methyl-1H-pyrazol-1-yl}methyl)pyridin-2-yl]-4-(2,2,2-trifluoroethyl)piperazine

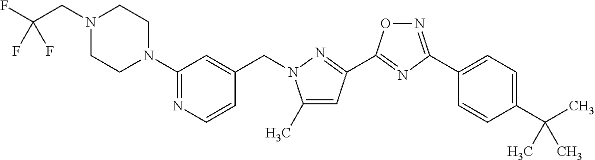 [SMILES: Cc1cc(-c2nc(-c3ccc(C(C)(C)C)cc3)no2)nn1Cc1ccnc(N2CCN(CC(F)(F)F)CC2)c1]

76 μl (0.546 mmol) of triethylamine and 74 μl (0.437 mmol) of trifluoromethanesulfonic acid anhydride were first added to a solution of 32 μl (0.437 mmol) of 2,2,2-trifluoroethanol in 2 ml of anhydrous methylene chloride at 0° C. After stirring at 0° C. for 2 h, a solution of 100 mg (0.219 mmol) of the compound from Example 74 in 1 ml of methylene chloride was added. Stirring was continued at RT. After 15 h, approx. 20 ml of water were added and the mixture was extracted with methylene chloride. The organic extract was washed with water and dried over anhydrous magnesium sulfate. After filtration, the solvent was removed on a rotary evaporator and the product was isolated by means of preparative HPLC (method N). 34 mg (28% of th., purity of approx. 98%) of the title compound were obtained.

¹H-NMR (400 MHz, CDCl₃, δ/ppm): 8.13 (2d, 2H+1H), 7.52 (d, 2H), 6.83 (s, 1H), 6.38 (d, 1H), 6.31 (s, 1H), 5.35 (s, 2H), 3.53-3.50 (m, 4H), 3.00 (quart, 2H), 2.76-2.73 (m, 4H), 2.28 (s, 3H), 1.37 (s, 9H).

LC/MS (method Q, ESIpos): Rₜ=2.70 min, m/z=540 [M+H]⁺.

### Example 79

### 1-{4-[(5-Methyl-3-{3-[3-methyl-4-(tetrahydro-2H-pyran-4-yl)phenyl]-1,2,4-oxadiazol-5-yl}-1H-pyrazol-1-yl)methyl]pyridin-2-yl}piperazine

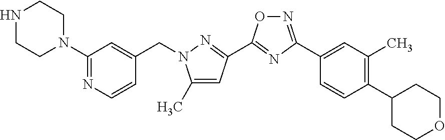 [SMILES: Cc1cc(-c2noc(-c3cc(C)n(Cc4ccnc(N5CCNCC5)c4)n3)n2)ccc1C1CCOCC1]

Analogously to the process described under Example 54, 160 mg (95% of th., purity of 95%) of the title compound were obtained from 145 mg (0.322 mmol) of the compound from Example 143A and 555 mg (6.45 mmol) of piperazine. In this case the crude product was purified not by means of preparative HPLC but by trituration with pentane.

¹H-NMR (400 MHz, CDCl₃, δ/ppm): 8.12 (d, 1H), 8.02 (s, 1H), 8.01 (d, 1H), 7.34 (d, 1H), 6.83 (s, 1H), 6.36 (d, 1H), 6.32 (s, 1H), 5.35 (s, 2H), 4.13-4.10 (m, 2H), 3.67-3.54 (m, 4H), 3.64 (dd, 4H), 3.07-3.00 (m, 1H), 2.94 (dd, 4H), 2.42 (s, 3H), 2.28 (s, 3H), 1.91-1.81 (m, 2H).

LC/MS (method D, ESIpos): Rₜ=1.72 min, m/z=500 [M+H]⁺.

### Example 80

### 1-{4-[(3-{3-[3-Chloro-4-(trifluoromethoxy)phenyl]-1,2,4-oxadiazol-5-yl}-5-methyl-1H-pyrazol-1-yl)methyl]pyridin-2-yl}piperazine

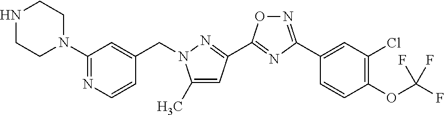 [SMILES: Cc1cc(-c2nc(-c3ccc(OC(F)(F)F)c(Cl)c3)no2)nn1Cc1ccnc(N2CCNCC2)c1]

Analogously to the process described under Example 16, 340 mg (0.723 mmol) of the compound from Example 144A and 1.24 g (14.5 mmol) of piperazine were reacted to give 114 mg (30% of th.) of the title compound.

¹H-NMR (400 MHz, DMSO-d₆, δ/ppm): 8.04 (d, 1H), 8.01-7.93 (m, 2H), 7.30 (d, 1H), 6.93 (s, 1H), 6.61 (s, 1H), 6.26 (d, 1H), 5.42 (s, 2H), 4.10 (s, broad, 1H), 3.35-3.25 (t, 4H), 2.80-2.70 (t, 4H), 2.32 (s, 3H).

LC/MS (method D, ESIpos): Rₜ=1.27 min, m/z=520/522 [M+H]⁺.

### Example 81

### 1-Cyclopropyl-4-{4-[(5-methyl-3-{3-[3-methyl-4-(tetrahydro-2H-pyran-4-yl)phenyl]-1,2,4-oxa-diazol-5-yl}-1H-pyrazol-1-yl)methyl]pyridin-2-yl}piperazine

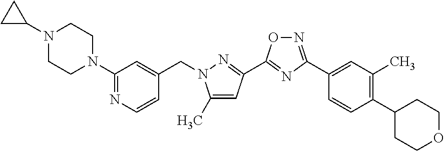 [SMILES: Cc1cc(-c2noc(-c3cc(C)n(Cc4ccnc(N5CCN(C6CC6)CC5)c4)n3)n2)ccc1C1CCOCC1]

Analogously to the process described under Example 71, 23 mg (13% of th., purity of 93%) of the title compound were obtained from 150 mg (0.30 mmol) of the compound from Example 79 and 362 μl (1.80 mmol) of 1-ethoxy-1-(trimethylsilyl)oxycyclopropane. The purification of the crude product was carried out by means of preparative HPLC (method N).

¹H-NMR (400 MHz, CDCl₃, δ/ppm): 8.12 (d, 1H), 8.02 (s, 1H), 8.01 (d, 1H), 7.34 (d, 1H), 6.83 (s, 1H), 6.33 (d, 1H), 6.32 (s, 1H), 5.34 (s, 2H), 4.15-4.09 (m, 2H), 3.60-3.53 (m, 2H), 3.50-3.44 (m, 4H), 3.08-2.99 (m, 1H), 2.70-2.67 (m, 4H), 2.42 (s, 3H), 2.28 (s, 3H), 1.92-1.80 (m, 2H), 1.75-1.69 (m, 2H), 1.65-1.59 (m, 1H), 0.50-0.42 (m, 4H).

LC/MS (method I, ESIpos): Rₜ=0.93 min, m/z=540 [M+H]⁺.

### Example 82

### N-{4-[5-(1-{[2-(4-Cyclopropylpiperazin-1-yl)pyridin-4-yl]methyl}-5-methyl-1H-pyrazol-3-yl)-1,2,4-oxadiazol-3-yl]benzyl}-N-isopropylpropan-2-amine

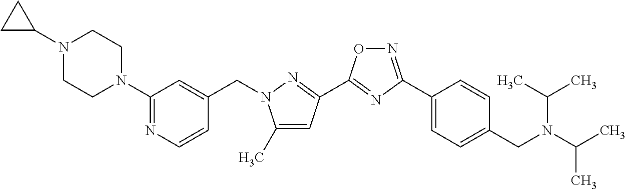 [SMILES: Cc1cc(-c2nc(-c3ccc(CN(C(C)C)C(C)C)cc3)no2)nn1Cc1ccnc(N2CCN(C3CC3)CC2)c1]

365 mg (1.00 mmol, purity of 93%) of the compound from Example 132A were initially introduced with 277 mg (1.10 mmol) of the compound from Example 135A into 10 ml of THF. The mixture was cooled to 0° C., 146 mg (1.30 mmol) of potassium tert-butylate were added and the mixture was stirred first at RT for 1 h and then under reflux for 24 h. After cooling to RT, the mixture was diluted with ethyl acetate and washed once with water and the aqueous phase was extracted once with ethyl acetate. The combined organic phases were washed once with saturated aqueous sodium chloride solution, dried over magnesium sulfate, filtered and concentrated. The residue was purified by means of preparative HPLC (method O). The combined product fractions were concentrated to a residual volume of water, saturated aqueous sodium bicarbonate solution was added and the mixture was extracted twice with ethyl acetate. The combined ethyl acetate phases were dried over magnesium sulfate, filtered and concentrated. The residue was finally dried in vacuo. 259 mg (47% of th.) of the title compound were obtained.

¹H-NMR (400 MHz, CDCl₃, δ/ppm): 8.13-8.10 (m, 3H), 7.50 (d, 2H), 6.82 (s, 1H), 6.36-6.30 (m, 2H), 5.35 (s, 2H), 3.70 (s, 2H), 3.45 (s, broad, 4H), 3.10-3.00 (m, 2H), 2.69 (s, broad, 4H), 3.30 (s, 3H), 1.67-1.60 (m, 1H), 1.05 (d, 12H), 0.50-0.45 (m, 4H).

LC/MS (method D, ESIpos): Rₜ=1.19 min, m/z=555 [M+H]⁺.

### Example 83

### 4-{4-[(5-Methyl-3-{3-[4-(trifluoromethoxy)phenyl]-1,2,4-oxadiazol-5-yl}-1H-pyrazol-1-yl)-methyl]pyridin-2-yl}morpholine

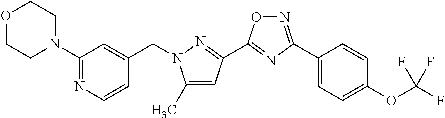 [SMILES: Cc1cc(-c2nc(-c3ccc(OC(F)(F)F)cc3)no2)nn1Cc1ccnc(N2CCOCC2)c1]

Analogously to the process described under Example 56, 156 mg (69% of th., purity of 98%) of the title compound were obtained from 200 mg (0.459 mmol) of the compound from Example 81A and 2.1 ml (23.9 mmol) of morpholine. The crude product was purified by means of preparative HPLC (method N).

¹H-NMR (400 MHz, CDCl₃, δ/ppm): 8.26 (d, 2H), 8.14 (d, 1H), 7.33 (d, 2H), 6.83 (s, 1H), 6.40 (d, 1H), 6.31 (s, 1H), 5.37 (s, 2H), 3.78 (dd, 4H), 3.46 (dd, 4H), 2.30 (s, 3H).

LC/MS (method D, ESIpos): Rₜ=2.25 min, m/z=587 [M+H]⁺.

### Example 84

### 1-{4-[(5-Methyl-3-{3-[4-(trifluoromethoxy)phenyl]-1,2,4-oxadiazol-5-yl}-1H-pyrazol-1-yl)-methyl]pyridin-2-yl}piperidin-4-ol

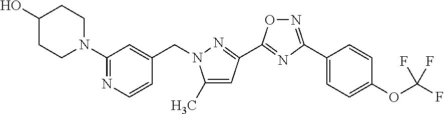 [SMILES: Cc1cc(-c2nc(-c3ccc(OC(F)(F)F)cc3)no2)nn1Cc1ccnc(N2CCC(O)CC2)c1]

Analogously to the process described under Example 56, 33 mg (14% of th.) of the title compound were obtained from 200 mg (0.459 mmol) of the compound from Example 81A and 464 mg (4.59 mmol) of 4-hydroxypiperidine. The crude product was purified by means of preparative HPLC (method N).

¹H-NMR (400 MHz, CDCl₃, δ/ppm): 8.25 (d, 2H), 8.12 (d, 1H), 7.33 (d, 2H), 6.83 (s, 1H), 6.36 (s, 1H), 6.32 (d, 1H), 5.34 (s, 2H), 4.02-3.96 (m, 2H), 3.94-3.88 (m, 1H), 3.17-3.10 (m, 2H), 2.29 (s, 3H), 1.98-1.91 (m, 2H), 1.59-1.51 (m, 2H).

LC/MS (method D, ESIpos): Rₜ=2.25 min, m/z=501 [M+H]⁺.

### Example 85

### 1-{4-[(5-Methyl-3-{3-[4-(trifluoromethoxy)phenyl]-1,2,4-oxadiazol-5-yl}-1H-pyrazol-1-yl)-methyl]pyridin-2-yl}piperidine-4-carbonitrile

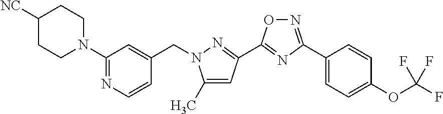 [SMILES: Cc1cc(-c2nc(-c3ccc(OC(F)(F)F)cc3)no2)nn1Cc1ccnc(N2CCC(C#N)CC2)c1]

A mixture of 120 mg (0.275 mmol) of the compound from Example 81A and 606 mg (5.51 mmol) of 4-cyanopiperidine was stirred at 160° C. in a microwave oven (CEM Discover, initial irradiation power 250 W) for 3 h. Approx. 4 ml of methanol were then added and the reaction mixture was purified directly by means of preparative HPLC (method N). The product fractions were combined and freed from the solvent on a rotary evaporator. The residue was triturated with approx. 5 ml of cyclohexane/ethyl acetate (20:1). After drying under a high vacuum, 103 mg (73% of th.) of the title compound were obtained.

¹H-NMR (400 MHz, CDCl₃, δ/ppm): 8.25 (d, 2H), 8.13 (d, 1H), 7.33 (d, 2H), 6.84 (s, 1H), 6.38 (d, 1H), 6.34 (s, 1H), 5.35 (s, 2H), 3.80-3.73 (m, 2H), 3.48-3.41 (m, 2H), 2.88-2.82 (m, 1H), 2.29 (s, 3H), 2.00-1.93 (m, 2H), 1.92-1.83 (m, 2H).

LC/MS (method F, ESIpos): Rₜ=1.28 min, m/z=510 [M+H]⁺.

### Example 86

### 1-Methyl-4-{4-[(5-methyl-3-{3-[4-(trifluoromethoxy)phenyl]-1,2,4-oxadiazol-5-yl}-1H-pyrazol-1-yl)methyl]pyridin-2-yl}piperazin-2-one

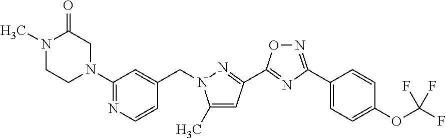 [SMILES: Cc1cc(-c2nc(-c3ccc(OC(F)(F)F)cc3)no2)nn1Cc1ccnc(N2CCN(C)C(=O)C2)c1]

Analogously to the process described under Example 56, 154 mg (65% of th.) of the title compound were obtained from 200 mg (0.459 mmol) of the compound from Example 81A and 524 mg (4.59 mmol) of 1-methylpiperazin-2-one [H. R. Buerki et al., Eur. J. Med. Chem. 1978 (13), 479-485]. The crude product was purified by means of preparative HPLC (method N).

¹H-NMR (400 MHz, CDCl₃, δ/ppm): 8.25 (d, 2H), 8.13 (d, 1H), 7.33 (d, 2H), 6.84 (s, 1H), 6.42 (d, 1H), 6.23 (s, 1H), 5.37 (s, 2H), 4.02 (s, 2H), 3.90 (dd, 2H), 3.43 (dd, 2H), 3.03 (s, 3H), 2.29 (s, 3H).

LC/MS (method F, ESIpos): Rₜ=1.24 min, m/z=514 [M+H]⁺.

### Example 87

### 1-(4-{4-[(5-Methyl-3-{3-[4-(trifluoromethoxy)phenyl]-1,2,4-oxadiazol-5-yl}-1H-pyrazol-1-yl)-methyl]pyridin-2-yl}piperazin-1-yl)ethanone

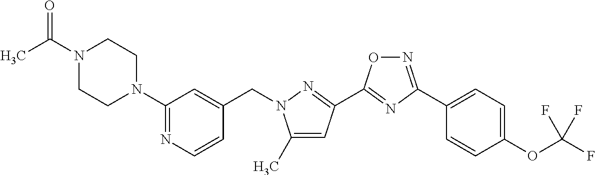 [SMILES: CC(=O)N1CCN(c2cc(Cn3nc(-c4nc(-c5ccc(OC(F)(F)F)cc5)no4)cc3C)ccn2)CC1]

128 μl (0.927 mmol) of triethylamine and 44 μl (0.618 mmol) of acetyl chloride were added to a solution of 300 mg (0.618 mmol) of the compound from Example 64 in 50 ml of anhydrous methylene chloride at 0° C. The reaction mixture was then stirred at RT for 16 h. 50 ml of saturated aqueous sodium bicarbonate solution were then added. After extraction by shaking, the organic phase which had been separated off was washed with water and then dried over anhydrous magnesium sulfate. After filtration, the solvent was removed on a rotary evaporator. The crude product was purified by means of preparative HPLC (method N). The product obtained was stirred with 3 ml of ethanol. After filtration and drying under a high vacuum, 234 mg (72% of th.) of the title compound were obtained.

¹H-NMR (400 MHz, CDCl₃, δ/ppm): 8.25 (d, 2H), 8.14 (d, 1H), 7.33 (d, 2H), 6.85 (s, 1H), 6.40 (d, 1H), 6.33 (s, 1H), 5.37 (s, 2H), 3.73-3.70 (m, 2H), 2.60-2.53 (m, 4H), 3.49-3.46 (m, 2H), 2.30 (s, 3H), 2.13 (s, 3H).

LC/MS (method Q, ESIpos): Rₜ=2.14 min, m/z=528 [M+H]⁺.

### Example 88

### 2-(4-Methylpiperidin-1-yl)-4-[(5-methyl-3-{3-[4-(trifluoromethoxy)phenyl]-1,2,4-oxadiazol-5-yl}-1H-pyrazol-1-yl)methyl]pyridine

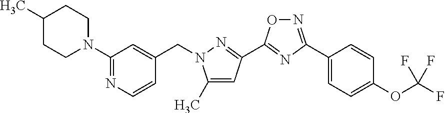 [SMILES: Cc1cc(-c2nc(-c3ccc(OC(F)(F)F)cc3)no2)nn1Cc1ccnc(N2CCC(C)CC2)c1]

Analogously to the process described under Example 56, 136 mg (59% of th.) of the title compound were obtained from 200 mg (0.459 mmol) of the compound from Example 81A and 2.8 ml (23.9 mmol) of 4-methylpiperidine. The crude product was purified by means of preparative HPLC (method N).

¹H-NMR (400 MHz, CDCl₃, δ/ppm): 8.25 (d, 2H), 8.10 (d, 1H), 7.33 (d, 2H), 6.83 (s, 1H), 6.33 (s, 1H), 6.28 (d, 1H), 5.33 (s, 2H), 4.22-4.15 (m, 2H), 2.82-2.74 (m, 2H), 2.29 (s, 3H), 1.72-1.67 (m, 2H), 1.63-1.53 (m, 1H), 1.22-1.13 (m, 2H), 0.95 (d, 3H).

LC/MS (method D, ESIpos): Rₜ=2.27 min, m/z=499 [M+H]⁺.

### Example 89

### 1-(Cyclopropylmethyl)-4-{4-[(5-methyl-3-{3-[4-(trifluoromethoxy)phenyl]-1,2,4-oxadiazol-5-yl}-1H-pyrazol-1-yl)methyl]pyridin-2-yl}piperazine

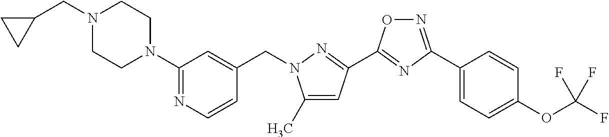 [SMILES: Cc1cc(-c2nc(-c3ccc(OC(F)(F)F)cc3)no2)nn1Cc1ccnc(N2CCN(CC3CC3)CC2)c1]

Analogously to the process described under Example 73, 52 mg (23% of th.) of the title compound were obtained from 200 mg (0.412 mmol) of the compound from Example 64 and 37 μl (0.494 mmol) of cyclopropanecarbaldehyde.

¹H-NMR (400 MHz, CDCl₃, δ/ppm): 8.25 (d, 2H), 8.12 (d, 1H), 7.33 (d, 2H), 6.83 (s, 1H), 6.33 (d, 1H), 6.32 (s, 1H), 5.35 (s, 2H), 3.52 (dd, 4H), 2.60 (dd, 4H), 2.29 (s, 3H), 2.28 (d, 2H), 0.93-0.83 (m, 1H), 0.54-0.51 (m, 2H), 0.13-0.10 (m, 2H).

LC/MS (method I, ESIpos): Rₜ=0.99 min, m/z=540 [M+H]⁺.

### Example 90

### 1-(4-{[5-Methyl-3-(3-{4-[1-(trifluoromethyl)cyclopropyl]phenyl}-1,2,4-oxadiazol-5-yl)-1H-pyrazol-1-yl]methyl}pyridin-2-yl)piperazine

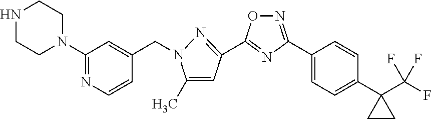 [SMILES: Cc1cc(-c2nc(-c3ccc(C4(C(F)(F)F)CC4)cc3)no2)nn1Cc1ccnc(N2CCNCC2)c1]

A mixture of 175 mg (0.381 mmol) of the compound from Example 145A and 656 mg (7.61 mmol) of piperazine was stirred at a bath temperature of 150° C. under argon overnight. After cooling to RT, ethyl acetate and water were added and the phases were separated. The aqueous phase was extracted three times with ethyl acetate, the combined ethyl acetate phases were concentrated and the residue was dried in vacuo to obtain 195 mg (98% of th., purity of 97%) of the title compound in this way.

LC/MS (method F, ESIpos): Rₜ=1.10 min, m/z=510 [M+H]⁺.

### Example 91

### 1-Cyclopropyl-4-(4-{[5-methyl-3-(3-{4-[1-(trifluoromethyl)cyclopropyl]phenyl}-1,2,4-oxadiazol-5-yl)-1H-pyrazol-1-yl]methyl}pyridin-2-yl)piperazine

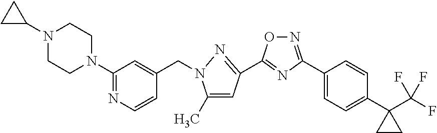 [SMILES: Cc1cc(-c2nc(-c3ccc(C4(C(F)(F)F)CC4)cc3)no2)nn1Cc1ccnc(N2CCN(C3CC3)CC2)c1]

195 mg (0.371 mmol, purity of 97%) of the compound from Example 90 were initially introduced into a mixture of 55 ml of methanol and 213 μl (3.71 mmol) of acetic acid at RT under argon and 50 mg of molecular sieve (3 Å) and 448 μl (2.23 mmol) of [(1-ethoxycyclopropyl)oxy](trimethyl)silane were added. After stirring at RT for 10 min, 70 mg (1.11 mmol) of sodium cyanoborohydride were added and the mixture was heated under reflux for 2 h. After cooling to RT, the solid present was filtered off and rinsed once with methanol and the filtrate was concentrated. The residue was purified by means of preparative HPLC (method O). The combined product fractions were concentrated to a residual volume of aqueous phase. Saturated aqueous sodium bicarbonate solution was added and the mixture was extracted twice with ethyl acetate. The combined ethyl acetate phases were dried over magnesium sulfate, filtered and concentrated. The residue was recrystallized from diethyl ether. After drying in vacuo, 86 mg (42% of th.) of the title compound were obtained.

¹H-NMR (400 MHz, CDCl₃, δ/ppm): 8.18 (d, 2H), 8.12 (d, 1H), 7.59 (d, 2H), 6.84 (s, 1H), 6.34 (s, 1H), 6.33 (s, 1H), 5.38 (s, 2H), 3.50-3.42 (m, 4H), 2.72-2.66 (m, 4H), 2.28 (s, 3H), 1.68-1.58 (m, 1H), 1.47-1.36 (m, 2H), 1.09 (s, 2H), 0.50-0.40 (m, 4H).

LC/MS (method F, ESIpos): Rₜ=1.17 min, m/z=550 [M+H]⁺.

### Example 92

### 1-Cyclopropyl-4-(4-{[5-methyl-3-(3-{4-[N-methyl-S-(trifluoromethyl)sulfonimidoyl]phenyl}-1,2,4-oxadiazol-5-yl)-1H-pyrazol-1-yl]methyl}pyridin-2-yl)piperazine

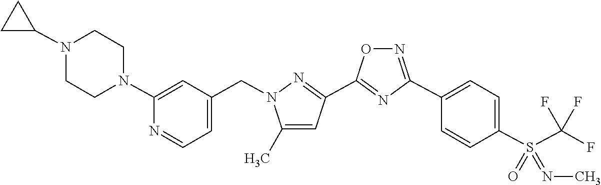 [SMILES: CN=S(=O)(c1ccc(-c2noc(-c3cc(C)n(Cc4ccnc(N5CCN(C6CC6)CC5)c4)n3)n2)cc1)C(F)(F)F]

80 mg (0.215 mmol) of the compound from Example 130A were initially introduced with 54 mg (0.215 mmol) of the compound from Example 135A into 1 ml of THF. The mixture was cooled to 0° C., 31 mg (0.280 mmol) of potassium tert-butylate were added and the mixture was stirred first at RT for 1 h and then under reflux for 24 h. It was subsequently concentrated and the residue was purified by preparative HPLC (method O) twice. The combined product fractions were concentrated to a residual volume of water and saturated aqueous sodium bicarbonate solution was added. The solid formed was filtered off, washed twice with water and dried in vacuo. 28 mg (22% of th.) of the title compound were obtained in this way.

¹H-NMR (400 MHz, CDCl₃, δ/ppm): 8.44 (s, broad, 2H), 8.30-8.00 (m, 3H), 6.86 (s, broad, 1H), 6.34 (s, broad, 2H), 5.36 (s, broad, 2H), 3.50 (s, broad, 4H), 3.12 (s, broad, 3H), 2.70 (s, broad, 4H), 2.30 (s, broad, 3H), 1.60 (s, broad, 1H), 0.50 (s, broad, 4H).

LC/MS (method I, ESIpos): Rₜ=0.97 min, m/z=587 [M+H]⁺.

### Example 93

### 1-Cyclopropyl-4-{4-[(3-{3-[3-fluoro-4-(trifluoromethoxy)phenyl]-1,2,4-oxadiazol-5-yl}-5-methyl-1H-pyrazol-1-yl)methyl]pyridin-2-yl}piperazine

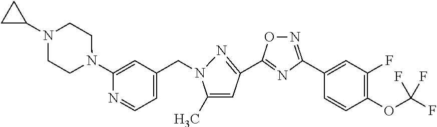 [SMILES: Cc1cc(-c2nc(-c3ccc(OC(F)(F)F)c(F)c3)no2)nn1Cc1ccnc(N2CCN(C3CC3)CC2)c1]

146 mg (1.30 mmol) of potassium tert-butylate were added to a mixture of 357 mg (1.0 mmol, purity of 92%) of the compound from Example 131A and 277 mg (1.10 mmol) of the compound from Example 135A in 10 ml of THF and the mixture was heated under reflux overnight, while stirring. After cooling to RT, the mixture was diluted with ethyl acetate and washed once with water. The aqueous phase was extracted once with ethyl acetate. The combined organic phases were washed once with saturated sodium chloride solution, dried over magnesium sulfate, filtered and concentrated. The residue was purified by means of preparative HPLC (method O). The combined product fractions were concentrated to a residual volume of aqueous phase, saturated aqueous sodium bicarbonate solution was added and the mixture was extracted twice with ethyl acetate. The combined ethyl acetate phases were dried over magnesium sulfate, filtered and concentrated. After the residue had been dried in vacuo, 269 mg (49% of th.) of the title compound were obtained.

¹H-NMR (400 MHz, CDCl₃, δ/ppm): 8.11 (d, 1H), 8.10-8.01 (m, 2H), 7.46-7.41 (t, 1H), 6.83 (s, 1H), 6.32 (s, 2H), 5.35 (s, 2H), 3.49-3.44 (m, 4H), 2.71-2.66 (m, 4H), 2.29 (s, 3H), 1.68-1.60 (m, 1H), 0.50-0.40 (m, 4H).

LC/MS (method D, ESIpos): Rₜ=1.97 min, m/z=544 [M+H]⁺.

### Example 94

### 4-[(5-Methyl-3-{3-[4-(trifluoromethoxy)phenyl]-1,2,4-oxadiazol-5-yl}-1H-pyrazol-1-yl)methyl]-2-(pyrrolidin-1-yl)pyridine

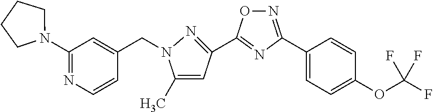 [SMILES: Cc1cc(-c2nc(-c3ccc(OC(F)(F)F)cc3)no2)nn1Cc1ccnc(N2CCCC2)c1]

Analogously to the process described under Example 56, 63 mg (28% of th.) of the title compound were obtained from 200 mg (0.459 mmol) of the compound from Example 81A and 770 μl (9.18 mmol) of pyrrolidine. For working up, when the reaction had ended the reaction mixture was first concentrated to dryness on a rotary evaporator and the residue was then stirred with acetonitrile. The solid thereby obtained was filtered off. The product was isolated from the filtrate by preparative HPLC (method N).

¹H-NMR (400 MHz, CDCl₃, δ/ppm): 8.26 (d, 2H), 8.10 (d, 1H), 7.34 (d, 2H), 6.83 (s, 1H), 6.27 (d, 1H), 6.02 (s, 1H), 5.35 (s, 2H), 3.41-3.36 (m, 4H), 2.28 (s, 3H), 1.99-1.97 (m, 4H).

LC/MS (method I, ESIpos): Rₜ=0.99 min, m/z=471 [M+H]⁺.

### Example 95

### 2-(Azetidin-1-yl)-4-[(5-methyl-3-{3-[4-(trifluoromethoxy)phenyl]-1,2,4-oxadiazol-5-yl}-1H-pyrazol-1-yl)methyl]pyridine

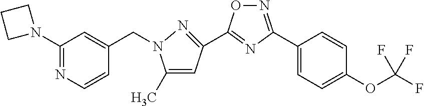 [SMILES: Cc1cc(-c2nc(-c3ccc(OC(F)(F)F)cc3)no2)nn1Cc1ccnc(N2CCC2)c1]

Analogously to the process described under Example 16, 150 mg (0.344 mmol) of the compound from Example 81A and 232 μl (3.44 mmol) of azetidine were reacted to give 66 mg (42% of th.) of the title compound.

¹H-NMR (400 MHz, CDCl₃, δ/ppm): 8.25 (d, 2H), 8.09 (d, 1H), 7.34 (d, 2H), 6.83 (s, 1H), 6.31 (d, 1H), 5.93 (s, 1H), 5.34 (s, 2H), 4.03-3.98 (m, 4H), 2.42-2.31 (m, 2H), 2.28 (s, 3H).

LC/MS (method I, ESIpos): Rₜ=0.96 min, m/z=457 [M+H]⁺.

### Example 96

### 4-[(5-Methyl-3-{3-[4-(pyrrolidin-1-yl)phenyl]-1,2,4-oxadiazol-5-yl}-1H-pyrazol-1-yl)methyl]-2-(pyrrolidin-1-yl)pyridine

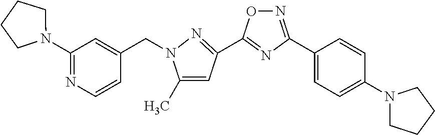 [SMILES: Cc1cc(-c2nc(-c3ccc(N4CCCC4)cc3)no2)nn1Cc1ccnc(N2CCCC2)c1]

Analogously to the process described under Example 56, 89 mg (40% of th.) of the title compound were obtained from 200 mg (0.459 mmol) of the compound from Example 81A and 770 μl (9.18 mmol) of pyrrolidine. For working up, when the reaction had ended the reaction mixture was first concentrated to dryness on a rotary evaporator and the residue was then stirred with acetonitrile. The product thereby remained in undissolved form and was separated off and dried under a high vacuum.

¹H-NMR (400 MHz, CDCl₃, δ/ppm): 8.09 (d, 1H), 8.04 (d, 2H), 6.80 (s, 1H), 6.60 (d, 2H), 6.27 (d, 1H), 6.00 (s, 1H), 5.34 (s, 2H), 3.40-3.34 (m, 8H), 2.27 (s, 3H), 2.05-2.01 (m, 4H), 1.99-1.95 (m, 4H).

LC/MS (method I, ESIpos): Rₜ=0.99 min, m/z=456 [M+H]⁺.

### Example 97

### 2-(4-tent-Butylpiperidin-1-yl)-4-[(5-methyl-3-{3-[4-(trifluoromethoxy)phenyl]-1,2,4-oxadiazol-5-yl}-1H-pyrazol-1-yl)methyl]pyridine

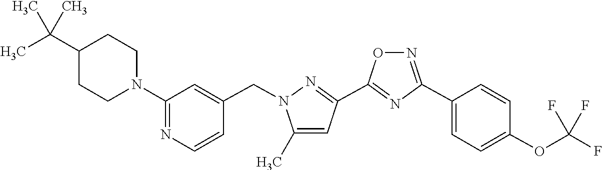 [SMILES: Cc1cc(-c2nc(-c3ccc(OC(F)(F)F)cc3)no2)nn1Cc1ccnc(N2CCC(C(C)(C)C)CC2)c1]

Analogously to the process described under Example 56, 99 mg (65% of th., purity of 98%) of the title compound were obtained from 120 mg (0.275 mmol) of the compound from Example 81A and 778 mg (5.51 mmol) of 4-tert-butylpiperidine. The product was isolated by means of preparative HPLC (method N) and finally stirred with pentane/diethyl ether (20:1).

¹H-NMR (400 MHz, CDCl₃, δ/ppm): 8.25 (d, 2H), 8.11 (d, 1H), 7.33 (d, 2H), 6.83 (s, 1H), 6.33 (s, 1H), 6.28 (d, 1H), 5.33 (s, 2H), 4.31-4.26 (m, 2H), 2.72-2.65 (m, 2H), 2.28 (s, 3H), 1.77-1.72 (m, 2H), 1.31-1.14 (m, 3H), 0.86 (s, 9H).

LC/MS (method I, ESIpos): Rₜ=1.33 min, m/z=541 [M+H]⁺.

### Example 98

### 1-{4-[(5-Methyl-3-{3-[4-(trifluoromethoxy)phenyl]-1,2,4-oxadiazol-5-yl}-1H-pyrazol-1-yl)-methyl]pyridin-2-yl}-4-(2,2,2-trifluoroethyl)-1,4-diazepan

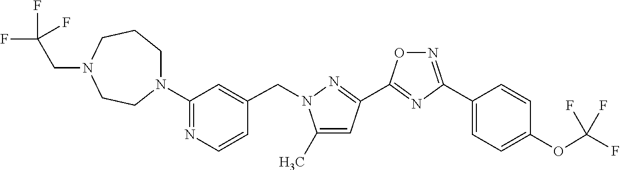 [SMILES: Cc1cc(-c2nc(-c3ccc(OC(F)(F)F)cc3)no2)nn1Cc1ccnc(N2CCCN(CC(F)(F)F)CC2)c1]

100 mg (0.23 mmol) of the compound from Example 81A and 209 mg (1.15 mmol) of 1-(2,2,2-trifluoroethyl)-1,4-diazepan were heated at 160° C. in a microwave oven for 3 h. After cooling to RT, the reaction mixture was purified directly by preparative HPLC (method P). The combined product fractions were concentrated on a rotary evaporator. After the residue had been dried in vacuo, 92 mg (65% of th.) of the title compound were obtained.

¹H-NMR (400 MHz, DMSO-d₆, δ/ppm): 8.20 (d, 2H), 8.00 (d, 1H), 7.59 (d, 2H), 6.95 (s, 1H), 6.44 (s, 1H), 6.20 (d, 1H), 5.43 (s, 2H), 3.67 (t, 2H), 3.57 (t, 2H), 3.27 (m, 2H), 2.92 (d, 2H), 2.75 (d, 2H), 2.33 (s, 3H), 1.80 (m, 2H).

LC/MS (method D, ESIpos): Rₜ=2.26 min, m/z=581 [M+H]⁺.

### Example 99

### 2-(1-Cyclopropylpiperidin-4-yl)-4-[(5-methyl-3-{3-[4-(trifluoromethoxy)phenyl]-1,2,4-oxadiazol-5-yl}-1H-pyrazol-1-yl)methyl]pyridine

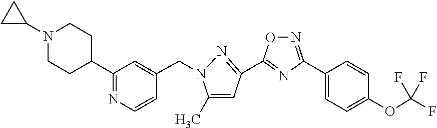 [SMILES: Cc1cc(-c2nc(-c3ccc(OC(F)(F)F)cc3)no2)nn1Cc1ccnc(C2CCN(C3CC3)CC2)c1]

130 mg (0.250 mmol) of the compound from Example 63 were dissolved in approx. 10 ml of methanol and converted into the free base by percolation over a bicarbonate cartridge (Polymerlabs, Stratospheres SPE, PL-HCO₃ MP SPE, capacity 0.9 mmol). After the solvent had been evaporated off, the residue was taken up again in 3.5 ml of methanol, and 143 μl (2.49 mmol) of glacial acetic acid, 301 μl (1.50 mmol) of 1-ethoxy-1-(trimethylsilyloxy)cyclopropane and 40 mg of dried, powdered molecular sieve (3 Å) were added. After stirring at RT for 10 min, 47 mg (0.749 mmol) of solid sodium cyanoborohydride were added. The reaction mixture was then heated under reflux for 4 h. After cooling to RT, the mixture was diluted with approx. 10 ml of methylene chloride and the undissolved material was filtered off. The filtrate was concentrated to dryness on a rotary evaporator and the residue was then dissolved again in approx. 4 ml of methanol. A prepurification of the product was carried out by means of preparative HPLC (method N). The product fractions were combined and freed from the solvent. The residue obtained was freed from the formic acid originating from the preparative HPLC by percolation over a bicarbonate cartridge (Polymerlabs, Stratospheres SPE, PL-HCO₃ MP SPE, capacity 0.9 mmol). A final fine purification was carried out by means of chromatography over silica gel (mobile phase: cyclohexane/ethyl acetate 10:1→1:1). 74 mg (54% of th., purity of 96%) of the title compound were obtained in this way.

¹H-NMR (400 MHz, CDCl₃, δ/ppm): 8.48 (d, 1H), 8.25 (d, 2H), 7.33 (d, 2H), 6.89 (s, 1H), 6.85 (s, 1H), 6.83 (d, 1H), 5.41 (s, 2H), 3.17-3.11 (m, 2H), 2.76-2.68 (m, 1H), 2.33-2.27 (m, 2H), 2.28 (s, 3H), 1.92-1.87 (m, 2H), 1.74-1.63 (m, 2H), 1.62-1.58 (m, 1H), 0.48-0.39 (m, 4H).

LC/MS (method I, ESIpos): Rₜ=0.97 min, m/z=525 [M+H]⁺.

### Example 100

### {3-[(5-Methyl-3-{3-[4-(trifluoromethoxy)phenyl]-1,2,4-oxadiazol-5-yl}-1H-pyrazol-1-yl)methyl]-phenyl}(1,2-oxazolidin-2-yl)methanone

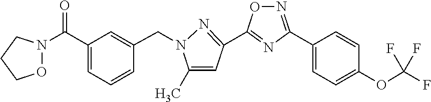 [SMILES: Cc1cc(-c2nc(-c3ccc(OC(F)(F)F)cc3)no2)nn1Cc1cccc(C(=O)N2CCCO2)c1]

Analogously to the process described under Example 32, 68 mg (60% of th.) of the title compound were obtained from 100 mg (0.225 mmol) of the compound from Example 93A and 49 mg (0.450 mmol) of 1,2-oxazolidine hydrochloride. In deviation from the instructions mentioned, a further equivalent of N,N-diisopropylethylamine was employed as the base here. Final percolation over a bicarbonate cartridge was omitted.

¹H-NMR (400 MHz, CDCl₃, δ/ppm): 8.25 (d, 2H), 7.73 (d, 1H), 7.62 (s, 1H), 7.39 (t, 1H), 7.33 (d, 2H), 7.25 (d, 1H), 6.81 (s, 1H), 5.49 (s, 2H), 3.95 (t, 2H), 3.88 (t, 2H), 2.33 (quint, 2H), 2.29 (s, 3H).

HPLC (method A): Rₜ=4.58 min.

MS (DCI, NH₃): m/z=500 [M+H]⁺, 517 [M+NH₄]⁺.

LC/MS (method F, ESIpos): Rₜ=1.39 min, m/z=500 [M+H]⁺.

### Example 101

### (4-Hydroxypiperidin-1-yl) {3-[(5-methyl-3-{3-[4-(trifluoromethoxy)phenyl]-1,2,4-oxadiazol-5-yl}-1H-pyrazol-1-yl)methyl]phenyl}methanone

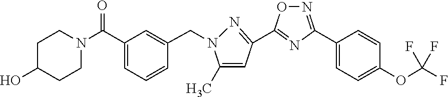 [SMILES: Cc1cc(-c2nc(-c3ccc(OC(F)(F)F)cc3)no2)nn1Cc1cccc(C(=O)N2CCC(O)CC2)c1]

Analogously to the process described under Example 32, 117 mg (98% of th.) of the title compound were obtained from 100 mg (0.225 mmol) of the compound from Example 93A and 46 mg (0.450 mmol) of 4-hydroxypiperidine. Final percolation over a bicarbonate cartridge was omitted here.

¹H-NMR (400 MHz, CDCl₃, δ/ppm): 8.25 (d, 2H), 7.39 (t, 1H), 7.33 (2d, 2H+1H), 7.20 (d, 1H), 7.19 (s, 1H), 6.82 (s, 1H), 5.48 (s, 2H), 4.15 (broad, 1H), 3.95 (broad, 1H), 3.59 (broad, 1H), 3.37 (broad, 1H), 3.14 (broad, 1H), 2.30 (s, 3H), 1.95 (broad, 1H), 1.79 (broad, 1H), 1.59 (broad, 2H), 1.45 (broad, 1H).

HPLC (method A): Rₜ=4.41 min.

MS (DCI, NH₃): m/z=528 [M+H]⁺, 545 [M+NH₄]⁺.

LC/MS (method I, ESIpos): Rₜ=1.12 min, m/z=528 [M+H]⁺.

### Example 102

### {3-[(5-Methyl-3-{3-[4-(trifluoromethoxy)phenyl]-1,2,4-oxadiazol-5-yl}-1H-pyrazol-1-yl)methyl]-phenyl}(morpholin-4-yl)methanone

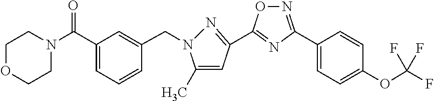 [SMILES: Cc1cc(-c2nc(-c3ccc(OC(F)(F)F)cc3)no2)nn1Cc1cccc(C(=O)N2CCOCC2)c1]

Analogously to the process described under Example 32, 76 mg (66% of th.) of the title compound were obtained from 100 mg (0.225 mmol) of the compound from Example 93A and 40 μl (0.450 mmol) of morpholine. The purification by preparative HPLC and the final percolation over a bicarbonate cartridge were omitted in this case; the product precipitated out on addition of water to the reaction mixture and was filtered off with suction and dried under a high vacuum.

¹H-NMR (400 MHz, CDCl₃, δ/ppm): 8.24 (d, 2H), 7.40 (t, 1H), 7.33 (2d, 2H+1H), 7.21 (d, 1H), 7.20 (s, 1H), 6.83 (s, 1H), 5.48 (s, 2H), 3.74 (broad, 4H), 3.60 (broad, 2H), 3.39 (broad, 2H), 2.31 (s, 3H).

HPLC (method A): Rₜ=4.54 min.

MS (DCI, NH₃): m/z=514 [M+H]⁺, 531 [M+NH₄]⁺.

LC/MS (method I, ESIpos): Rₜ=1.19 min, m/z=514 [M+H]⁺.

### Example 103

### {3-[(5-Methyl-3-{3-[4-(trifluoromethoxy)phenyl]-1,2,4-oxadiazol-5-yl}-1H-pyrazol-1-yl)methyl]-phenyl}(pyrrolidin-1-yl)methanone

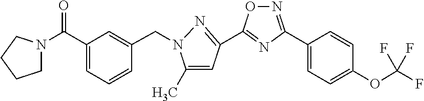 [SMILES: Cc1cc(-c2nc(-c3ccc(OC(F)(F)F)cc3)no2)nn1Cc1cccc(C(=O)N2CCCC2)c1]

Analogously to the process described under Example 32, 72 mg (64% of th.) of the title compound were obtained from 100 mg (0.225 mmol) of the compound from Example 93A and 38 μl (0.450 mmol) of pyrrolidine. Final percolation over a bicarbonate cartridge was omitted here.

¹H-NMR (400 MHz, CDCl₃, δ/ppm): 8.25 (d, 2H), 7.46 (d, 1H), 7.38 (t, 1H), 7.33 (d, 2H), 7.31 (s, 1H), 7.19 (d, 1H), 6.82 (s, 1H), 5.47 (s, 2H), 3.61 (t, 2H), 3.35 (t, 2H), 2.29 (s, 3H), 1.94 (quint, 2H), 1.85 (quint, 2H).

HPLC (method A): Rₜ=4.68 min.

MS (ESIpos): m/z=498 [M+H]⁺, 995 [2M+H]⁺.

LC/MS (method F, ESIpos): Rₜ=1.43 min, m/z=498 [M+H]⁺.

### Example 104

Azetidin-1-yl {3-[(5-methyl-3-{3-[4-(trifluoromethoxy)phenyl]-1,2,4-oxadiazol-5-yl}-1H-pyrazol-1-yl)methyl]phenyl}methanone

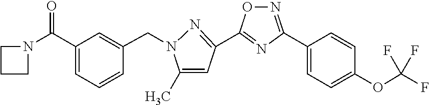 [SMILES: Cc1cc(-c2nc(-c3ccc(OC(F)(F)F)cc3)no2)nn1Cc1cccc(C(=O)N2CCC2)c1]

Analogously to the process described under Example 32, 84 mg (78% of th.) of the title compound were obtained from 100 mg (0.225 mmol) of the compound from Example 93A and 79 μl (0.450 mmol) of azetidine. Final percolation over a bicarbonate cartridge was omitted here.

¹H-NMR (400 MHz, CDCl₃, δ/ppm): 8.25 (d, 2H), 7.55 (d, 1H), 7.45 (s, 1H), 7.39 (t, 1H), 7.32 (s, 2H), 7.24 (d, 1H), 6.82 (s, 1H), 5.48 (s, 2H), 4.24-4.18 (m, 4H), 2.32 (quint, 2H), 2.29 (s, 3H).

HPLC (method A): Rₜ=4.62 min.

MS (DCI, NH₃): m/z=484 [M+H]⁺, 501 [M+NH₄]⁺.

LC/MS (method F, ESIpos): Rₜ=1.39 min, m/z=484 [M+H]⁺.

### Example 105

### (3-Fluoroazetidin-1-yl){3-[(5-methyl-3-{3-[4-(trifluoromethoxy)phenyl]-1,2,4-oxadiazol-5-yl}-1H-pyrazol-1-yl)methyl]phenyl}methanone

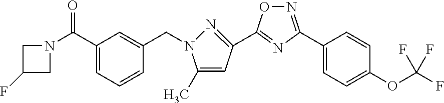 [SMILES: Cc1cc(-c2nc(-c3ccc(OC(F)(F)F)cc3)no2)nn1Cc1cccc(C(=O)N2CC(F)C2)c1]

Analogously to the process described under Example 32, 95 mg (84% of th.) of the title compound were obtained from 100 mg (0.225 mmol) of the compound from Example 93A and 50 mg (0.450 mmol) of 3-fluoroazetidine hydrochloride [B. Hulin et al., Bioorg. Med. Chem. Lett. 2005, 15 (21), 4770-4773]. In deviation from the instructions mentioned, a further equivalent of N,N-diisopropylethylamine was employed as the base here. Final percolation over a bicarbonate cartridge was omitted.

¹H-NMR (400 MHz, CDCl₃, δ/ppm): 8.24 (d, 2H), 7.74 (d, 1H), 7.47 (s, 1H), 7.41 (t, 1H), 7.34 (d, 2H), 7.29 (d, 1H), 6.83 (s, 1H), 5.48 (s, 2H), 5.41-5.22 (m, 1H), 4.50-4.39 (m, 2H), 4.38-4.24 (m, 2H), 2.30 (s, 3H).

HPLC (method A): Rₜ=4.59 min.

MS (ESIpos): m/z=502 [M+H]⁺, 1003 [2M+H]⁺.

LC/MS (method F, ESIpos): Rₜ=1.40 min, m/z=502 [M+H]⁺.

### Example 106

### (3-Methoxyazetidin-1-yl){3-[(5-methyl-3-{3-[4-(trifluoromethoxy)phenyl]-1,2,4-oxadiazol-5-yl}-1H-pyrazol-1-yl)methyl]phenyl}methanone

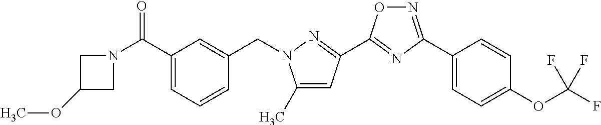 [SMILES: COC1CN(C(=O)c2cccc(Cn3nc(-c4nc(-c5ccc(OC(F)(F)F)cc5)no4)cc3C)c2)C1]

Analogously to the process described under Example 32, 72 mg (60% of th., purity of 96%) of the title compound were obtained from 100 mg (0.225 mmol) of the compound from Example 93A and 56 mg (0.450 mmol) of 3-methoxyazetidine hydrochloride [L. Provins et al., Bioorg. Med. Chem. Lett. 2007, 17 (11), 3077-3080]. In deviation from the instructions mentioned, a further equivalent of N,N-diisopropylethylamine was employed as the base here. Final percolation over a bicarbonate cartridge was omitted.

¹H-NMR (400 MHz, CDCl₃, δ/ppm): 8.25 (d, 2H), 7.55 (d, 1H), 7.44 (s, 1H), 7.39 (t, 1H), 7.33 (d, 2H), 7.25 (d, 1H), 6.83 (s, 1H), 5.48 (s, 2H), 4.37-4.29 (m, 2H), 4.22-4.17 (m, 1H), 4.10-4.01 (m, 2H), 3.26 (s, 3H), 2.30 (s, 3H).

HPLC (method A): Rₜ=4.58 min.

MS (ESIpos): m/z=514 [M+H]⁺, 1027 [2M+H]⁺.

LC/MS (method F, ESIpos): Rₜ=1.38 min, m/z=514 [M+H]⁺.

### Example 107

### (3-Methylazetidin-1-yl) {3-[(5-methyl-3-{3-[4-(trifluoromethoxy)phenyl]-1,2,4-oxadiazol-5-yl}-1H-pyrazol-1-yl)methyl]phenyl}methanone

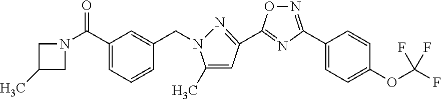 [SMILES: Cc1cc(-c2nc(-c3ccc(OC(F)(F)F)cc3)no2)nn1Cc1cccc(C(=O)N2CC(C)C2)c1]

Analogously to the process described under Example 32, 72 mg (60% of th., purity of 96%) of the title compound were obtained from 100 mg (0.225 mmol) of the compound from Example 93A and 48 mg (0.450 mmol) of 3-methylazetidine hydrochloride [L. Provins et al., Bioorg. Med. Chem. Lett. 2007, 17 (11), 3077-3080]. In deviation from the instructions mentioned, a further equivalent of N,N-diisopropylethylamine was employed as the base here. Final percolation over a bicarbonate cartridge was omitted.

¹H-NMR (400 MHz, CDCl₃, δ/ppm): 8.25 (d, 2H), 7.55 (d, 1H), 7.45 (s, 1H), 7.38 (t, 1H), 7.33 (d, 2H), 7.23 (d, 1H), 6.82 (s, 1H), 5.48 (s, 2H), 4.32-4.27 (m, 2H), 3.79-3.71 (m, 2H), 2.79-2.69 (m, 1H), 2.29 (s, 3H), 1.23 (d, 3H).

LC/MS (method F, ESIpos): Rₜ=1.45 min, m/z=498 [M+H]⁺.

### Example 108

### Ethyl 1-{4-[5-(5-methyl-1-{3-[(4-methylpiperazin-1-yl)carbonyl]benzyl}-1H-pyrazol-3-yl)-1,2,4-oxadiazol-3-yl]phenyl}cyclobutanecarboxylate

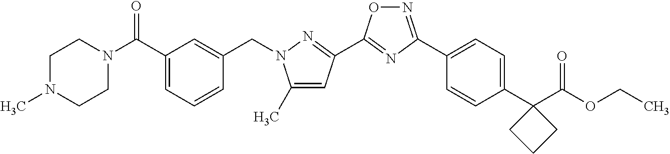 [SMILES: CCOC(=O)C1(c2ccc(-c3noc(-c4cc(C)n(Cc5cccc(C(=O)N6CCN(C)CC6)c5)n4)n3)cc2)CCC1]

86 mg (0.450 mmol) of EDC and 69 mg (0.450 mmol) of HOBt were added to a solution of 155 mg (0.409 mmol) of the hydrochloride of the compound from Example 105A in 2 ml of anhydrous DMF and the mixture was stirred at RT for 30 min. A solution of 118 mg (0.450 mmol) of the compound from Example 109A in 2 ml of anhydrous DMF was then added and stirring was continued at RT for 15 h. After this time, the reaction batch was immersed in an oil bath preheated to 140° C. and left in this for 1 h. After cooling to RT, the reaction mixture was separated directly into its components by means of preparative HPLC (method N). The product fractions were combined and concentrated to dryness on a rotary evaporator. The residue obtained was dissolved in approx. 5 ml of methanol and the solution was passed over a bicarbonate cartridge (Polymerlabs, Stratospheres SPE, PL-HCO₃ MP SPE, capacity 0.9 mmol) in order to remove adhering formic acid from the HPLC purification. After concentration and drying, 58 mg (24% of th., purity of 95%) of the title compound were obtained.

¹H-NMR (400 MHz, CDCl₃, δ/ppm): 8.15 (d, 2H), 7.42 (d, 2H), 7.38 (t, 1H), 7.33 (d, 1H), 7.19 (d, 1H), 7.15 (s, 1H), 6.83 (s, 1H), 5.47 (s, 2H), 4.12 (quart, 2H), 3.76 (broad, 2H), 3.37 (broad, 2H), 2.91-2.83 (m, 2H), 2.58-2.50 (m, 2H), 2.43 (broad, 2H), 2.30 (s, 3H), 2.28 (broad, 2H), 2.24 (s, 3H), 2.13-2.02 (m, 1H), 1.94-1.85 (m, 1H), 1.17 (t, 3H).

LC/MS (method F, ESIpos): Rₜ=1.13 min, m/z=569 [M+H]⁺.

### Example 109

### (4-Methylpiperazin-1-yl)(3-{[5-methyl-3-(3-{4-[(trifluoromethyl)sulfanyl]phenyl}-1,2,4-oxadiazol-5-yl)-1H-pyrazol-1-yl]methyl}phenyl)methanone

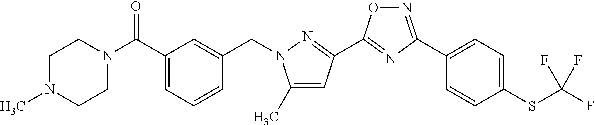 [SMILES: Cc1cc(-c2nc(-c3ccc(SC(F)(F)F)cc3)no2)nn1Cc1cccc(C(=O)N2CCN(C)CC2)c1]

89 mg (0.465 mmol) of EDC, 71 mg (0.465 mmol) of HOBt and 59 μl (0.422 mmol) of triethylamine were added to a solution of 160 mg (0.422 mmol) of the hydrochloride of the compound from Example 105A in 2 ml of anhydrous DMF and the mixture was stirred at RT for 30 min. A solution of 120 mg (0.507 mmol) of the compound from Example 15A in 2 ml of anhydrous DMF was then added and stirring was continued at RT for 1 h. After this time, the reaction batch was immersed in an oil bath preheated to 140° C. and left in this for 1 h. After cooling to RT, the reaction mixture was separated directly into its components by means of preparative HPLC (method N). The product fractions were combined and concentrated on a rotary evaporator to about half the original volume. A pH of approx. 8-9 was then established by addition of solid sodium bicarbonate. The mixture was extracted three times with approx. 20 ml of ethyl acetate each time. The combined organic extracts were washed successively with water and saturated sodium chloride solution. After drying over anhydrous magnesium sulfate and filtration, the solvent was removed on a rotary evaporator. 48 mg (21% of th., purity of approx. 95%) of the title compound were obtained.

¹H-NMR (400 MHz, DMSO-d₆, δ/ppm): 8.21 (d, 2H), 7.93 (d, 2H), 7.45 (t, 1H), 7.32 (d, 1H), 7.30 (d, 1H), 7.15 (s, 1H), 6.96 (s, 1H), 5.55 (s, 2H), 3.55 (broad, 2H), 3.24 (broad, 2H), 2.34 (s, 3H), 2.32 (broad, 2H), 2.18 (broad, 2H), 2.11 (s, 3H).

LC/MS (method D, ESIpos): Rₜ=2.00 min, m/z=543 [M+H]⁺.

### Example 110

### (3-{[3-(3-{4-[1-(Methoxymethyl)cyclobutyl]phenyl}-1,2,4-oxadiazol-5-yl)-5-methyl-1H-pyrazol-1-yl]methyl}phenyl)(4-methylpiperazin-1-yl)methanone

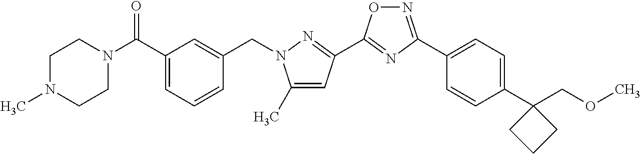

100 mg (0.523 mmol) of EDC and 80 mg (0.523 mmol) of HOBt were added to a solution of 180 mg (0.475 mmol) of the hydrochloride of the compound from Example 105A in 3 ml of anhydrous DMF and the mixture was stirred at RT for 30 min. A solution of 122 mg (0.523 mmol) of the compound from Example 110A in 2 ml of anhydrous DMF was then added and stirring was continued at RT for 15 h. After this time, the reaction batch was immersed in an oil bath preheated to 140° C. and left in this for 30 min. After cooling to RT, the reaction mixture was separated directly into its components by means of preparative HPLC (method N). The product fractions were combined and concentrated to dryness on a rotary evaporator. The residue obtained was dissolved in approx. 5 ml of methanol and the solution was passed over a bicarbonate cartridge (Polymerlabs, Stratospheres SPE, PL-HCO₃ MP SPE, capacity 0.9 mmol) in order to remove adhering formic acid from the HPLC purification. After concentration and drying, 67 mg (25% of th., purity of 95%) of the title compound were obtained.

¹H-NMR (400 MHz, CDCl₃, δ/ppm): 8.13 (d, 2H), 7.38 (t, 1H), 7.33 (d, 1H), 7.29 (d, 2H), 7.19 (d, 1H), 7.14 (s, 1H), 6.82 (s, 1H), 5.47 (s, 2H), 3.75 (broad, 2H), 3.54 (s, 2H), 3.36 (broad, 2H), 3.28 (s, 3H), 2.50-2.25 (m, 8H), 2.29 (s, 3H), 2.24 (s, 3H), 2.15-2.03 (m, 1H), 1.93-1.83 (m, 1H).

LC/MS (method I, ESIpos): Rₜ=1.01 min, m/z=541 [M+H]⁺.

### Example 111

### {3-[(3-{3-[4-(1-Fluorocyclobutyl)phenyl]-1,2,4-oxadiazol-5-yl}-5-methyl-1H-pyrazol-1-yl)-methyl]phenyl}(4-methylpiperazin-1-yl)methanone

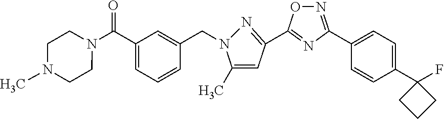 [SMILES: Cc1cc(-c2nc(-c3ccc(C4(F)CCC4)cc3)no2)nn1Cc1cccc(C(=O)N2CCN(C)CC2)c1]

Analogously to the process described under Example 41, 57 mg (38% of th.) of the title compound were obtained from 100 mg (0.292 mmol) of the compound from Example 105A and 73 mg (0.350 mmol) of the compound from Example 26A.

¹H-NMR (400 MHz, CDCl₃, δ/ppm): 8.23 (d, 2H), 7.59 (d, 2H), 7.39 (t, 1H), 7.34 (d, 1H), 7.20 (d, 1H), 7.16 (s, 1H), 6.84 (s, 1H), 5.48 (s, 2H), 3.76 (broad, 2H), 3.36 (broad, 2H), 2.77-2.55 (m, 4H), 2.44 (broad, 2H), 2.30 (s, 3H), 2.28 (broad, 2H), 2.25 (s, 3H), 2.19-2.07 (m, 1H), 1.87-1.75 (m, 1H).

HPLC (method A): Rₜ=4.24 min.

MS (DCI, NH₃): m/z=515 [M+H]⁺.

### Example 112

### (4-Methylpiperazin-1-yl){3-[(5-methyl-3-{3-[4-(1,1,1-trifluoro-2-methoxypropan-2-yl)phenyl]-1,2,4-oxadiazol-5-yl}-1H-pyrazol-1-yl)methyl]phenyl}methanone (racemate)

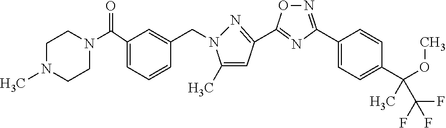 [SMILES: COC(C)(c1ccc(-c2noc(-c3cc(C)n(Cc4cccc(C(=O)N5CCN(C)CC5)c4)n3)n2)cc1)C(F)(F)F]

Analogously to the process described under Example 110, 500 mg (1.32 mmol) of the hydrochloride of the compound from Example 105A and 381 mg (1.45 mmol) of the compound from Example 111A were reacted to give 190 mg (24% of th., purity of 95%) of the title compound.

¹H-NMR (400 MHz, CDCl₃, δ/ppm): 8.23 (d, 2H), 7.64 (d, 2H), 7.39 (t, 1H), 7.33 (d, 1H), 7.20 (d, 1H), 7.16 (s, 1H), 6.83 (s, 1H), 5.48 (s, 2H), 3.75 (broad, 2H), 3.36 (broad, 2H), 3.27 (s, 3H), 2.44 (broad, 2H), 2.30 (s, 3H), 2.29 (broad, 2H), 2.26 (s, 3H), 1.82 (s, 3H).

LC/MS (method I, ESIpos): Rₜ=0.95 min, m/z=569 [M+H]⁺.

### Example 113

### (4-Methylpiperazin-1-yl) {3-[(5-methyl-3-{3-[4-(1,1,1-trifluoro-2-methoxypropan-2-yl)phenyl]-1,2,4-oxadiazol-5-yl}-1H-pyrazol-1-yl)methyl]phenyl}methanone (enantiomer 1)

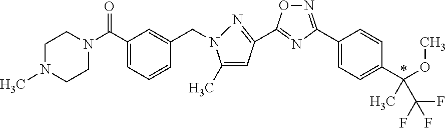 [SMILES: COC(C)(c1ccc(-c2noc(-c3cc(C)n(Cc4cccc(C(=O)N5CCN(C)CC5)c4)n3)n2)cc1)C(F)(F)F]

160 mg (0.281 mmol) of the racemic compound from Example 112 were dissolved in a mixture of 4 ml of isopropanol and 11 ml of isohexane and separated into the enantiomers by chromatography on a chiral phase [column material: Daicel Chiralpak AD-H, 5 μm, 250 mm×20 mm; injection volume: 0.3 ml; flow rate: 15 ml/min; temperature: 40° C.; UV detection: 220 nm; mobile phase: 50% isohexane, 49.8% isopropanol, 0.2% diethylamine]. 72 mg (90% of th., ee >98.5%) of the title compound (enantiomer 1) and 76 mg (95% of th., ee >99.0%) of the other enantiomer (Example 114) were obtained.

Analytical HPLC [Daicel Chiracel AD-H, 5 μm, 250 mm×4.6 mm; mobile phase: 40% isohexane, 59.8% isopropanol, 0.2% diethylamine; flow rate: 1 ml/min; temperature: 40° C.]: Rₜ=5.27 min.

### Example 114

### (4-Methylpiperazin-1-yl) {3-[(5-methyl-3-{3-[4-(1,1,1-trifluoro-2-methoxypropan-2-yl)phenyl]-1,2,4-oxadiazol-5-yl}-1H-pyrazol-1-yl)methyl]phenyl}methanone (enantiomer 2)

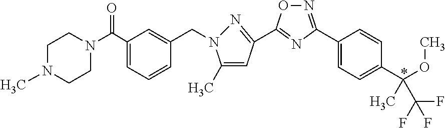 [SMILES: COC(C)(c1ccc(-c2noc(-c3cc(C)n(Cc4cccc(C(=O)N5CCN(C)CC5)c4)n3)n2)cc1)C(F)(F)F]

160 mg (0.281 mmol) of the racemic compound from Example 112 were dissolved in a mixture of 4 ml of isopropanol and 11 ml of isohexane and separated into the enantiomers by chromatography on a chiral phase [column material: Daicel Chiralpak AD-H, 5 μm, 250 mm×20 mm; injection volume: 0.3 ml; flow rate: 15 ml/min; temperature: 40° C.; UV detection: 220 nm; mobile phase: 50% isohexane, 49.8% isopropanol, 0.2% diethylamine]. 76 mg (95% of th., ee >99.0%) of the title compound (enantiomer 2) and 72 mg (90% of th., ee >98.5%) of the other enantiomer (Example 113) were obtained.

Analytical HPLC [Daicel Chiracel AD-H, 5 μm, 250 mm×4.6 mm; mobile phase: 40% isohexane, 59.8% isopropanol, 0.2% diethylamine; flow rate: 1 ml/min; temperature: 40° C.]: Rₜ=5.68 min.

### Example 115

### {3-[(3-{3-[3-Fluoro-4-(tetrahydro-2H-pyran-4-yl)phenyl]-1,2,4-oxadiazol-5-yl}-5-methyl-1H-pyrazol-1-yl)methyl]phenyl}(4-methylpiperazin-1-yl)methanone

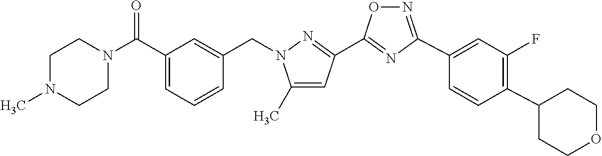 [SMILES: Cc1cc(-c2nc(-c3ccc(C4CCOCC4)c(F)c3)no2)nn1Cc1cccc(C(=O)N2CCN(C)CC2)c1]

Analogously to the process described under Example 110, 230 mg (0.607 mmol) of the hydrochloride of the compound from Example 105A and 159 mg (0.668 mmol) of the compound from Example 112A were reacted to give 43 mg (13% of th., purity of 98%) of the title compound.

¹H-NMR (400 MHz, CDCl₃, δ/ppm): 7.96 (d, 1H), 7.87 (d, 1H), 7.41-7.33 (m, 3H), 7.20 (d, 1H), 7.16 (s, 1H), 6.82 (s, 1H), 5.47 (s, 2H), 4.12-4.08 (m, 2H), 3.76 (broad, 2H), 3.61-3.54 (m, 2H), 3.36 (broad, 2H), 3.22-3.14 (m, 1H), 2.43 (broad, 2H), 2.30 (s, 3H), 2.28 (broad, 2H), 2.24 (s, 3H), 1.93-1.76 (m, 4H).

LC/MS (method I, ESIpos): Rₜ=0.89 min, m/z=545 [M+H]⁺.

### Example 116

### [3-({3-[3-(4-Chlorophenyl)-1,2,4-oxadiazol-5-yl]-5-methyl-1H-pyrazol-1-yl}methyl)phenyl]-(4-methylpiperazin-1-yl)methanone

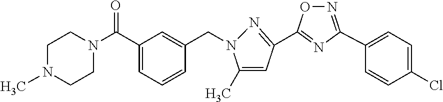 [SMILES: Cc1cc(-c2nc(-c3ccc(Cl)cc3)no2)nn1Cc1cccc(C(=O)N2CCN(C)CC2)c1]

Analogously to the process described under Example 109, 40 mg (20% of th., purity of 98%) of the title compound were prepared from 160 mg (0.422 mmol) of the hydrochloride of the compound from Example 105A and 86 mg (0.507 mmol) of 4-chloro-N-hydroxybenzamidine. After purification by preparative HPLC, the product was dissolved in approx. 5 ml of methanol and the solution was passed over a bicarbonate cartridge (Polymerlabs, Stratospheres SPE, PL-HCO₃ MP SPE, capacity 0.9 mmol) in order to remove adhering formic acid.

¹H-NMR (400 MHz, DMSO-d₆, δ/ppm): 8.07 (d, 2H), 7.67 (d, 2H), 7.46 (t, 1H), 7.32 (d, 1H), 7.30 (d, 1H), 7.14 (s, 1H), 6.93 (s, 1H), 5.54 (s, 2H), 3.56 (broad, 2H), 3.23 (broad, 2H), 2.34 (s, 3H), 2.30 (broad, 2H), 2.18 (broad, 2H), 2.11 (s, 3H).

LC/MS (method D, ESIpos): Rₜ=1.82 min, m/z=477 [M+H]⁺.

### Example 117

### (4-Methylpiperazin-1-yl){3-[(5-methyl-3-{3-[4-(tetrahydro-2H-pyran-4-yl)phenyl]-1,2,4-oxa-diazol-5-yl}-1H-pyrazol-1-yl)methyl]phenyl}methanone

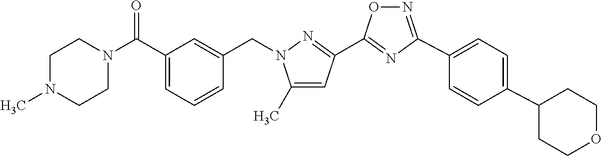 [SMILES: Cc1cc(-c2nc(-c3ccc(C4CCOCC4)cc3)no2)nn1Cc1cccc(C(=O)N2CCN(C)CC2)c1]

Analogously to the process described under Example 110, 27 mg (15% of th., purity of 96%) of the title compound were obtained from 125 mg (0.330 mmol) of the hydrochloride of the compound from Example 105A and 80 mg (0.363 mmol) of the compound from Example 113A.

¹H-NMR (400 MHz, CDCl₃, δ/ppm): 8.04 (d, 2H), 7.40-7.30 (m, 4H), 7.20 (d, 1H), 7.15 (s, 1H), 6.82 (s, 1H), 5.47 (s, 2H), 4.12-4.08 (m, 2H), 3.75 (broad, 2H), 3.58-3.52 (m, 2H), 3.36 (broad, 2H), 2.88-2.80 (m, 1H), 2.43 (broad, 2H), 2.30 (s, 3H), 2.28 (broad, 2H), 2.25 (s, 3H), 1.91-1.77 (m, 4H).

LC/MS (method F, ESIpos): Rₜ=0.97 min, m/z=527 [M+H]⁺.

### Example 118

### (3-{[3-(3-{4-[1-(2-Fluoroethyl)cyclobutyl]phenyl}-1,2,4-oxadiazol-5-yl)-5-methyl-1H-pyrazol-1-yl]methyl}phenyl)(4-methylpiperazin-1-yl)methanone

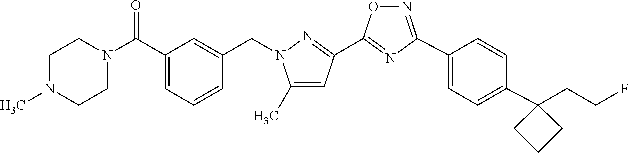 [SMILES: Cc1cc(-c2nc(-c3ccc(C4(CCF)CCC4)cc3)no2)nn1Cc1cccc(C(=O)N2CCN(C)CC2)c1]

Analogously to the process described under Example 110, 36 mg (17% of th., purity of 95%) of the title compound were obtained from 140 mg (0.370 mmol) of the hydrochloride of the compound from Example 105A and 96 mg (0.406 mmol) of the compound from Example 113A.

¹H-NMR (400 MHz, CDCl₃, δ/ppm): 8.14 (d, 2H), 7.41-7.33 (m, 2H), 7.30-7.23 (m, 2H), 7.20 (d, 1H), 7.16 (s, 1H), 6.83 (s, 1H), 5.47 (s, 2H), 4.31 (td, 2H), 3.75 (broad, 2H), 3.37 (broad, 2H), 2.50-2.40 (m, 4H), 2.33-2.09 (m, 7H), 2.30 (s, 3H), 2.25 (s, 3H), 1.93-1.84 (m, 1H).

LC/MS (method I, ESIpos): Rₜ=1.01 min, m/z=543 [M+H]⁺.

### Example 119

### (4-Methylpiperazin-1-yl) {3-[(5-methyl-3-{3-[3-(trifluoromethoxy)phenyl]-1,2,4-oxadiazol-5-yl}-1H-pyrazol-1-yl)methyl]phenyl}methanone hydrochloride

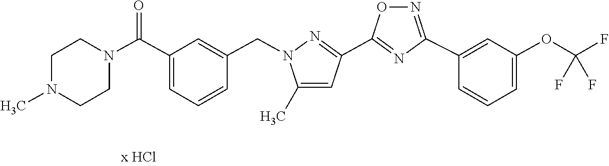 [SMILES: Cc1cc(-c2nc(-c3cccc(OC(F)(F)F)c3)no2)nn1Cc1cccc(C(=O)N2CCN(C)CC2)c1]

89 mg (0.465 mmol) of EDC, 71 mg (0.465 mmol) of HOBt and 59 μl (0.422 mmol) of triethylamine were added successively to a solution of 160 mg (0.422 mmol) of the hydrochloride of the compound from Example 105A in 2 ml of anhydrous DMF. After stirring at RT for 30 min, a solution of 112 mg (0.507 mmol) of 3-trifluoromethoxy-N-hydroxybenzamidine in 2 ml of anhydrous DMF was added. The reaction mixture was stirred first at RT for 1 h and then at 140° C. for 1 h. After cooling to RT, the reaction mixture was separated directly into its components via preparative HPLC (method N). The product fractions were combined and evaporated to dryness. The residue obtained was dissolved in approx. 3 ml of methanol and the solution was freed from adhering formic acid by percolation over a bicarbonate cartridge (Polymerlabs, Stratospheres SPE, PL-HCO₃ MP SPE, capacity 0.9 mmol). The product was then purified again via a filtration with suction (silica gel, mobile phase: methylene chloride/methanol 20:1). After evaporation of the product fraction, the residue was dissolved in approx. 2 ml of methylene chloride and approx. 5 ml of a 4 M solution of hydrogen chloride in dioxane was added. After evaporation to dryness, the residue was dissolved in methylene chloride again and a 4 M solution of hydrogen chloride in dioxane was again added. After renewed evaporation and drying under a high vacuum, 61 mg (26% of th., purity of 90%) of the title compound were obtained.

¹H-NMR (400 MHz, DMSO-d₆, δ/ppm): 10.38 (broad, 1H), 8.11 (d, 1H), 7.95 (s, 1H), 7.77 (t, 1H), 7.65 (d, 1H), 7.49 (t, 1H), 7.41 (d, 1H), 7.33 (d, 1H), 7.30 (s, 1H), 5.56 (s, 2H), 3.73-3.64 (m, 4H), 3.50-3.44 (m, 2H), 3.41-3.28 (m, 2H), 3.12-3.01 (m, 2H), 2.78 (s, 3H), 2.37 (s, 3H).

LC/MS (method D, ESIpos): Rₜ=1.92 min, m/z=527 [M+H]⁺.

### Example 120

### {3-[(5-Methyl-3-{3-[4-(tetrahydro-2H-pyran-4-yl)phenyl]-1,2,4-oxadiazol-5-yl}-1H-pyrazol-1-yl)-methyl]phenyl}(morpholin-4-yl)methanone

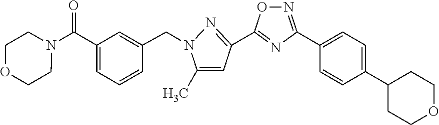 [SMILES: Cc1cc(-c2nc(-c3ccc(C4CCOCC4)cc3)no2)nn1Cc1cccc(C(=O)N2CCOCC2)c1]

Analogously to the process described under Example 32, 81 mg (88% of th.) of the title compound were obtained from 80 mg (0.180 mmol) of the compound from Example 147A and 31 μl (0.360 mmol) of morpholine. Final percolation over a bicarbonate cartridge was omitted here.

¹H-NMR (400 MHz, CDCl₃, δ/ppm): 8.14 (d, 2H), 7.41-7.32 (m, 4H), 7.21 (d, 1H), 7.20 (s, 1H), 6.83 (s, 1H), 5.47 (s, 2H), 4.12-4.08 (m, 2H), 3.80-3.70 (broad, 4H), 3.61 (broad, 2H), 3.54 (dt, 2H), 3.40 (broad, 2H), 2.87-2.79 (m, 1H), 2.30 (s, 3H), 1.92-1.78 (m, 4H).

LC/MS (method F, ESIpos): Rₜ=1.20 min, m/z=514 [M+H]⁺.

### Example 121

### (4-Methylpiperazin-1-yl){3-[(5-methyl-3-{3-[4-(1,1,1-trifluoro-2-hydroxypropan-2-yl)phenyl]-1,2,4-oxadiazol-5-yl}-1H-pyrazol-1-yl)methyl]phenyl}methanone (racemate)

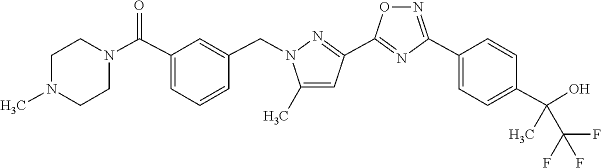 [SMILES: Cc1cc(-c2nc(-c3ccc(C(C)(O)C(F)(F)F)cc3)no2)nn1Cc1cccc(C(=O)N2CCN(C)CC2)c1]

Analogously to the process described under Example 110, 500 mg (1.32 mmol) of the hydrochloride of the compound from Example 105A and 360 mg (1.45 mmol) of the compound from Example 114A were reacted to give 72 mg (10% of th., purity of 97%) of the title compound. In deviation from the instructions mentioned, the reaction mixture was worked up as follows: The solvent DMF was first mostly removed on a rotary evaporator. Approx. 50 ml of water were added to the residue and the mixture was extracted three times with approx. 50 ml of ethyl acetate each time. The combined organic extracts were washed with saturated sodium chloride solution and dried over anhydrous magnesium sulfate. After filtration and evaporation of the solvent, the crude product was prepurified by means of MPLC (approx. 100 g of silica gel, mobile phase: cyclohexane/ethyl acetate 1:1). The product fractions were combined and freed from the solvent, and then subsequently purified by means of preparative HPLC, as described.

¹H-NMR (400 MHz, CDCl₃, δ/ppm): 8.23 (d, 2H), 7.72 (d, 2H), 7.39 (t, 1H), 7.34 (d, 1H), 7.20 (d, 1H), 7.16 (s, 1H), 6.83 (s, 1H), 5.48 (s, 2H), 3.76 (broad, 2H), 3.37 (broad, 2H), 2.78 (broad, 1H), 2.43 (broad, 2H), 2.31 (s, 3H), 2.29 (broad, 2H), 2.24 (s, 3H), 1.82 (s, 3H).

LC/MS (method I, ESIpos): Rₜ=0.85 min, m/z=555 [M+H]⁺.

### Example 122

### (4-Methylpiperazin-1-yl) {3-[(5-methyl-3-{3-[4-(1,1,1-trifluoro-2-hydroxypropan-2-yl)phenyl]-1,2,4-oxadiazol-5-yl}-1H-pyrazol-1-yl)methyl]phenyl}methanone (enantiomer 1)

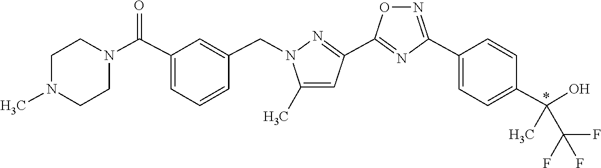 [SMILES: Cc1cc(-c2nc(-c3ccc(C(C)(O)C(F)(F)F)cc3)no2)nn1Cc1cccc(C(=O)N2CCN(C)CC2)c1]

62 mg (0.108 mmol) of the racemic compound from Example 121 were dissolved in 1 ml of ethanol and separated into the enantiomers by chromatography on a chiral phase [column material: Daicel Chiralpak AD-H, 5 μm, 250 mm×20 mm; injection volume: 0.5 ml; flow rate: 15 ml/min; temperature: 40° C.; UV detection: 220 nm; mobile phase: 50% isohexane, 49.8% ethanol, 0.2% diethylamine]. 24 mg (80% of th., ee >99.5%) of the title compound (enantiomer 1) and 27 mg (90% of th., ee >99.5%) of the other enantiomer (Example 123) were obtained.

Analytical HPLC [Daicel Chiralpak AD-H, 5 μm, 250 mm×4.6 mm; mobile phase: 40% isohexane, 59.8% ethanol, 0.2% diethylamine; flow rate: 1 ml/min; temperature: 40° C.]: Rₜ=7.20 min.

### Example 123

### (4-Methylpiperazin-1-yl){3-[(5-methyl-3-{3-[4-(1,1,1-trifluoro-2-hydroxypropan-2-yl)phenyl]-1,2,4-oxadiazol-5-yl}-1H-pyrazol-1-yl)methyl]phenyl}methanone (enantiomer 2)

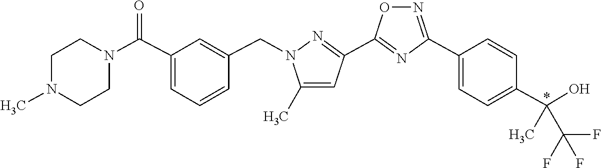 [SMILES: Cc1cc(-c2nc(-c3ccc(C(C)(O)C(F)(F)F)cc3)no2)nn1Cc1cccc(C(=O)N2CCN(C)CC2)c1]

62 mg (0.108 mmol) of the racemic compound from Example 121 were dissolved in 1 ml of ethanol and separated into the enantiomers by chromatography on a chiral phase [column material: Daicel Chiralpak AD-H, 5 μm, 250 mm×20 mm; injection volume: 0.5 ml; flow rate: 15 ml/min; temperature: 40° C.; UV detection: 220 nm; mobile phase: 50% isohexane, 49.8% ethanol, 0.2% diethylamine]. 27 mg (90% of th., ee >99.5%) of the title compound (enantiomer 2) and 24 mg (80% of th., ee >99.5%) of the other enantiomer (Example 122) were obtained.

Analytical HPLC [Daicel Chiralpak AD-H, 5 μm, 250 mm×4.6 mm; mobile phase: 40% isohexane, 59.8% ethanol, 0.2% diethylamine; flow rate: 1 ml/min; temperature: 40° C.]: Rₜ=9.26 min.

### Example 124

### {3-[(3-{3-[4-(1-Methoxycyclobutyl)phenyl]-1,2,4-oxadiazol-5-yl}-5-methyl-1H-pyrazol-1-yl)-methyl]phenyl}(4-methylpiperazin-1-yl)methanone

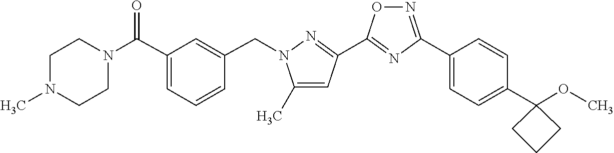

Analogously to the process described under Example 41, 66 mg (43% of th.) of the title compound were obtained from 100 mg (0.292 mmol) of the compound from Example 105A and 77 mg (0.350 mmol) of the compound from Example 25A.

¹H-NMR (400 MHz, CDCl₃, δ/ppm): 8.21 (d, 2H), 7.56 (d, 2H), 7.39 (t, 1H), 7.33 (d, 1H), 7.20 (d, 1H), 7.17 (s, 1H), 6.83 (s, 1H), 5.48 (s, 2H), 3.76 (broad, 2H), 3.37 (broad, 2H), 2.97 (s, 3H), 2.47-2.38 (m, 6H), 2.30 (s, 3H), 2.27 (broad, 2H), 2.26 (s, 3H), 2.02-1.93 (m, 1H), 1.78-1.67 (m, 1H).

HPLC (method A): Rₜ=4.14 min.

MS (DCI, NH₃): m/z=527 [M+H]⁺.

### Example 125

### [3-({5-Methyl-3-[3-(4-methylphenyl)-1,2,4-oxadiazol-5-yl]-1H-pyrazol-1-yl}methyl)phenyl]-(4-methylpiperazin-1-yl)methanone

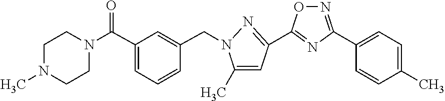

Analogously to the process described under Example 119, 48 mg (25% of th., purity of 97%) of the title compound were obtained from 160 mg (0.422 mmol) of the hydrochloride of the compound from Example 105A and 76 mg (0.507 mmol) of 4-methyl-N-hydroxybenzamidine. In deviation from the instructions mentioned, after percolation over a bicarbonate cartridge a subsequent chromatography over silica gel and conversion into the corresponding hydrochloride were omitted here.

¹H-NMR (400 MHz, DMSO-d₆, δ/ppm): 7.96 (d, 2H), 7.46 (t, 1H), 7.40 (d, 2H), 7.32 (d, 1H), 7.30 (d, 1H), 7.13 (s, 1H), 6.93 (s, 1H), 5.54 (s, 2H), 3.56 (broad, 2H), 3.23 (broad, 2H), 2.40 (s, 3H), 2.34 (s, 3H), 2.30 (broad, 2H), 2.18 (broad, 2H), 2.11 (s, 3H).

LC/MS (method I, ESIpos): Rₜ=0.87 min, m/z=457 [M+H]⁺.

### Example 126

### [3-({3-[3-(4-Fluorophenyl)-1,2,4-oxadiazol-5-yl]-5-methyl-1H-pyrazol-1-yl}methyl)phenyl]-(4-methylpiperazin-1-yl)methanone

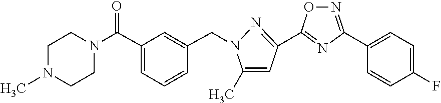 [SMILES: Cc1cc(-c2nc(-c3ccc(F)cc3)no2)nn1Cc1cccc(C(=O)N2CCN(C)CC2)c1]

Analogously to the process described under Example 119, 45 mg (23% of th., purity of 98%) of the title compound were obtained from 160 mg (0.422 mmol) of the hydrochloride of the compound from Example 105A and 78 mg (0.507 mmol) of 4-fluoro-N-hydroxybenzamidine. In deviation from the instructions mentioned, after percolation over a bicarbonate cartridge a subsequent chromatography over silica gel and conversion into the corresponding hydrochloride were omitted here.

¹H-NMR (400 MHz, DMSO-d₆, δ/ppm): 8.12 (dd, 2H), 7.47-7.41 (m, 3H), 7.32 (d, 1H), 7.30 (d, 1H), 7.15 (s, 1H), 6.94 (s, 1H), 5.55 (s, 2H), 3.55 (broad, 2H), 3.26 (broad, 2H), 2.34 (s, 3H), 2.29 (broad, 2H), 2.21 (broad, 2H), 2.12 (s, 3H).

LC/MS (method I, ESIpos): Rₜ=0.85 min, m/z=461 [M+H]⁺.

### Example 127

### (1-{4-[5-(5-Methyl-1-{3-[(4-methylpiperazin-1-yl)carbonyl]benzyl}-1H-pyrazol-3-yl)-1,2,4-oxa-diazol-3-yl]phenyl}cyclobutyl)methyl acetate formate

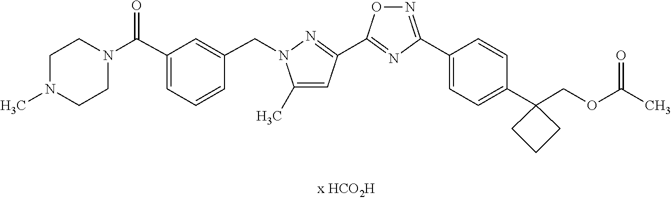 [SMILES: CC(=O)OCC1(c2ccc(-c3noc(-c4cc(C)n(Cc5cccc(C(=O)N6CCN(C)CC6)c5)n4)n3)cc2)CCC1]

Analogously to the process described under Example 110, 131 mg (26% of th., purity of 95%) of the title compound were obtained from 300 mg (0.792 mmol) of the hydrochloride of the compound from Example 105A and 287 mg (0.871 mmol) of the compound from Example 115A. Final percolation over a bicarbonate cartridge was not carried out in this case.

¹H-NMR (400 MHz, CDCl₃, δ/ppm): 8.18 (s, 1H), 8.13 (d, 2H), 7.40 (t, 1H), 7.34 (d, 1H), 7.28 (d, 2H), 7.23 (d, 1H), 7.18 (s, 1H), 6.83 (s, 1H), 5.97 (s, 2H), 4.28 (s, 2H), 3.86 (broad, 2H), 3.51 (broad, 2H), 2.69 (broad, 2H), 2.47-2.38 (m, 2H), 2.39 (s, 3H), 2.33-2.27 (m, 2H), 2.30 (s, 3H), 2.22-2.08 (m, 1H), 1.99 (s, 3H), 1.97-1.88 (m, 1H).

LC/MS (method D, ESIpos): Rₜ=1.90 min, m/z=569 [M+H]⁺.

### Example 128

### (3-{[3-(3-{4-[1-(2-Hydroxyethyl)cyclobutyl]phenyl}-1,2,4-oxadiazol-5-yl)-5-methyl-1H-pyrazol-1-yl]methyl}phenyl)(4-methylpiperazin-1-yl)methanone

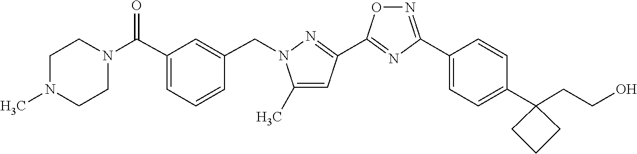 [SMILES: Cc1cc(-c2nc(-c3ccc(C4(CCO)CCC4)cc3)no2)nn1Cc1cccc(C(=O)N2CCN(C)CC2)c1]

111 mg (0.581 mmol) of EDC, 89 mg (0.581 mmol) of HOBt and 110 μl (0.792 mmol) of triethylamine were added to a solution of 200 mg (0.528 mmol) of the hydrochloride of the compound from Example 105A in 5 ml of anhydrous DMF and the mixture was stirred at RT for 30 min. A solution of 160 mg (0.581 mmol) of the compound from Example 116A in 2 ml of anhydrous DMF was then added and stirring was continued at RT for 1 h. After this time, the reaction batch was immersed in an oil bath preheated to 140° C. and left in this for 1 h. After cooling to RT, the solvent was mostly stripped off on a rotary evaporator. 50 ml of water were added to the residue obtained and the mixture was extracted three times with approx. 50 ml of ethyl acetate each time. The combined organic extracts were washed with saturated sodium chloride solution and dried over anhydrous magnesium sulfate. After filtration, the solvent was removed on a rotary evaporator and the residue was separated into its components by means of preparative HPLC (method N). Slightly contaminated fractions of the title compound (13 mg) and of the corresponding acetate (27 mg) were obtained. The fraction mentioned last was dissolved in 1 ml of ethanol and 100 μl of 1 M sodium hydroxide solution were added. After stirring at RT for 1 h, the mixture was neutralized by addition of 90 μl of 1 M hydrochloric acid and concentrated to dryness on a rotary evaporator. The residue was combined with the 13 mg of the product fraction obtained above and the mixture was then purified again by means of MPLC (silica gel, mobile phase: cyclohexane/ethyl acetate 20:1→1:1). 26 mg (9% of th., purity of 93%) of the title compound were obtained in this way.

¹H-NMR (400 MHz, CDCl₃, δ/ppm): 8.13 (d, 2H), 7.39 (t, 1H), 7.33 (d, 1H), 7.25 (d, 2H), 7.20 (d, 1H), 7.15 (s, 1H), 6.83 (s, 1H), 5.47 (s, 2H), 3.75 (broad, 2H), 3.47 (t, 2H), 3.36 (broad, 2H), 2.47-2.39 (m, 4H), 2.29 (s, 3H), 2.28 (broad, 2H), 2.25 (s, 3H), 2.24 (broad, 2H), 2.19-2.10 (m, 1H), 2.14 (t, 2H), 1.92-1.83 (m, 1H).

LC/MS (method I, ESIpos): Rₜ=0.86 min, m/z=541 [M+H]⁺.

### Example 129

### (3-{[3-(3-{4-[1-(Hydroxymethyl)cyclobutyl]phenyl}-1,2,4-oxadiazol-5-yl)-5-methyl-1H-pyrazol-1-yl]methyl}phenyl)(4-methylpiperazin-1-yl)methanone

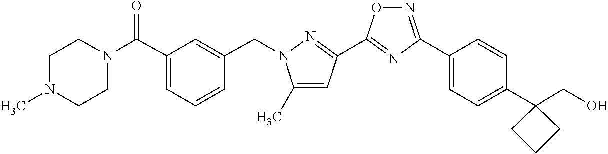 [SMILES: Cc1cc(-c2nc(-c3ccc(C4(CO)CCC4)cc3)no2)nn1Cc1cccc(C(=O)N2CCN(C)CC2)c1]

0.5 ml of 1 M sodium hydroxide solution was added to a solution of 125 mg (0.203 mmol) of the compound from Example 127 in 5 ml of ethanol and the mixture was stirred at RT for 30 min. The reaction mixture was then separated directly into its components by means of preparative HPLC (method N). The product fractions were combined and concentrated to dryness on a rotary evaporator. The residue was dissolved in approx. 5 ml of methanol and the solution was passed over a bicarbonate cartridge (Polymerlabs, Stratospheres SPE, PL-HCO₃ MP SPE, capacity 0.9 mmol) in order to remove adhering formic acid from the HPLC purification. After concentration and drying, 93 mg (85% of th.) of the title compound were obtained.

¹H-NMR (400 MHz, CDCl₃, δ/ppm): 8.16 (d, 2H), 7.39 (t, 1H), 7.33 (d, 1H), 7.27 (d, 2H), 7.20 (d, 1H), 7.15 (s, 1H), 6.83 (s, 1H), 5.48 (s, 2H), 3.80 (d, 2H), 3.77 (broad, 2H), 3.35 (broad, 2H), 2.49-2.33 (m, 4H), 2.29 (s, 3H), 2.28 (broad, 4H), 2.25 (s, 3H), 2.17-2.06 (m, 1H), 1.97-1.88 (m, 1H), 1.29 (t, 1H).

LC/MS (method I, ESIpos): Rₜ=0.84 min, m/z=527 [M+H]⁺.

### Example 130

### {3-[(5-Methyl-3-{3-[3-methyl-4-(tetrahydro-2H-pyran-4-yl)phenyl]-1,2,4-oxadiazol-5-yl}-1H-pyrazol-1-yl)methyl]phenyl}(4-methylpiperazin-1-yl)methanone

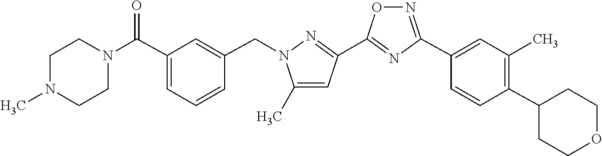 [SMILES: Cc1cc(-c2noc(-c3cc(C)n(Cc4cccc(C(=O)N5CCN(C)CC5)c4)n3)n2)ccc1C1CCOCC1]

Analogously to the process described under Example 110, 220 mg (0.581 mmol) of the hydrochloride of the compound from Example 105A and 150 mg (0.639 mmol) of the compound from Example 108A were reacted to give 57 mg (17% of th.) of the title compound.

¹H-NMR (400 MHz, CDCl₃, δ/ppm): 8.02-8.00 (m, 2H), 7.41-7.33 (m, 3H), 7.20 (d, 1H), 7.15 (s, 1H), 6.83 (s, 1H), 5.47 (s, 2H), 4.13-4.10 (m, 2H), 3.77 (broad, 2H), 3.60-3.54 (m, 2H), 3.37 (broad, 2H), 3.07-3.00 (m, 1H), 2.45 (broad, 2H), 2.43 (s, 3H), 2.30 (s, 3H), 2.29 (broad, 2H), 2.27 (s, 3H), 1.92-1.81 (m, 2H), 1.73-1.69 (m, 2H).

LC/MS (method I, ESIpos): Rₜ=0.89 min, m/z=541 [M+H]⁺.

### Example 131

### N,N-Dimethyl-2-(1-{4-[5-(5-methyl-1-{3-[(4-methylpiperazin-1-yl)carbonyl]benzyl}-1H-pyrazol-3-yl)-1,2,4-oxadiazol-3-yl]phenyl}cyclobutyl)acetamide

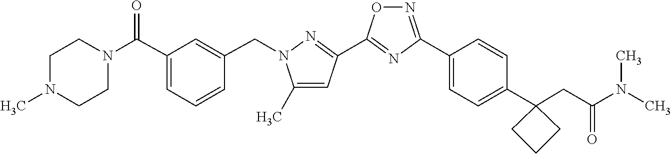

Analogously to the process described under Example 121, 300 mg (0.792 mmol) of the hydrochloride of the compound from Example 105A and 300 mg (0.871 mmol) of the compound from Example 117A were reacted to give 68 mg (15% of th.) of the title compound. After percolation over a bicarbonate cartridge to remove adhering formic acid from the HPLC purification, the product was finally purified by stirring with ethanol.

¹H-NMR (400 MHz, CDCl₃, δ/ppm): 8.11 (d, 2H), 7.38 (t, 1H), 7.33 (d, 1H), 7.31 (d, 2H), 7.20 (d, 1H), 7.15 (s, 1H), 6.82 (s, 1H), 5.47 (s, 2H), 3.76 (broad, 2H), 3.36 (broad, 2H), 2.83 (s, 2H), 2.73 (s, 3H), 2.60-2.52 (m, 2H), 2.51-2.40 (m, 4H), 2.35 (s, 3H), 2.30 (s, 3H), 2.28 (broad, 2H), 2.25 (s, 3H), 2.22-2.10 (m, 1H), 1.93-1.83 (m, 1H).

LC/MS (method I, ESIpos): Rₜ=0.85 min, m/z=582 [M+H]⁺.

### Example 132

### (3-{[3-(3-{4-[(Diisopropylamino)methyl]phenyl}-1,2,4-oxadiazol-5-yl)-5-methyl-1H-pyrazol-1-yl]methyl}phenyl)(4-methylpiperazin-1-yl)methanone

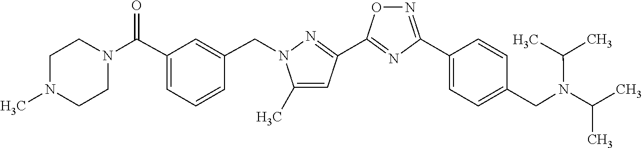 [SMILES: Cc1cc(-c2nc(-c3ccc(CN(C(C)C)C(C)C)cc3)no2)nn1Cc1cccc(C(=O)N2CCN(C)CC2)c1]

Analogously to the process described under Example 41, 100 mg (0.292 mmol) of the compound from Example 105A and 73 mg (0.292 mmol) of the compound from Example 118A were reacted to give 45 mg (28% of th.) of the title compound. Before purification of the reaction mixture by preparative HPLC (method O), the DMSO contained in the mixture was removed via a freeze drying.

¹H-NMR (400 MHz, CDCl₃, δ/ppm): 8.03 (d, 2H), 7.50-7.40 (m, 2H), 7.33-7.23 (m, 2H), 7.12 (d, 1H), 7.08 (s, 1H), 6.75 (s, 1H), 5.40 (s, 2H), 3.75-3.60 (m, broad, 4H), 3.35-3.25 (m, broad, 2H), 3.06-2.93 (m, broad, 2H), 2.42-2.30 (m, broad, 2H), 2.30-2.18 (m, broad, 2H), 2.22 (s, 3H), 2.20 (s, 3H), 1.08-0.89 (m, 12H).

LC/MS (method I, ESIpos): Rₜ=0.62 min, m/z=556 [M+H]⁺.

### Example 133

### (4-Methylpiperazin-1-yl) {3-[(5-methyl-3-{3-[4-(trifluoromethyl)phenyl]-1,2,4-oxadiazol-5-yl}-1H-pyrazol-1-yl)methyl]phenyl}methanone

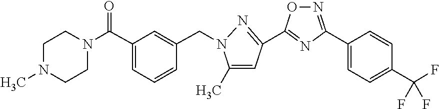 [SMILES: Cc1cc(-c2nc(-c3ccc(C(F)(F)F)cc3)no2)nn1Cc1cccc(C(=O)N2CCN(C)CC2)c1]

Analogously to the process described under Example 41, 100 mg (0.292 mmol) of the compound from Example 105A and 60 mg (0.292 mmol) of N′-hydroxy-4-(trifluoromethyl)-benzenecarboxamide amide were reacted to give 56 mg (38% of th.) of the title compound. Before purification of the reaction mixture by preparative HPLC (method O), the DMSO contained in the mixture was removed via a freeze drying.

¹H-NMR (400 MHz, CDCl₃, δ/ppm): 8.32 (d, 2H), 7.77 (d, 2H), 7.41-7.33 (m, 2H), 7.22-7.16 (m, 2H), 6.83 (s, 1H), 5.49 (s, 2H), 3.77 (s, broad, 2H), 3.37 (s, broad, 2H), 2.43 (s, broad, 2H), 2.31 (s, 3H), 2.33-2.23 (m, 2H), 2.28 (s, 3H).

LC/MS (method I, ESIpos): Rₜ=0.93 min, m/z=511 [M+H]⁺.

### Example 134

### (4-Methylpiperazin-1-yl) {3-[(5-methyl-3-{3-[4-(trimethylsilyl)phenyl]-1,2,4-oxadiazol-5-yl}-1H-pyrazol-1-yl)methyl]phenyl}methanone

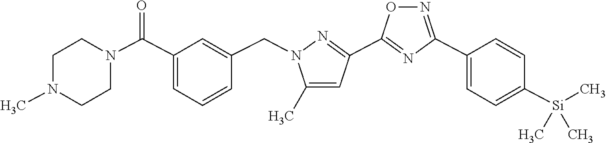

100 mg (0.292 mmol) of the compound from Example 105A were initially introduced into 3 ml of methylene chloride at 0° C., one drop of DMF was added and 76 μl (0.876 mmol) of oxalyl chloride were then added dropwise. The mixture was stirred at RT for 1 h and subsequently concentrated and the residue was dried in vacuo. The residue was then dissolved again in 2 ml of methylene chloride and the solution was added to a mixture of 68 mg (0.292 mmol, purity of 90%) of the compound from Example 17A and 81 μl (0.584 mmol) of triethylamine in 1 ml of methylene chloride at 0° C. The mixture was stirred at RT for 1 h and concentrated again and the residue was dried in vacuo. The residue was then dissolved in 3 ml of DMSO and the solution was heated at 120° C. in a microwave apparatus (CEM Discover, initial irradiation power 250 W) for 30 min. After cooling to RT, the reaction mixture was purified directly by means of preparative HPLC (method O). The product fractions were combined and concentrated to a residual volume of water. Saturated aqueous sodium bicarbonate solution was added and the mixture was extracted twice with ethyl acetate. The combined ethyl acetate phases were dried over magnesium sulfate, filtered and concentrated. After the residue had been dried in vacuo, 76 mg (50% of th.) of the title compound were obtained.

¹H-NMR (400 MHz, CDCl₃, δ/ppm): 8.18 (d, 2H), 7.65 (d, 2H), 7.41-7.32 (m, 2H), 7.21 (d, 1H), 7.17 (s, 1H), 6.82 (s, 1H), 5.49 (s, 2H), 3.78 (s, broad, 2H), 3.38 (s, broad, 2H), 2.45 (s, broad, 2H), 2.31 (s, 3H), 2.33-2.23 (m, 2H), 2.28 (s, 3H), 0.31 (s, 9H).

LC/MS (method I, ESIpos): Rₜ=1.03 min, m/z=515 [M+H]⁺.

### Example 135

### (3-{[5-Methyl-3-(3-{4-[N-methyl-S-(trifluoromethyl)sulfonimidoyl]phenyl}-1,2,4-oxadiazol-5-yl)-1H-pyrazol-1-yl]methyl}phenyl)(4-methylpiperazin-1-yl)methanone (racemate)

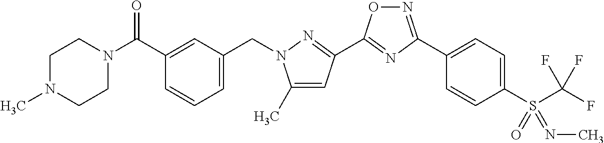 [SMILES: CN=S(=O)(c1ccc(-c2noc(-c3cc(C)n(Cc4cccc(C(=O)N5CCN(C)CC5)c4)n3)n2)cc1)C(F)(F)F]

162 mg (0.427 mmol) of the compound from Example 105A were initially introduced into 4 ml of methylene chloride, 0.74 ml (8.53 mmol) of oxalyl chloride was added and the mixture was stirred at RT for 1 h. The mixture was subsequently concentrated and the residue was dried in vacuo. The residue was then dissolved in 2 ml of methylene chloride, a solution of 120 mg (0.427 mmol) of the compound from Example 119A and 0.18 ml (1.28 mmol) of triethylamine in 1 ml of methylene chloride was added and the mixture was stirred at RT for 1 h. The mixture was then concentrated and the residue was dried in vacuo. The residue was then dissolved in 3 ml of DMSO and the solution was heated at 120° C. for 1.5 h, while stirring. After cooling to RT, the reaction mixture was purified directly by means of preparative HPLC (method O). The combined product fractions were concentrated to a small residual volume of aqueous phase, saturated aqueous sodium bicarbonate solution was added and the mixture was extracted three times with ethyl acetate. The combined ethyl acetate phases were dried over sodium sulfate, filtered and concentrated. After drying the residue, 48 mg (19% of th.) of the title compound were obtained.

¹H-NMR (400 MHz, CDCl₃, δ/ppm): 8.44 (d, 2H), 8.21 (d, 2H), 7.44-7.30 (m, 2H), 7.23-7.13 (m, 2H), 6.85 (s, 1H), 5.48 (s, 2H), 3.76 (s, broad, 2H), 3.37 (s, broad, 2H), 3.12 (s, 3H), 2.50-2.36 (m, 2H), 2.35-2.25 (m, broad, 2H), 2.30 (s, 3H), 2.27 (s, 3H).

LC/MS (method I, ESIpos): Rₜ=0.94 min, m/z=588 [M+H]⁺.

### Example 136

### {3-[(3-{3-[3-Chloro-4-(trifluoromethoxy)phenyl]-1,2,4-oxadiazol-5-yl}-5-methyl-1H-pyrazol-1-yl)methyl]phenyl}(4-methylpiperazin-1-yl)methanone

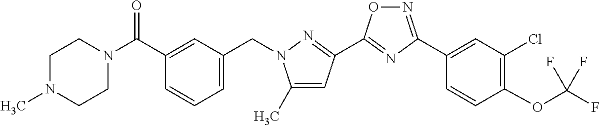 [SMILES: Cc1cc(-c2nc(-c3ccc(OC(F)(F)F)c(Cl)c3)no2)nn1Cc1cccc(C(=O)N2CCN(C)CC2)c1]

Analogously to the process described under Example 41, 200 mg (0.528 mmol) of the compound from Example 105A and 134 mg (0.528 mmol) of the compound from Example 120A were reacted to give 11.2 mg (4% of th.) of the title compound.

¹H-NMR (400 MHz, CDCl₃, δ/ppm): 8.37 (d, 1H), 8.19-8.05 (m, 1H), 7.49-7.30 (m, 3H), 7.23-7.11 (m, 2H), 6.83 (s, 1H), 5.48 (s, 2H), 3.80 (s, broad, 2H), 3.42 (s, broad, 2H), 2.60-2.30 (m, broad, 4H), 2.32 (s, 3H), 2.30 (s, 3H).

LC/MS (method I, ESIpos): Rₜ=1.01 min, m/z=561/563 [M+H]⁺.

### Example 137

### (4-Methylpiperazin-1-yl)(3-{[5-methyl-3-(3-{4-[1-(trifluoromethyl)cyclopropyl]phenyl}-1,2,4-oxadiazol-5-yl)-1H-pyrazol-1-yl]methyl}phenyl)methanone

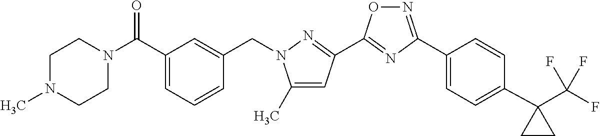 [SMILES: Cc1cc(-c2nc(-c3ccc(C4(C(F)(F)F)CC4)cc3)no2)nn1Cc1cccc(C(=O)N2CCN(C)CC2)c1]

450 mg (1.19 mmol) of the compound from Example 105A were initially introduced into 12 ml of methylene chloride at 0° C., one drop of DMF was added and 311 μl (3.56 mmol) of oxalyl chloride were then added dropwise. The mixture was stirred at RT for 1 h and subsequently concentrated and the residue was dried in vacuo. The residue was then taken up in 8 ml of methylene chloride and this solution was added to a mixture of 290 mg (1.19 mmol) of the compound from Example 121A and 331 μl (2.38 mmol) of triethylamine in 4 ml of methylene chloride at 0° C. The mixture was stirred at RT for 1 h and then concentrated and the residue was dried in vacuo. The residue was then dissolved in 12 ml of DMSO and the mixture was heated at 120° C. in a microwave apparatus (CEM Discover, initial irradiation power 250 W) for 30 min. After cooling, the reaction mixture was purified directly by means of preparative HPLC (method O). The combined product fractions were concentrated to a residual volume of aqueous phase. Saturated aqueous sodium bicarbonate solution was added and the mixture was extracted twice with ethyl acetate. The combined ethyl acetate phases were dried over magnesium sulfate, filtered and concentrated. After the residue had been dried in vacuo, 109 mg (17% of th.) of the title compound were obtained.

¹H-NMR (400 MHz, CDCl₃, δ/ppm): 8.10 (d, 2H), 7.52 (d, 2H), 7.38-7.23 (m, 2H), 7.18-7.05 (m, 2H), 6.76 (s, 1H), 5.40 (s, 2H), 3.70 (s, broad, 2H), 3.30 (s, broad, 2H), 2.49-2.07 (m, 4H), 2.22 (s, 3H), 2.19 (s, 3H), 1.34 (s, broad, 2H), 1.02 (s, broad, 2H).

LC/MS (method I, ESIpos): Rₜ=0.97 min, m/z=551 [M+H]⁺.

### Example 138

### [3-({3-[5-(4-Cyclopropylphenyl)-1,2,4-oxadiazol-3-yl]-5-methyl-1H-pyrazol-1-yl}methyl)phenyl]-(4-methylpiperazin-1-yl)methanone

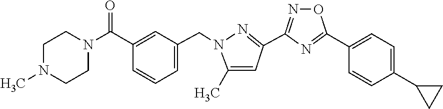 [SMILES: Cc1cc(-c2noc(-c3ccc(C4CC4)cc3)n2)nn1Cc1cccc(C(=O)N2CCN(C)CC2)c1]

68 mg (0.421 mmol) of 4-cyclopropylbenzoic acid were initially introduced into 2 ml of methylene chloride, one drop of DMF was added and the mixture was cooled to 0° C. 160 mg (1.26 mmol) of oxalyl chloride were then added at this temperature and the mixture was subsequently stirred at 40° C. for 20 min. 5 ml of methylene chloride were then added, the mixture was concentrated and the residue was dried in vacuo. The residue was taken up in 3 ml of methylene chloride and the mixture was added to a mixture of 150 mg (0.421 mmol) of the compound from Example 148A and 140 μl of triethylamine in 5 ml of methylene chloride at RT. The mixture was stirred at RT for 3 h and concentrated again and the residue was dried in vacuo. The residue was then dissolved in 2 ml of dry DMSO and the solution was heated at 140° C. in a microwave apparatus (CEM Discover, initial irradiation power 250 W) for 1 h. After cooling to RT, 60 ml of water were added and the mixture was extracted three times with 30 ml of ethyl acetate each time. The combined ethyl acetate phases were washed once with saturated sodium chloride solution, dried over magnesium sulfate, filtered and concentrated. The residue was taken up in acetonitrile and purified by means of preparative HPLC (method O). The combined product fractions were concentrated to a residual volume of aqueous phase, saturated aqueous sodium bicarbonate solution was added and the mixture was extracted three times with approx. 30 ml of methylene chloride each time. The combined methylene chloride phases were washed once with water, dried over magnesium sulfate, filtered and concentrated. After the residue had been dried in vacuo, 65 mg (32% of th.) of the title compound were obtained.

¹H-NMR (400 MHz, CDCl₃, δ/ppm): 8.13 (d, 2H), 7.44-7.30 (m, 2H), 7.23-7.15 (m, 3H), 7.12 (s, 1H), 6.74 (s, 1H), 5.47 (s, 2H), 3.82-3.58 (m, 4H), 3.45-3.28 (s, broad, 2H), 2.54-2.34 (m, 2H), 2.28 (s, 3H), 2.22 (s, 3H), 2.04-1.90 (m, 1H), 1.14-1.01 (m, 2H), 0.85-0.74 (m, 2H).

LC/MS (method I, ESIpos): Rₜ=0.91 min, m/z=483 [M+H]⁺.

### Example 139

### (3-{[3-(3-{4-[4-(Fluoromethyl)tetrahydro-2H-pyran-4-yl]phenyl}-1,2,4-oxadiazol-5-yl)-5-methyl-1H-pyrazol-1-yl]methyl}phenyl)(4-methylpiperazin-1-yl)methanone

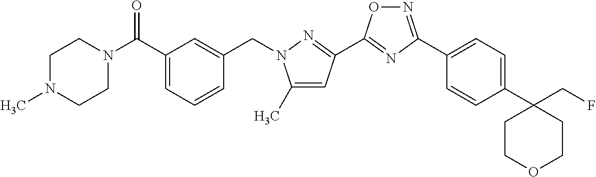 [SMILES: Cc1cc(-c2nc(-c3ccc(C4(CF)CCOCC4)cc3)no2)nn1Cc1cccc(C(=O)N2CCN(C)CC2)c1]

74 μl (0.53 mmol) of triethylamine, 51 mg (0.26 mmol) of EDC and 40 mg (0.26 mmol) of HOBt were added successively to 100 mg (0.26 mmol) of the hydrochloride of the compound from Example 105A in 2.6 ml of DMF. After stirring at RT for 10 minutes, 66 mg (0.26 mmol) of the compound from Example 123A were added and the mixture was first stirred at RT for a further 10 min and then heated at 140° C. for 30 min. After cooling, the reaction mixture was purified directly by means of preparative HPLC (method P). 7.0 mg (5% of th.) of the title compound were obtained.

¹H-NMR (400 MHz, DMSO-d₆, δ/ppm): 8.01 (d, 2H), 7.44 (m, 3H), 7.32 (t, 2H), 7.15 (s, 1H), 6.94 (s, 1H), 5.55 (s, 2H), 3.72 (d, broad, 2H), 3.62-3.45 (m, broad, 4H), 3.24 (m, broad, 2H), 3.05 (d, 2H), 2.34 (s, 3H), 2.37-2.12 (m, broad, 4H), 2.12 (s, 3H), 1.84-1.65 (m, 2H), 1.59 (t, 2H).

LC/MS (method D, ESIpos): Rₜ=1.75 min, m/z=559 [M+H]⁺.

### Example 140

### 1-({3-[(5-Methyl-3-{3-[4-(1,1,1-trifluoro-2-methylpropan-2-yl)phenyl]-1,2,4-oxadiazol-5-yl}-1H-pyrazol-1-yl)methyl]phenyl}sulfonyl)piperidin-4-ol

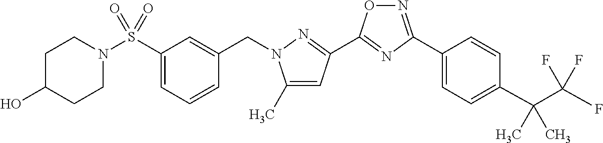 [SMILES: Cc1cc(-c2nc(-c3ccc(C(C)(C)C(F)(F)F)cc3)no2)nn1Cc1cccc(S(=O)(=O)N2CCC(O)CC2)c1]

A solution of 162 mg (0.34 mmol) of the compound from Example 136A in 1 ml of THF was added to a solution of 95 mg (0.28 mmol) of the compound from Example 29A and 35 mg (0.31 mmol) of potassium tert-butylate in 3 ml of THF, while cooling in an ice bath, and the mixture was then stirred at RT overnight. It was then diluted with ethyl acetate, and magnesium sulfate was added. After filtration, the filtrate was freed from the solvent on a rotary evaporator and the residue obtained was purified by means of preparative HPLC (method P). 77 mg (38% of th.) of the title compound were obtained.

¹H-NMR (400 MHz, DMSO-d₆, δ/ppm): 8.09 (d, 2H), 7.77 (d, 2H), 7.70-7.60 (m, 3H), 7.49 (d, 1H), 6.96 (s, 1H), 5.66 (s, 2H), 4.65 (d, 1H), 3.50 (m, 1H), 3.11 (m, 2H), 2.69 (m, 2H), 2.35 (s, 3H), 1.69 (m, 2H), 1.61 (s, 6H), 1.40 (m, 2H).

LC/MS (method I): Rₜ=1.24 min, m/z=590 [M+H]⁺.

### Example 141

### 1-Methyl-4-({3-[(5-methyl-3-{3-[4-(1,1,1-trifluoro-2-methylpropan-2-yl)phenyl]-1,2,4-oxadiazol-5-yl}-1H-pyrazol-1-yl)methyl]phenyl}sulfonyl)piperazine

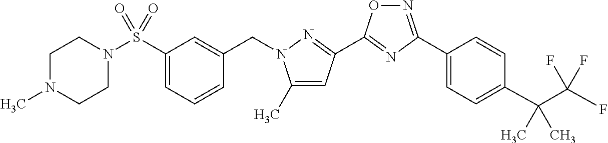 [SMILES: Cc1cc(-c2nc(-c3ccc(C(C)(C)C(F)(F)F)cc3)no2)nn1Cc1cccc(S(=O)(=O)N2CCN(C)CC2)c1]

Analogously to the process described under Example 140, 126 mg (43% of th.) of the title compound were obtained from 140 mg (0.42 mmol) of the compound from Example 29A and 1.0 ml (approx. 0.5 mmol) of the intermediate solution in THF obtained in Example 137A.

¹H-NMR (400 MHz, DMSO-d₆, δ/ppm): 8.09 (d, 2H), 7.77 (d, 2H), 7.69-7.64 (m, 2H), 7.57 (s, 1H), 7.53 (d, 1H), 6.97 (s, 1H), 5.66 (s, 2H), 2.84 (s, 4H), 2.35 (s, 3H), 2.29 (s, 4H), 2.07 (s, 3H), 1.61 (s, 6H).

LC/MS (method I): Rₜ=1.02 min, m/z=589 [M+H]⁺.

### Example 142

### 1-Methyl-4-({3-[(5-methyl-3-{3-[4-(trifluoromethoxy)phenyl]-1,2,4-oxadiazol-5-yl}-1H-pyrazol-1-yl)methyl]phenyl}sulfonyl)piperazine

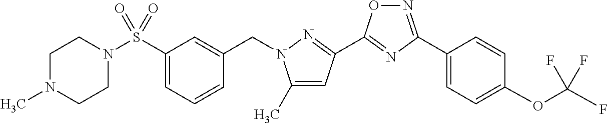 [SMILES: Cc1cc(-c2nc(-c3ccc(OC(F)(F)F)cc3)no2)nn1Cc1cccc(S(=O)(=O)N2CCN(C)CC2)c1]

Analogously to the process described under Example 140, 75 mg (27% of th.) of the title compound were obtained from 129 mg (0.42 mmol) of the compound from Example 28A and 1.0 ml (approx. 0.5 mmol) of the intermediate solution in THF obtained in Example 137A.

¹H-NMR (400 MHz, DMSO-d₆, δ/ppm): 8.20 (d, 2H), 7.69-7.64 (m, 2H), 7.61 (s, 1H), 7.58 (d, 2H), 7.53 (d, 1H), 6.96 (s, 1H), 5.66 (s, 2H), 2.84 (s, 4H), 2.36 (s, 3H), 2.29 (s, 4H), 2.07 (s, 3H).

LC/MS (method I): Rₜ=0.98 min, m/z=563 [M+H]⁺.

### Example 143

### [3-({4-[3-(4-tert-Butylphenyl)-1,2,4-oxadiazol-5-yl]-2-methyl-1H-pyrrol-1-yl}methyl)phenyl]-(4-methylpiperazin-1-yl)methanone

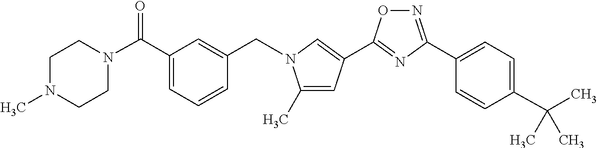 [SMILES: Cc1cc(-c2nc(-c3ccc(C(C)(C)C)cc3)no2)cn1Cc1cccc(C(=O)N2CCN(C)CC2)c1]

100 mg (0.309 mmol) of a 21% strength solution of sodium ethanolate in mineral ethanol were added to a suspension of 100 mg (0.281 mmol) of the compound from Example 106A/step 3 and 60 mg (0.309 mmol) of 4-tert-butyl-N′-hydroxybenzamidine in 3 ml of ethanol. The mixture was heated at 160° C. in a microwave oven (CEM Discover, initial irradiation power 250 W) for 30 min.

After cooling to RT, the reaction batch was separated directly into its components by means of preparative HPLC (method N). The product fractions were combined and concentrated to dryness on a rotary evaporator. The residue obtained was dissolved in approx. 5 ml of methanol and the solution was passed over a bicarbonate cartridge (Polymerlabs, Stratospheres SPE, PL-HCO₃ MP SPE, capacity 0.9 mmol) in order to remove adhering formic acid from the HPLC purification. After concentration and drying, 6.4 mg (5% of th.) of the title compound were obtained.

¹H-NMR (400 MHz, CDCl₃, δ/ppm): 8.03 (d, 2H), 7.49 (d, 2H), 7.48 (s, 1H), 7.39 (t, 1H), 7.33 (d, 1H), 7.09 (d, 1H), 7.08 (s, 1H), 6.58 (s, 1H), 5.11 (s, 2H), 3.76 (broad, 2H), 3.37 (broad, 2H), 2.44 (broad, 2H), 2.28 (broad, 2H), 2.26 (s, 3H), 2.19 (s, 3H), 1.35 (s, 9H).

LC/MS (method I, ESIpos): Rₜ=1.06 min, m/z=498 [M+H]⁺.

### Example 144

### (4-Methylpiperazin-1-yl){3-[(2-methyl-4-{3-[4-(1,1,1-trifluoro-2-methylpropan-2-yl)phenyl]-1,2,4-oxadiazol-5-yl}-1H-pyrrol-1-yl)methyl]phenyl}methanone

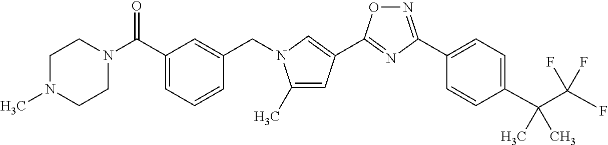 [SMILES: Cc1cc(-c2nc(-c3ccc(C(C)(C)C(F)(F)F)cc3)no2)cn1Cc1cccc(C(=O)N2CCN(C)CC2)c1]

Analogously to the process described under Example 41, 100 mg (0.293 mmol) of the compound from Example 106A and 79 mg (0.322 mmol) of the compound from Example 1A were reacted to give 21 mg (13% of th.) of the title compound.

¹H-NMR (400 MHz, CDCl₃, δ/ppm): 8.10 (d, 2H), 7.60 (d, 2H), 7.48 (s, 1H), 7.39 (t, 1H), 7.33 (d, 1H), 7.10 (d, 1H), 7.09 (s, 1H), 6.59 (s, 1H), 5.12 (s, 2H), 3.77 (broad, 2H), 3.37 (broad, 2H), 2.44 (broad, 2H), 2.28 (broad, 2H), 2.26 (s, 3H), 2.19 (s, 3H), 1.62 (s, 6H).

LC/MS (method F, ESIpos): Rₜ=1.21 min, m/z=552 [M+H]⁺.

### Example 145

### (4-Methylpiperazin-1-yl) {3-[(2-methyl-4-{3-[4-(trimethylsilyl)phenyl]-1,2,4-oxadiazol-5-yl}-1H-pyrrol-1-yl)methyl]phenyl}methanone

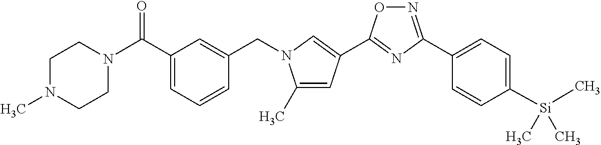

Analogously to the process described under Example 41, 100 mg (0.293 mmol) of the compound from Example 106A and 67 mg (0.322 mmol) of the compound from Example 17A were reacted to give 11 mg (7.4% of th.) of the title compound.

¹H-NMR (400 MHz, CDCl₃, δ/ppm): 8.08 (d, 2H), 7.62 (d, 2H), 7.49 (s, 1H), 7.39 (t, 1H), 7.33 (d, 1H), 7.10 (d, 1H), 7.09 (s, 1H), 6.59 (s, 1H), 5.12 (s, 2H), 3.76 (broad, 2H), 3.37 (broad, 2H), 2.43 (broad, 2H), 2.29 (broad, 2H), 2.25 (s, 3H), 2.19 (s, 3H), 0.30 (s, 9H).

LC/MS (method F, ESIpos): Rₜ=1.27 min, m/z=514 [M+H]⁺.

### Example 146

### {3-[(4-{3-[4-(4-Fluorotetrahydro-2H-pyran-4-yl)phenyl]-1,2,4-oxadiazol-5-yl}-2-methyl-1H-pyrrol-1-yl)methyl]phenyl}(4-methylpiperazin-1-yl)methanone

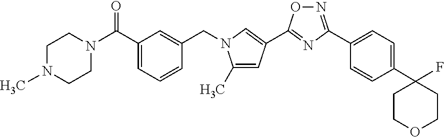 [SMILES: Cc1cc(-c2nc(-c3ccc(C4(F)CCOCC4)cc3)no2)cn1Cc1cccc(C(=O)N2CCN(C)CC2)c1]

Analogously to the process described under Example 41, 120 mg (0.351 mmol) of the compound from Example 106A and 92 mg (0.387 mmol) of the compound from Example 7A were reacted to give 20 mg (10% of th.) of the title compound.

¹H-NMR (400 MHz, CDCl₃, δ/ppm): 8.14 (d, 2H), 7.50 (d, 2H), 7.49 (s, 1H), 7.39 (t, 1H), 7.33 (d, 1H), 7.10 (d, 1H), 7.09 (s, 1H), 6.59 (s, 1H), 5.12 (s, 2H), 3.99-3.86 (m, 4H), 3.76 (broad, 2H), 3.37 (broad, 2H), 2.44 (broad, 2H), 2.29 (broad, 2H), 2.26 (s, 3H), 2.23-2.10 (m, 2H), 2.18 (s, 3H), 1.97-1.91 (m, 2H).

LC/MS (method I, ESIpos): Rₜ=0.90 min, m/z=544 [M+H]⁺.

### Example 147

### (4-Methylpiperazin-1-yl) {3-[(2-methyl-4-{3-[4-(trifluoromethyl)phenyl]-1,2,4-oxadiazol-5-yl}-1H-pyrrol-1-yl)methyl]phenyl}methanone

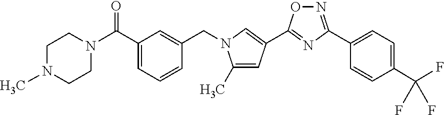 [SMILES: Cc1cc(-c2nc(-c3ccc(C(F)(F)F)cc3)no2)cn1Cc1cccc(C(=O)N2CCN(C)CC2)c1]

Analogously to the process described under Example 41, 120 mg (0.351 mmol) of the compound from Example 106A and 79 mg (0.387 mmol) of 4-trifluoromethyl-N′-hydroxybenzamidine were reacted to give 15 mg (8.4% of th.) of the title compound.

¹H-NMR (400 MHz, CDCl₃, δ/ppm): 8.25 (d, 2H), 7.74 (d, 2H), 7.49 (s, 1H), 7.40 (t, 1H), 7.33 (d, 1H), 7.11-7.08 (m, 2H), 6.59 (s, 1H), 5.13 (s, 2H), 3.77 (broad, 2H), 3.37 (broad, 2H), 2.43 (broad, 2H), 2.29 (broad, 2H), 2.27 (s, 3H), 2.20 (s, 3H).

LC/MS (method I, ESIpos): Rₜ=0.99 min, m/z=510 [M+H]⁺.

### Example 148

### 1-Methyl-4-{3-[(5-methyl-3-{3-[4-(trifluoromethoxy)phenyl]-1,2,4-oxadiazol-5-yl}-1H-pyrazol-1-yl)methyl]phenoxy}piperidine

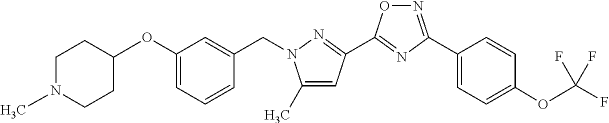 [SMILES: Cc1cc(-c2nc(-c3ccc(OC(F)(F)F)cc3)no2)nn1Cc1cccc(OC2CCN(C)CC2)c1]

First 30 mg (0.264 mmol) of 4-hydroxy-1-methylpiperidine and, after 5 min, 100 mg (0.240 mmol) of the compound from Example 95A were added to a solution of 69 mg (0.264 mmol) of triphenylphosphine and 52 μl (0.264 mmol) of diisopropyl azodicarboxylate (DIAD) in 3 ml of anhydrous THF. After the reaction mixture had been stirred at RT for 16 h, the same amount of DIAD was again added. After a further 5 days at RT, a further 30 mg (0.264 mmol) of 4-hydroxy-1-methylpiperidine were added. After 16 h again at RT, 1 ml of water and approx. 3 ml of DMF were added to the reaction mixture. This solution was separated directly into its components by means of preparative HPLC (method N). The product fractions were combined and freed from the solvent on a rotary evaporator. The product obtained in this way was subsequently purified again by means of MPLC (silica gel, mobile phase: methylene chloride/methanol 10:1). 32 mg (26% of th.) of the title compound were obtained.

¹H-NMR (400 MHz, CDCl₃, δ/ppm): 8.25 (d, 2H), 7.33 (d, 2H), 7.22 (t, 1H), 6.82 (d, 1H), 6.81 (s, 1H), 6.72 (d, 1H), 6.71 (s, 1H), 5.40 (s, 2H), 4.31-4.24 (m, 1H), 2.69 (broad, 2H), 2.30 (s, 3H), 2.29 (broad, 2H), 2.28 (s, 3H), 1.98 (broad, 2H), 1.81 (broad, 2H).

HPLC (method A): Rₜ=4.41 min.

LC/MS (method I, ESIpos): Rₜ=0.99 min, m/z=514 [M+H]⁺.

### Example 149

### 2-[4-(Methoxymethyl)piperidin-1-yl]-4-[(5-methyl-3-{3-[4-(trifluoromethoxy)phenyl]-1,2,4-oxa-diazol-5-yl}-1H-pyrazol-1-yl)methyl]pyridine

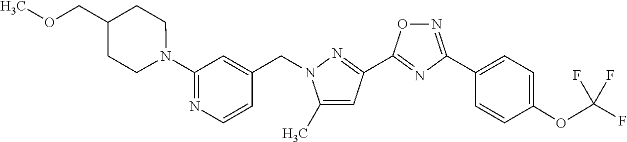 [SMILES: COCC1CCN(c2cc(Cn3nc(-c4nc(-c5ccc(OC(F)(F)F)cc5)no4)cc3C)ccn2)CC1]

445 mg (3.44 mmol) of 4-(methoxymethyl)pyridine and 100 mg (0.229 mmol) of the compound from Example 81A were heated at 160° C. in a microwave oven (Biotage Initiator 2.5, automatic control of the irradiation power) for 3 h without addition of a solvent. After cooling to RT, the reaction mixture was taken up in approx. 2 ml of methanol. This solution was separated directly into its components by means of preparative HPLC (method N). The product fractions were combined and freed from the solvent and the residue was stirred with pentane. 77 mg (60% of th., purity of approx. 94%) of the title compound were obtained.

¹H-NMR (400 MHz, CDCl₃, δ/ppm): 8.25 (d, 2H), 8.11 (d, 1H), 7.33 (d, 2H), 6.83 (s, 1H), 6.35 (s, 1H), 6.29 (d, 1H), 5.33 (s, 2H), 4.26-4.20 (m, 2H), 3.33 (s, 3H), 3.23 (d, 2H), 2.83-2.77 (m, 2H), 2.29 (s, 3H), 1.88-1.77 (m, 3H), 1.29-1.18 (m, 2H).

LC/MS (method I, ESIpos): Rₜ=1.09 min, m/z=529 [M+H]⁺.

### Example 150

### 2-(4-Methoxypiperidin-1-yl)-4-[(5-methyl-3-{3-[4-(trifluoromethoxy)phenyl]-1,2,4-oxadiazol-5-yl}-1H-pyrazol-1-yl)methyl]pyridine

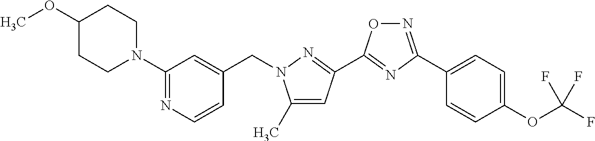 [SMILES: COC1CCN(c2cc(Cn3nc(-c4nc(-c5ccc(OC(F)(F)F)cc5)no4)cc3C)ccn2)CC1]

1.06 g (9.18 mmol) of 4-methoxypyridine and 200 mg (0.229 mmol) of the compound from Example 81A were heated at 160° C. in a microwave oven (Biotage Initiator 2.5, automatic control of the irradiation power) for 3 h without addition of a solvent. After cooling to RT, approx. 50 ml of water were added to the reaction mixture and the mixture was extracted three times with approx. 50 ml of ethyl acetate each time. The combined organic extracts were washed successively with water and saturated sodium chloride solution. After drying over anhydrous magnesium sulfate, the mixture was filtered and the filtrate was freed from the solvent on a rotary evaporator. The crude product was purified by means of MPLC (approx. 50 g of silica gel, mobile phase: cyclohexane/ethyl acetate 3:1→1:1). 167 mg (70% of th.) of the title compound were obtained.

¹H-NMR (400 MHz, CDCl₃, δ/ppm): 8.26 (d, 2H), 8.11 (d, 1H), 7.33 (d, 2H), 6.83 (s, 1H), 6.36 (s, 1H), 6.31 (d, 1H), 5.33 (s, 2H), 3.93-3.87 (m, 2H), 3.44-3.38 (m, 1H), 3.37 (s, 3H), 3.21-3.14 (m, 2H), 2.29 (s, 3H), 1.97-1.90 (m, 2H), 1.63-1.54 (m, 2H).

LC/MS (method I, ESIpos): Rₜ=1.10 min, m/z=515 [M+H]⁺.

### Example 151

### 6-{4-[(5-Methyl-3-{3-[4-(trifluoromethoxy)phenyl]-1,2,4-oxadiazol-5-yl}-1H-pyrazol-1-yl)-methyl]pyridin-2-yl}-2-oxa-6-azaspiro[3.3]heptane

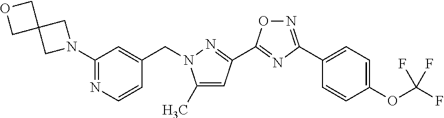 [SMILES: Cc1cc(-c2nc(-c3ccc(OC(F)(F)F)cc3)no2)nn1Cc1ccnc(N2CC3(COC3)C2)c1]

662 mg (2.30 mmol) of 2-oxa-6-azaspiro[3.3]heptane hemioxalate [M. Roger-Evans et al., Angew. Chem. Intl. Ed. Engl. 2008, 47 (24), 4512-4515], 100 mg (0.229 mmol) of the compound from Example 81A and 0.8 ml (4.59 mmol) of N,N-diisopropylethylamine were dissolved in 2.5 ml of methanol and the solution was first automatically controlled to 140° C. in a microwave oven (Biotage Initiator 2.5, automatic control of the irradiation power). When this temperature was reached, the temperature was increased to 160° C. by manual control. After 15 h at 160° C., the mixture was allowed to cool to RT. The reaction mixture was diluted with a further approx. 3 ml of methanol and separated directly into its components via preparative HPLC (method N). The product fractions were combined and freed from the solvent on a rotary evaporator. The residue was then chromatographed over a Chromabond cartridge for further purification (1.5 g of silica gel, mobile phase: cyclohexane/ethyl acetate 1:1→1:5). 22 mg (19% of th.) of the title compound were obtained in this way.

¹H-NMR (400 MHz, CDCl₃, δ/ppm): 8.25 (d, 2H), 8.09 (d, 1H), 7.34 (d, 2H), 6.83 (s, 1H), 6.37 (d, 1H), 5.95 (s, 1H), 5.33 (s, 2H), 4.81 (s, 4H), 4.13 (s, 4H), 2.28 (s, 3H).

LC/MS (method D, ESIpos): Rₜ=1.89 min, m/z=499 [M+H]⁺.

### Example 152

### 2-(1-{4-[(5-Methyl-3-{3-[4-(trifluoromethoxy)phenyl]-1,2,4-oxadiazol-5-yl}-1H-pyrazol-1-yl)-methyl]pyridin-2-yl}piperidin-4-yl)propan-2-ol

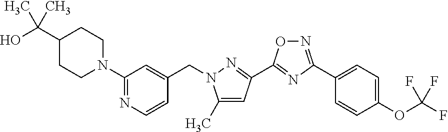 [SMILES: Cc1cc(-c2nc(-c3ccc(OC(F)(F)F)cc3)no2)nn1Cc1ccnc(N2CCC(C(C)(C)O)CC2)c1]

Analogously to the process described under Example 149, 493 mg (3.44 mmol) of 2-(piperidin-4-yl)propan-2-ol and 100 mg (0.229 mmol) of the compound from Example 81A were reacted to give 40 mg (32% of th.) of the title compound. The product obtained after purification via preparative HPLC was finally stirred with ethanol (instead of pentane).

¹H-NMR (400 MHz, CDCl₃, δ/ppm): 8.25 (d, 2H), 8.11 (d, 1H), 7.33 (d, 2H), 6.83 (s, 1H), 6.35 (s, 1H), 6.30 (d, 1H), 5.33 (s, 2H), 4.36-4.30 (m, 2H), 2.77-2.70 (m, 2H), 2.29 (s, 3H), 1.87-1.80 (m, 2H), 1.53-1.47 (m, 1H), 1.38-1.28 (m, 2H), 1.23 (s, broad, 1H), 1.18 (s, 6H).

LC/MS (method I, ESIpos): Rₜ=1.03 min, m/z=543 [M+H]⁺.

### B. EVALUATION OF THE PHARMACOLOGICAL ACTIVITY

The pharmacological activity of the compounds according to the invention can be demonstrated by in vitro and in vivo studies such as are known to the person skilled in the art. The usefulness of the substances according to the invention can be illustrated by way of example by in vitro (tumour) cell experiments and in vivo tumour models such as are described below. The connection between an inhibition of the HIF transcription activity and the inhibition of tumour growth is demonstrated by numerous studies described in the literature (cf. e.g. Warburg, 1956; Semenza, 2007).

### B-1. HIF-Luciferase Assay

HCT 116 cells were transfected in a stable manner with a plasmid which contained a luciferase reporter under the control of an HIF-responsive sequence. These cells were sown in microtitre plates [20,000 cells/cavity in RPMI 1640 medium with 10% foetal calf serum (FCS) and 100 μg/ml of hygromycin]. Incubation was carried out overnight under standard conditions (5% CO₂, 21% O₂, 37° C., moistened). The following morning the cells were incubated with various concentrations of the test substances (0-10 μmol/l) in a hypoxia chamber (1% O₂). After 24 h, Bright Glo reagent (Promega, Wis., USA) was added in accordance with the manufacturer'/s instructions, and after 5 min the luminescence was measured. Cells which were incubated under normoxia served as background controls.

The IC₅₀ values from this assay for representative embodiment examples are listed in the following table:

| Example no. | IC₅₀ [nmol/l] |
|---|---|
| 16 | 4 |
| 18 | 5 |
| 21 | 10 |
| 35 | 2 |
| 41 | 6 |
| 45 | 10 |
| 52 | 3 |
| 65 | 0.6 |
| 71 | 1 |
| 72 | 1 |
| 75 | 1 |
| 77 | 1 |
| 78 | 0.5 |
| 85 | 2 |
| 86 | 4 |
| 91 | 0.6 |
| 93 | 0.8 |
| 100 | 2.5 |
| 119 | 20 |
| 137 | 3 |
| 140 | 4 |
| 150 | 3 |

### B-2. Suppression of HIF Target Genes In Vitro

Human bronchial carcinoma cells (H460 and A549) were incubated for 16 h with variable concentrations of the test substances (1 nM to 10 μM) under normoxic conditions and under a 1% oxygen partial pressure (see HIF-luciferase assay). The total RNA was isolated from the cells and transcribed into cDNA and the mRNA expression of HIF target genes was analyzed in real time PCR. Active test substances already lower the mRNA expression of the HIF target genes compared with untreated cells under normoxic conditions, but above all under hypoxic conditions.

### B-3. Human Xenograft and Syngenic Tumour Models

Human tumour xenograft models in immunodeficient mice and syngenic tumour mouse models were used for evaluation of the substances. For this, tumour cells were cultured in vitro and implanted subcutaneously, or tumour xenotransplant pieces were transplanted further subcutaneously. The animals were treated by oral, subcutaneous or intraperitoneal therapy after the tumour was established. The activity of the test substances was analyzed in monotherapy and in combination therapy with other pharmacological active substance. The tumour inhibitory potency of the test substance on tumours of advanced size (approx. 100 mm²) was moreover characterized. The state of health of the animals was checked daily, and the treatments were performed in accordance with animal protection regulations. The tumour area was measured with slide gauges (length L, breadth B=shorter dimension). The tumour volume was calculated from the formula (L×B²)/2. The inhibition in tumour growth was determined at the end of the study as the T/C ratio of the tumour areas and tumour weights and as the TGI value (tumour growth inhibition, calculated from the formula [1−(T/C)]×100) (T=tumour size in the treated group; C=tumour size in the untreated control group).

The influence of the test substances on the tumour vessel architecture and the blood flow within the tumour was identified with the aid of computer microtomography and ultrasound microstudies on treated and untreated tumour-carrying mice.

### B-4. Determination of Pharmacokinetic Parameters Following Intravenous and Peroral Administration

The substance to investigated was administered to animals (e.g. mice or rats) intravenously as a solution (e.g. in corresponding plasma with a small addition of DMSO or in a PEG/ethanol/water mixture), and peroral administration took place as a solution (e.g. in a Solutol/ethanol/water or PEG/ethanol/water mixture) or as a suspension (e.g. in tylose), in each case via a stomach tube. After administration of the substance, blood was taken from the animals at specified points in time. This was heparinized, and plasma was then obtained therefrom by centrifugation. The substance was quantified analytically in the plasma via LC-MS/MS. From the plasma concentration/time plots determined in this way, the pharmacokinetic parameters, such as AUC (area under the concentration/time curve), C. (maximum plasma concentration), T_{1/2} (half life), V_{SS} (distribution volume) and CL (clearance), and the absolute and the relative bioavailability (i.v./p.o. comparison or comparison of suspension to solution after p.o. administration), were calculated using an internal standard and with the aid of a validated computer program.

### C. EMBODIMENT EXAMPLES FOR PHARMACEUTICAL COMPOSITIONS

The compounds according to the invention can be converted into pharmaceutical formulations as follows.

### Tablet:

### Composition:

100 mg of the compound according to the invention, 50 mg of lactose (monohydrate), 50 mg of maize starch (native), 10 mg of polyvinylpyrrolidone (PVP 25) (BASF, Ludwigshafen, Germany) and 2 mg magnesium stearate.

Tablet weight 212 mg. Diameter 8 mm, radius of curvature 12 mm

### Preparation:

The mixture of compound according to the invention, lactose and starch is granulated with a 5% strength solution (w/w) of the PVP in water. After drying, the granules are mixed with the magnesium stearate for 5 minutes. This mixture is pressed with a conventional tablet press (for tablet format see above). A pressing force of 15 kN is used as the recommended value for the pressing.

### Suspension for Oral Administration:

### Composition:

1,000 mg of the compound according to the invention, 1,000 mg of ethanol (96%), 400 mg of Rhodigel® (xanthan gum from FMC, Pennsylvania, USA) and 99 g of water.

10 ml of oral suspension correspond to an individual dose of 100 mg of the compound according to the invention.

### Preparation:

The Rhodigel is suspended in ethanol and the compound according to the invention is added to the suspension. The water is added with stirring. The mixture is stirred for approx. 6 h until swelling of the Rhodigel has ended.

### Solution for Oral Administration:

### Composition:

500 mg of the compound according to the invention, 2.5 g of polysorbate and 97 g of polyethylene glycol 400.20 g of oral solution correspond to an individual dose of 100 mg of the compound according to the invention.

### Preparation:

The compound according to the invention is suspended in the mixture of polyethylene glycol and polysorbate, while stirring. The stirring operation is continued until solution of the compound according to the invention is complete.

i.v. Solution:

The compound according to the invention is dissolved in a concentration below the saturation solubility in a physiologically acceptable solvent (e.g. isotonic saline solution, glucose solution 5% and/or PEG 400 solution 30%). The solution is subjected to sterile filtration and is transferred into sterile and pyrogen-free injection containers.

### D. LITERATURE REFERENCES

Globocan 2002 Report
IARC International Agency for Research on Cancer: Globocan 2002, http://www-dep.iarc.fr/globocan/downloads.htm
American Cancer Society, Cancer Facts and Figures 2005
American Cancer Society: Cancer Facts and Figures 2007, http://www.cancer.org/docroot/STT/content/STT_{—}1x_Cancer_Facts_Figures_{—}2007. asp
Gibbs J B, 2000
Gibbs J B: Mechanism-based target identification and drug discovery in cancer research, Science 2000, 287 (5460), 1969-1973.
Semenza and Wang, 1992
Semenza G L, Wang G L: A nuclear factor induced by hypoxia via de novo protein synthesis binds to the human erythropoietin gene enhancer at a site required for transcriptional activation, Mol. Cell. Biol. 1992, 12 (12), 5447-5454.
Wang and Semenza, 1995
Wang G L, Semenza G L: Purification and characterization of hypoxia-inducible factor 1, J. Biol. Chem. 1995, 270 (3), 1230-1237.
Wang, Jiang et al., 1995
Wang G L, Jiang B H, Rue E A, Semenza G L: Hypoxia-inducible factor 1 is a basic-helix-loop-helix-PAS heterodimer regulated by cellular O₂ tension, PNAS 1995, 92 (12), 5510-5514.
Jiang, Rue et al., 1996
Jiang B H, Rue E, Wang G L, Roe R, Semenza G L: Dimerization, DNA binding, and transactivation properties of hypoxia-inducible factor 1, J. Biol. Chem. 1996, 271 (30), 17771-17778.
Makino, Cao et al., 2001
Makino Y, Cao R, Svensson K, Bertilsson G, Asman M, Tanaka H, Cao Y, Poellinger L: Nature 2001, 414 (6863), 550-554.
Jiang, Semenza et al., 1996
Jiang B H, Semenza G L, Bauer C, Marti H H: Hypoxia-inducible factor 1 levels vary exponentially over a physiologically relevant range of O₂ tension, Am. J. Physiol. 1996, 271, 1172-1180.
Maxwell, Wiesener et al., 1999
Maxwell P H, Wiesener M S, Chang G W, Clifford S C, Vaux E C, Cockman M E, Wykoff C C, Ratcliffe P J: The tumour suppressor protein VHL targets hypoxia-inducible factors for oxygen-dependent proteolysis, Nature 1999, 399 (6733), 271-275.
Hirota and Semenza, 2006
Hirota K, Semenza G L: Regulation of angiogenesis by hypoxia-inducible factor 1, Crit. Rev. Oncol. Hematol. 2006, 59 (1), 15-26.
Chen, Zhao et al., 2003
Chen J, Zhao S, Nakada K, Kuge Y, Tamaki N, Okada F, Wang J, Shindo M, Higashino F, Takeda K, Asaka M, Katoh H, Sugiyama T, Hosokawa M, Kobayashi M: Dominant-negative hypoxia-inducible factor-1 alpha reduces tumorigenicity of pancreatic cancer cells through the suppression of glucose metabolism, Am. J. Pathol. 2003, 162 (4), 1283-1291.
Stoeltzing, McCarty et al., 2004
Stoeltzing 0, McCarty M F, Wey J S, Fan F, Liu W, Belcheva A, Bucana C D, Semenza G L, Ellis L M: Role of hypoxia-inducible factor-1 alpha in gastric cancer cell growth, angiogenesis, and vessel maturation, J. Natl. Cancer Inst. 2004, 96 (12), 946-956.
Li, Lin et al., 2005
Li L, Lin X, Stayer M, Shoemaker A, Semizarov D, Fesik S W, Shen Y: Evaluating hypoxia-inducible factor-1alpha as a cancer therapeutic target via inducible RNA interference in vivo, Cancer Res. 2005, 65 (16), 7249-7258.
Mizukami, Jo et al., 2005
Mizukami Y, Jo W S, Duerr E M, Gala M, Li J, Zhang X, Zimmer M A, Iliopoulos O, Zukerberg L R, Kohgo Y, Lynch M P, Rueda B R, Chung D C: Induction of interleukin-8 preserves the angiogenic response in HIF-1alpha-deficient colon cancer cells, Nat. Med. 2005, 11 (9), 992-997.
Li, Shi et al., 2006
Li J, Shi M, Cao Y, Yuan W, Pang T, Li B, Sun Z, Chen L, Zhao R C: Knockdown of hypoxia-inducible factor-1 alpha in breast carcinoma MCF-7 cells results in reduced tumor growth and increased sensitivity to methotrexate, Biochem. Biophys. Res. Commun. 2006, 342, 1341-1351.
Semenza, 2007
Semenza G L: Drug Discov. Today 2007, 12 (19-20), 853-859.
Weidemann and Johnson, 2008
Weidemann A, Johnson R S: Cell Death and Differentiation 2008, 15, 621-627.
Aiello et al., 1994
Aiello et al.: New Engl. J. Med. 1994, 331, 1480.
Peer et al., 1995
Peer et al.: Lab. Invest. 1995, 72, 638.
Lopez et al., 1996
Lopez et al.: Invest. Ophthalmol. Vis. Sci. 1996, 37, 855.
Warburg, 1956
Warburg O.: Science 1956, 123 (3191), 309-314.

1. Compound of the formula (I) 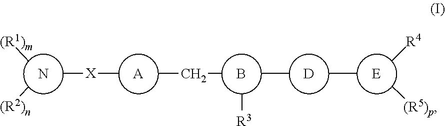
in which
the ring 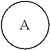
represents a phenyl or pyridyl ring,
the ring 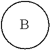
with the substituent R³ represents a heteroaryl ring of the formula 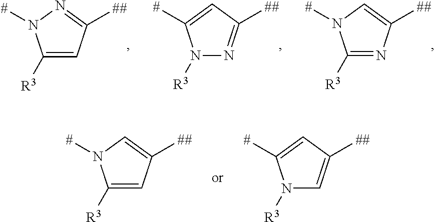
wherein
# designates the linkage point with the adjacent CH₂ group
and
## designates the linkage point with the ring 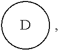
the ring 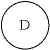
represents a heteroaryl ring of the formula 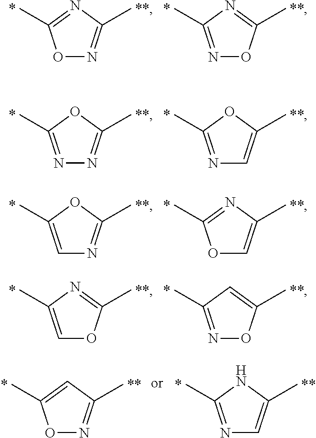
wherein
* designates the linkage point with the ring 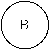
and
** designates the linkage point with the ring 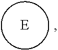
the ring 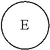
represents a phenyl or pyridyl ring,
the ring 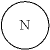
represents a saturated 4- to 10-membered aza-heterocycle, which contains at least one N atom as a ring member and in addition can contain one or two further hetero ring members from the series N, O, S and/or S(O)₂,
X represents a bond or ♦—(CH₂)_{q}—N(R⁶)—♦♦, ♦—N(R⁶)—(CH₂)_{q}—♦♦, —O—, —S—, —C(═O)—, —S(═O)₂—, ♦—C(═O)—N(R⁶)—♦♦ or ♦—N(R⁶)—C(═O)—♦♦, wherein
♦ designates the linkage point with the ring 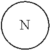
and
♦♦ designates the linkage point with the ring 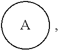
q denotes the number 0, 1 or 2
and
R⁶ denotes hydrogen, (C₁-C₆)-alkyl or (C₃-C₆)-cycloalkyl,
wherein (C₁-C₆)-alkyl and (C₃-C₆)-cycloalkyl can each be substituted by hydroxyl or (C₁-C₄)-alkoxy,
R¹ represents a substituent bonded to a carbon atom of the ring 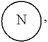
chosen from the series fluorine, cyano, (C₁-C₆)-alkyl, hydroxyl, (C₁-C₆)-alkoxy, oxo, amino, mono-(C₁-C₆)-alkylamino, di-(C₁-C₆)-alkylamino and (C₃-C₆)-cycloalkyl,
wherein (C₁-C₆)-alkyl in its turn can be substituted up to three times by fluorine and up to two times in an identical or different manner by a radical chosen from the series hydroxyl, (C₁-C₄)-alkoxy, amino, mono-(C₁-C₄)-alkylamino and di-(C₁-C₄)-alkylamino
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
m represents the number 0, 1, 2, 3 or 4,
wherein in the case where the substituent R⁵ occurs several times, its meanings can be identical or different,
R² represents a substituent bonded to a nitrogen atom of the ring 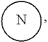
chosen from the series (C₁-C₆)-alkyl, (C₁-C₆)-alkylcarbonyl, (C₁-C₆)-alkoxycarbonyl, (C₁-C₆)-alkylsulfonyl and (C₃-C₆)-cycloalkyl,
wherein the alkyl group in (C₁-C₆)-alkyl, (C₁-C₆)-alkylcarbonyl, (C₁-C₆)-alkoxycarbonyl and (C₁-C₆)-alkylsulfonyl in its turn can be substituted up to three times by fluorine and up to two times in an identical or different manner by a radical chosen from the series hydroxyl, (C₁-C₄)-alkoxy, amino, mono-(C₁-C₄)-alkylamino, di-(C₁-C₄)-alkylamino (C₃-C₆)-cycloalkyl and 4- to 6-membered heterocyclyl
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
n represents the number 0 or 1 or also, if the aza-heterocycle 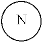
contains further N atoms as ring members, the number 2,
wherein in the case where the substituent R² occurs twice, its meanings can be identical or different,
R³ represents methyl, ethyl or trifluoromethyl,
R⁴ represents hydrogen or a substituent chosen from the series halogen, cyano, pentafluorothio, (C₁-C₆)-alkyl, tri-(C₁-C₄)-alkylsilyl, —OR⁷, —NR⁷R⁸, —N(R⁷)—C(═O)—R⁸, —N(R⁷)—C(═O)—OR⁸, —N(R⁷)—S(═O)₂—R⁸, —C(═O)—OR⁷, —C(═O)—NR⁷R⁸, —SR⁷, —S(═O)—R⁷, —S(═O)₂—R⁷, —S(═O)₂—NR⁷R⁸, —S(═O)(═NH)—R⁷, —S(═O)(═NCH₃)—R⁷, (C₃-C₆)-cycloalkyl, 4- to 6-membered heterocyclyl and 5- or 6-membered heteroaryl,
wherein (C₁-C₆)-alkyl in its turn can be substituted up to three times by fluorine and up to two times in an identical or different manner by a radical chosen from the series —OR⁷, —NR⁷R⁸, —N(R⁷)—C(═O)—R⁸, —N(R⁷)—C(═O)—OR⁸, —C(═O)—OR⁷, —C(═O)—NR⁷R⁸, (C₃-C₆)-cycloalkyl, 4- to 6-membered heterocyclyl and 5- or 6-membered heteroaryl
and wherein
the cycloalkyl and heterocyclyl groups mentioned in their turn can be substituted up to two times in an identical or different manner by a radical chosen from the series fluorine, (C₁-C₄)-alkyl, hydroxyl, (C₁-C₄)-alkoxy, trifluoromethoxy, oxo, amino, mono-(C₁-C₄)-alkylamino, di-(C₁-C₄)-alkylamino, (C₁-C₄)-alkylcarbonylamino, (C₁-C₄)-alkoxycarbonylamino, (C₁-C₄)-alkylcarbonyl and (C₁-C₄)-alkoxycarbonyl
and
the heteroaryl groups mentioned in their turn can be substituted up to two times in an identical or different manner by a radical chosen from the series fluorine, chlorine, cyano, (C₁-C₄)-alkyl, (C₁-C₄)-alkoxy and trifluoromethoxy
wherein the (C₁-C₄)-alkyl substituents mentioned herein in their turn can be substituted by hydroxyl, (C₁-C₄)-alkoxy, trifluoromethoxy, (C₁-C₄)-alkylcarbonyloxy, aminocarbonyl, mono-(C₁-C₄)-alkylaminocarbonyl or di-(C₁-C₄)-alkylaminocarbonyl or up to three times by fluorine,
and wherein
R⁷ and R⁸ independently of each other for each individual occurrence denote hydrogen, (C₁-C₆)-alkyl, (C₃-C₆)-cycloalkyl or 4- to 6-membered heterocyclyl,
wherein (C₁-C₆)-alkyl can be substituted up to three times by fluorine and up to two times in an identical or different manner by a radical chosen from the series hydroxyl, (C₁-C₄)-alkoxy, trifluoromethoxy, amino, mono-(C₁-C₄)-alkylamino, di-(C₁-C₄)-alkylamino, (C₁-C₄)-alkoxycarbonyl, (C₃-C₆)-cycloalkyl and 4- to 6-membered heterocyclyl
and
the cycloalkyl and heterocyclyl groups mentioned can be substituted up to two times in an identical or different manner by a radical chosen from the series fluorine, (C₁-C₄)-alkyl, trifluoromethyl, hydroxyl, (C₁-C₄)-alkoxy, trifluoromethoxy, oxo, amino, mono-(C₁-C₄)-alkylamino, di-(C₁-C₄)-alkylamino, (C₁-C₄)-alkylcarbonyl and (C₁-C₄)-alkoxycarbonyl,
or
R⁷ and R⁸ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo, amino, mono-(C₁-C₄)-alkylamino, di-(C₁-C₄)-alkylamino, (C₁-C₄)-alkylcarbonyl and (C₁-C₄)-alkoxycarbonyl,
R⁵ represents a substituent chosen from the series fluorine, chlorine, cyano, methyl, trifluoromethyl and hydroxyl
and
p represents the number 0, 1 or 2,
wherein in the case where the substituent R⁵ occurs twice, its meanings can be identical or different,
or a salt thereof.

2. Compound of the formula (I) according to claim 1, in which
the ring 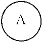
represents a phenyl or pyridyl ring and the adjacent groups X and CH₂ are bonded to ring carbon atoms 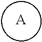
in 1, 3 or 1,4 relation to one another
and
the ring 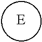
with the substituents R⁴ and R⁵ represents a phenyl ring of the formula 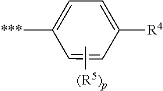
wherein
*** designates the linkage point with the ring 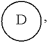
or a salt thereof.

3. Compound of the formula (I) according to claim 1, in which
the ring 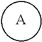
represents a pyridyl ring and the adjacent groups X and CH₂ are bonded to ring carbon atoms of this pyridyl ring in 1, 3 or 1,4 relation to one another
and
the ring 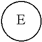
with the substituents R⁴ and R⁵ represents a phenyl ring of the formula 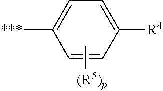
wherein
*** designates the linkage point with the ring 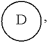
or a salt thereof.

4. Compound of the formula (I) according to claim 1, in which
the ring 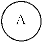
represents a phenyl ring and the adjacent groups X and CH₂ are bonded to this phenyl ring in 1, 3 or 1,4 relation to one another,
the ring 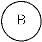
with the substituent R³ represents a heteroaryl ring of the formula 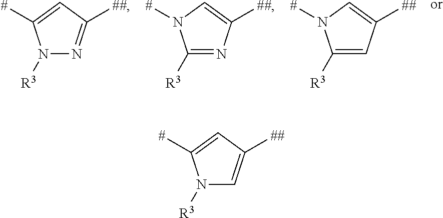
wherein
# designates the linkage point with the adjacent CH₂ group
and
## designates the linkage point with the ring 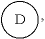
and
the ring 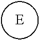
with the substituents R⁴ and R⁵ represents a phenyl ring of the formula 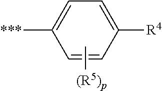
wherein
*** designates the linkage point with the ring 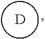
or a salt thereof.

5. Compound of the formula (I) according to claim 1, in which
the ring 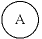
represents a phenyl ring and the adjacent groups X and CH₂ are bonded to this phenyl ring in 1, 3 or 1,4 relation to one another,
the ring 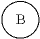
with the substituent R³ represents a heteroaryl ring of the formula 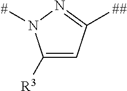
wherein
# designates the linkage point with the adjacent CH₂ group
and
## designates the linkage point with the ring 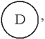
the ring 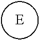
with the substituents R⁴ and R⁵ represents a phenyl ring of the formula 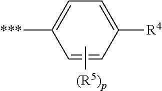
wherein
*** designates the linkage point with the ring 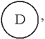
R¹ represents a substituent bonded to a carbon atom of the ring 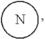
chosen from the series cyano, (C₁-C₆)-alkyl, oxo and (C₃-C₆)-cycloalkyl,
wherein (C₁-C₆)-alkyl in its turn can be substituted up to three times by fluorine and up to two times in an identical or different manner by a radical chosen from the series hydroxyl, (C₁-C₄)-alkoxy, amino, mono-(C₁-C₄)-alkylamino and di-(C₁-C₄)-alkylamino
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
R² represents a substituent bonded to a nitrogen atom of the ring 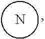
chosen from the series (C₁-C₆)-alkyl, (C₁-C₆)-alkylcarbonyl, (C₁-C₆)-alkoxycarbonyl, (C₁-C₄-alkylsulfonyl and (C₃-C₆)-cycloalkyl,
wherein the alkyl group in (C₁-C₆)-alkyl, (C₁-C₆)-alkylcarbonyl, (C₁-C₆)-alkoxycarbonyl and (C₁-C₆)-alkylsulfonyl in its turn can be substituted up to three times by fluorine and up to two times in an identical or different manner by a radical chosen from the series hydroxyl, (C₁-C₄)-alkoxy, amino, mono-(C₁-C₄)-alkylamino, di-(C₁-C₄)-alkylamino (C₃-C₆)-cycloalkyl and 4- to 6-membered heterocyclyl
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
m represents the number 0, 1, 2, 3 or 4,
wherein in the case where the substituent R⁵ occurs several times, its meanings can be identical or different,
and
n represents the number 0 or 1 or also, if the aza-heterocycle 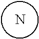
contains further N atoms as ring members, the number 2,
wherein in the case where the substituent R² occurs twice, its meanings can be identical or different,
wherein the sum of m and n does not equal the number 0,
or a salt thereof.

6. Compound of the formula (I) according to claim 1, in which
the ring 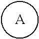
represents a pyridyl ring and the adjacent groups X and CH₂ are bonded to ring carbon atoms of this pyridyl ring in 1, 3 or 1,4 relation to one another,
the ring 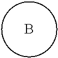
with the substituent R³ represents a heteroaryl ring of the formula 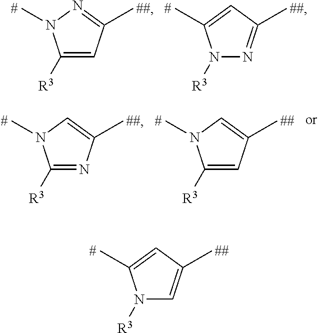
wherein
# designates the linkage point with the adjacent CH₂ group
and
## designates the linkage point with the ring 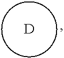
the ring 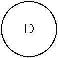
represents a heteroaryl ring of the formula 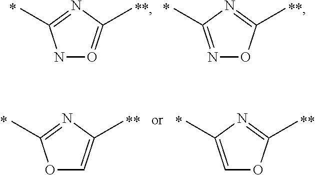
wherein
* designates the linkage point with the ring 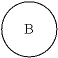
and
** designates the linkage point with the ring 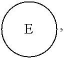
the ring 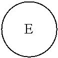
with the substituents R⁴ and R⁵ represents a phenyl ring of the formula 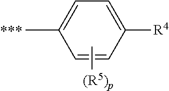
wherein
*** designates the linkage point with the ring 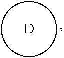
the ring 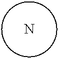
represents a saturated 4- to 10-membered aza-heterocycle, which contains at least one N atom as a ring member and in addition can contain a further hetero ring member from the series N, O, S or S(O)₂,
X represents a bond or ♦—(CH₂)_{q}—N(R⁶)—♦♦, —O—, —S—, —C(═O)—, —S(═O)₂— or ♦—N(R⁶)—C(═O)—♦♦, wherein
♦ designates the linkage point with the ring 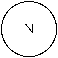
and
♦♦ designates the linkage point with the ring 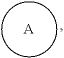
q denotes the number 0, 1 or 2
and
R⁶ denotes hydrogen, (C₁-C₄)-alkyl or (C₃-C₆)-cycloalkyl,
R¹ represents a substituent bonded to a carbon atom of the ring 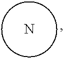
chosen from the series fluorine, cyano, (C₁-C₄)-alkyl, hydroxyl, (C₁-C₄)-alkoxy, oxo, amino, mono-(C₁-C₄)-alkylamino, di-(C₁-C₄)-alkylamino and (C₃-C₆)-cycloalkyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
m represents the number 0, 1 or 2,
wherein in the case where the substituent R¹ occurs twice, its meanings can be identical or different,
R² represents a substituent bonded to a nitrogen atom of the ring 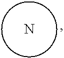
chosen from the series (C₁-C₄)-alkyl, (C₁-C₄)-alkylcarbonyl, (C₁-C₄)-alkoxycarbonyl, (C₁-C₄)-alkylsulfonyl and (C₃-C₆)-cycloalkyl,
wherein the alkyl group in (C₁-C₄)-alkyl, (C₁-C₄)-alkylcarbonyl, (C₁-C₄)-alkoxycarbonyl and (C₁-C₄)-alkylsulfonyl in its turn can be substituted by a radical chosen from the series hydroxyl, (C₁-C₄)-alkoxy, amino, mono-(C₁-C₄)-alkylamino, di-(C₁-C₄)-alkylamino (C₃-C₆)-cycloalkyl and 4- to 6-membered heterocyclyl and up to three times by fluorine
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
n represents the number 0 or 1,
R³ represents methyl, ethyl or trifluoromethyl,
R⁴ represents a substituent chosen from the series fluorine, chlorine, cyano, pentafluorothio, (C₁-C₆)-alkyl, tri-(C₁-C₄)-alkylsilyl, —OR⁷, —NR⁷R⁸, —SR⁷, —S(═O)—R⁷, —S(═O)₂—R⁷, —S(═O)(═NH)—R⁷, —S(═O)(═NCH₃)—R⁷, (C₃-C₆)-cycloalkyl and 4- to 6-membered heterocyclyl,
wherein (C₁-C₆)-alkyl in its turn can be substituted up to three times by fluorine and up to two times in an identical or different manner by a radical chosen from the series —OR⁷, —NR⁷R⁸, —N(R⁷)—C(═O)—R⁸, —C(═O)—NR⁷R⁸, (C₃-C₆)-cycloalkyl, 4- to 6-membered heterocyclyl and 5- or 6-membered heteroaryl
and wherein
the cycloalkyl and heterocyclyl groups mentioned in their turn can be substituted up to two times in an identical or different manner by a radical chosen from the series fluorine, (C₁-C₄)-alkyl, hydroxyl, (C₁-C₄)-alkoxy, trifluoromethoxy, oxo and (C₁-C₄)-alkylcarbonyl
and
the heteroaryl group mentioned in its turn can be substituted up to two times in an identical or different manner by a radical chosen from the series fluorine, chlorine, cyano, (C₁-C₄-alkyl, (C₁-C₄)-alkoxy and trifluoromethoxy
wherein the (C₁-C₄)-alkyl substituents mentioned herein in their turn can be substituted by hydroxyl, methoxy, trifluoromethoxy, ethoxy, acetoxy, aminocarbonyl, methylaminocarbonyl or dimethylaminocarbonyl or up to three times by fluorine,
and wherein
R⁷ and R⁸ independently of each other for each individual occurrence denote hydrogen, (C₁-C₄)-alkyl, (C₃-C₆)-cycloalkyl or 4- to 6-membered heterocyclyl,
wherein (C₁-C₄)-alkyl can be substituted up to three times by fluorine and up to two times in an identical or different manner by a radical chosen from the series hydroxyl, (C₁-C₄)-alkoxy, trifluoromethoxy, (C₃-C₆)-cycloalkyl and 4- to 6-membered heterocyclyl
and
the cycloalkyl and heterocyclyl groups mentioned can be substituted up to two times in an identical or different manner by a radical chosen from the series fluorine, (C₁-C₄)-alkyl, trifluoromethyl, hydroxyl, (C₁-C₄)-alkoxy, trifluoromethoxy, oxo and (C₁-C₄)-alkylcarbonyl
or
R⁷ and R⁸ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo and (C₁-C₄)-alkylcarbonyl,
R⁵ represents a substituent chosen from the series fluorine, chlorine and methyl
and
p represents the number 0 or 1,
or a salt thereof.

7. Compound of the formula (I) according to claim 1, in which
the ring 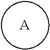
represents a phenyl ring and the adjacent groups X and CH₂ are bonded to this phenyl ring in 1, 3 or 1,4 relation to one another,
the ring 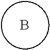
with the substituent R³ represents a heteroaryl ring of the formula 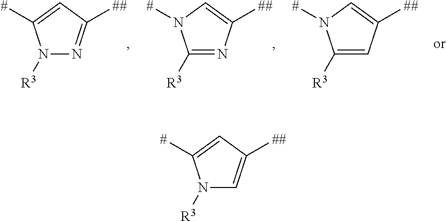
wherein
# designates the linkage point with the adjacent CH₂ group
and
## designates the linkage point with the ring 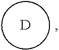
the ring 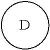
represents a heteroaryl ring of the formula 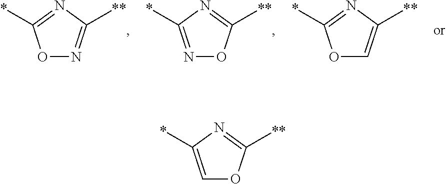
wherein
* designates the linkage point with the ring 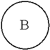
and
** designates the linkage point with the ring 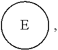
the ring 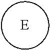
with the substituents R⁴ and R⁵ represents a phenyl ring of the formula 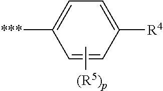
wherein
*** designates the linkage point with the ring 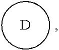
the ring 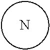
represents a saturated 4- to 10-membered aza-heterocycle, which contains at least one N atom as a ring member and in addition can contain a further hetero ring member from the series N, O, S or S(O)₂,
X represents a bond or ♦—(CH₂)_{q}—N(R⁶)—♦♦, —O—, —S—, —C(═O)—, —S(═O)₂— or ♦—N(R⁶)—C(═O)—♦♦, wherein
♦ designates the linkage point with the ring 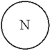
and
♦♦ designates the linkage point with the ring 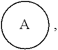
q denotes the number 0, 1 or 2
and
R⁶ denotes hydrogen, (C₁-C₄)-alkyl or (C₃-C₆)-cycloalkyl,
R¹ represents a substituent bonded to a carbon atom of the ring 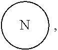
chosen from the series fluorine, cyano, (C₁-C₄)-alkyl, hydroxyl, (C₁-C₄)-alkoxy, oxo, amino, mono-(C₁-C₄)-alkylamino, di-(C₁-C₄)-alkylamino and (C₃-C₆)-cycloalkyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
m represents the number 0, 1 or 2,
wherein in the case where the substituent R¹ occurs twice, its meanings can be identical or different,
R² represents a substituent bonded to a nitrogen atom of the ring 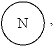
chosen from the series (C₁-C₄)-alkyl, (C₁-C₄)-alkylcarbonyl, (C₁-C₄)-alkoxycarbonyl, (C₁-C₄)-alkylsulfonyl and (C₃-C₆)-cycloalkyl,
wherein the alkyl group in (C₁-C₄)-alkyl, (C₁-C₄)-alkylcarbonyl, (C₁-C₄)-alkoxycarbonyl and (C₁-C₄)-alkylsulfonyl in its turn can be substituted by a radical chosen from the series hydroxyl, (C₁-C₄)-alkoxy, amino, mono-(C₁-C₄)-alkylamino, di-(C₁-C₄)-alkylamino (C₃-C₆)-cycloalkyl and 4- to 6-membered heterocyclyl and up to three times by fluorine
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
n represents the number 0 or 1,
R³ represents methyl, ethyl or trifluoromethyl,
R⁴ represents a substituent chosen from the series fluorine, chlorine, cyano, pentafluorothio, (C₁-C₆)-alkyl, tri-(C₁-C₄)-alkylsilyl, —OR⁷, —NR⁷R⁸, —SR⁷, —S(═O)—R⁷, —S(═O)₂—R⁷, —S(═O)(═NH)—R⁷, —S(═O)(═NCH₃)—R⁷, (C₃-C₆)-cycloalkyl and 4- to 6-membered heterocyclyl,
wherein (C₁-C₆)-alkyl in its turn can be substituted up to three times by fluorine and up to two times in an identical or different manner by a radical chosen from the series —OR⁷, NR⁷R⁸, —N(R⁷)—C(═O)—R⁸, —C(═O)—NR⁷R⁸, (C₃-C₆)-cycloalkyl, 4- to 6-membered heterocyclyl and 5- or 6-membered heteroaryl
and wherein
the cycloalkyl and heterocyclyl groups mentioned in their turn can be substituted up to two times in an identical or different manner by a radical chosen from the series fluorine, (C₁-C₄)-alkyl, hydroxyl, (C₁-C₄)-alkoxy, trifluoromethoxy, oxo and (C₁-C₄)-alkylcarbonyl
and
the heteroaryl group mentioned in its turn can be substituted up to two times in an identical or different manner by a radical chosen from the series fluorine, chlorine, cyano, (C₁-C₄)-alkyl, (C₁-C₄)-alkoxy and trifluoromethoxy
wherein the (C₁-C₄)-alkyl substituents mentioned herein in their turn can be substituted by hydroxyl, methoxy, trifluoromethoxy, ethoxy, acetoxy, aminocarbonyl, methylaminocarbonyl or dimethylaminocarbonyl or up to three times by fluorine,
and wherein
R⁷ and R⁸ independently of each other for each individual occurrence denote hydrogen, (C₁-C₄)-alkyl, (C₃-C₆)-cycloalkyl or 4- to 6-membered heterocyclyl,
wherein (C₁-C₄)-alkyl can be substituted up to three times by fluorine and up to two times in an identical or different manner by a radical chosen from the series hydroxyl, (C₁-C₄)-alkoxy, trifluoromethoxy, (C₃-C₆)-cycloalkyl and 4- to 6-membered heterocyclyl
and
the cycloalkyl and heterocyclyl groups mentioned can be substituted up to two times in an identical or different manner by a radical chosen from the series fluorine, (C₁-C₄)-alkyl, trifluoromethyl, hydroxyl, (C₁-C₄)-alkoxy, trifluoromethoxy, oxo and (C₁-C₄)-alkylcarbonyl
or
R⁷ and R⁸ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo and (C₁-C₄)-alkylcarbonyl,
R⁵ represents a substituent chosen from the series fluorine, chlorine and methyl
and
p represents the number 0 or 1,
or a salt thereof.

8. Compound of the formula (I) according to claim 1, in which
the ring 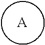
represents a phenyl ring and the adjacent groups X and CH₂ are bonded to this phenyl ring in 1, 3 or 1,4 relation to one another,
the ring 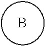
with the substituent R³ represents a heteroaryl ring of the formula 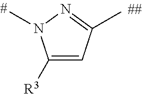
wherein
# designates the linkage point with the adjacent CH₂ group
and
## designates the linkage point with the ring 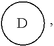
the ring 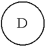
represents a heteroaryl ring of the formula 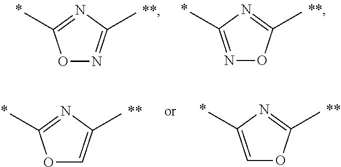
wherein
* designates the linkage point with the ring 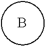
and
** designates the linkage point with the ring 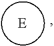
the ring 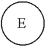
with the substituents R⁴ and R⁵ represents a phenyl ring of the formula 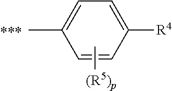
wherein
*** designates the linkage point with the ring 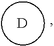
the ring 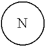
represents a saturated 4- to 10-membered aza-heterocycle, which contains at least one N atom as a ring member and in addition can contain a further hetero ring member from the series N, O, S or S(O)₂,
X represents a bond or ♦—(CH₂)_{q}—N(R⁶)—♦♦, —O—, —S—, —C(═O)—, —S(═O)₂— or ♦—N(R⁶)—C(═O)—♦♦, wherein
♦ designates the linkage point with the ring 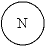
and
♦♦ designates the linkage point with the ring 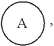
q denotes the number 0, 1 or 2
and
R⁶ denotes hydrogen, (C₁-C₄)-alkyl or (C₃-C₆)-cycloalkyl,
R¹ represents a substituent bonded to a carbon atom of the ring 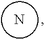
chosen from the series cyano, (C₁-C₄)-alkyl, oxo and (C₃-C₆)-cycloalkyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
R² represents a substituent bonded to a nitrogen atom of the ring 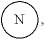
chosen from the series (C₁-C₄)-alkyl, (C₁-C₄)-alkylcarbonyl, (C₁-C₄)-alkoxycarbonyl, (C₁-C₄)-alkylsulfonyl and (C₃-C₆)-cycloalkyl,
wherein the alkyl group in (C₁-C₄)-alkyl, (C₁-C₄)-alkylcarbonyl, (C₁-C₄)-alkoxycarbonyl and (C₁-C₄)-alkylsulfonyl in its turn can be substituted by a radical chosen from the series hydroxyl, (C₁-C₄)-alkoxy, amino, mono-(C₁-C₄)-alkylamino, di-(C₁-C₄)-alkylamino (C₃-C₆)-cycloalkyl and 4- to 6-membered heterocyclyl and up to three times by fluorine
and
(C₃-C₆)-cycloalkyl in its turn can be substituted up to two times in an identical or different manner by a radical chosen from the series fluorine, (C₁-C₄)-alkyl, hydroxyl, (C₁-C₄)-alkoxy, amino, mono-(C₁-C₄)-alkylamino and di-(C₁-C₄)-alkylamino,
m represents the number 0, 1 or 2,
wherein in the case where the substituent R¹ occurs twice, its meanings can be identical or different,
n represents the number 0 or 1,
wherein the sum of m and n equals the number 1, 2 or 3,
R³ represents methyl, ethyl or trifluoromethyl,
R⁴ represents a substituent chosen from the series fluorine, chlorine, cyano, pentafluorothio, (C₁-C₆)-alkyl, tri-(C₁-C₄)-alkylsilyl, —OR⁷, —NR⁷R⁸, —SR⁷, —S(═O)—R⁷, —S(═O)₂—R⁷, —S(═O)(═NH)—R⁷, —S(═O)(═NCH₃)—R⁷, (C₃-C₆)-cycloalkyl and 4- to 6-membered heterocyclyl,
wherein (C₁-C₆)-alkyl in its turn can be substituted up to three times by fluorine and up to two times in an identical or different manner by a radical chosen from the series —OR⁷, —NR⁷R⁸, —N(R⁷)—C(═O)—R⁸, —C(═O)—NR⁷R⁸, (C₃-C₆)-cycloalkyl, 4- to 6-membered heterocyclyl and 5- or 6-membered heteroaryl
and wherein
the cycloalkyl and heterocyclyl groups mentioned in their turn can be substituted up to two times in an identical or different manner by a radical chosen from the series fluorine, (C₁-C₄)-alkyl, hydroxyl, (C₁-C₄)-alkoxy, trifluoromethoxy, oxo and (C₁-C₄)-alkylcarbonyl
and
the heteroaryl group mentioned in its turn can be substituted up to two times in an identical or different manner by a radical chosen from the series fluorine, chlorine, cyano, (C₁-C₄)-alkyl, (C₁-C₄)-alkoxy and trifluoromethoxy
wherein the (C₁-C₄)-alkyl substituents mentioned herein in their turn can be substituted by hydroxyl, methoxy, trifluoromethoxy, ethoxy, acetoxy, aminocarbonyl, methylaminocarbonyl or dimethylaminocarbonyl or up to three times by fluorine,
and wherein
R⁷ and R⁸ independently of each other for each individual occurrence denote hydrogen, (C₁-C₄)-alkyl, (C₃-C₆)-cycloalkyl or 4- to 6-membered heterocyclyl,
wherein (C₁-C₄)-alkyl can be substituted up to three times by fluorine and up to two times in an identical or different manner by a radical chosen from the series hydroxyl, (C₁-C₄)-alkoxy, trifluoromethoxy, (C₃-C₆)-cycloalkyl and 4- to 6-membered heterocyclyl
and
the cycloalkyl and heterocyclyl groups mentioned can be substituted up to two times in an identical or different manner by a radical chosen from the series fluorine, (C₁-C₄)-alkyl, trifluoromethyl, hydroxyl, (C₁-C₄)-alkoxy, trifluoromethoxy, oxo and (C₁-C₄)-alkylcarbonyl
or
R⁷ and R⁸ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo and (C₁-C₄)-alkylcarbonyl,
R⁵ represents a substituent chosen from the series fluorine, chlorine and methyl
and
p represents the number 0 or 1,
or a salt thereof.

9. Compound of the formula (I) according to claim 1, in which
the ring 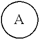
represents a pyridyl ring of the formula 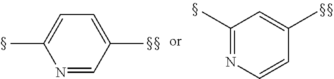
wherein
§ designates the linkage point with the adjacent group X
and
§§ designates the linkage point with the adjacent CH₂ group,
the ring 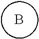
with the substituent R³ represents a heteroaryl ring of the formula 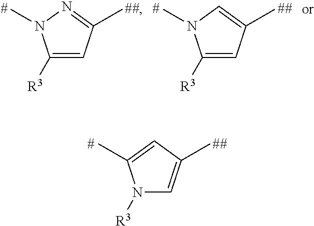
wherein
# designates the linkage point with the adjacent CH₂ group
and
## designates the linkage point with the ring 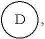
the ring 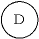
represents a heteroaryl ring of the formula 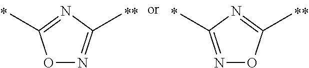
wherein
* designates the linkage point with the ring 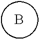
and
** designates the linkage point with the ring 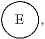
the ring 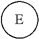
with the substituents R⁴ and R⁵ represents a phenyl ring of the formula 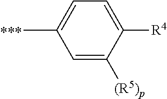
wherein
*** designates the linkage point with the ring 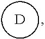
the ring 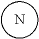
represents a saturated 4- to 10-membered aza-heterocycle, which contains at least one N atom as a ring member and in addition can contain a further hetero ring member from the series N, O, S or S(O)₂,
X represents a bond or ♦—(CH₂)_{q}—N(R⁶)—♦♦, —C(═O)— or ♦—N(R⁶)—C(═O)—♦♦, wherein
♦ designates the linkage point with the ring 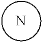
and
♦♦ designates the linkage point with the ring 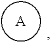
q denotes the number 0 or 1
and
R⁶ denotes hydrogen, methyl, ethyl, isopropyl, cyclopropyl or cyclobutyl,
R¹ represents a substituent bonded to a carbon atom of the ring 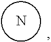
chosen from the series fluorine, cyano, (C₁-C₄)-alkyl, hydroxyl, (C₁-C₄)-alkoxy, oxo, amino, mono-(C₁-C₄)-alkylamino, di-(C₁-C₄)-alkylamino, cyclopropyl and cyclobutyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine,
m represents the number 0 or 1,
R² represents a substituent bonded to a nitrogen atom of the ring 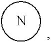
chosen from the series (C₁-C₄-alkyl, (C₁-C₄)-alkylcarbonyl, (C₁-C₄)-alkoxycarbonyl, (C₁-C₄)-alkylsulfonyl, cyclopropyl and cyclobutyl,
wherein the alkyl group in (C₁-C₄-alkyl, (C₁-C₄)-alkylcarbonyl, (C₁-C₄)-alkoxycarbonyl and (C₁-C₄)-alkylsulfonyl in its turn can be substituted by a radical chosen from the series hydroxyl, (C₁-C₄)-alkoxy, amino, mono-(C₁-C₄)-alkylamino, di-(C₁-C₄)-alkylamino, (C₃-C₅)-cycloalkyl and 4- or 5-membered heterocyclyl and up to three times by fluorine
n represents the number 0 or 1,
R³ represents methyl,
R⁴ represents a substituent chosen from the series chlorine, pentafluorothio, (C₁-C₆)-alkyl, trimethylsilyl, —OR⁷, —SR⁷, —S(═O)—R⁷, —S(═O)₂—R⁷, —S(═O)(═NCH₃)—CF₃, (C₃-C₆)-cycloalkyl and 4- to 6-membered heterocyclyl,
wherein (C₁-C₆)-alkyl in its turn can be substituted by a radical chosen from the series —OR⁷, —NR⁷R⁸, —C(═O)—NR⁷R⁸, (C₃-C₆)-cycloalkyl and 4- to 6-membered heterocyclyl and up to three time by fluorine
and
the cycloalkyl and heterocyclyl groups mentioned in their turn can be substituted up to two times in an identical or different manner by a radical chosen from the series fluorine, (C₁-C₄-alkyl, trifluoromethyl, (C₁-C₄)-alkoxy, trifluoromethoxy and oxo,
wherein the (C₁-C₄-alkyl substituent in its turn can be substituted by methoxy, trifluoromethoxy or ethoxy,
and wherein
R⁷ and R⁸ independently of each other for each individual occurrence denote hydrogen, (C₁-C₄)-alkyl or (C₃-C₆)-cycloalkyl,
wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
and
the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy and trifluoromethoxy,
or
R⁷ and R⁸ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo and (C₁-C₄)-alkylcarbonyl,
R⁵ represents fluorine,
and
p represents the number 0 or 1,
or a salt thereof.

10. Compound of the formula (I) according to claim 1, in which
the ring 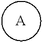
represents a phenyl ring and the adjacent groups X and CH₂ are bonded to this phenyl ring in 1, 3 or 1,4 relation to one another,
the ring 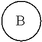
with the substituent R³ represents a heteroaryl ring of the formula 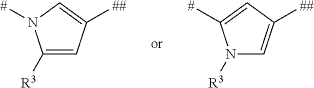
wherein
# designates the linkage point with the adjacent CH₂ group
and
## designates the linkage point with the ring 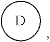
the ring 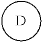
represents a heteroaryl ring of the formula 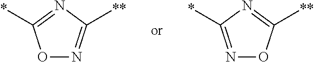
wherein
* designates the linkage point with the ring 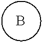
and
** designates the linkage point with the ring 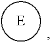
the ring 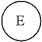
with the substituents R⁴ and R⁵ represents a phenyl ring of the formula 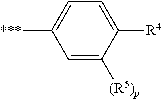
wherein
*** designates the linkage point with the ring 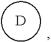
the ring 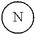
represents a saturated 4- to 10-membered aza-heterocycle, which contains at least one N atom as a ring member and in addition can contain a further hetero ring member from the series N, O, S or S(O)₂,
X represents a bond or ♦—(CH₂)_{q}—N(R⁶)—♦♦, —C(═O)— or ♦—N(R⁶)—C(═O)—♦♦, wherein
♦ designates the linkage point with the ring 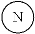
and
♦♦ designates the linkage point with the ring 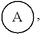
q denotes the number 0 or 1
and
R⁶ denotes hydrogen, methyl, ethyl, isopropyl, cyclopropyl or cyclobutyl,
R¹ represents a substituent bonded to a carbon atom of the ring 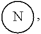
chosen from the series fluorine, cyano, (C₁-C₄)-alkyl, hydroxyl, (C₁-C₄)-alkoxy, oxo, amino, mono-(C₁-C₄)-alkylamino, di-(C₁-C₄)-alkylamino, cyclopropyl and cyclobutyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine,
m represents the number 0 or 1,
R² represents a substituent bonded to a nitrogen atom of the ring 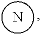
chosen from the series (C₁-C₄)-alkyl, (C₁-C₄)-alkylcarbonyl, (C₁-C₄)-alkoxycarbonyl, (C₁-C₄)-alkylsulfonyl, cyclopropyl and cyclobutyl,
wherein the alkyl group in (C₁-C₄)-alkyl, (C₁-C₄)-alkylcarbonyl, (C₁-C₄)-alkoxycarbonyl and (C₁-C₄)-alkylsulfonyl in its turn can be substituted by a radical chosen from the series hydroxyl, (C₁-C₄)-alkoxy, amino, mono-(C₁-C₄)-alkylamino, di-(C₁-C₄)-alkylamino (C₃-C₅)-cycloalkyl and 4- or 5-membered heterocyclyl and up to three times by fluorine,
n represents the number 0 or 1,
R³ represents methyl,
R⁴ represents a substituent chosen from the series chlorine, pentafluorothio, (C₁-C₆)-alkyl, trimethylsilyl, —OR⁷, —SR⁷, —S(═O)—R⁷, —S(═O)₂—R⁷, —S(═O)(═NCH₃)—CF₃, (C₃-C₆)-cycloalkyl and 4- to 6-membered heterocyclyl,
wherein (C₁-C₆)-alkyl in its turn can be substituted by a radical chosen from the series —OR⁷, —NR⁷R⁸, —C(═O)—NR⁷R⁸, (C₃-C₆)-cycloalkyl and 4- to 6-membered heterocyclyl and up to three time by fluorine
and
the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
wherein the (C₁-C₄)-alkyl substituent mentioned in its turn can be substituted by methoxy, trifluoromethoxy or ethoxy,
and wherein
R⁷ and R⁸ independently of each other for each individual occurrence denote hydrogen, (C₁-C₄)-alkyl or (C₃-C₆)-cycloalkyl,
wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
and
the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy and trifluoromethoxy,
or
R⁷ and R⁸ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo and (C₁-C₄)-alkylcarbonyl,
R⁵ represents fluorine,
and
p represents the number 0 or 1,
or a salt thereof.

11. Compound of the formula (I) according to claim 1, in which
the ring 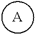
represents a phenyl ring and the adjacent groups X and CH₂ are bonded to this phenyl ring in 1, 3 or 1,4 relation to one another,
the ring 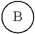
with the substituent R³ represents a heteroaryl ring of the formula 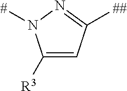
wherein
# designates the linkage point with the adjacent CH₂ group
and
## designates the linkage point with the ring 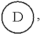
the ring 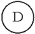
represents a heteroaryl ring of the formula 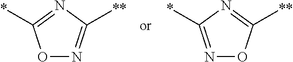
wherein
* designates the linkage point with the ring 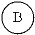
and
** designates the linkage point with the ring 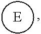
the ring 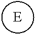
with the substituents R⁴ and R⁵ represents a phenyl ring of the formula 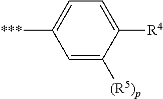
wherein
*** designates the linkage point with the ring 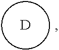
the ring 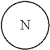
represents a saturated 4- to 10-membered aza-heterocycle, which contains at least one N atom as a ring member and in addition can contain a further hetero ring member from the series N, O, S or S(O)₂,
X represents a bond or ♦—(CH₂)_{q}—N(R⁶)—♦♦, —C(═O)— or ♦—N(R⁶)—C(═O)—♦♦, wherein
♦ designates the linkage point with the ring 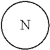
and
♦♦ designates the linkage point with the ring 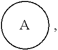
q denotes the number 0 or 1
and
R⁶ denotes hydrogen, methyl, ethyl, isopropyl, cyclopropyl or cyclobutyl,
R¹ represents a substituent bonded to a carbon atom of the ring 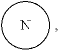
chosen from the series cyano, (C₁-C₄)-alkyl, oxo, cyclopropyl and cyclobutyl,
wherein (C₁-C₄)-alkyl in its turn can be substituted by a radical chosen from the series hydroxyl, (C₁-C₄)-alkoxy, amino, mono-(C₁-C₄)-alkylamino and di-(C₁-C₄)-alkylamino and up to three times by fluorine,
R² represents a substituent bonded to a nitrogen atom of the ring 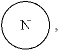
chosen from the series (C₁-C₄)-alkyl, (C₁-C₄)-alkylcarbonyl, (C₁-C₄)-alkoxycarbonyl, (C₁-C₄)-alkylsulfonyl, cyclopropyl and cyclobutyl,
wherein the alkyl group in (C₁-C₄)-alkyl, (C₁-C₄)-alkylcarbonyl, (C₁-C₄)-alkoxycarbonyl and (C₁-C₄)-alkylsulfonyl in its turn can be substituted by a radical chosen from the series hydroxyl, (C₁-C₄)-alkoxy, amino, mono-(C₁-C₄)-alkylamino, di-(C₁-C₄)-alkylamino, (C₃-C₅)-cycloalkyl and 4- or 5-membered heterocyclyl and up to three times by fluorine,
m represents the number 0 or 1,
n represents the number 0 or 1,
wherein the sum of m and n equals the number 1 or 2,
R³ represents methyl,
R⁴ represents a substituent chosen from the series chlorine, pentafluorothio, (C₁-C₆)-alkyl, trimethylsilyl, —OR⁷, —SR⁷, —S(═O)—R⁷, —S(═O)₂—R⁷, —S(═O)(═NCH₃)—CF₃, (C₃-C₆)-cycloalkyl and 4- to 6-membered heterocyclyl,
wherein (C₁-C₆)-alkyl in its turn can be substituted by a radical chosen from the series —OR⁷, —NR⁷R⁸, —C(═O)—NR⁷R⁸, (C₃-C₆)-cycloalkyl and 4- to 6-membered heterocyclyl and up to three time by fluorine
and
the cycloalkyl and heterocyclyl groups mentioned in their turn can be substituted up to two times in an identical or different manner by a radical chosen from the series fluorine, (C₁-C₄)-alkyl, trifluoromethyl, (C₁-C₄)-alkoxy, trifluoromethoxy and oxo,
wherein the (C₁-C₄)-alkyl substituent mentioned in its turn can be substituted by methoxy, trifluoromethoxy or ethoxy,
and wherein
R⁷ and R⁸ independently of each other for each individual occurrence denote hydrogen, (C₁-C₄)-alkyl or (C₃-C₆)-cycloalkyl,
wherein (C₁-C₄)-alkyl can be substituted by a radical chosen from the series hydroxyl, (C₁-C₄)-alkoxy, trifluoromethoxy and (C₃-C₆)-cycloalkyl and up to three times by fluorine
and
the cycloalkyl groups mentioned can be substituted up to two times in an identical or different manner by a radical chosen from the series fluorine, (C₁-C₄)-alkyl, trifluoromethyl, hydroxyl, (C₁-C₄)-alkoxy and trifluoromethoxy,
or
R⁷ and R⁸ in the case where both are bonded to a nitrogen atom form a 4- to 6-membered heterocycle together with this nitrogen atom, which can contain a further ring hetero atom from the series N, O, S or S(O)₂ and which can be substituted up to two times in an identical or different manner by a radical chosen from the series fluorine, (C₁-C₄)-alkyl, trifluoromethyl, hydroxyl, (C₁-C₄)-alkoxy, oxo and (C₁-C₄)-alkylcarbonyl,
R⁵ represents fluorine,
and
p represents the number 0 or 1,
or a salt thereof.

12. (canceled)

13. (canceled)

14. (canceled)

15. (canceled)

16. (canceled)

17. A pharmaceutical composition comprising compound as defined in claim 1 in combination with one or more inert, non-toxic, pharmaceutically suitable auxiliary substances.

18. A pharmaceutical composition comprising compound as defined in claim 1 in combination with one or more further active compounds.

19. (canceled)

20. (canceled)

21. Method for the treatment and/or prevention of cancer diseases or tumour diseases comprising administering an effective amount of at least one compound as defined in claim 1 to a human or animal in need thereof.

22. Method for the treatment and/or prevention of ischaemic cardiovascular diseases, cardiac insufficiency, cardiac infarction, arrhythmia, stroke, pulmonary hypertension, fibrotic diseases of the kidney and lung, psoriasis, diabetic retinopathy, macular degeneration, rheumatic arthritis and Chugwash polycythaemia comprising administering an effective amount of at least one compound as defined in claim 1 to a human or animal in need thereof.

23. Process for the preparation of compounds of the formula (I-D) 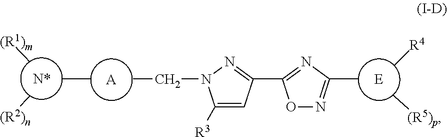
in which the rings A and E and R¹, R², R³, R⁴, R⁵, m, n and p each have the meanings given in claim 1,
and
the ring N* represents a ring N which is bonded to the ring A via a ring nitrogen atom and is as defined in claim 1,
characterized in that an N′-hydroxyamidine of the formula (IX) 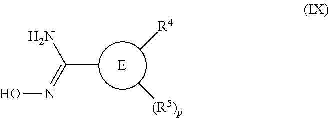
in which the ring E and R⁴, R⁵ and p have the meanings given above,
first can either be
[A] subjected to a condensation reaction with a pyrazolecarboxylic acid of the formula (XXVII) 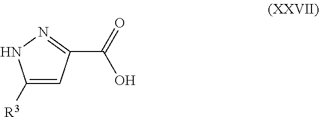
in which R³ has the meaning given above,
to give a 1,2,4-oxadiazole derivative of the formula (XXVIII) 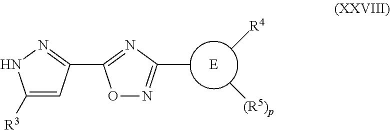
in which the ring E and R³, R⁴, R⁵ and p have the meanings given above,
and this is then alkylated in the presence of a base with a compound of the formula (III) 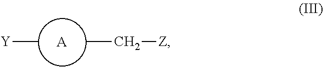
in which the ring A has the meaning given above,
Y represents chlorine, bromine or iodine
and
Z represents chlorine, bromine, iodine, mesylate, triflate or tosylate,
to give a compound of the formula (XXIX) 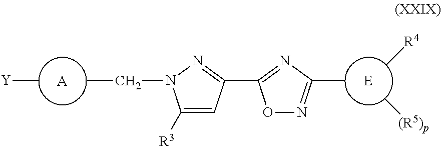
in which the rings A and E and R³, R⁴, R⁵, p and Y have the meanings given above,
or
[B] subjected to a condensation reaction with a pyrazolecarboxylic acid of the formula (XXX) 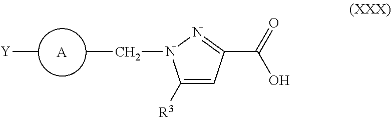
in which the ring A and R³ have the meanings given above,
and
Y represents chlorine, bromine or iodine,
to give the compound of the formula (XXIX) 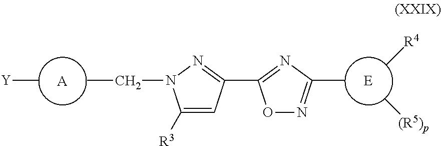
in which the rings A and E and R³, R⁴, R⁵, p and Y have the abovementioned meanings,
and the compound of the formula (XXIX) obtained in this way in then reacted, optionally in the presence of a palladium catalyst and/or a base, with a compound of the formula (XXXI) 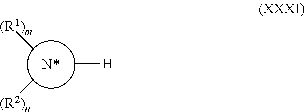
in which the ring N* and R¹, R², m and n have the meanings given above and the hydrogen atom shown is bonded to a nitrogen atom of the ring N*.
